(19) United States
(12) Patent Application Publication
(10) Pub. No.: US 2025/0154136 A1
(43) Pub. Date: May 15, 2025

(54) COMPOUND, AND ORGANIC ELECTROLUMINESCENT MATERIAL AND ORGANIC ELECTROLUMINESCENT DEVICE COMPRISING THE SAME

(71) Applicant: DuPont Specialty Materials Korea Ltd., Chungcheongnam-do (KR)

(72) Inventors: Doo-Hyeon MOON, Gyeonggi-do (KR); Mi-Ja LEE, Gyeonggi-do (KR); Tae-Jun HAN, Gyeonggi-do (KR); Kyoung-Jin PARK, Gyeonggi-do (KR); Eun-Young LEE, Gyeonggi-do (KR)

(21) Appl. No.: 18/928,865

(22) Filed: Oct. 28, 2024

(30) Foreign Application Priority Data
Nov. 2, 2023 (KR) 10-2023-0149744
Sep. 24, 2024 (KR) 10-2024-0128657

(51) Int. Cl. C07D 405/14; C07B 59/00; C07D 409/14; H10K 85/60

(52) U.S. Cl. CPC C07D 405/14; C07B 59/004; C07D 409/14; H10K 85/622; H10K 85/654; H10K 85/656; H10K 85/6572; H10K 85/6574; H10K 85/6576; C07B 2200/05

(57) ABSTRACT

The present disclosure relates to a compound, and an organic electroluminescent material and an organic electroluminescent device comprising the same. By comprising the compound and/or the organic electroluminescent material according to the present disclosure, an organic electroluminescent device can be provided which has low driving voltage and/or high luminous efficiency and/or long lifespan characteristics compared to conventional organic electroluminescent devices.

### TECHNICAL FIELD

The present disclosure relates to a compound, and an organic electroluminescent material and an organic electroluminescent device comprising the same.

### BACKGROUND ART

The TPD/Alq₃ bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. Therefore, an OLED having high luminous efficiency and/or long lifespan characteristics is required for long-term use and high display resolution. Korean Patent Application Laid-Open Nos. 2011-0052540, 2011-0075690, 2019-0113663, and 2015-0061975 disclose an organic light-emitting device that uses a nitrogen-containing heteroaryl such as triazine and/or a carbazole derivative substituted with dibenzofuran, dibenzothiophene, etc. as a host material for the light-emitting layer, but do not specifically disclose an organic electroluminescent device having improved performance, such as low driving voltage and/or long lifespan characteristics, due to the inclusion of a compound having a specific substituent at a specific position described in the present disclosure and an organic electroluminescent material including the same.

### DISCLOSURE OF THE INVENTION

### Technical Problem

The object of the present disclosure is, firstly, to provide a compound effective for producing an organic electroluminescent device having low driving voltage and/or long lifespan characteristics and, secondly, to provide an organic electroluminescent material comprising the compound, and an organic electroluminescent device comprising the compound and/or the organic electroluminescent material according to the present disclosure.

### Solution to Problem

As a result of intensive studies to solve the technical problem above, the present inventors found that the aforementioned object can be achieved by a compound represented by the following Formula 1, and/or an organic electroluminescent material comprising a compound represented by the following Formula 1 and a compound represented by the following Formula 2 or 3, thereby completing the present invention.

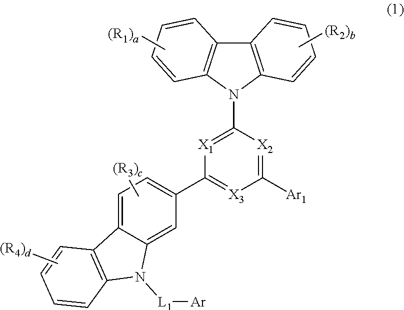

In Formula 1,
X₁ to X₃ each independently represent CR₅ or N, provided that at least one of X₁ to X₃ is N;
Ar₁ represents hydrogen, deuterium, halogen, cyano, amino, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, —Si(R₁₁)(R₁₂)(R₁₃), or —N(R₁₄)(R₁₅);
R₁₁ to R₁₅ each independently represent a substituted or unsubstituted (C₁-C₃₀)alkyl, a substituted or unsubstituted (C₂-C₃₀)alkenyl, a substituted or unsubstituted (C₆-C₃₀)aryl, or a substituted or unsubstituted (3- to 30-membered)heteroaryl;
L₁ represents a single bond, a substituted or unsubstituted (C₆-C₃₀)arylene, or a substituted or unsubstituted (3- to 30-membered)heteroarylene;
Ar represents a substituted or unsubstituted (C₆-C₃₀)aryl or a substituted or unsubstituted (3- to 30-membered)heteroaryl;
R₁ to R₅ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s); and
a, b, and d each independently represent an integer of 1 to 4, c represents an integer of 1 to 3, and when a to d are an integer of 2 or more, each of R₁ to R₄ may be the same as or different from each other.

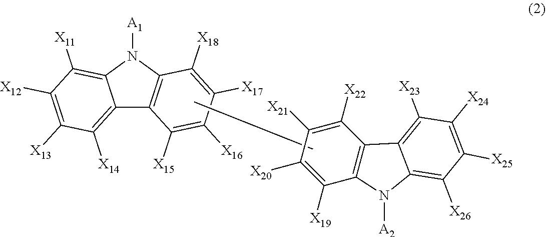

In Formula 2,
A₁ and A₂ each independently represent a substituted or unsubstituted (C₆-C₃₀)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are linked to each other to form a single bond; and
X₁₁ to X₁₄, X₂₃ to X₂₆, and X₁₅ to X₂₂ which do not form a single bond each independently represent hydrogen, deuterium, a substituted or unsubstituted (C₆-C₃₀)aryl, or a substituted or unsubstituted (3- to 30-membered)heteroaryl; or may be linked to the adjacent substituents to form a ring(s).

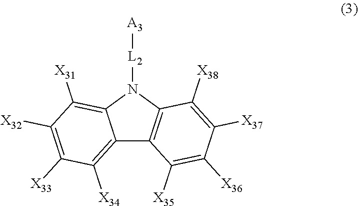

In Formula 3,
A₃ represents a substituted or unsubstituted (C₆-C₃₀)aryl, a substituted or unsubstituted dibenzofuranyl, a substituted or unsubstituted dibenzothiophenyl, a substituted or unsubstituted dibenzoselenophenyl, or a substituted or unsubstituted carbazolyl;
L₂ represents a single bond or a substituted or unsubstituted (C₆-C₃₀)arylene; and
a pair of adjacent X₃₅ to X₃₈ are linked to the following Formula 3-A to form a ring, and X₃₅ to X₃₈, which do not form a ring(s), and X₃₁ to X₃₄ each independently represent hydrogen, deuterium, a substituted or unsubstituted (C₆-C₃₀)aryl, or a substituted or unsubstituted (3- to 30-membered)heteroaryl.

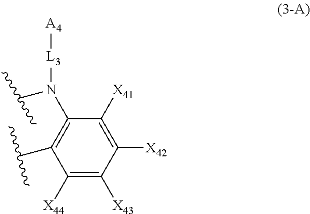

In Formula 3-A,
A₄ represents a substituted or unsubstituted (C₆-C₃₀)aryl, a substituted or unsubstituted dibenzofuranyl, a substituted or unsubstituted dibenzothiophenyl, a substituted or unsubstituted dibenzoselenophenyl, or a substituted or unsubstituted carbazolyl;
L₃ represents a single bond or a substituted or unsubstituted (C₆-C₃₀)arylene; and
X₄₁ to X₄₄ each independently represent hydrogen, deuterium, a substituted or unsubstituted (C₆-C₃₀)aryl, or a substituted or unsubstituted (3- to 30-membered)heteroaryl.

### Advantageous Effects of Invention

By comprising a compound according to the present disclosure and/or an organic electroluminescent material comprising the same, an organic electroluminescent device having low driving voltage and/or high luminous efficiency and/or long lifespan properties can be provided.

### MODE FOR THE INVENTION

Hereinafter, the present disclosure will be described in detail. However, the following description is intended to explain the invention, and is not meant in any way to restrict the scope of the invention.

The present disclosure relates to a compound represented by Formula 1 above, an organic electroluminescent material comprising the compound, and an organic electroluminescent device comprising the compound and/or the organic electroluminescent material.

The organic electroluminescent material according to the present disclosure comprises a compound represented by Formula 1 and a compound represented by Formula 2 or 3.

Herein, the term “organic electroluminescent compound” in the present disclosure means a compound that may be used in an organic electroluminescent device, and this may be comprised in any material layer constituting an organic electroluminescent device, as necessary.

Herein, the term “organic electroluminescent material” means a material that may be used in an organic electroluminescent device, and this may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (containing host and dopant materials), an electron buffer material, a hole-blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron-blocking layer, a light-emitting layer, an electron buffer layer, a hole-blocking layer, an electron transport layer, and an electron injection layer. As such, at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C₁-C₃₀)alkyl(ene)” is meant to be a linear or branched alkyl having 1 to 30 carbon atoms constituting the chain, in which the number of carbon atoms is preferably 1 to 20, more preferably 1 to 10. The above alkyl may include methyl, ethyl, n-propyl, isopropyl, n-butyl, isobutyl, tert-butyl, sec-butyl, etc. Herein, “(C₃-C₃₀)cycloalkyl(ene)” is meant to be a mono- or polycyclic hydrocarbon having 3 to 30 ring backbone carbon atoms, in which the number of carbon atoms is preferably 3 to 20, more preferably 3 to 7. The above cycloalkyl may include cyclopropyl, cyclobutyl, cyclopentyl, cyclohexyl, cyclopentylmethyl, cyclohexylmethyl, etc. The “(3- to 7-membered)heterocycloalkyl” in the present disclosure is meant to be a cycloalkyl having 3 to 7 ring backbone atoms, preferably 5 to 7 ring backbone atoms and at least one heteroatom selected from the group consisting of B, N, O, S, Si, and P, preferably O, S, and N, and includes tetrahydrofuran, pyrrolidine, thiolan, tetrahydropyran, etc. The “(C₆-C₃₀)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The “(3- to 30-membered)heteroaryl(ene)” in the present disclosure is an aryl having 3 to 30 ring backbone atoms including at least one heteroatom selected from the group consisting of B, N, O, S, Si, P, Se, and Ge in which the number of the ring backbone carbon atoms is preferably 5 to 25. The number of the heteroatoms in the heteroaryl is preferably 1 to 4. The above heteroaryl may be a monocyclic ring, or a fused ring condensed with at least one benzene ring, and may be partially saturated. Also, the above heteroaryl herein may be one formed by linking at least one heteroaryl or aryl group to a heteroaryl group via a single bond(s). Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified as a heteroaryl(ene) with electronic properties or a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, a substituted or unsubstituted dibenzothiophenyl, or a substituted or unsubstituted dibenzoselenophenyl. Herein, “a fused ring of (C₃-C₃₀) aliphatic ring and (C₆-C₃₀) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of the (C₃-C₃₀) aliphatic ring and the (C₆-C₃₀) aromatic ring may be replaced with at least one heteroatom selected from B, N, O, S, Si, and P, preferably at least one heteroatom selected from N, O, and S. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, and preferably this may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, the term “substituted” in the expression “substituted or unsubstituted” means that a hydrogen atom in a certain functional group is replaced with another atom or functional group, i.e., a substituent. Unless otherwise specified, the substituents may not be limited to hydrogen at positions where the substituents may be substituted, and when two or more hydrogen atoms are each replaced with a substituent in a functional group, the substituents may be the same as or different from each other. For example, “a substituent to which two or more substituents are connected” may be pyridine-triazine. That is, pyridine-triazine may be heteroaryl or may be interpreted as one substituent in which two heteroaryls are connected. Preferably, the substituted alkyl, the substituted alkenyl, the substituted cycloalkyl, the substituted heterocycloalkyl, the substituted aryl(ene), the substituted heteroaryl(ene), the substituted alkoxy, the substituted trialkylsilyl, the substituted dialkylarylsilyl, the substituted alkyldiarylsilyl, and the substituted triarylsilyl in the formulas of the present disclosure each independently are substituted with least one selected from the group consisting of: deuterium, halogen, cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3- to 30-membered)heteroaryl unsubstituted or substituted with at least one (C₆-C₃₀)aryl, (C₆-C₃₀)aryl unsubstituted or substituted with at least one of (C₁-C₃₀)alkyl and (3- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, tri(C₆-C₃₀)arylgermanyl, amino, mono- or di(C₁-C₃₀)alkylamino, mono- or di(C₂-C₃₀)alkenylamino, mono- or di(C₆-C₃₀)arylamino unsubstituted or substituted with (C₁-C₃₀)alkyl, mono- or di(3- to 30-membered)heteroarylamino, (C₁-C₃₀)alkyl(C₂-C₃₀)alkenylamino, (C₁-C₃₀)alkyl(C₆-C₃₀)arylamino, (C₁-C₃₀)alkyl(3- to 30-membered)heteroarylamino, (C₂-C₃₀)alkenyl(C₆-C₃₀)arylamino, (C₂-C₃₀)alkenyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, etc. For example, the substituents may be substituted with deuterium, methyl, tert-butyl, phenyl, or naphthyl, etc.

When a substituent is not shown in the chemical formula or the compound structure of the present disclosure, it may signify that all positions that may be present as substituents are hydrogen or deuterium. That is, in the case of deuterium, an isotope of hydrogen, some of the hydrogen atoms may be deuterium, which is an isotope; and in this case, the content of deuterium may be 0% to 100%. In the case where the substituent is not shown in the chemical formula or the compound structure of the present disclosure, when deuterium is not explicitly excluded, hydrogen and deuterium may be mixed and used in the compound, such as when the content of deuterium is 0%, the content of hydrogen is 100%, and all substituents are hydrogen. The deuterium is an element having a deuteron composed of one proton and one neutron as an atomic nucleus, which is one of the isotopes of hydrogen, and may be represented by hydrogen-2, and the element symbol may be D or ²H. Although the isotope has the same atomic number (Z), an isotope having a different mass number (A) means the same number of protons and the number of neutrons may also be interpreted as an element having different numbers.

Herein, “combinations thereof” signifies that one or more components of the corresponding list are combined to form a known or chemically stable arrangement that a person skilled in the art could conceive of from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formula of the present disclosure, when multiple substituents are indicated by the same symbol, each of these substituents represented by the same symbol may be the identical or different from one another.

Hereinafter, the compound according to one embodiment will be described.

The compound according to one embodiment of the present disclosure is represented by the following Formula 1.

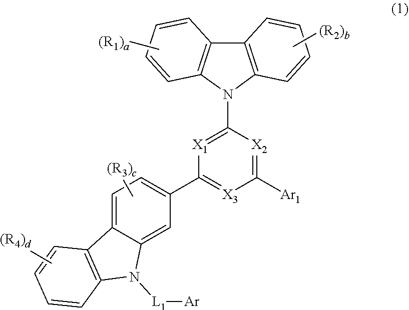

In Formula 1,
X₁ to X₃ each independently represent CR₅ or N, provided that at least one of X₁ to X₃ is N;
Ar₁ represents hydrogen, deuterium, halogen, cyano, amino, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, —Si(R₁₁)(R₁₂)(R₁₃), or —N(R₁₄)(R₁₅);
R₁₁ to R₁₅ each independently represent a substituted or unsubstituted (C₁-C₃₀)alkyl, a substituted or unsubstituted (C₂-C₃₀)alkenyl, a substituted or unsubstituted (C₆-C₃₀)aryl, or a substituted or unsubstituted (3- to 30-membered)heteroaryl;
L₁ represents a single bond, a substituted or unsubstituted (C₆-C₃₀)arylene, or a substituted or unsubstituted (3- to 30-membered)heteroarylene;
R₁ to R₅ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s);
Ar represents a substituted or unsubstituted (C₆-C₃₀)aryl or a substituted or unsubstituted (3- to 30-membered)heteroaryl; and
a, b, and d each independently represent an integer of 1 to 4, c represents an integer of 1 to 3, and when a to d are an integer of 2 or more, each of R₁ to R₄ may be the same as or different from each other.

In one embodiment, at least two of X₁ to X₃ may be N.

In one embodiment, all of X₁ to X₃ may be N.

In one embodiment, Ar₁ may be a substituted or unsubstituted (C₆-C₃₀)aryl or a substituted or unsubstituted (5- to 30-membered)heteroaryl, preferably (C₆-C₂₅)aryl unsubstituted or substituted with (C₁-C₃₀)alkyl or a substituted or unsubstituted (5- to 25-membered)heteroaryl, more preferably (C₆-C₁₈)aryl unsubstituted or substituted with (C₁-C₁₀)alkyl or a substituted or unsubstituted (5- to 18-membered)heteroaryl. For example, Ar₁ may be phenyl unsubstituted or substituted with at least one of methyl, dimethyl, and tert-butyl, a substituted or unsubstituted p-biphenyl, a substituted or unsubstituted m-biphenyl, a substituted or unsubstituted o-biphenyl, a substituted or unsubstituted m-terphenyl, a substituted or unsubstituted p-terphenyl, naphthyl unsubstituted or substituted with phenyl, a substituted or unsubstituted phenanthrenyl, a substituted or unsubstituted chrysenyl, a substituted or unsubstituted triphenylenyl, a substituted or unsubstituted fluoranthenyl, or a substituted or unsubstituted dibenzofuranyl. For example, the substituents may be further substituted with at least one deuterium.

In one embodiment, L₁ may be a single bond or a substituted or unsubstituted (C₆-C₃₀)arylene, preferably a single bond or a substituted or unsubstituted (C₆-C₂₅)arylene, more preferably a single bond or a substituted or unsubstituted (C₆-C₁₈)arylene. For example, L₁ may be a single bond, phenylene unsubstituted or substituted with phenyl, or a substituted or unsubstituted biphenylene.

In one embodiment, R₁ to R₅ each independently may be hydrogen, deuterium, halogen, cyano, or a substituted or unsubstituted (C₆-C₃₀)aryl. For example, R₁, R₂, and R₅ each independently may be hydrogen or deuterium, and R₃ and R₄ each independently may be hydrogen, deuterium, or a substituted or unsubstituted phenyl.

In one embodiment, Ar may be a substituted or unsubstituted (C₆-C₃₀)aryl or a substituted or unsubstituted (5- to 30-membered)heteroaryl, preferably a substituted or unsubstituted (5- to 30-membered)heteroaryl having a hole property. For example, Ar may be dibenzofuranyl unsubstituted or substituted with methyl or phenyl, a substituted or unsubstituted dibenzothiophenyl, or a substituted or unsubstituted dibenzoselenophenyl.

According to one embodiment, the compound represented by Formula 1 may be represented by the following Formula 1-1.

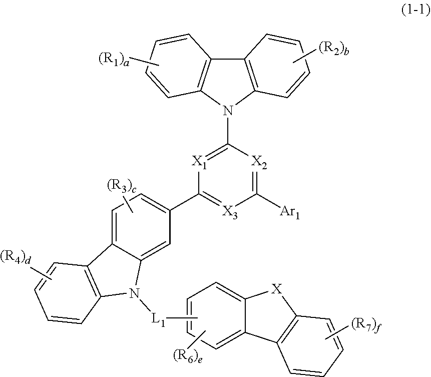

In Formula 1-1,
R₁ to R₄, X₁ to X₃, Ar₁, L₁, and a to d are as defined in Formula 1;
X represents O, S, or Se;
R₆ and R₇ are as defined as R₁ in Formula 1; and
e represents an integer of 1 to 3, f represents an integer of 1 to 4, and when e and f are an integer of 2 or more, each of R₆ and each of R₇ may be the same as or different from each other.

According to one embodiment, the compound represented by Formula 1 may be more specifically illustrated by the following compounds, but is not limited thereto:

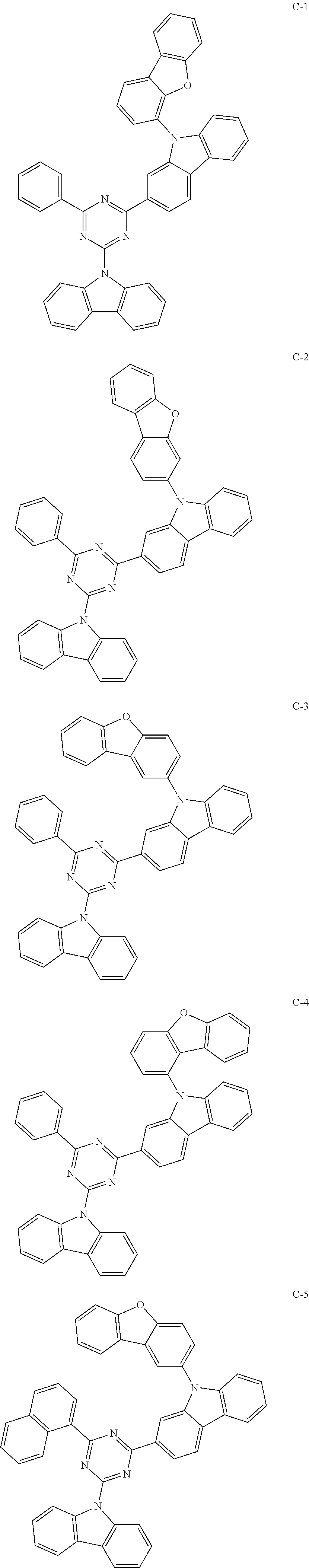

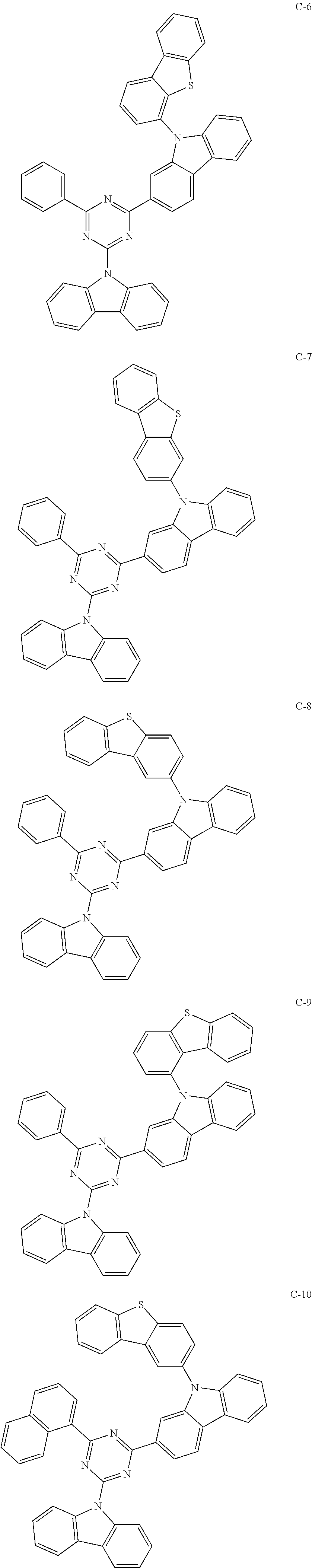

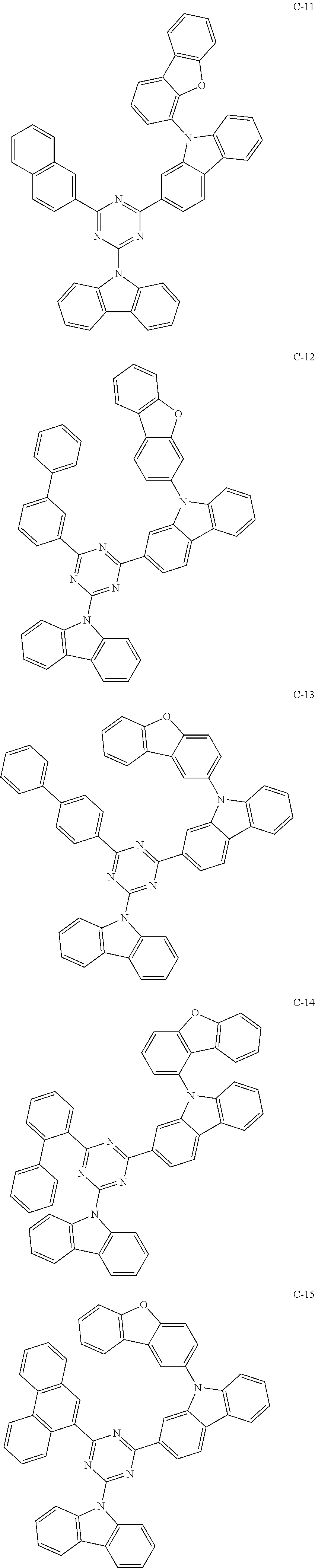

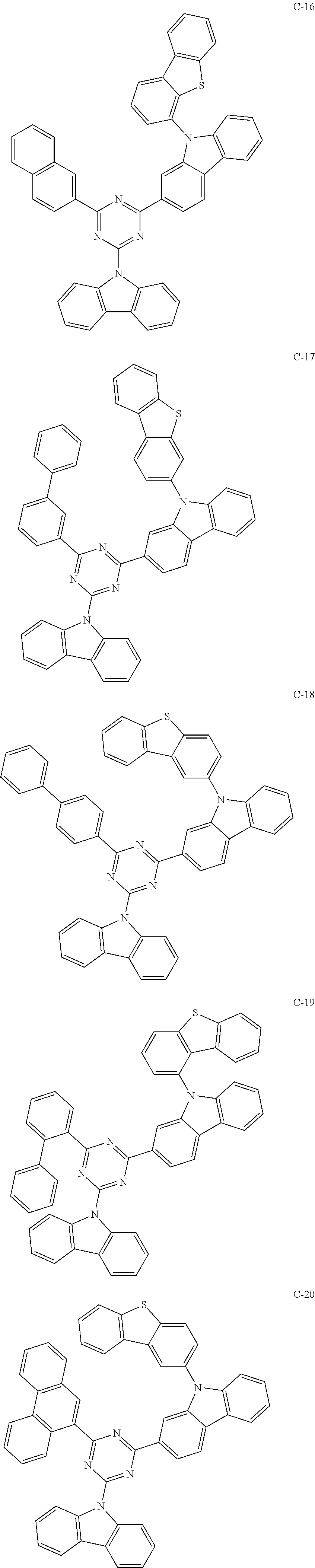

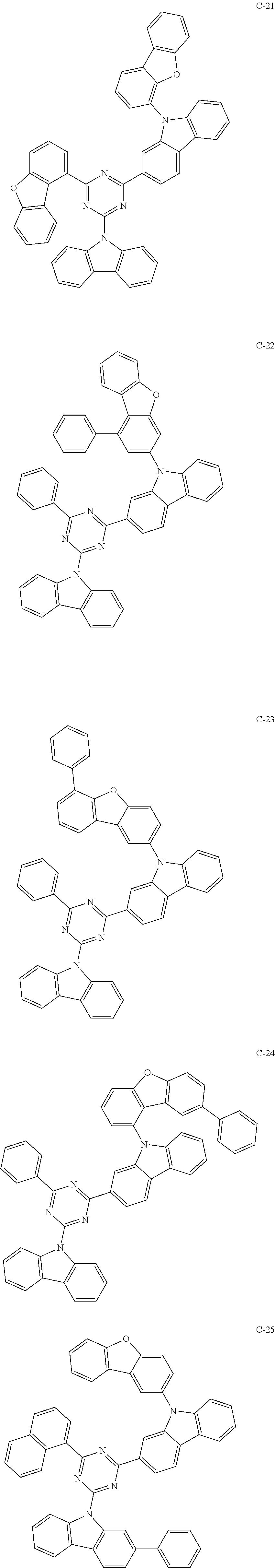

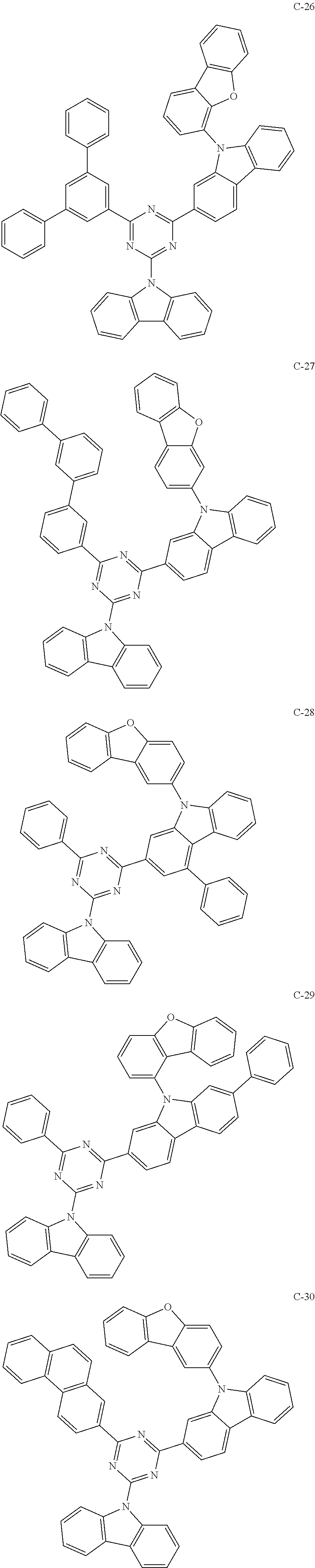

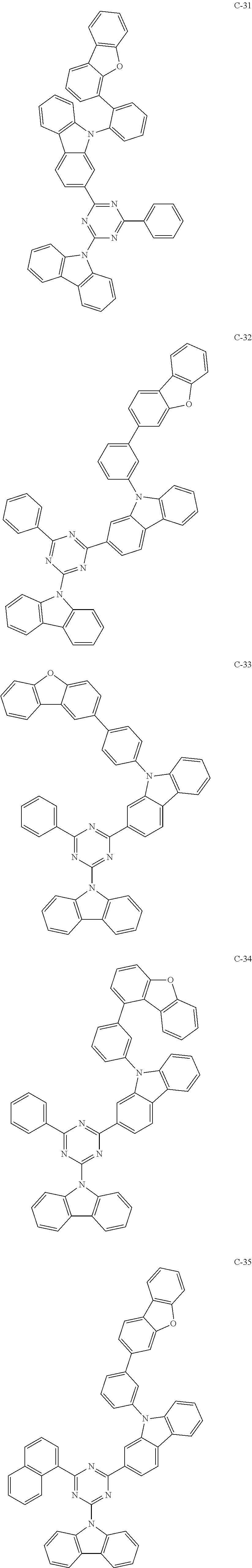 [SMILES: c1ccc(-c2nc(-c3ccc4c5ccccc5n(-c5ccccc5-c5cccc6c5oc5ccccc56)c4c3)nc(-n3c4ccccc4c4ccccc43)n2)cc1 ; c1ccc(-c2nc(-c3ccc4c5ccccc5n(-c5cccc(-c6ccc7c(c6)oc6ccccc67)c5)c4c3)nc(-n3c4ccccc4c4ccccc43)n2)cc1 ; c1ccc(-c2nc(-c3ccc4c5ccccc5n(-c5ccc(-c6ccc7oc8ccccc8c7c6)cc5)c4c3)nc(-n3c4ccccc4c4ccccc43)n2)cc1 ; c1ccc(-c2nc(-c3ccc4c5ccccc5n(-c5cccc(-c6cccc7oc8ccccc8c67)c5)c4c3)nc(-n3c4ccccc4c4ccccc43)n2)cc1 ; c1cc(-c2ccc3c(c2)oc2ccccc23)cc(-n2c3ccccc3c3ccc(-c4nc(-c5cccc6ccccc56)nc(-n5c6ccccc6c6ccccc65)n4)cc32)c1]

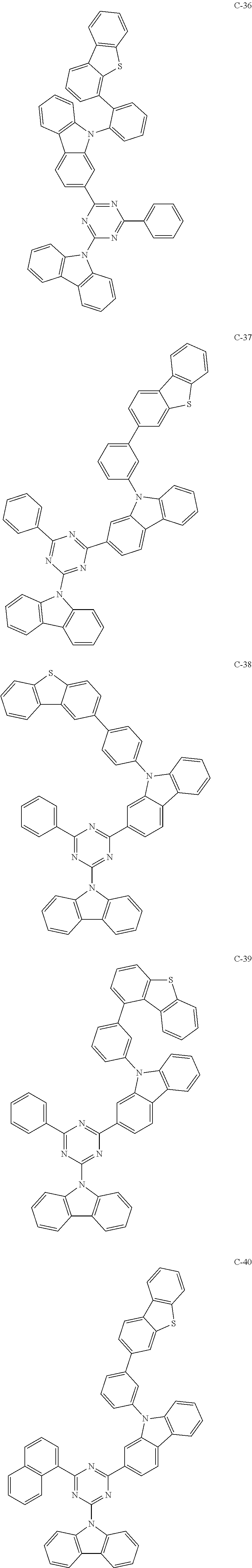 [SMILES: c1ccc(-c2nc(-c3ccc4c5ccccc5n(-c5ccccc5-c5cccc6c5sc5ccccc56)c4c3)nc(-n3c4ccccc4c4ccccc43)n2)cc1 ; c1ccc(-c2nc(-c3ccc4c5ccccc5n(-c5cccc(-c6ccc7c(c6)sc6ccccc67)c5)c4c3)nc(-n3c4ccccc4c4ccccc43)n2)cc1 ; c1ccc(-c2nc(-c3ccc4c5ccccc5n(-c5ccc(-c6ccc7sc8ccccc8c7c6)cc5)c4c3)nc(-n3c4ccccc4c4ccccc43)n2)cc1 ; c1ccc(-c2nc(-c3ccc4c5ccccc5n(-c5cccc(-c6cccc7sc8ccccc8c67)c5)c4c3)nc(-n3c4ccccc4c4ccccc43)n2)cc1 ; c1cc(-c2ccc3c(c2)sc2ccccc23)cc(-n2c3ccccc3c3ccc(-c4nc(-c5cccc6ccccc56)nc(-n5c6ccccc6c6ccccc65)n4)cc32)c1]

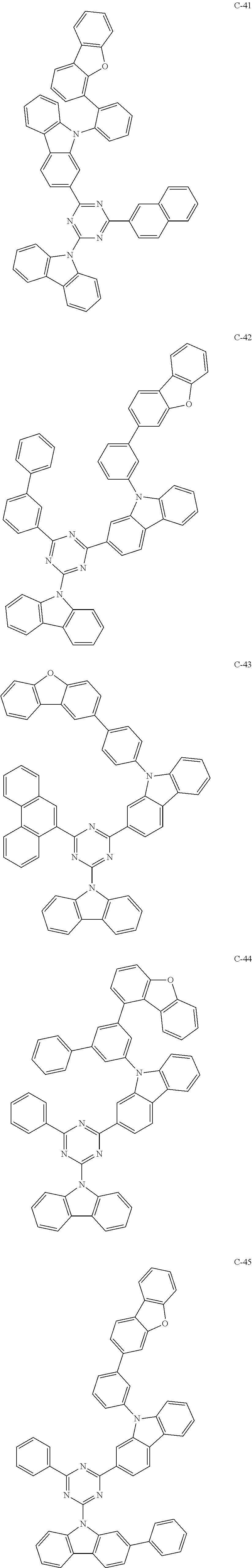

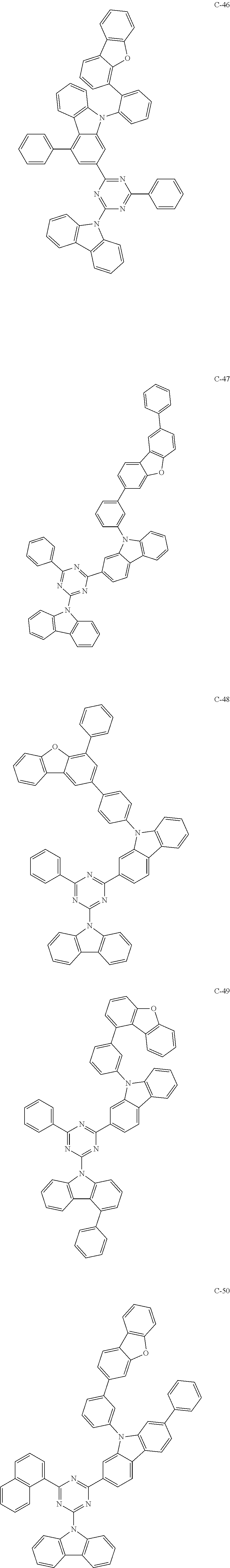

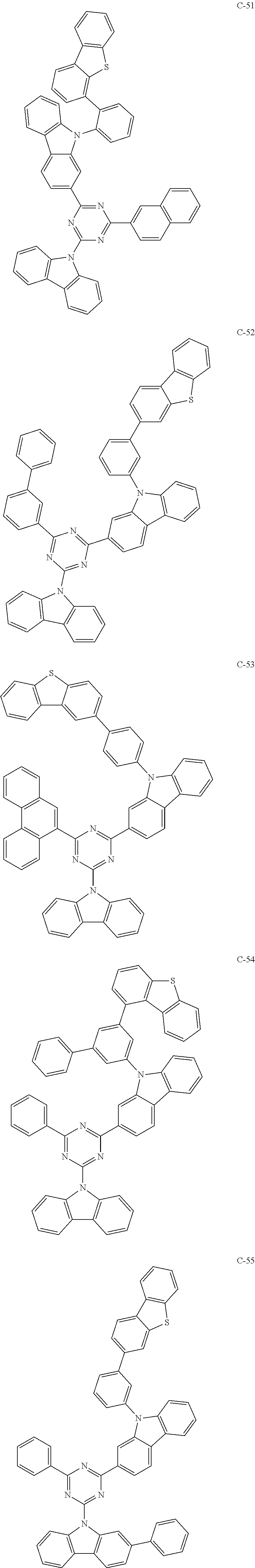

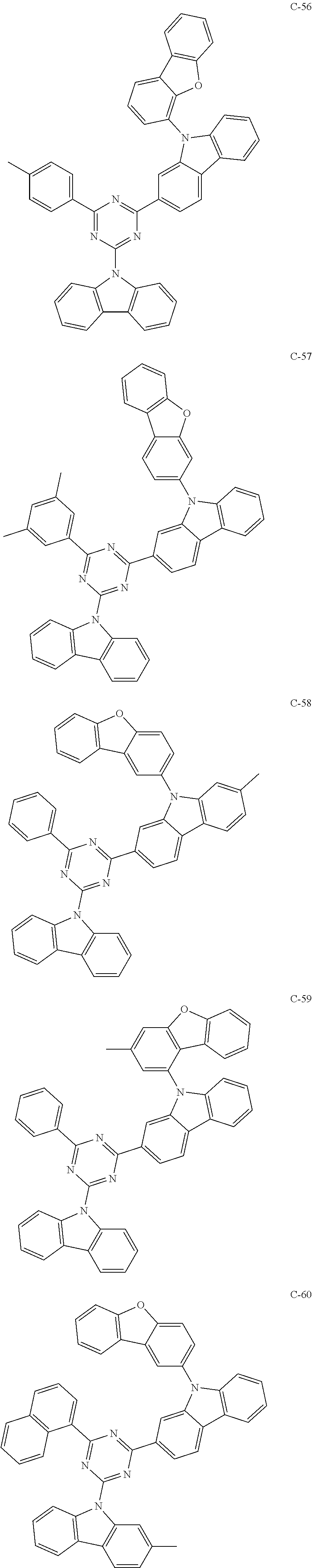

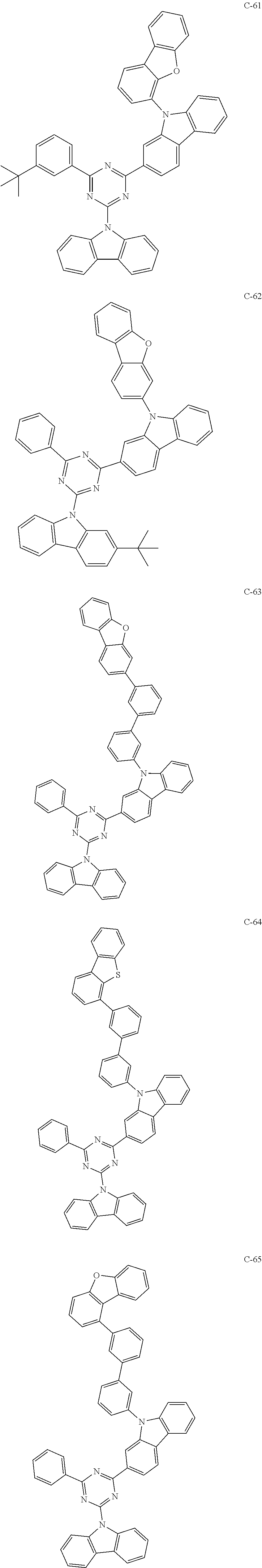

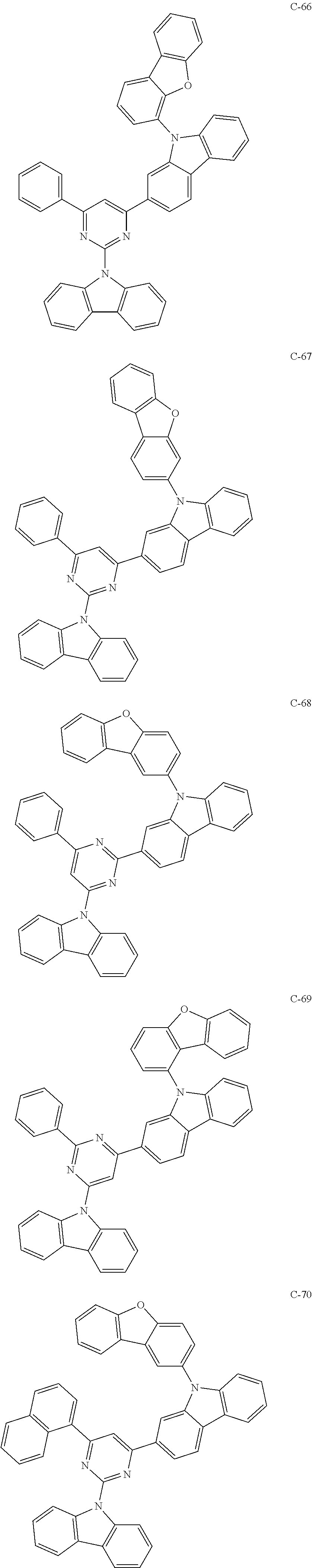

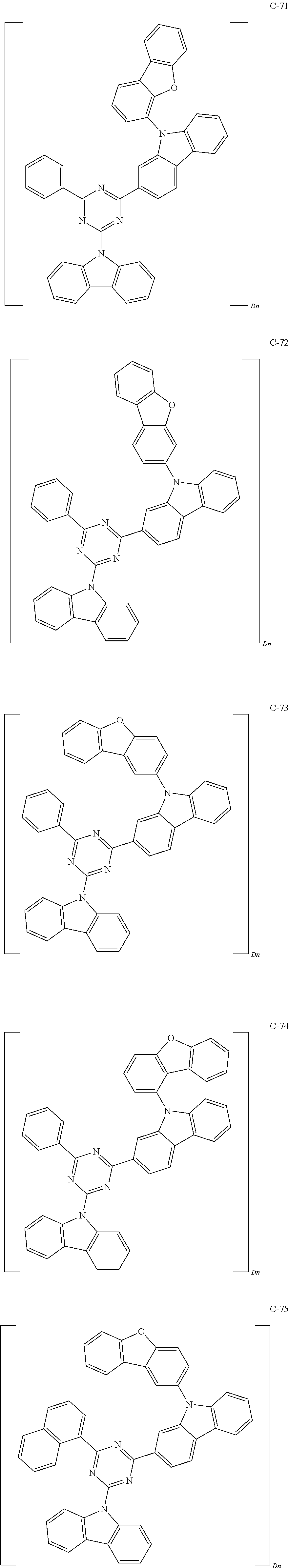

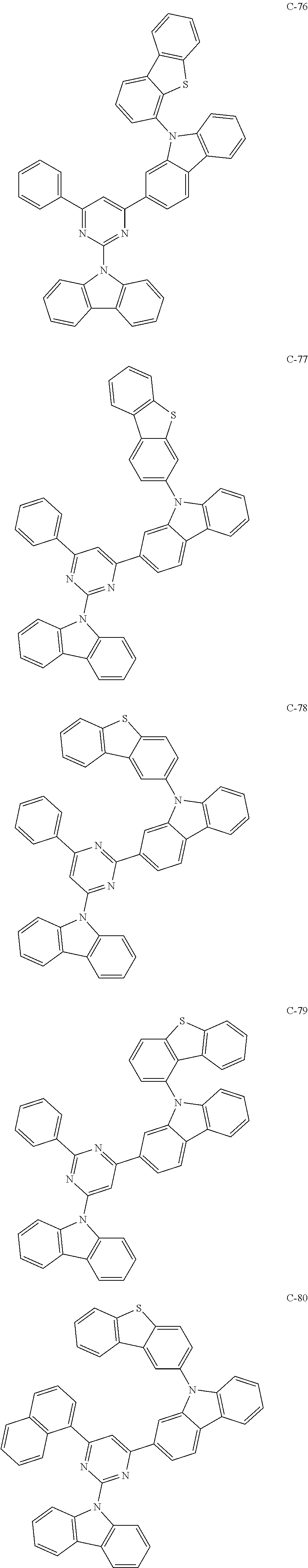

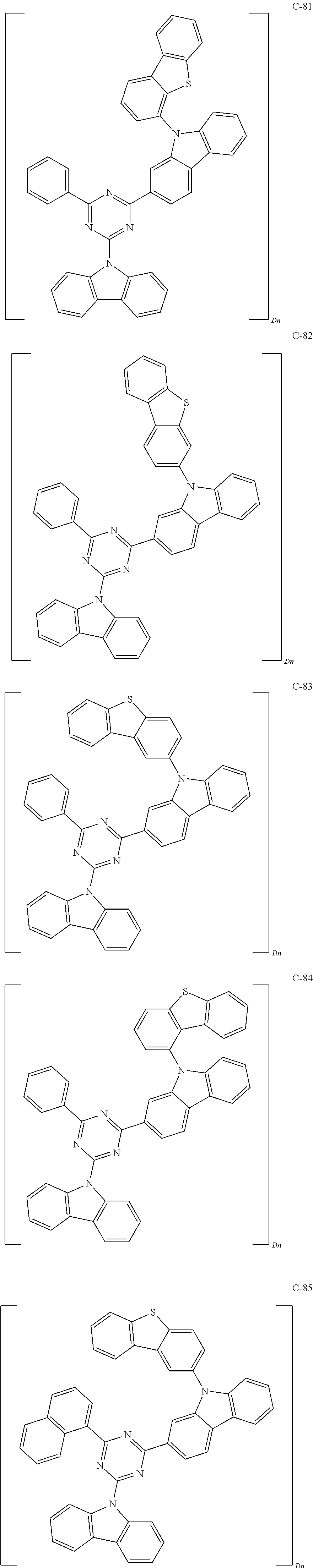

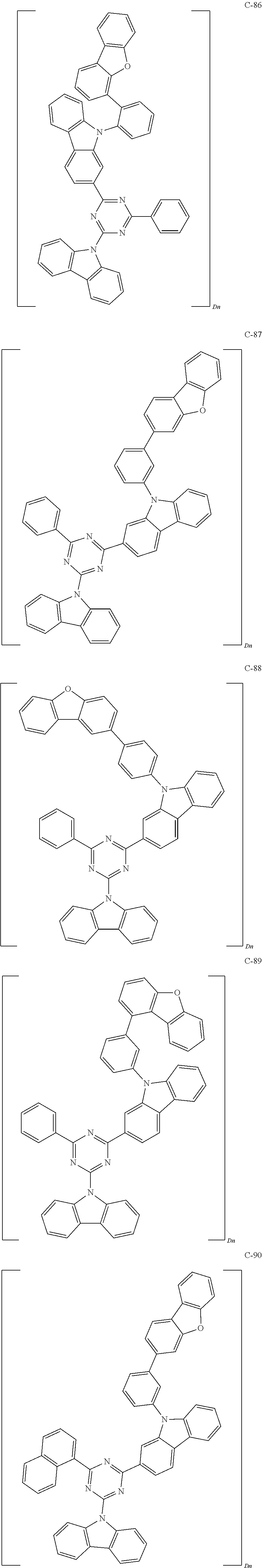

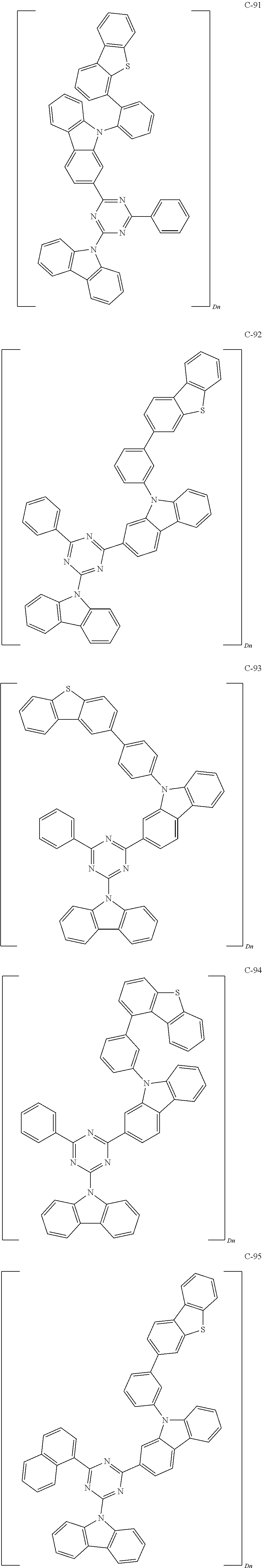

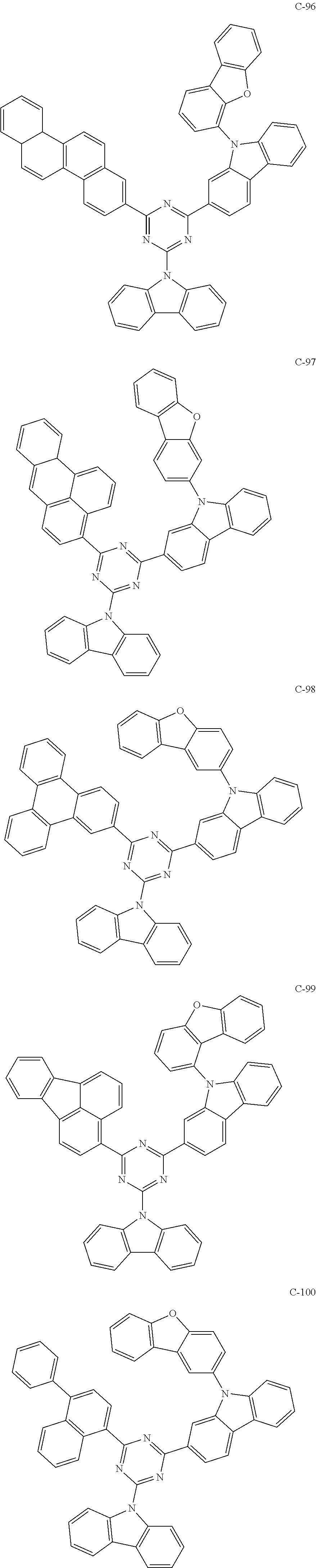

Wherein “Dₙ” in the above compounds means that n number of hydrogens is replaced by deuterium, wherein n represents an integer of 1 or more, and the upper limit of n is determined by the number of hydrogens that can be substituted in each compound.

The compound represented by Formula 1 according to the present disclosure may be prepared by synthetic methods known to those skilled in the art.

According to another embodiment, the present disclosure provides an organic electroluminescent material comprising the compound represented by Formula 1 above and a compound represented by the following Formula 2.

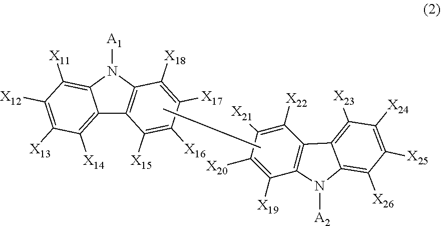

In Formula 2,
A₁ and A₂ each independently represent a substituted or unsubstituted (C₆-C₃₀)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are linked to each other to form a single bond; and
X₁₁ to X₁₄, X₂₃ to X₂₆, and X₁₅ to X₂₂ which do not form a single bond each independently represent hydrogen, deuterium, a substituted or unsubstituted (C₆-C₃₀)aryl, or a substituted or unsubstituted (3- to 30-membered)heteroaryl; or may be linked to the adjacent substituents to form a ring(s).

In one embodiment, A₁ and A₂ each independently may be a substituted or unsubstituted (C₆-C₂₅)aryl, a substituted or unsubstituted dibenzofuranyl, a substituted or unsubstituted dibenzothiophenyl, or a substituted or unsubstituted carbazolyl. For example, A₁ and A₂ each independently may be phenyl unsubstituted or substituted with deuterium; biphenyl unsubstituted or substituted with deuterium; terphenyl unsubstituted or substituted with deuterium; naphthyl unsubstituted or substituted with deuterium; fluorenyl unsubstituted or substituted with at least one of deuterium, (C₁-C₃₀)alkyl, and (C₆-C₃₀)aryl; benzofluorenyl unsubstituted or substituted with at least one of deuterium, (C₁-C₃₀)alkyl, and (C₆-C₃₀)aryl; triphenylenyl unsubstituted or substituted with deuterium; fluoranthenyl unsubstituted or substituted with deuterium; phenanthrenyl unsubstituted or substituted with deuterium; dibenzofuranyl unsubstituted or substituted with deuterium; dibenzothiophenyl unsubstituted or substituted with deuterium; carbazolyl unsubstituted or substituted with deuterium; or a combination thereof. For example, A₁ and A₂ each independently may be a substituted or unsubstituted phenyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted o-terphenyl, naphthyl unsubstituted or substituted with phenyl, a substituted or unsubstituted fluorenyl, benzofluorenyl unsubstituted or substituted with phenyl, a substituted or unsubstituted triphenylenyl, a substituted or unsubstituted fluoranthenyl, a substituted or unsubstituted phenanthrenyl, benzophenanthrenyl unsubstituted or substituted with phenyl, dibenzofuranyl unsubstituted or substituted with phenyl, dibenzothiophenyl unsubstituted or substituted with phenyl, or carbazolyl unsubstituted or substituted with phenyl or naphthyl.

Wherein, A₁ and A₂ each independently may be substituted with at least one of deuterium, (C₆-C₃₀)aryl, and (3- to 30-membered)heteroaryl, for example, may be substituted with at least one of deuterium, (C₆-C₁₈)aryl, and (5- to 20-membered)heteroaryl. For example, A₁ and A₂ each independently may be substituted with at least one of phenyl, naphthyl, triphenylenyl, dibenzofuranyl, and dibenzothiophenyl, and may be further substituted with at least one deuterium.

According to one embodiment, the compound represented by Formula 2 may be represented by any one of the following Formulas 2-1 to 2-8.

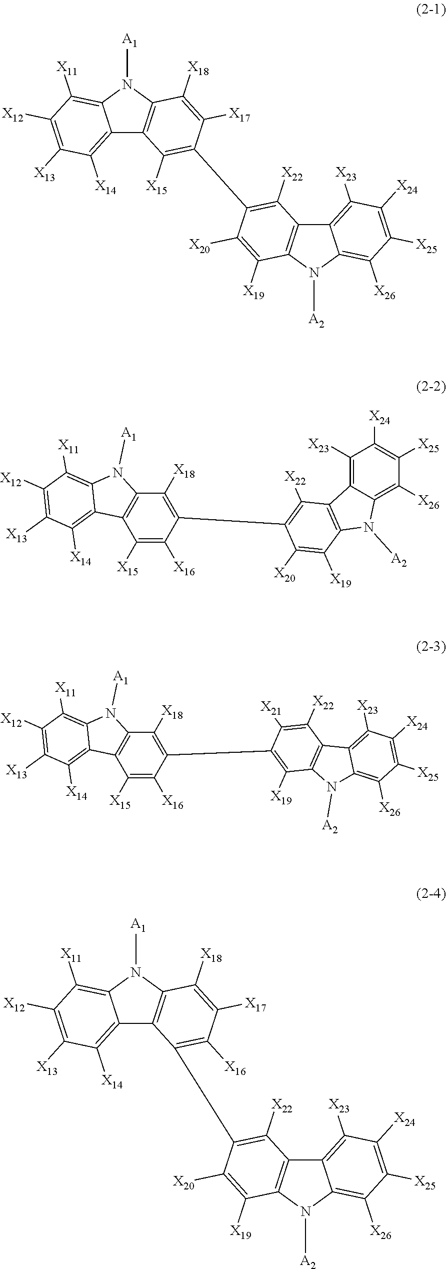

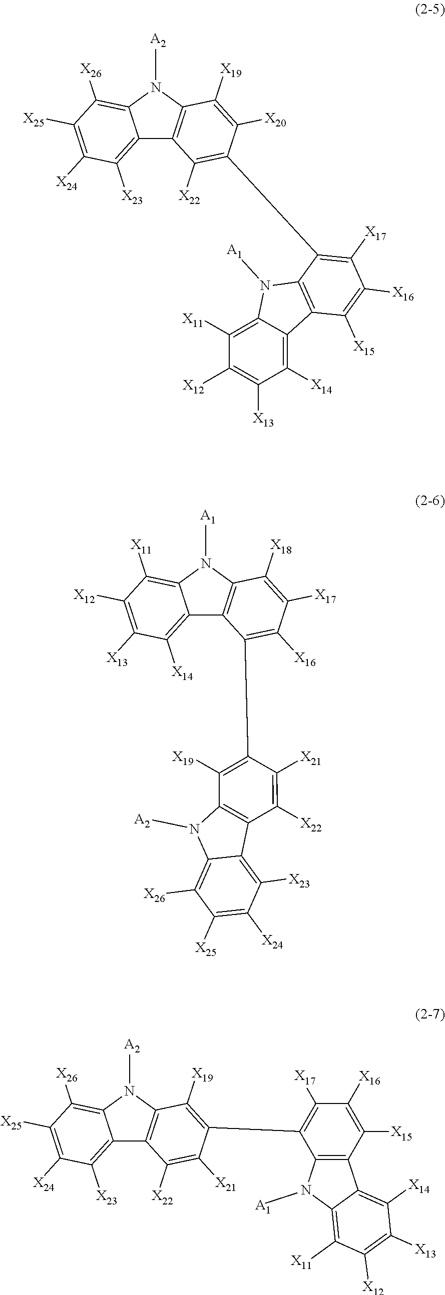

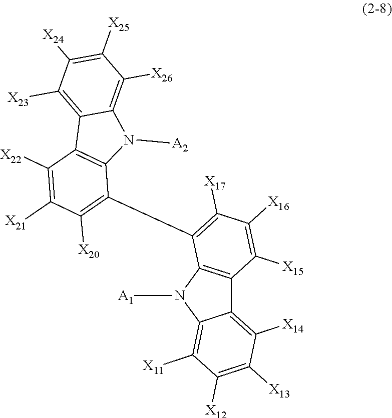

In Formulas 2-1 to 2-8,
A₁, A₂ and X₁₁ to X₂₆ are as defined in Formula 2.

In one embodiment, X₁₁ to X₁₄, X₂₃ to X₂₆, and X₁₅ to X₂₂ which do not form a single bond each independently may be hydrogen, deuterium, a substituted or unsubstituted (C₆-C₃₀)aryl, or a substituted or unsubstituted (5- to 30-membered)heteroaryl, or may be linked to the adjacent substituents to form a ring(s), preferably hydrogen, deuterium, a substituted or unsubstituted (C₆-C₂₅)aryl, or a substituted or unsubstituted (5- to 25-membered)heteroaryl, more preferably hydrogen or deuterium. For example, at least four of X₁₁ to X₁₄, X₂₃ to X₂₆, and X₁₅ to X₂₂ which do not form a single bond, may be deuterium.

In one embodiment, at least one of X₁₁, X₁₈, X₁₉, and X₂₆ may be deuterium.

In one embodiment, at least two of X₁₁, X₁₈, X₁₉, and X₂₆ may be deuterium.

In one embodiment, at least three of X₁₁, X₁₈, X₁₉, and X₂₆ may be deuterium.

In one embodiment, all of X₁₁, X₁₈, X₁₉, and X₂₆ may be deuterium.

In one embodiment, when X₁₆ and X₂₁ are linked to each other to form a single bond, all of X₁₁, X₁₈, X₁₉, and X₂₆ may be deuterium, and, when X₁₈ and X₁₉ are linked to each other to form a single bond, X₁₁ and X₂₆ may be deuterium.

In one embodiment, the deuterium substitution rate of X₁₁ to X₂₆ may be 25% to 100%, for example, 35% to 100%, for example, 45% to 100%, or, for example, 55% to 100%. The upper limit of the deuterium substitution rate may be 100%, but may also be less than 100%, for example, about 99%.

According to one embodiment, in the compound represented by Formula 2, the deuterium substitution rate may be 40% to 100%, for example, 50% to 100%, for example, 60% to 100%, or, for example, 70% to 100%. The upper limit of the deuterium substitution rate may be 100%, but may also be less than 100%, for example, about 99%. That is, the compound represented by Formula 2 can be a compound in which all hydrogens at substitutable positions are substituted with deuterium, or a compound in which hydrogens at substitutable positions are partially substituted with deuterium.

When the compound represented by Formula 2 contains deuterium substituted according to the above-mentioned number or ratio, the bond dissociation energy according to deuteration increases, thereby increasing the stability of the compound represented by Formula 2. When such a compound represented by Formula 2 is used in an organic electroluminescent device, improved lifespan characteristics may be exhibited.

According to one embodiment, the compound represented by Formula 2 may be more specifically illustrated by the following compounds, but is not limited thereto.

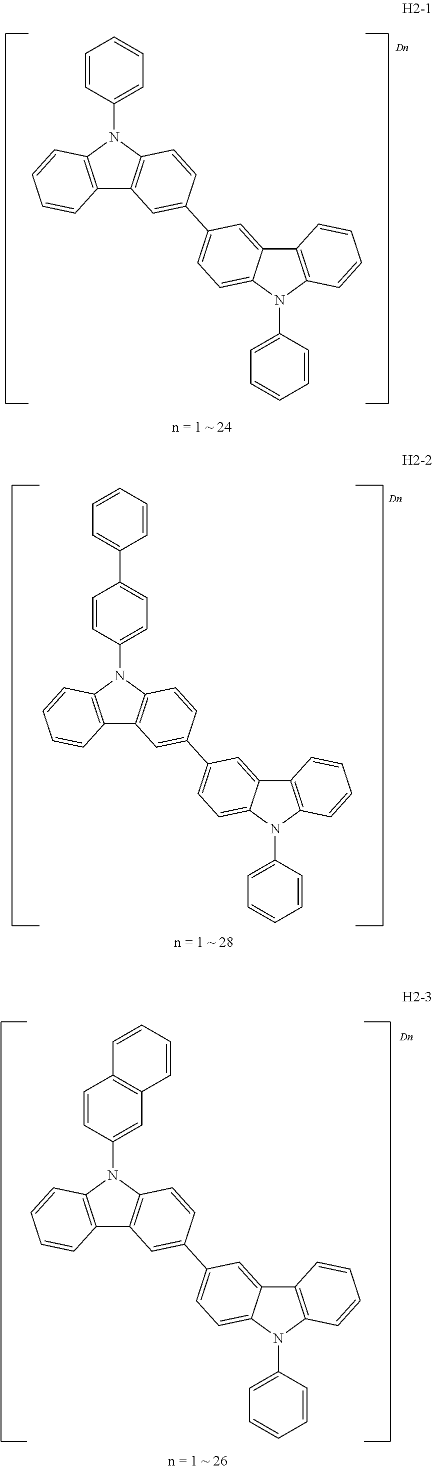

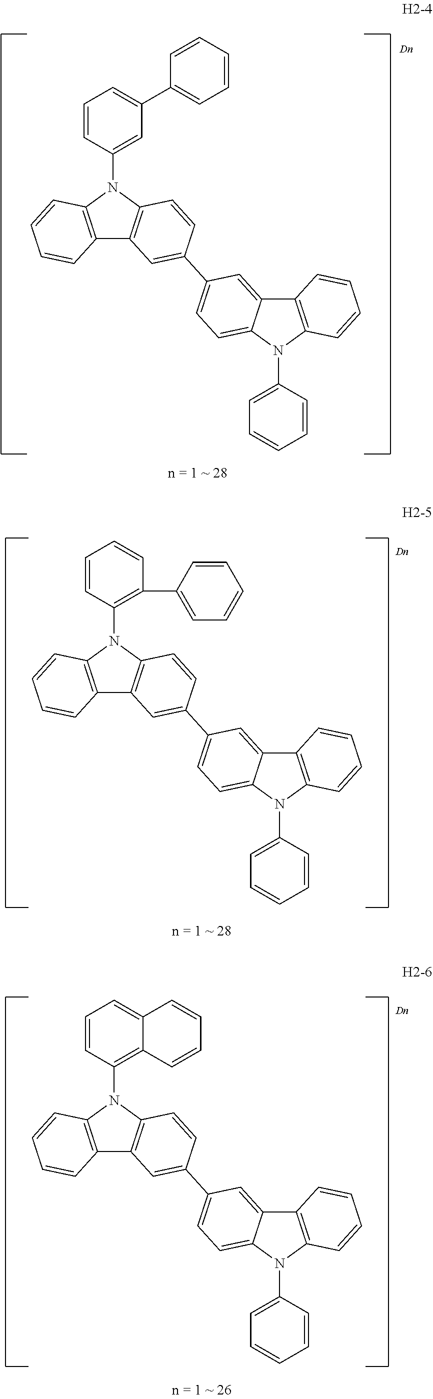

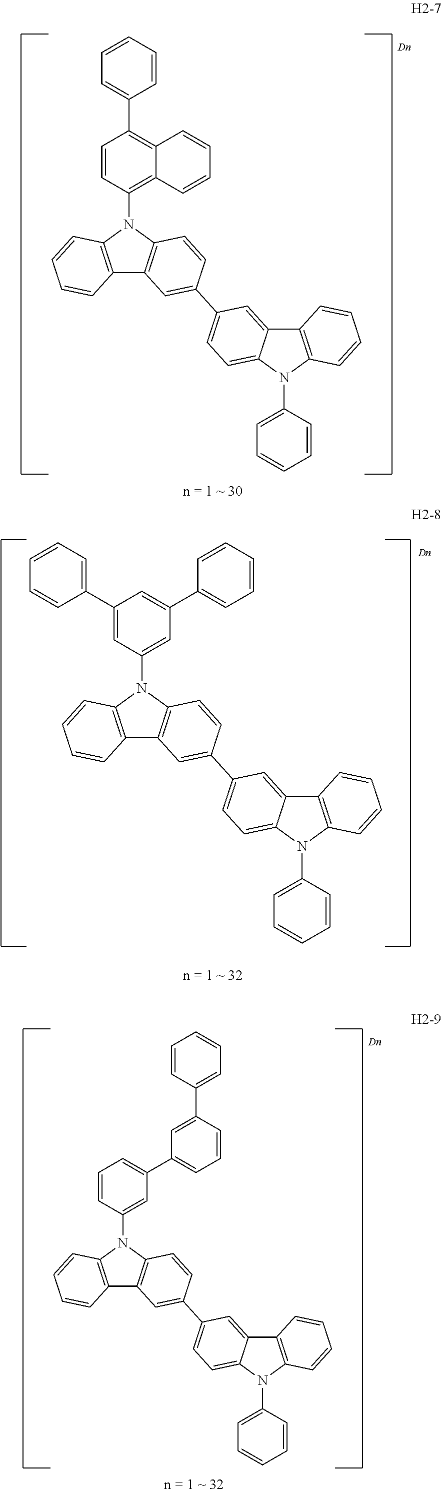

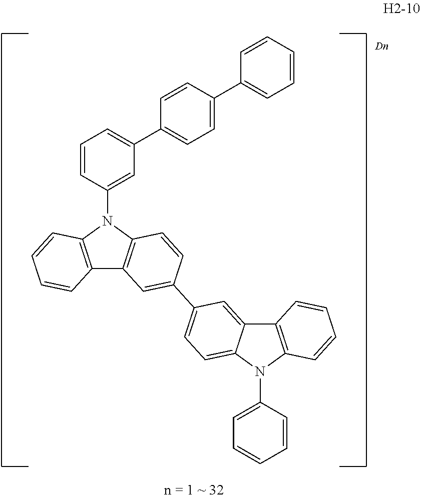

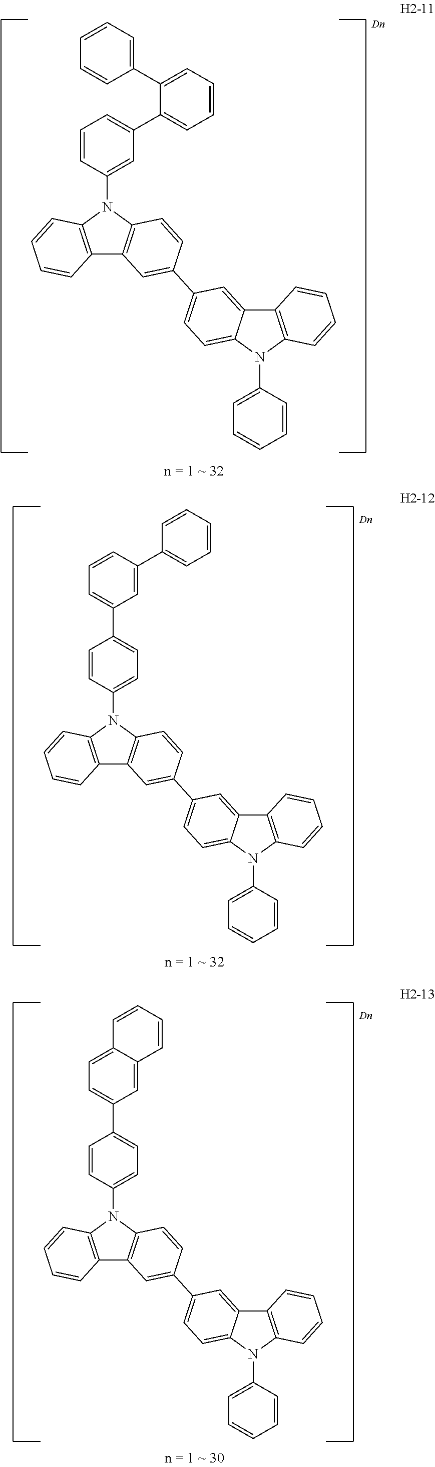

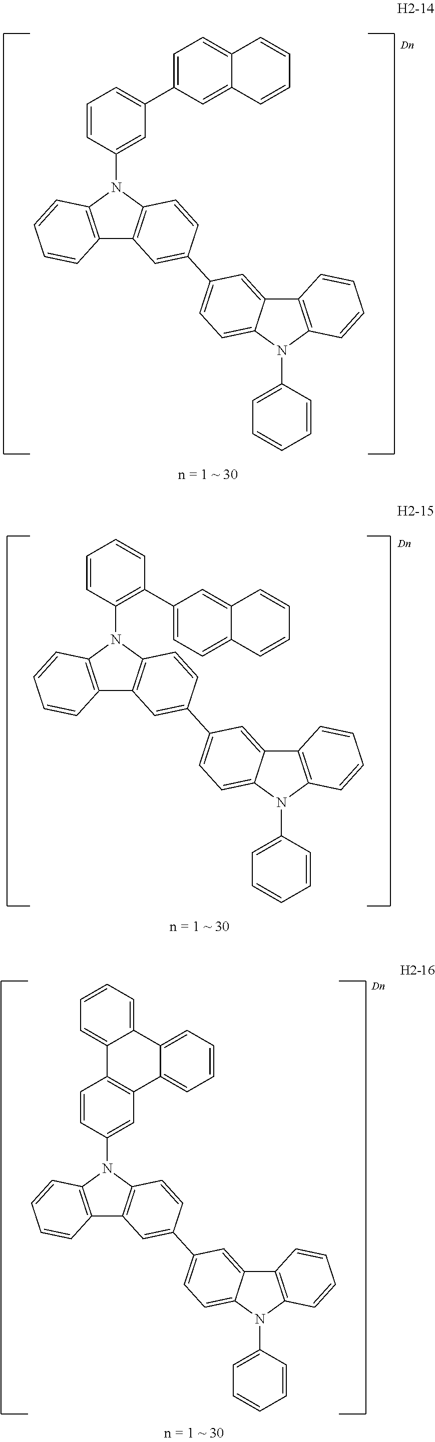

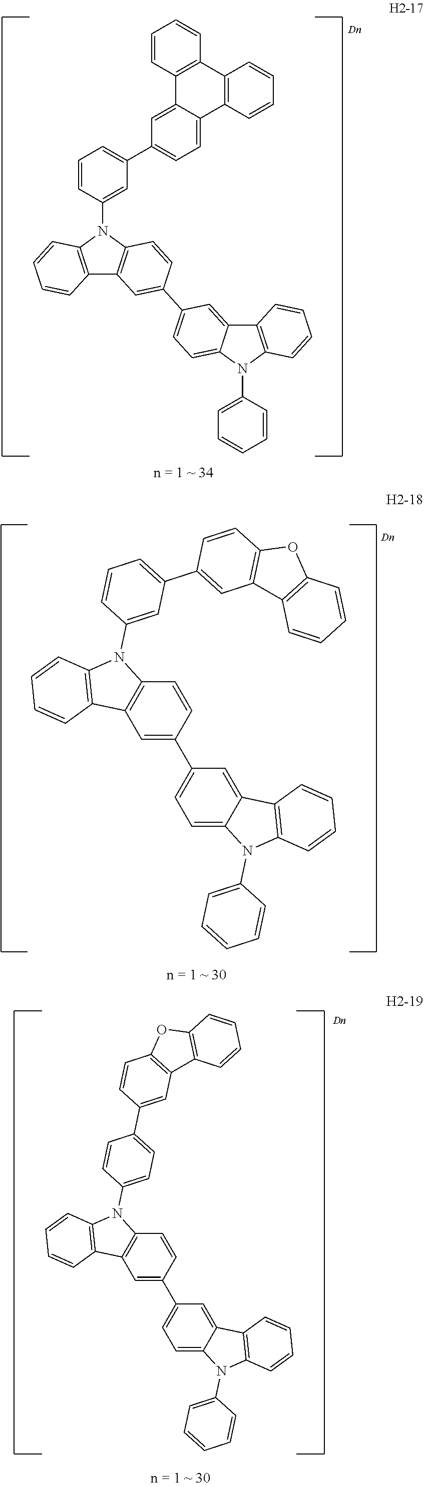

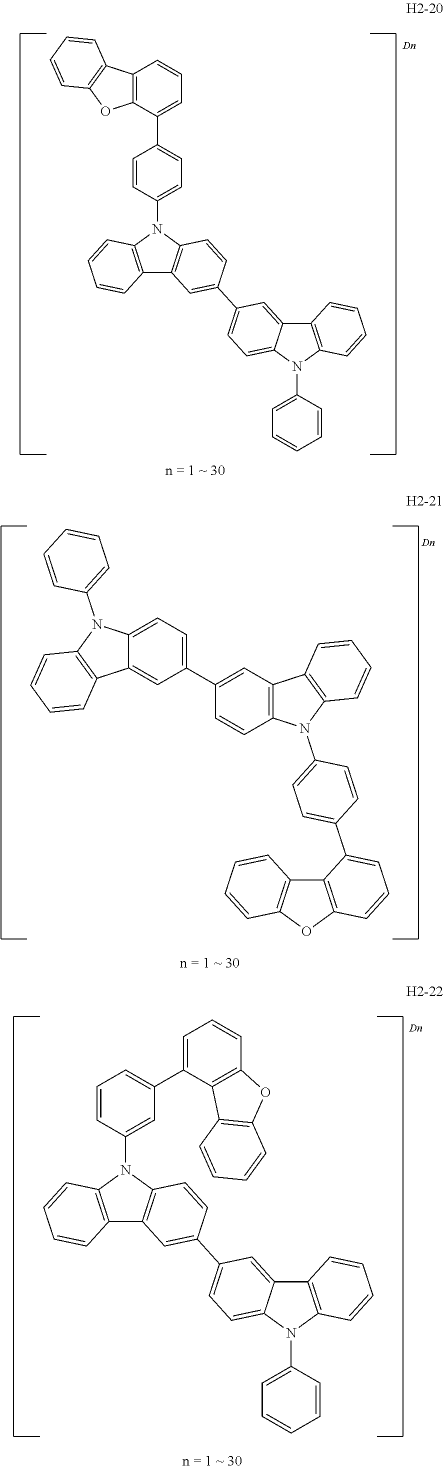

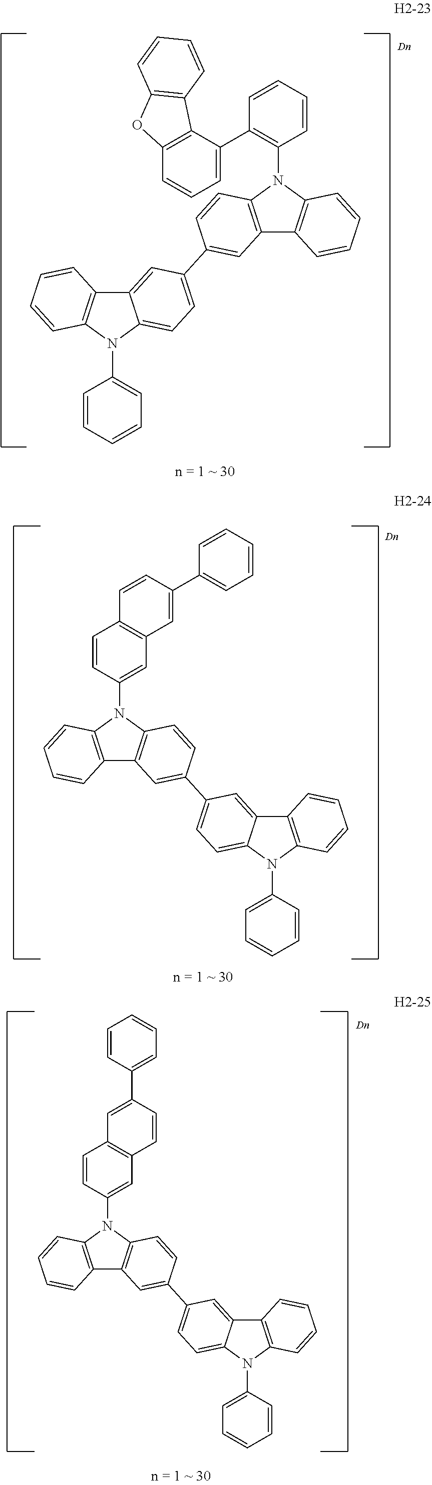

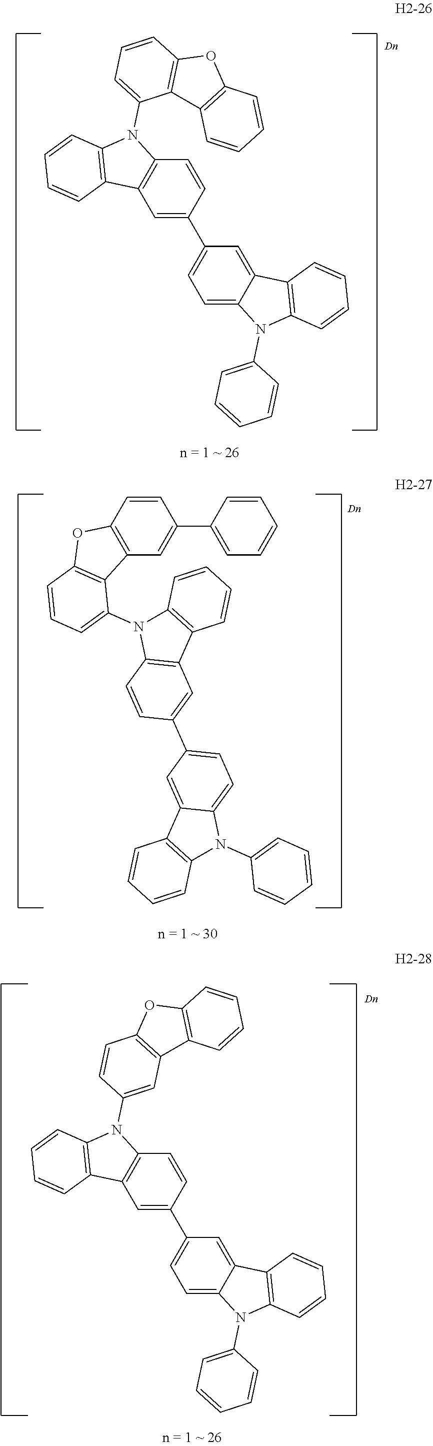

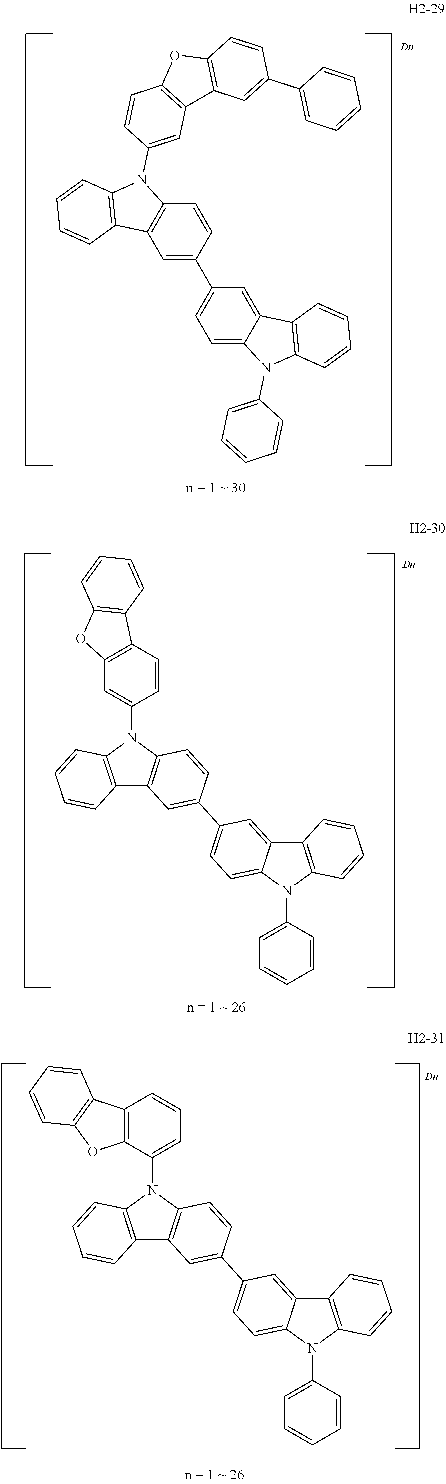

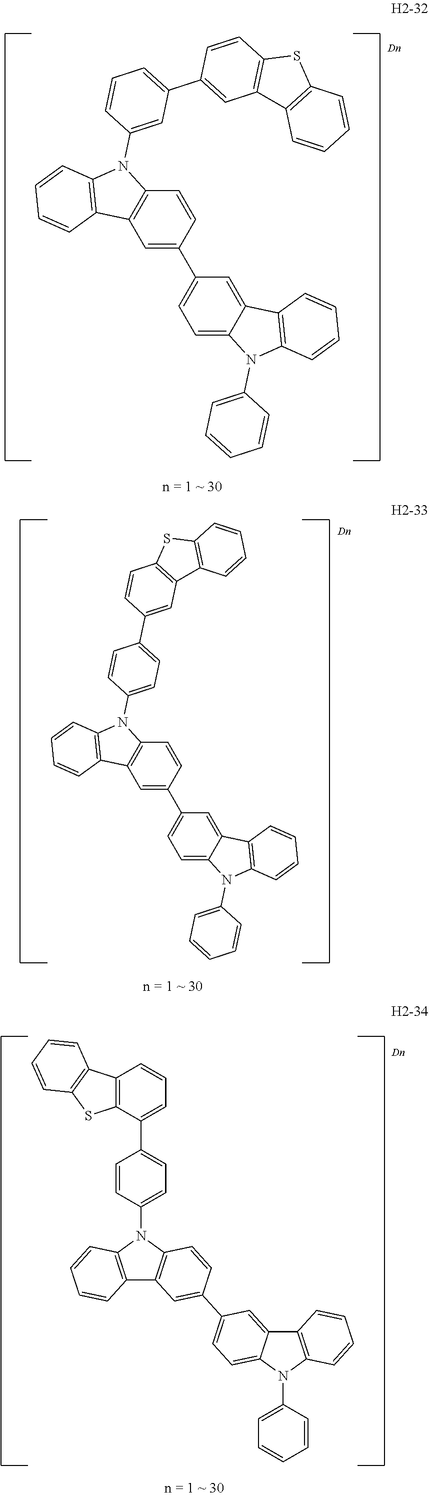

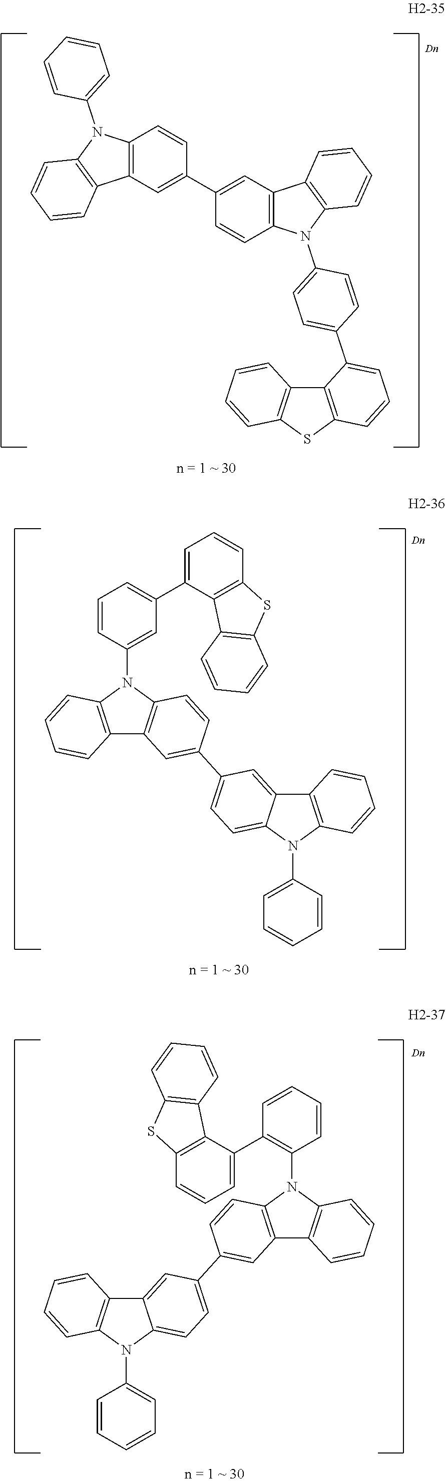

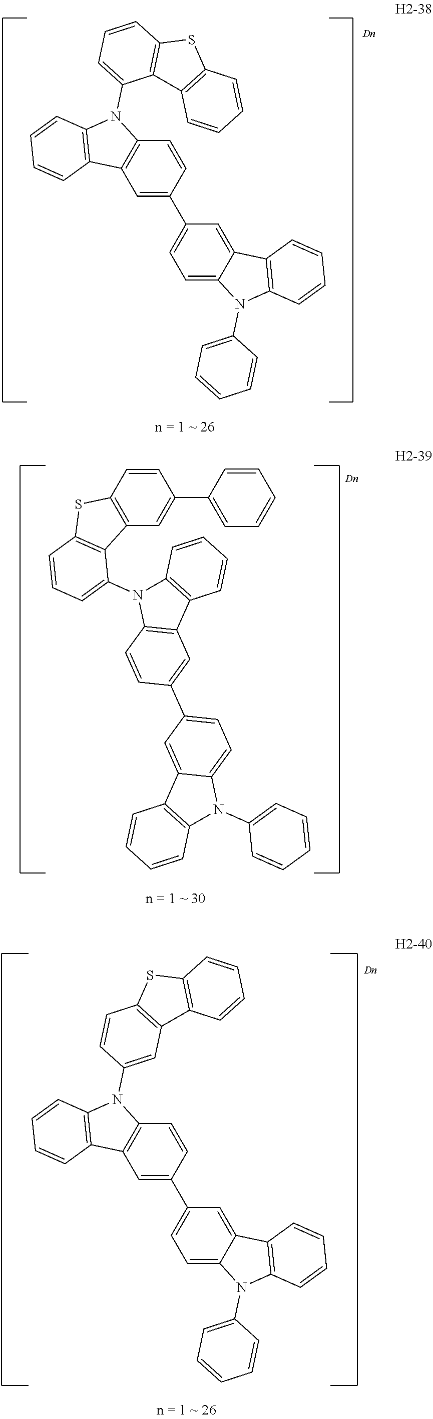

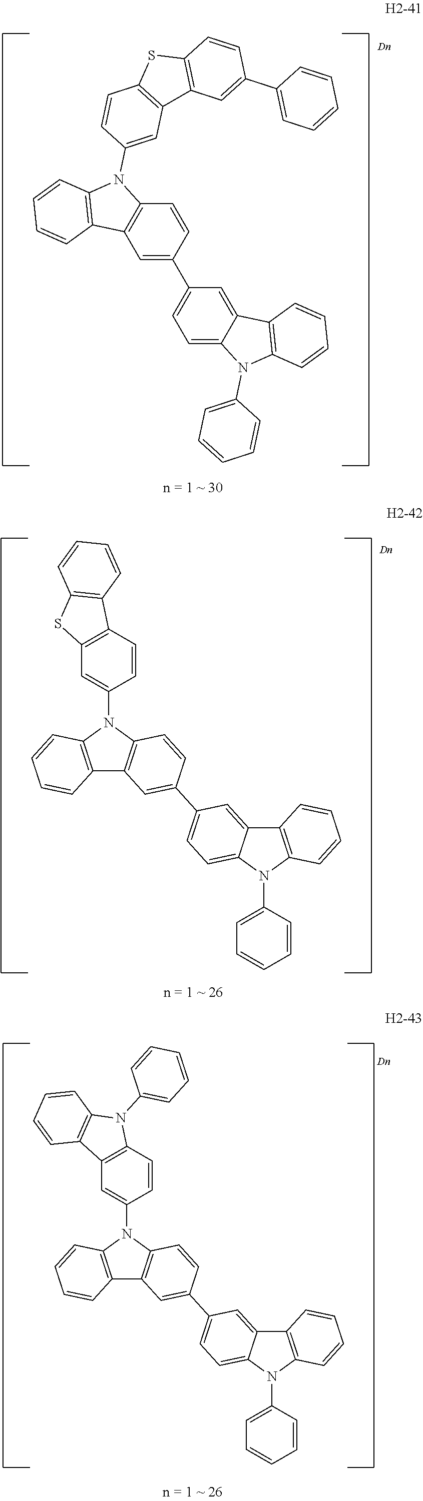

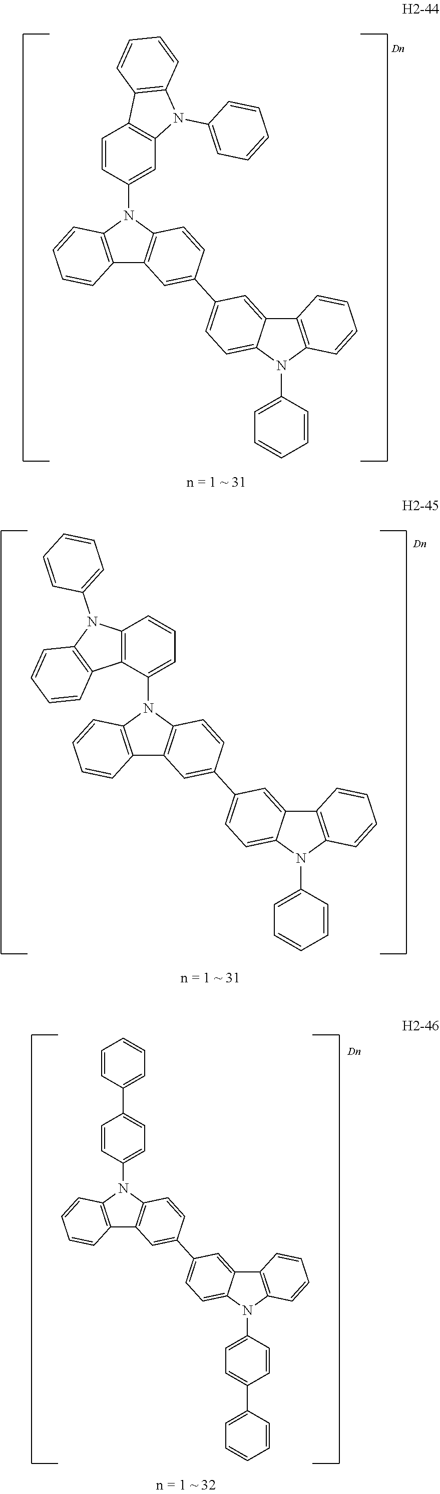

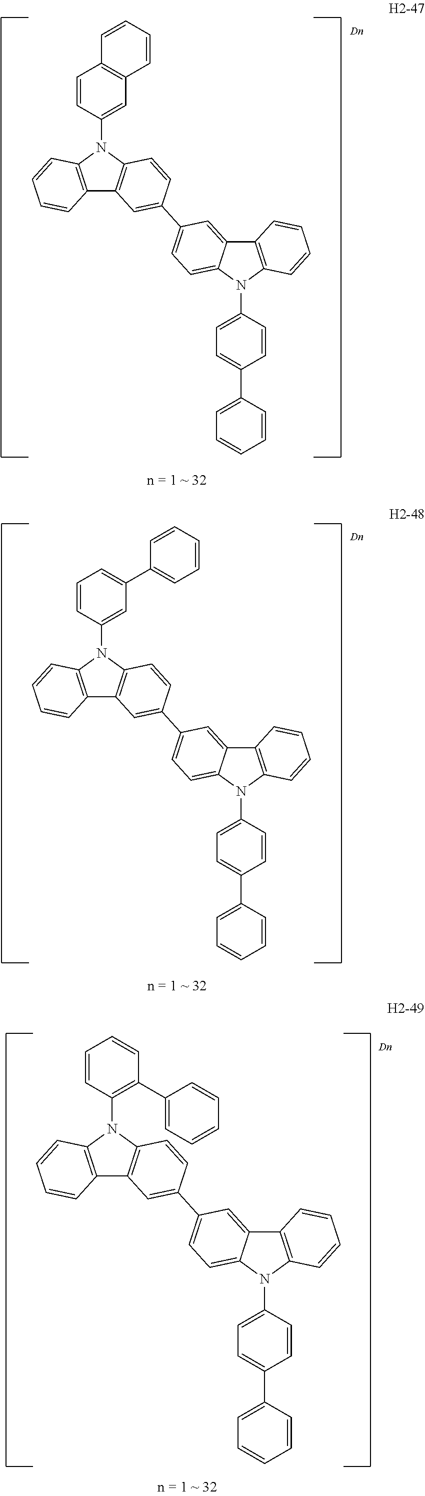

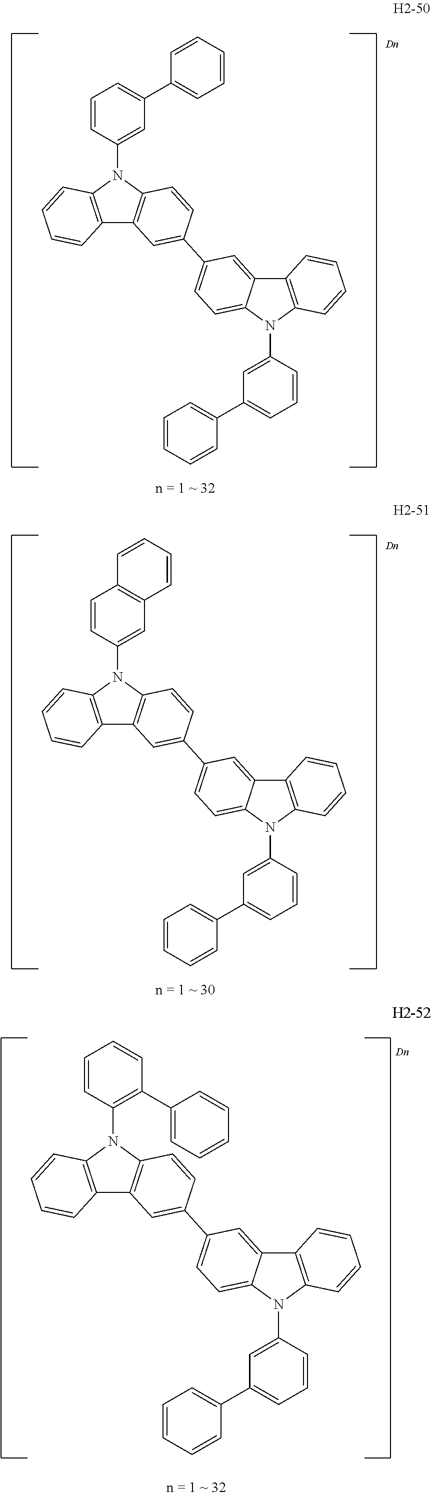

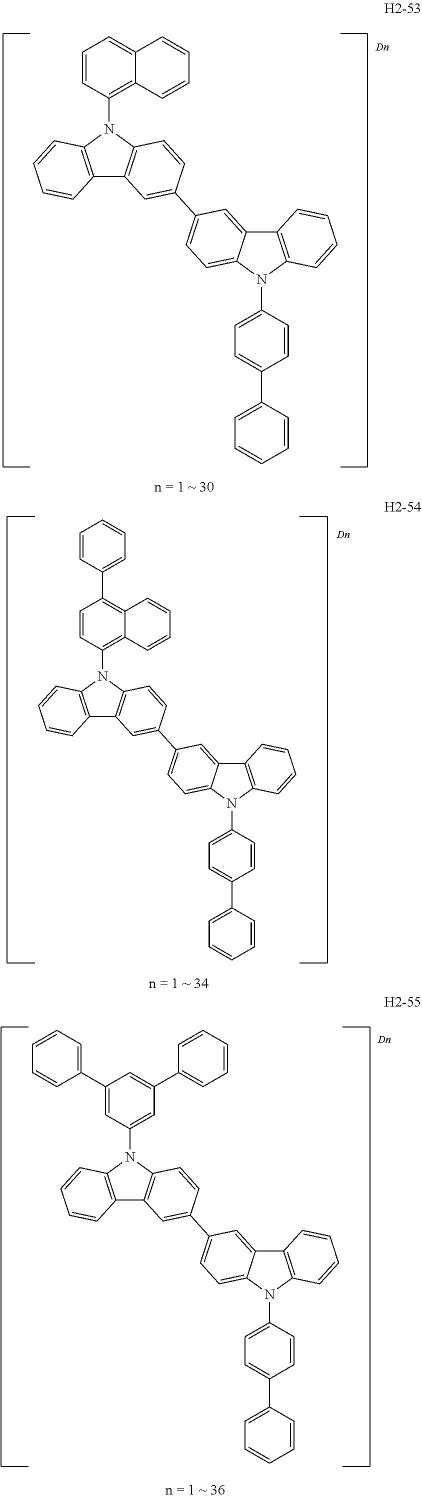

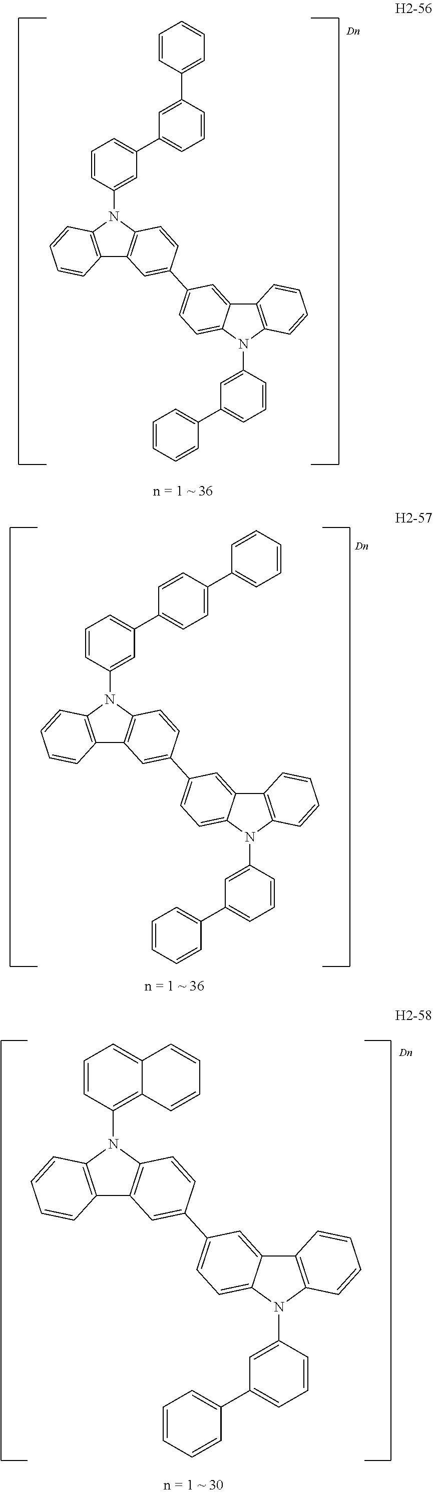

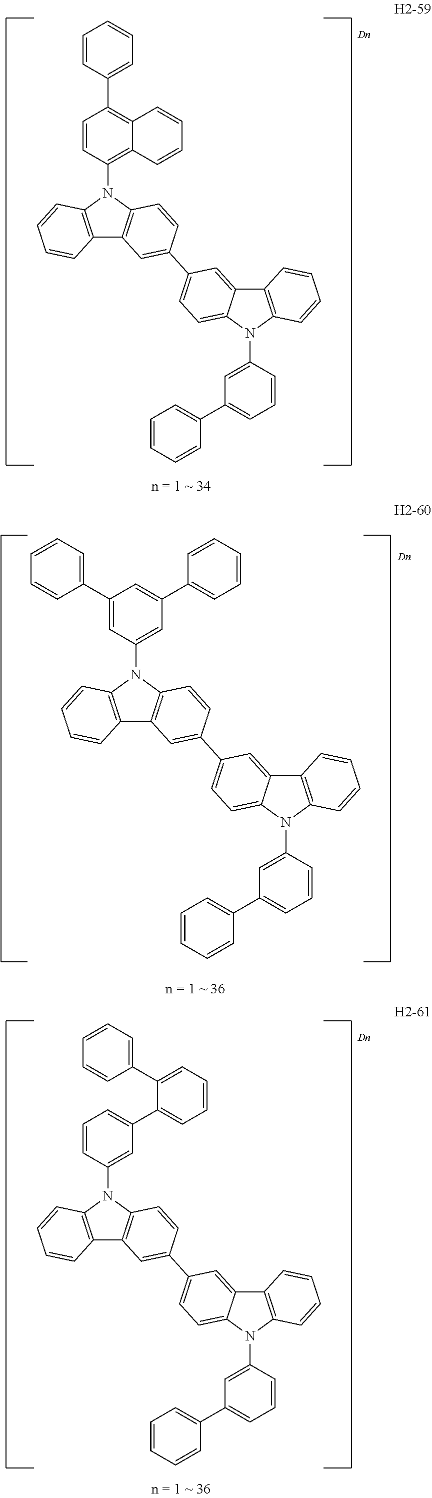

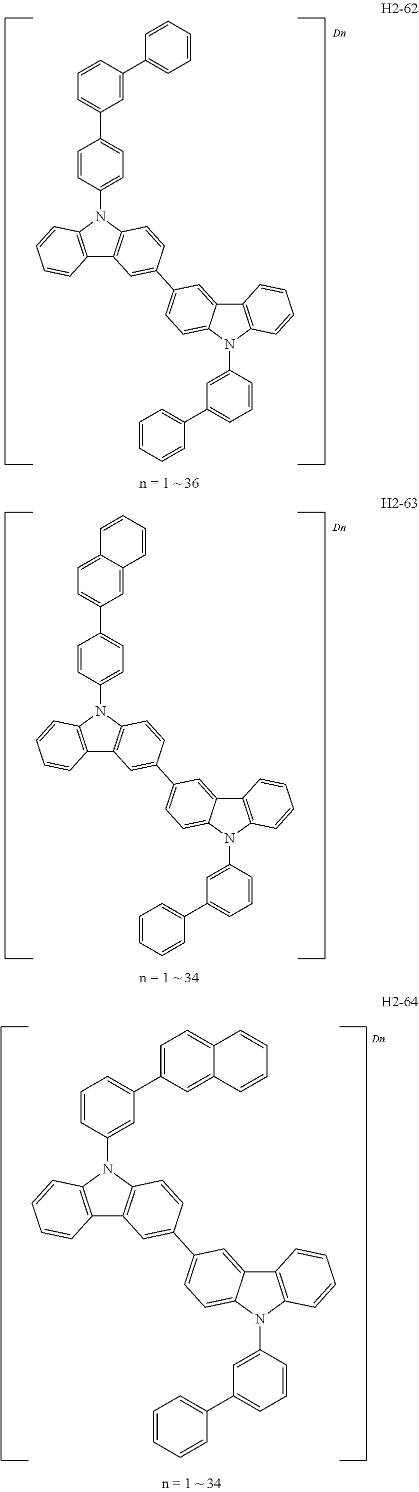

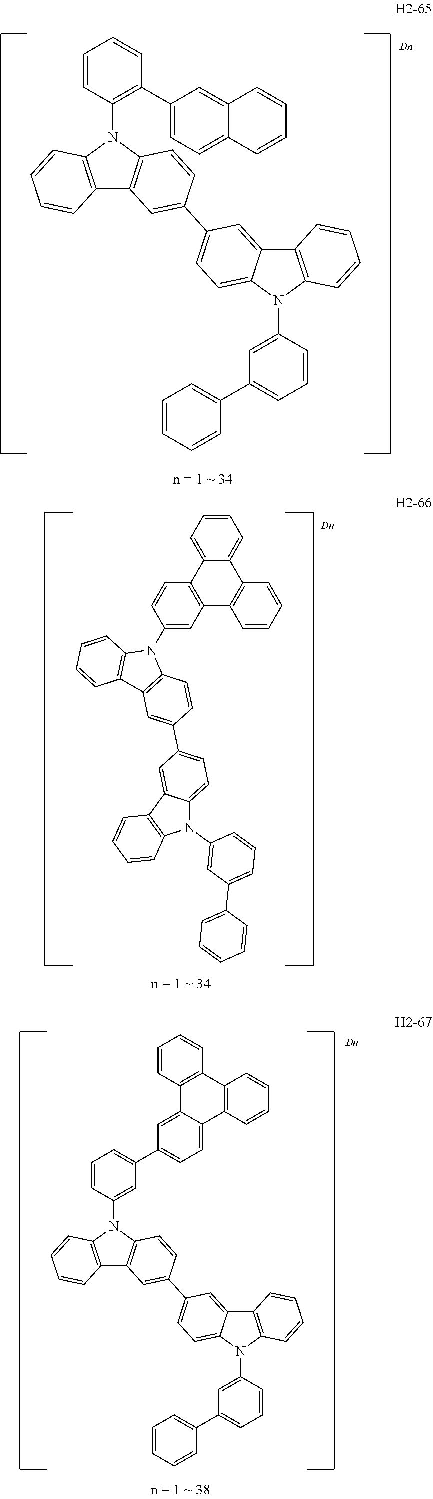

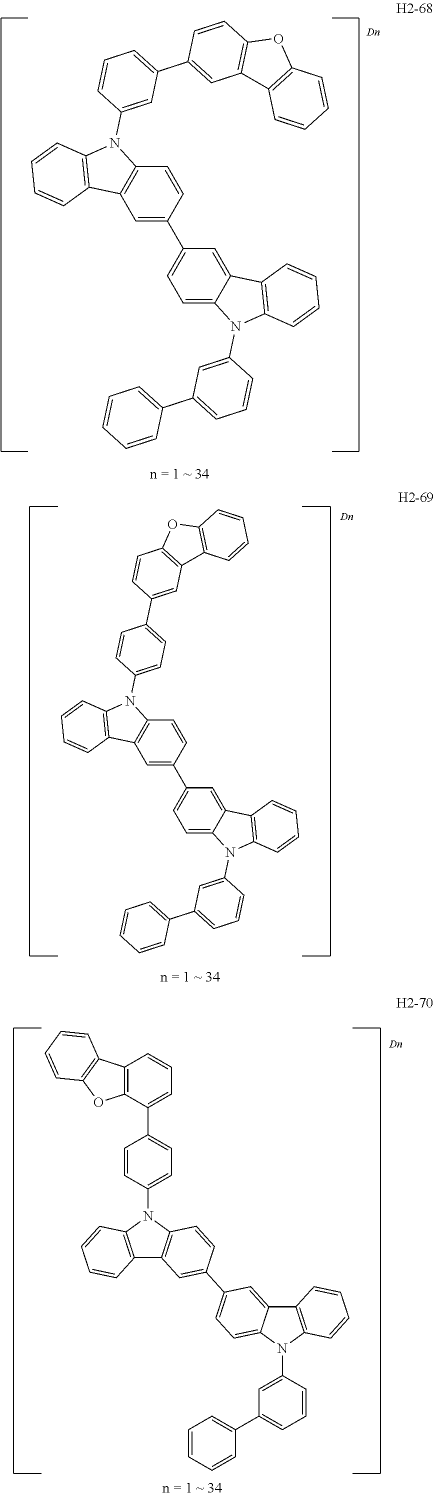

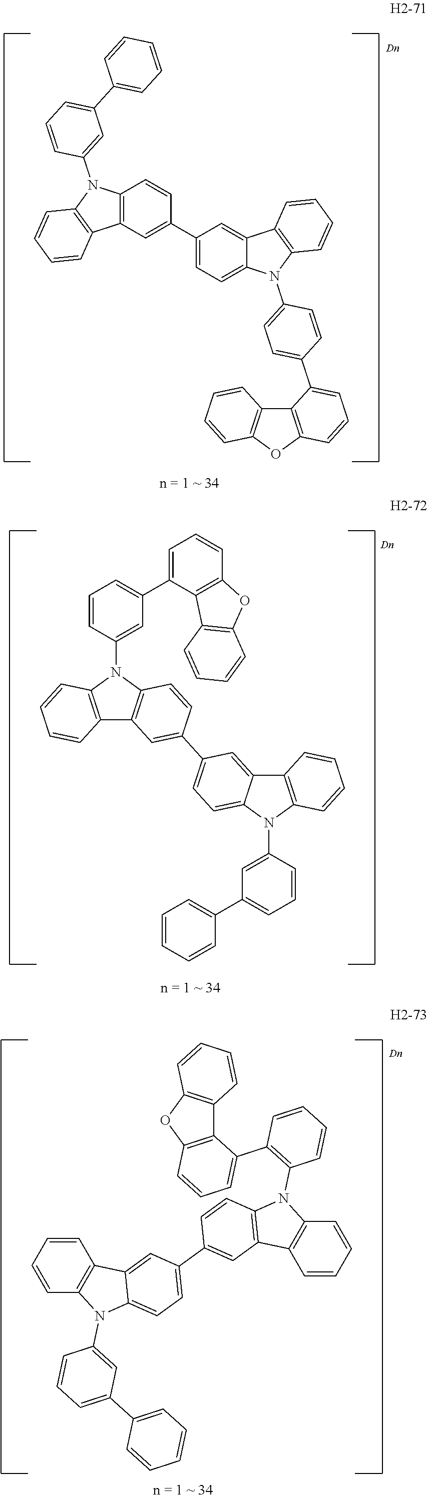

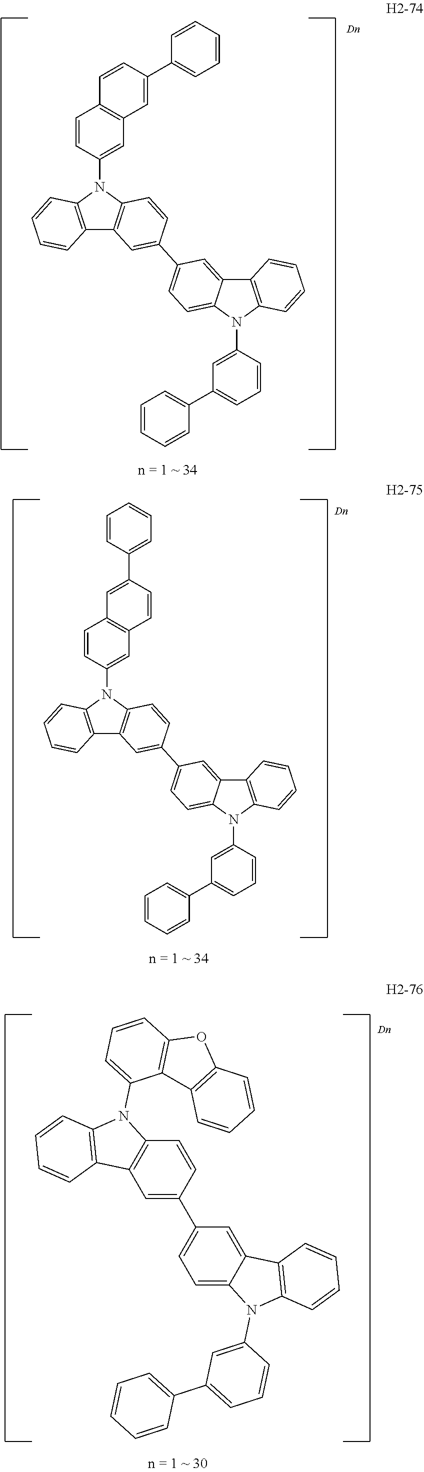

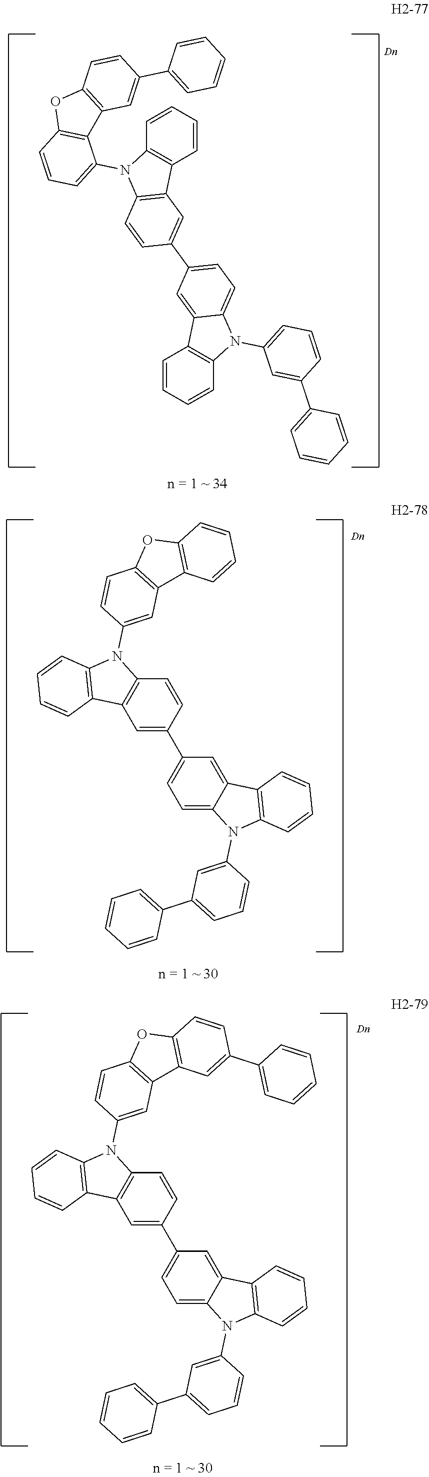

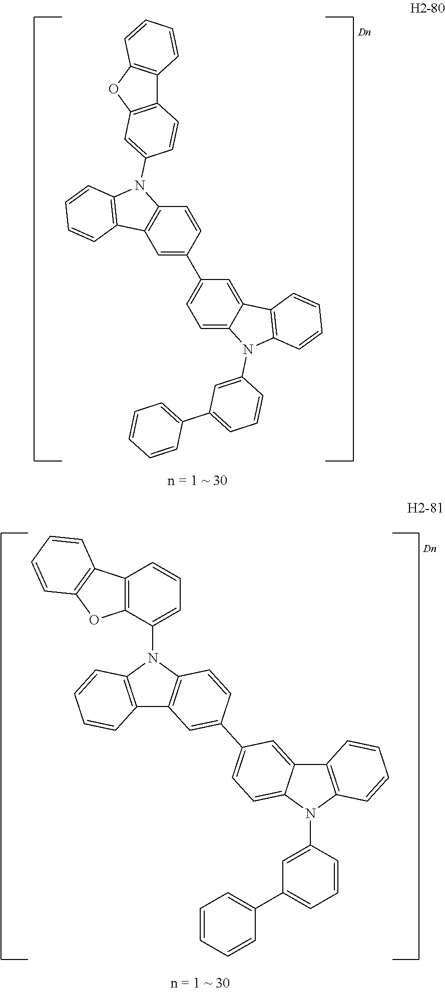

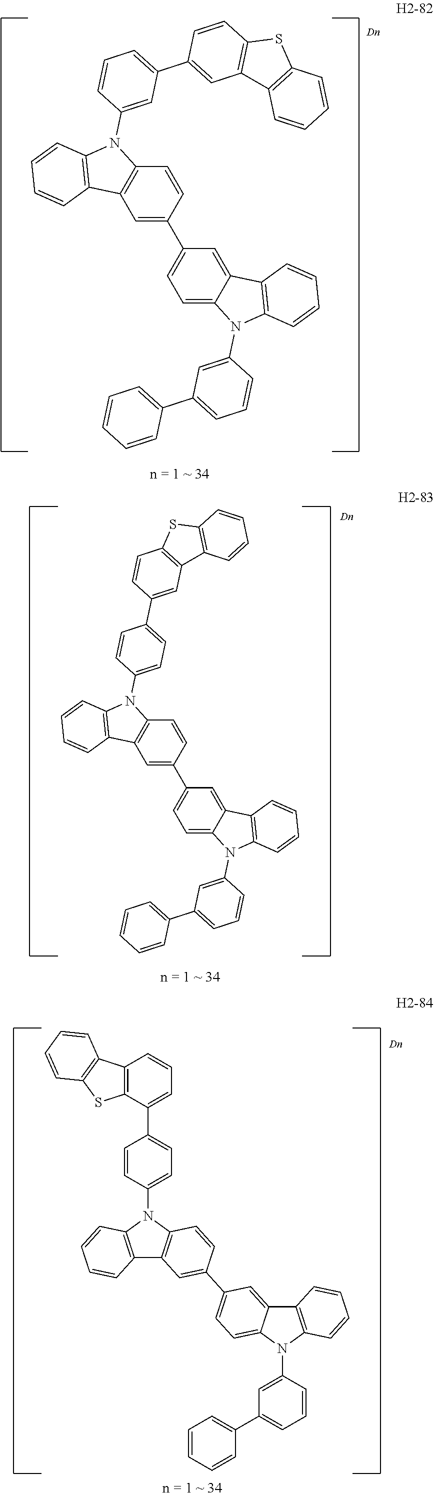

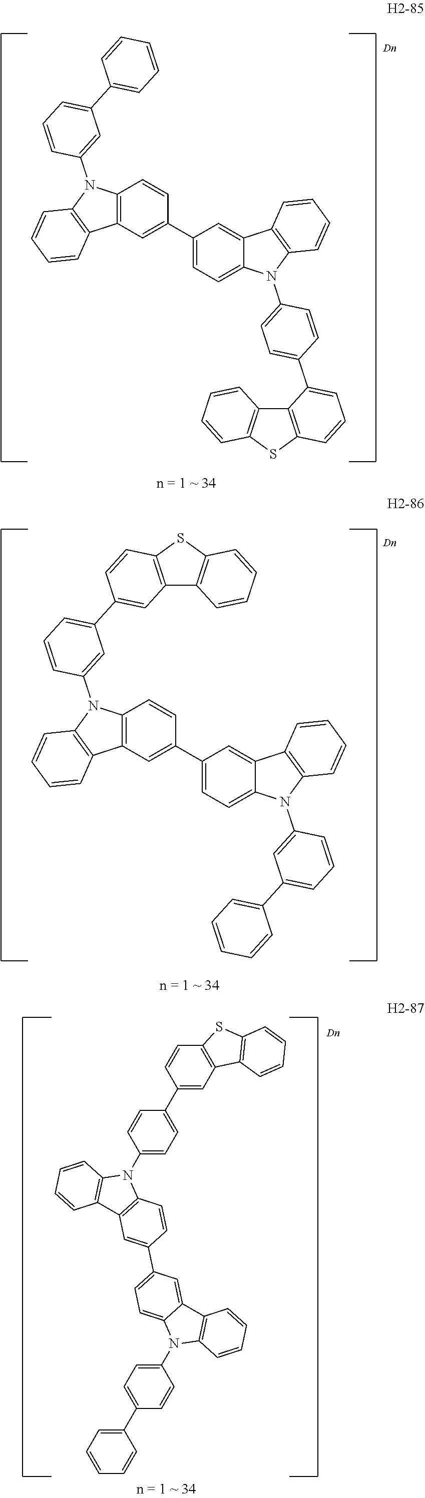

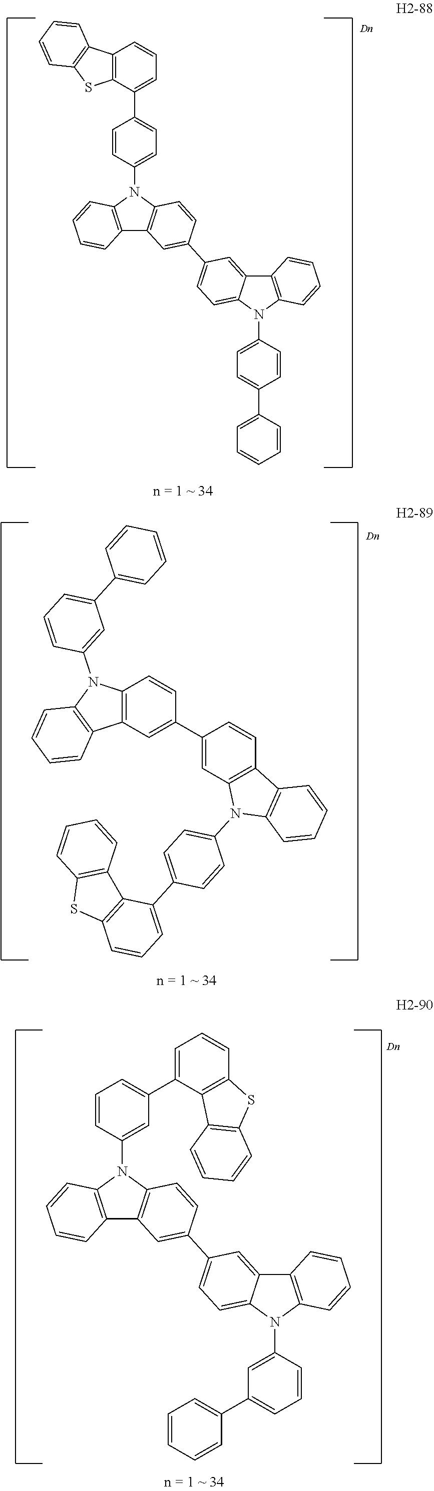

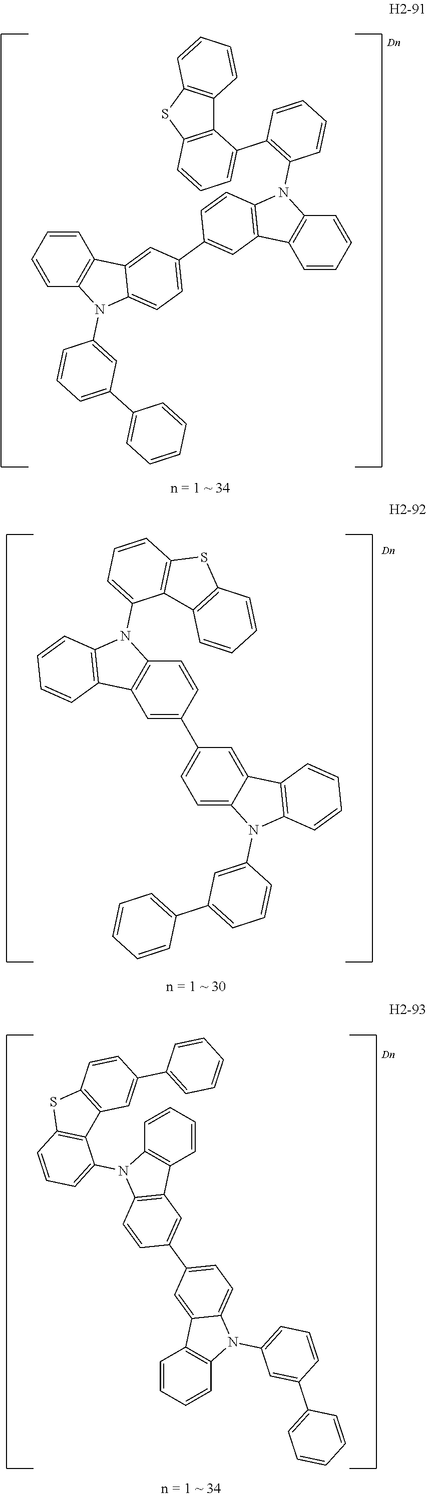

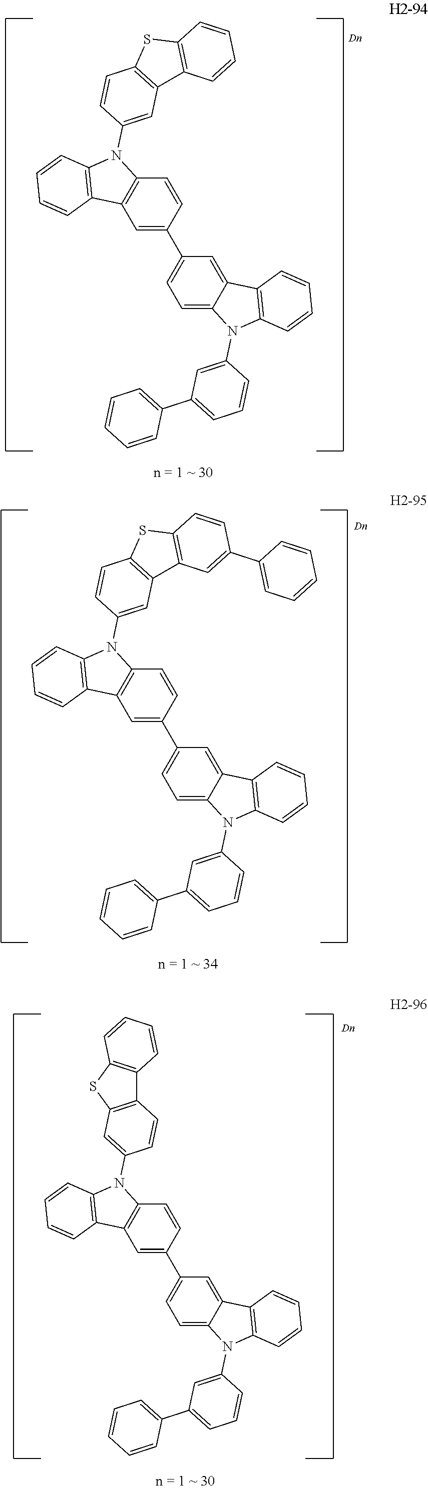

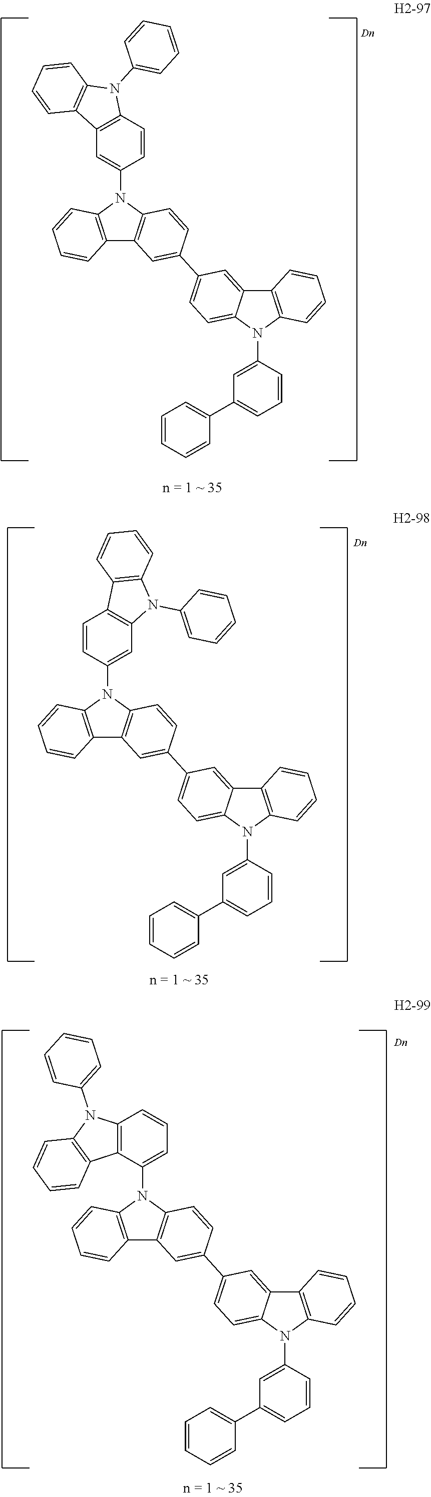

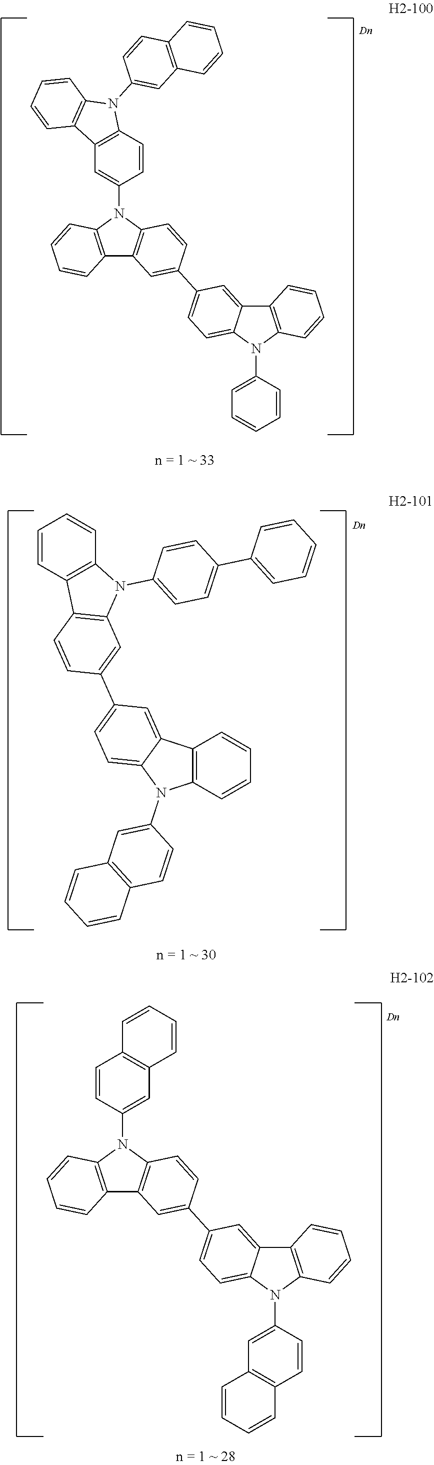

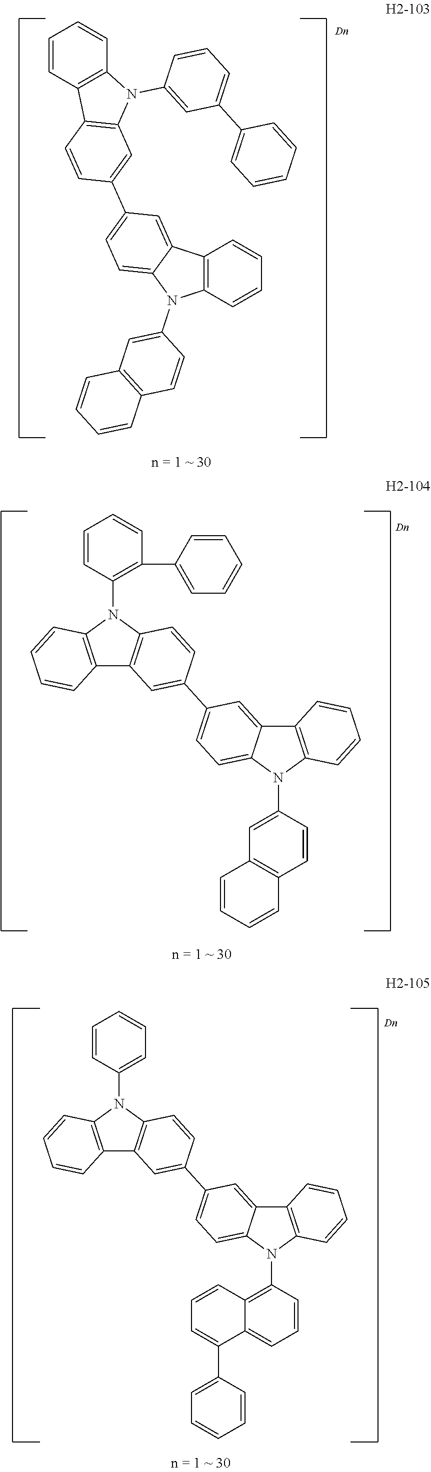

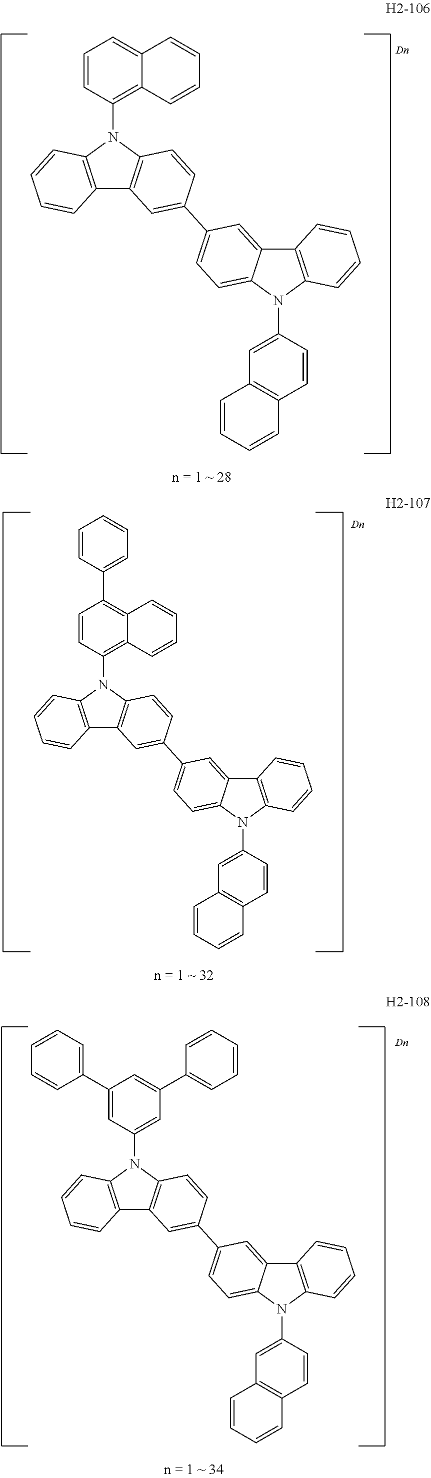

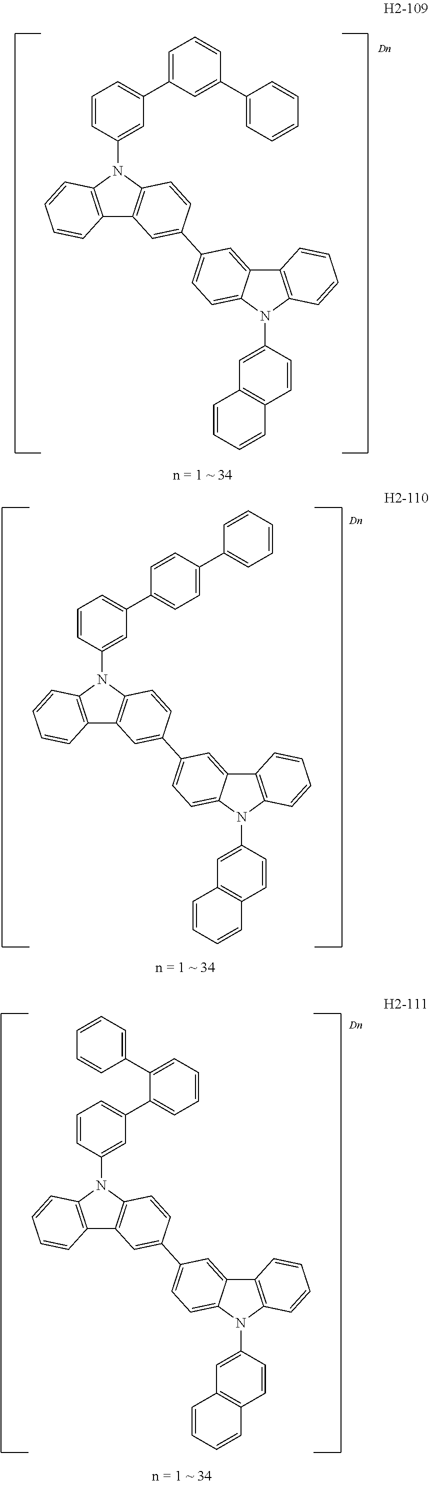

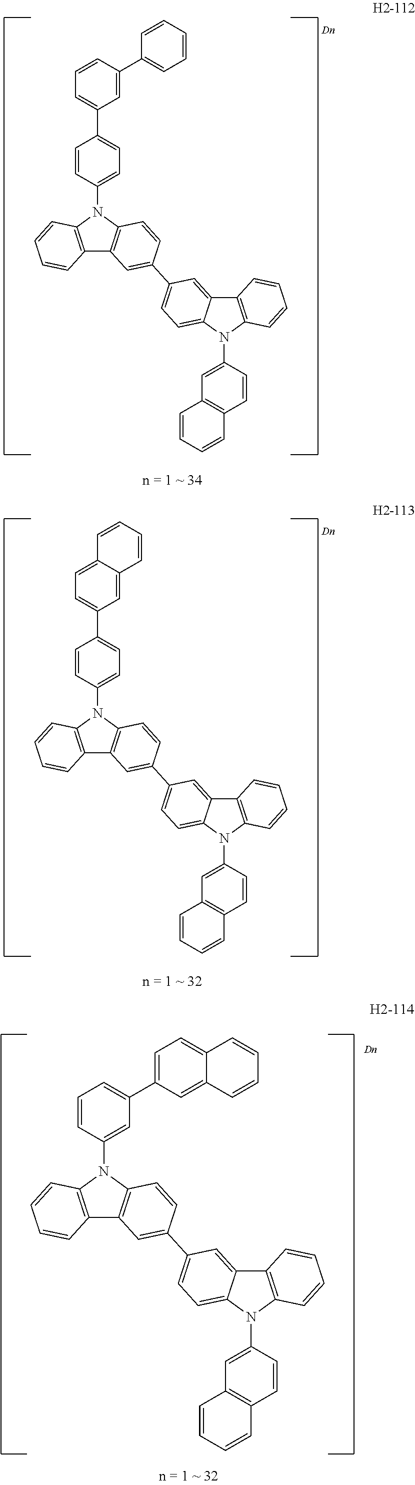

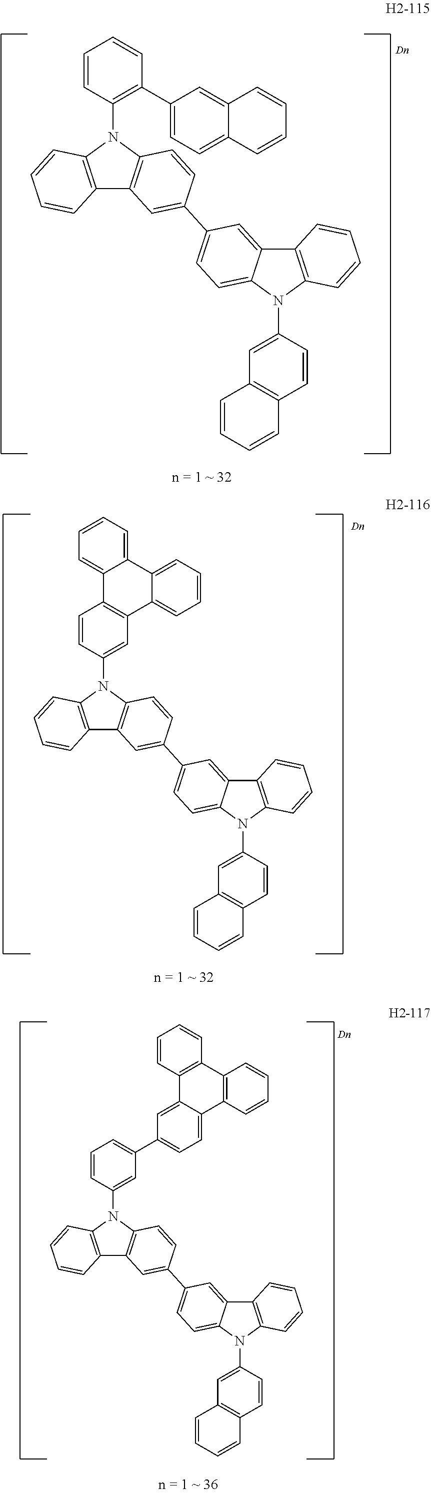

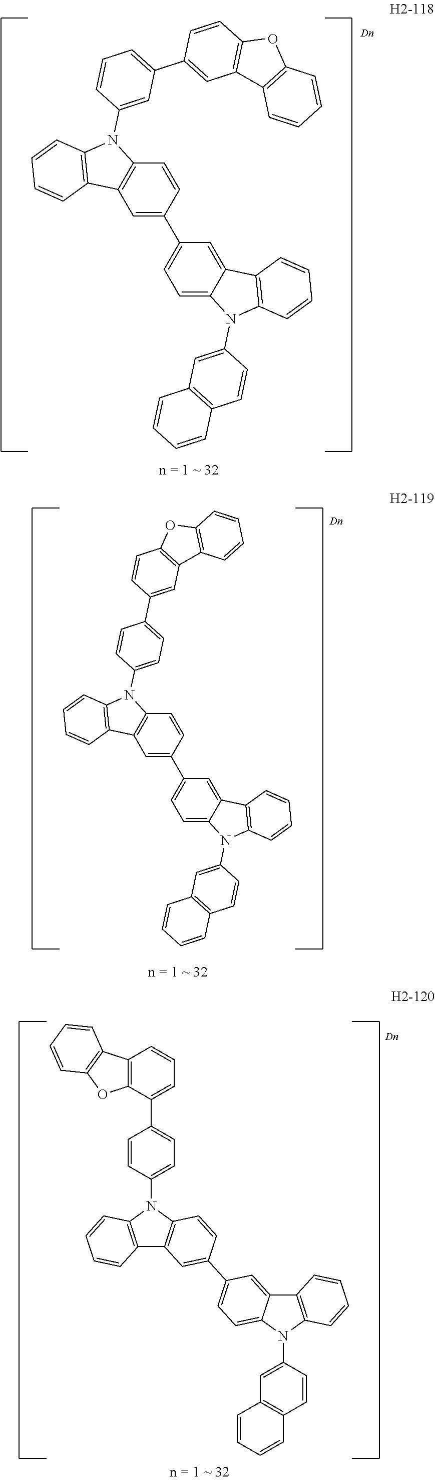

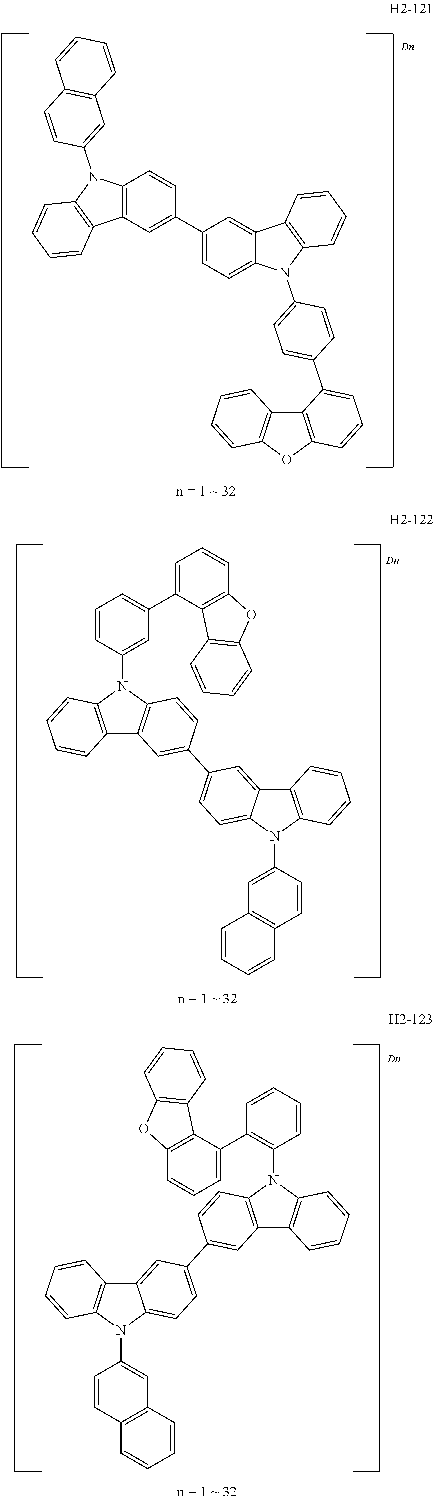

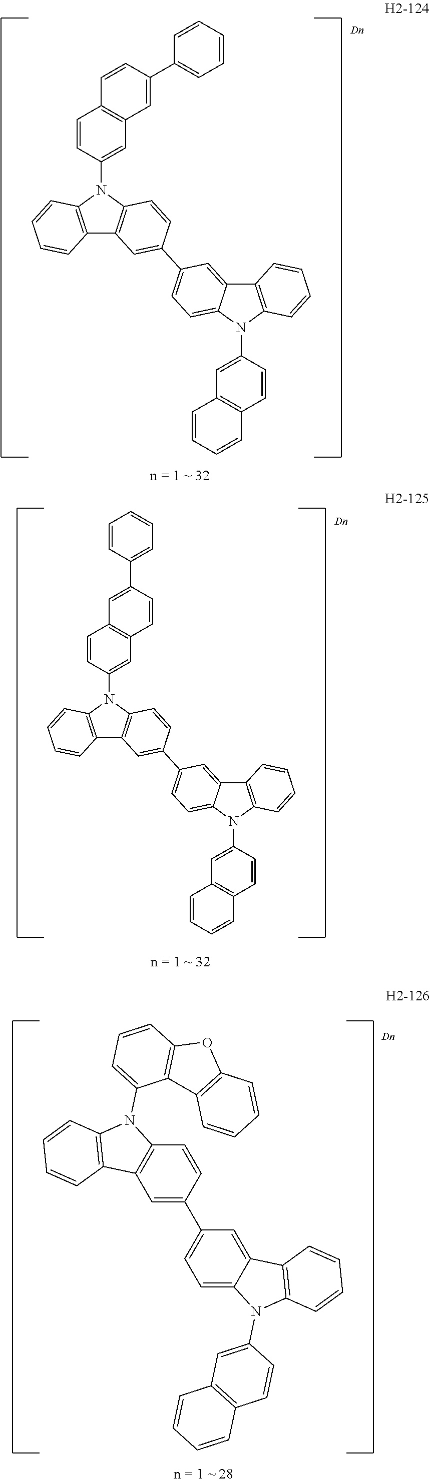

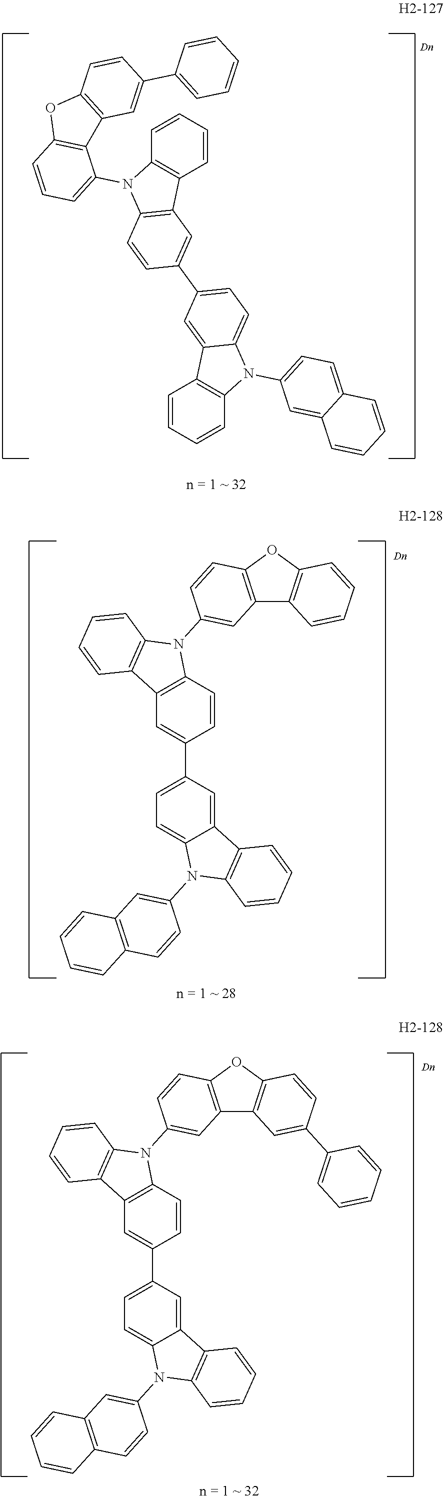

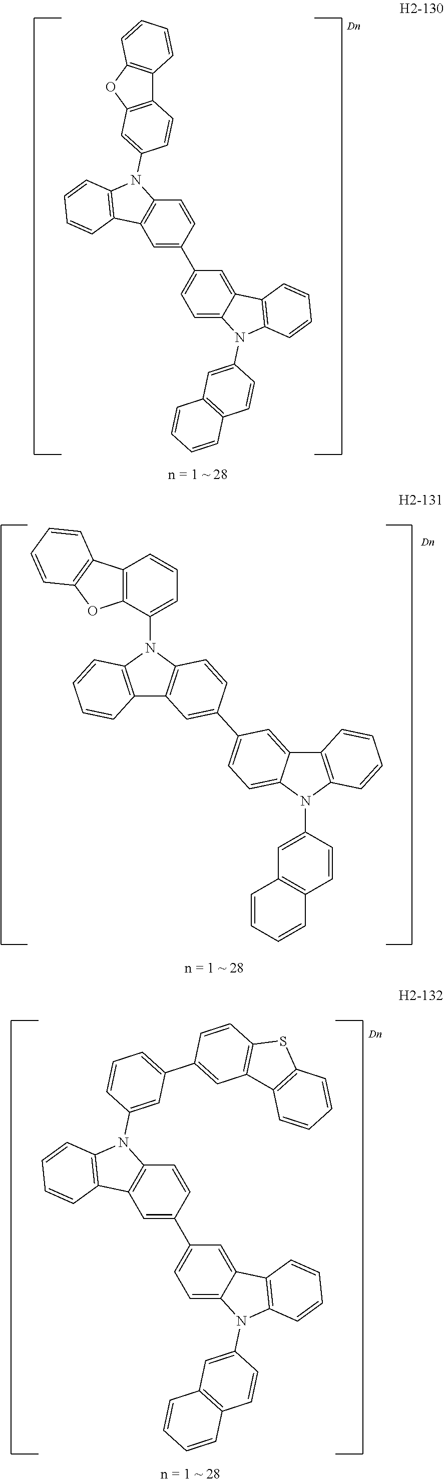

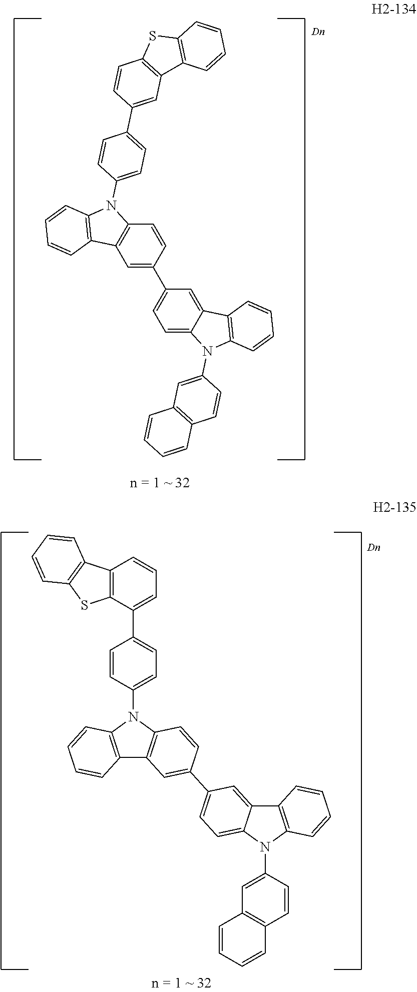

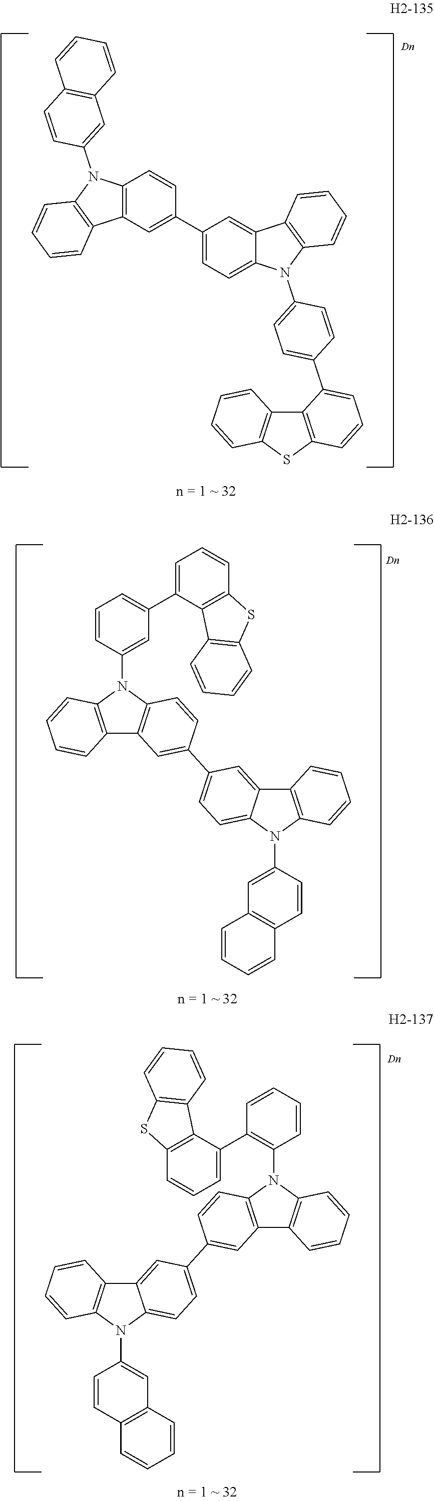

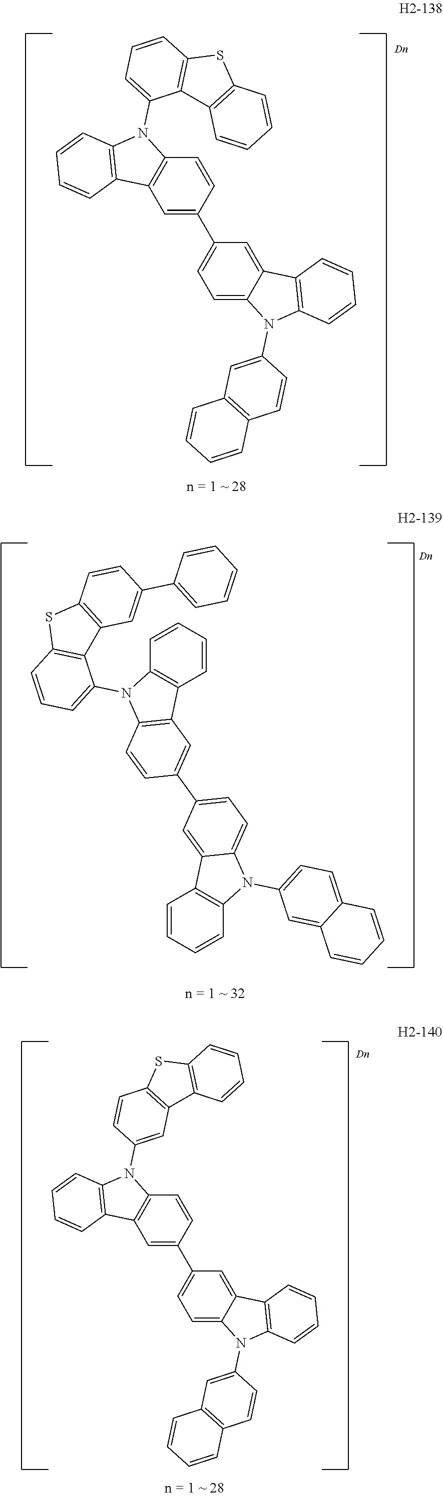

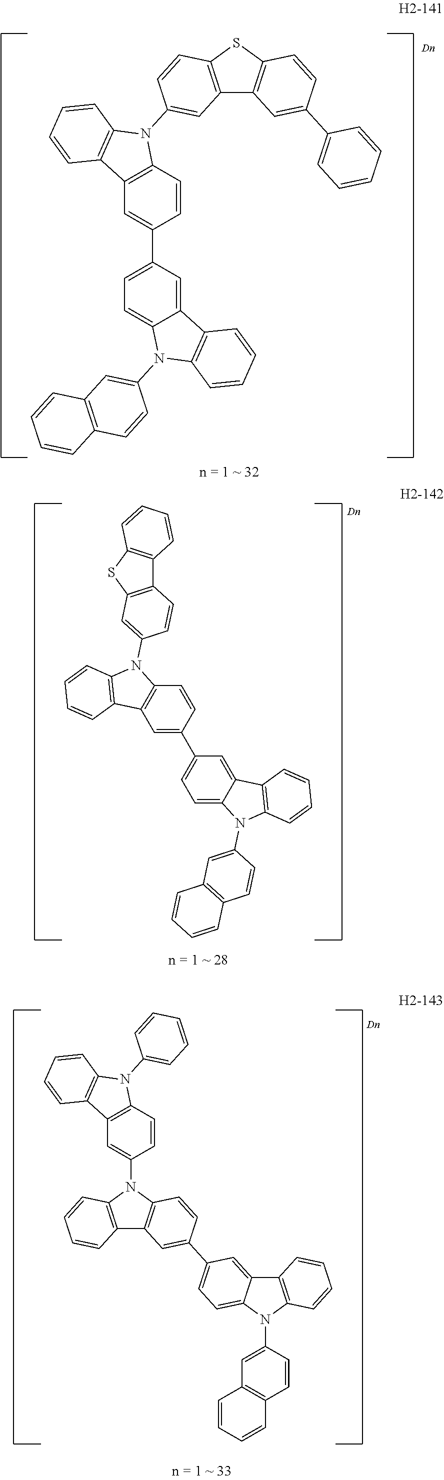

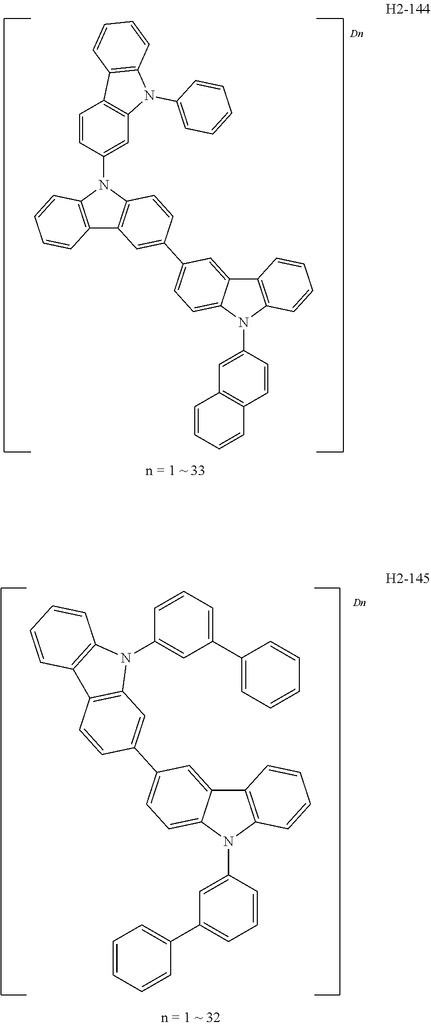

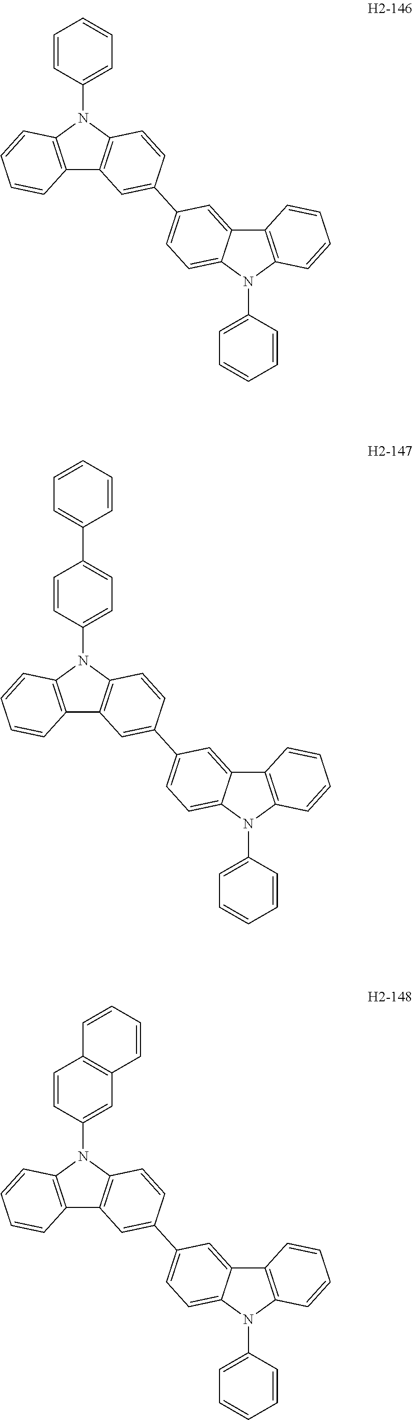 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1]

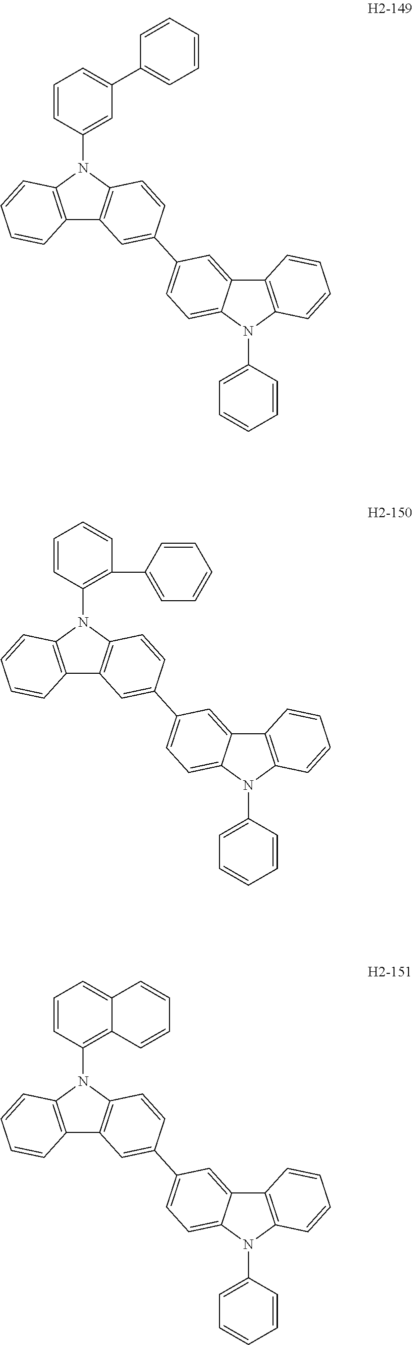 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1]

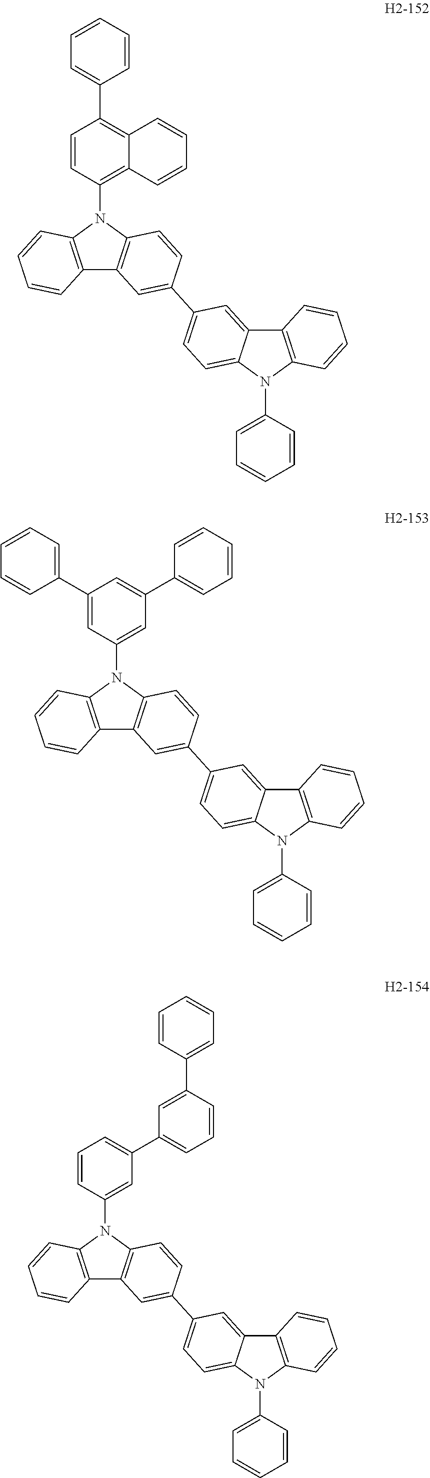 [SMILES: c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1]

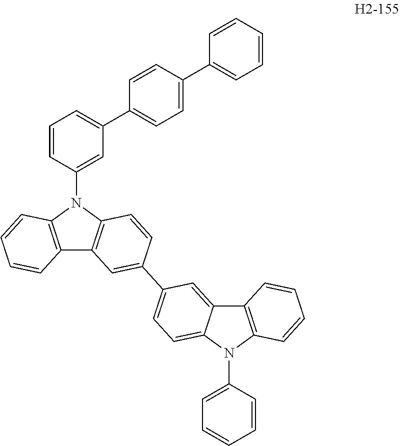 [SMILES: c1ccc(-c2ccc(-c3cccc(-n4c5ccccc5c5cc(-c6ccc7c(c6)c6ccccc6n7-c6ccccc6)ccc54)c3)cc2)cc1]

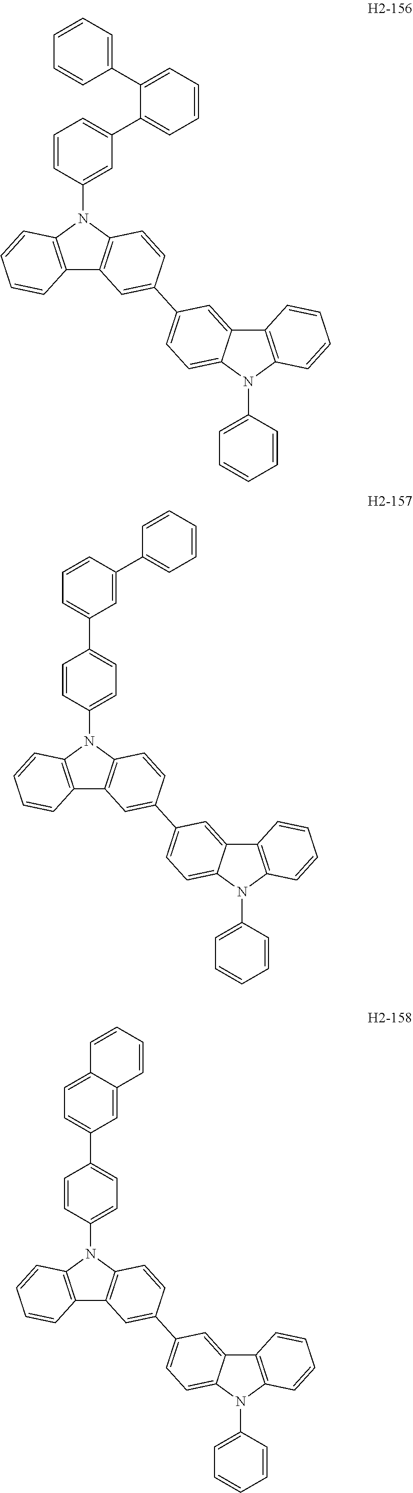 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5ccc6ccccc6c5)cc4)ccc32)cc1]

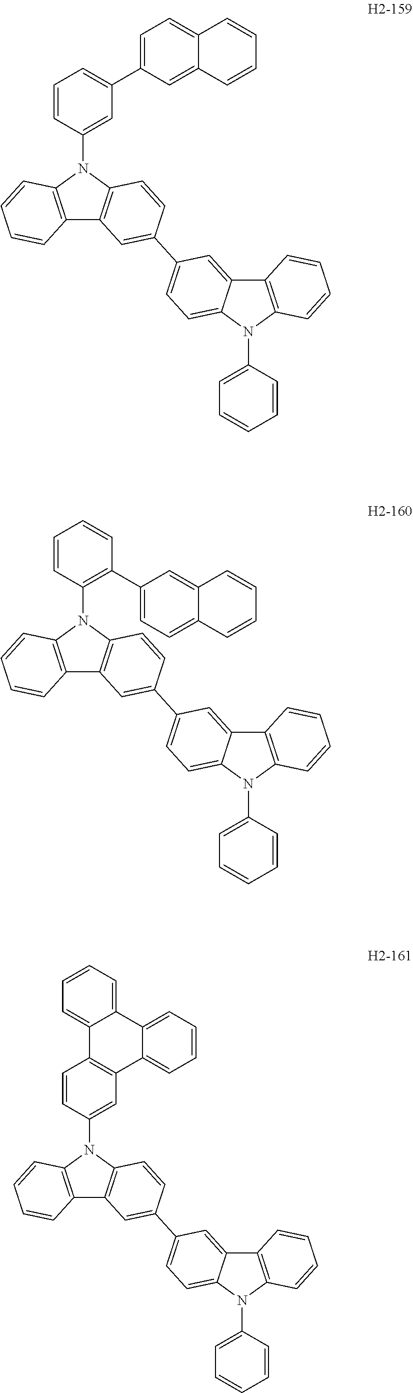 [SMILES: c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

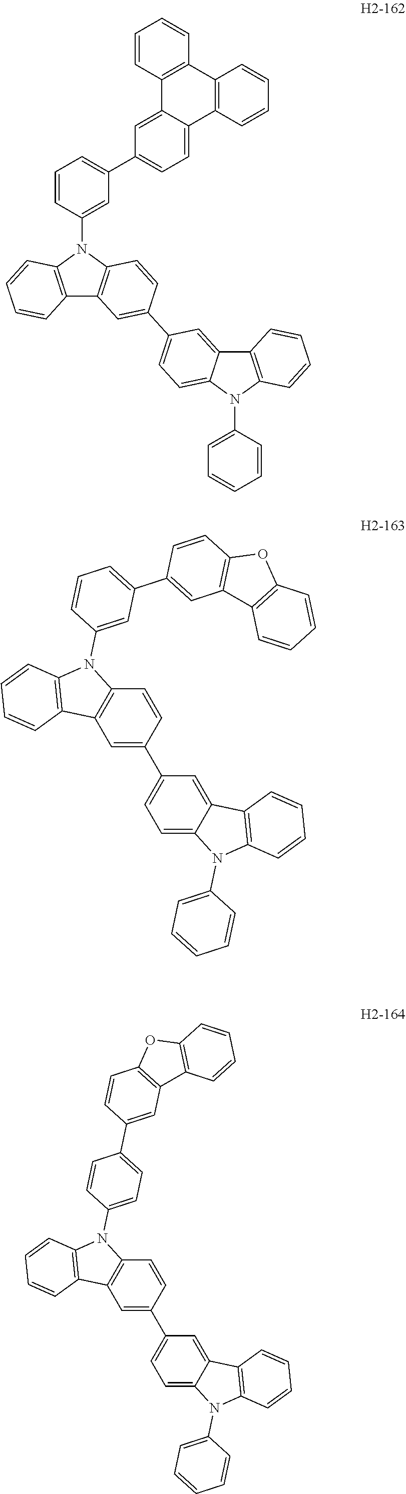 [SMILES: c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1]

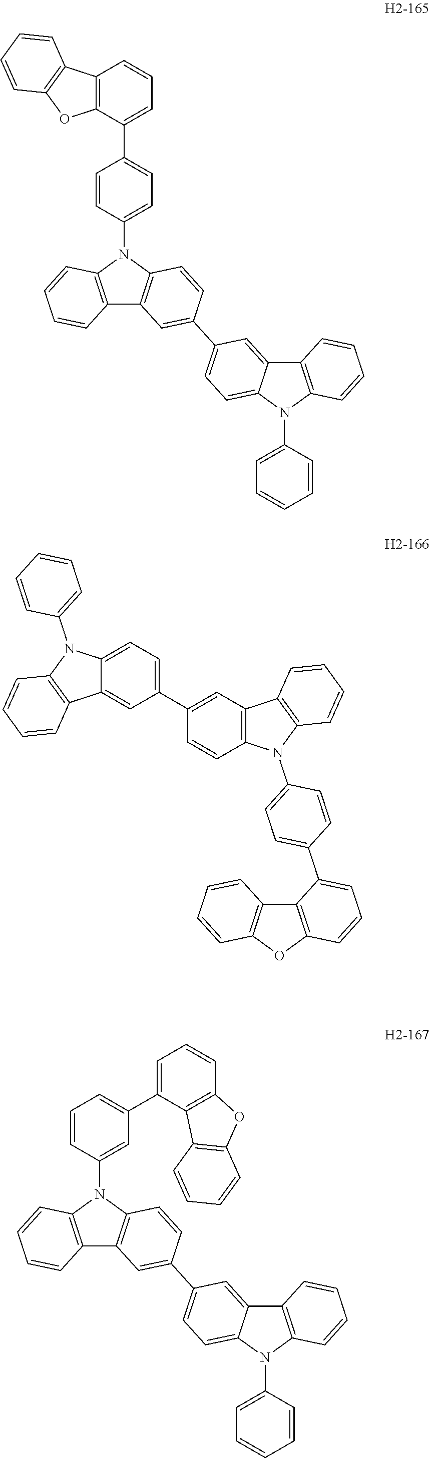 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1]

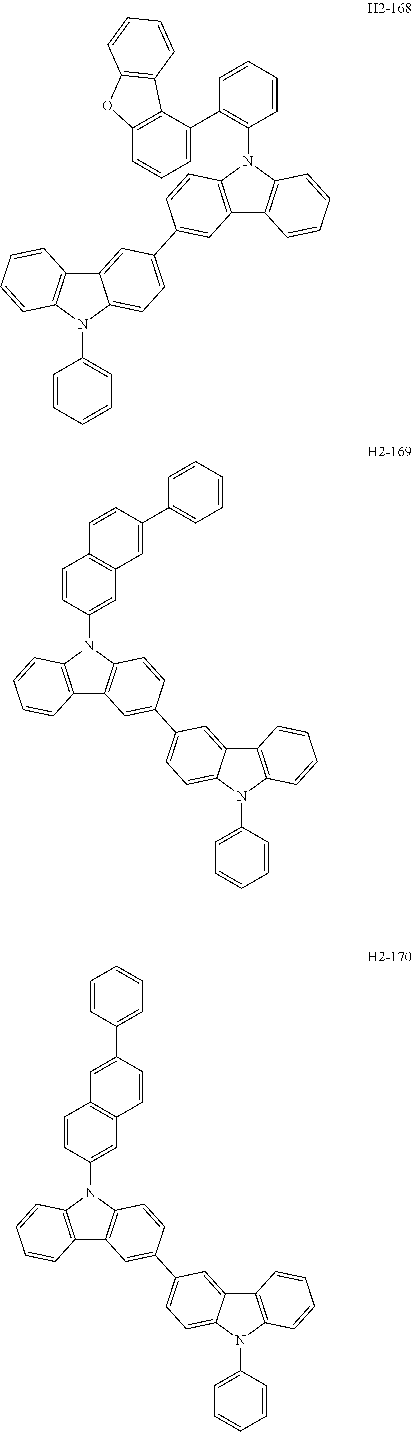 [SMILES: c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1 ; c1ccc(-c2ccc3cc(-n4c5ccccc5c5cc(-c6ccc7c(c6)c6ccccc6n7-c6ccccc6)ccc54)ccc3c2)cc1]

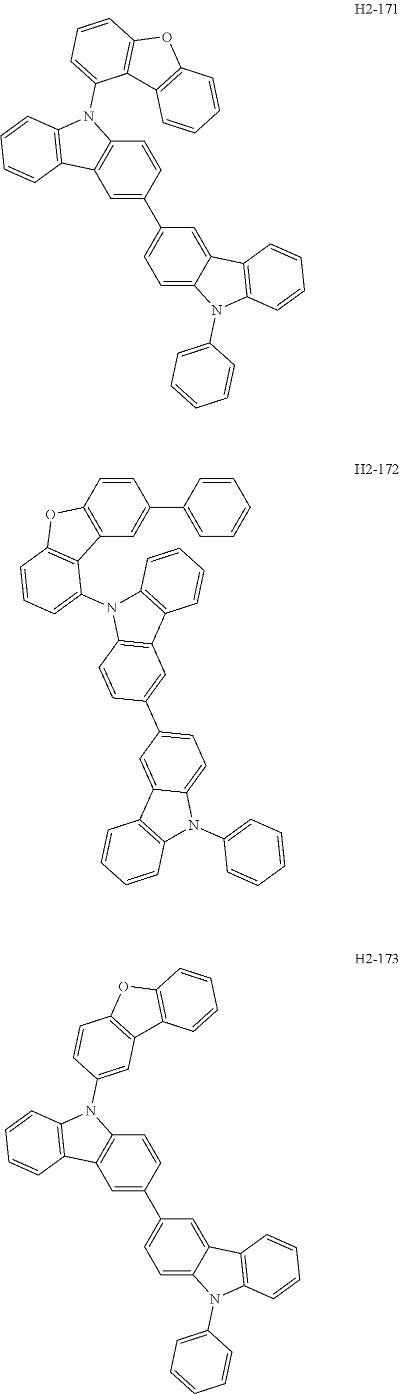 [SMILES: c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5oc6ccccc6c5c4)ccc32)cc1]

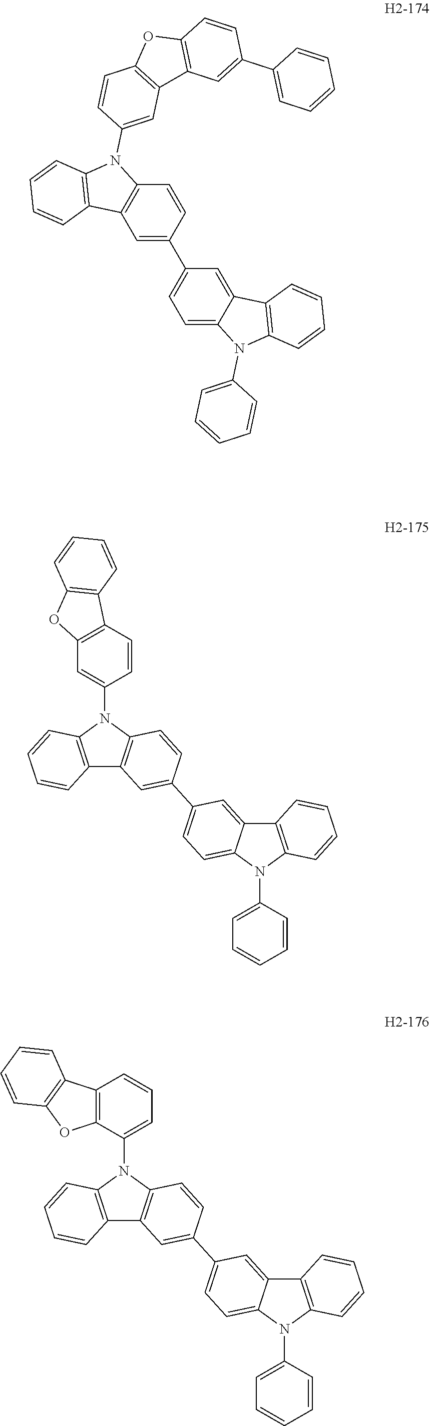 [SMILES: c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5c4oc4ccccc45)ccc32)cc1]

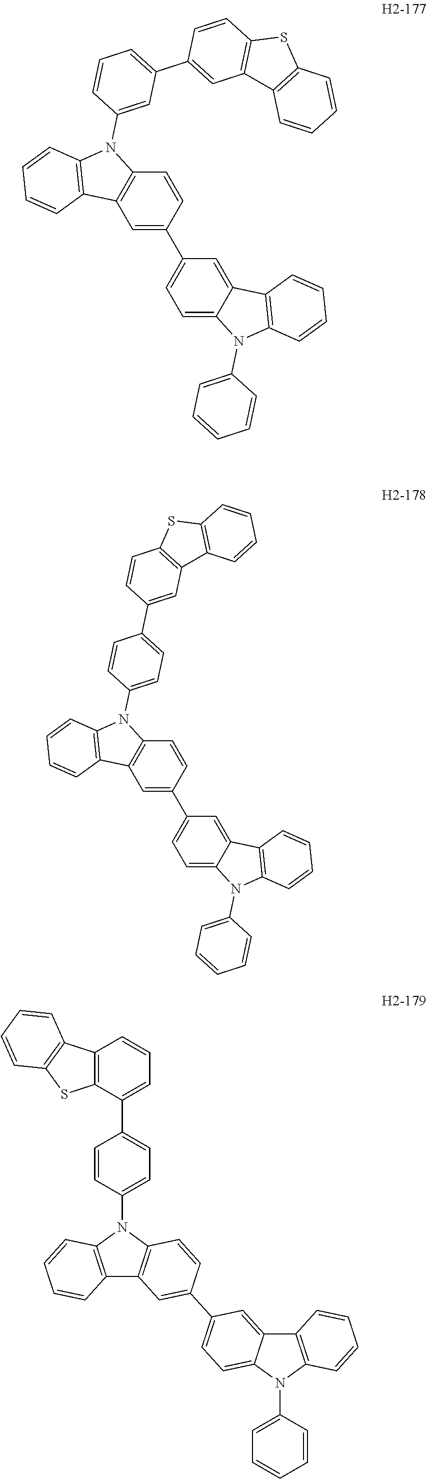 [SMILES: c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

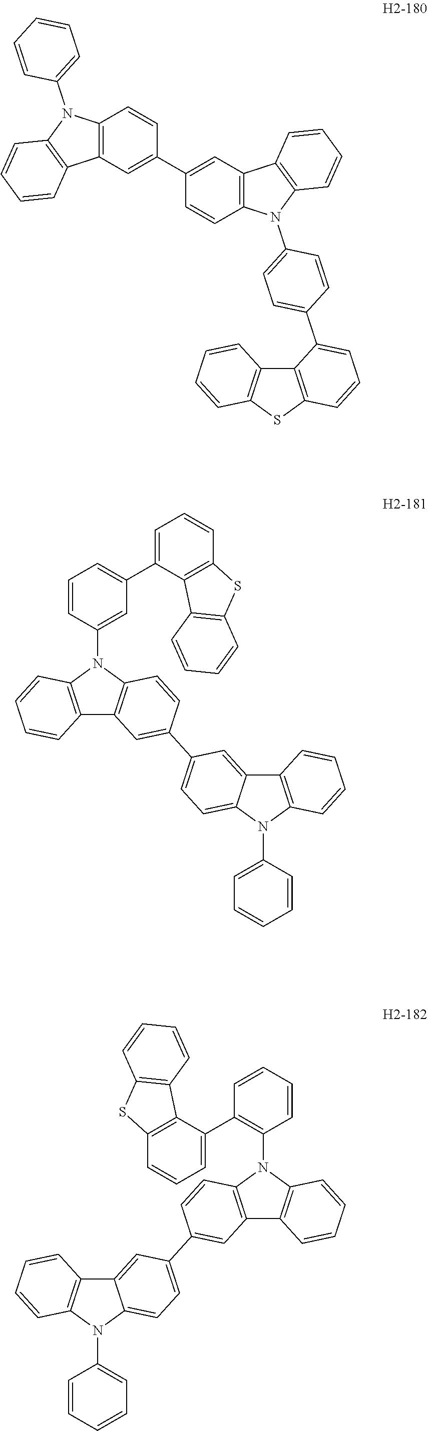 [SMILES: c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1]

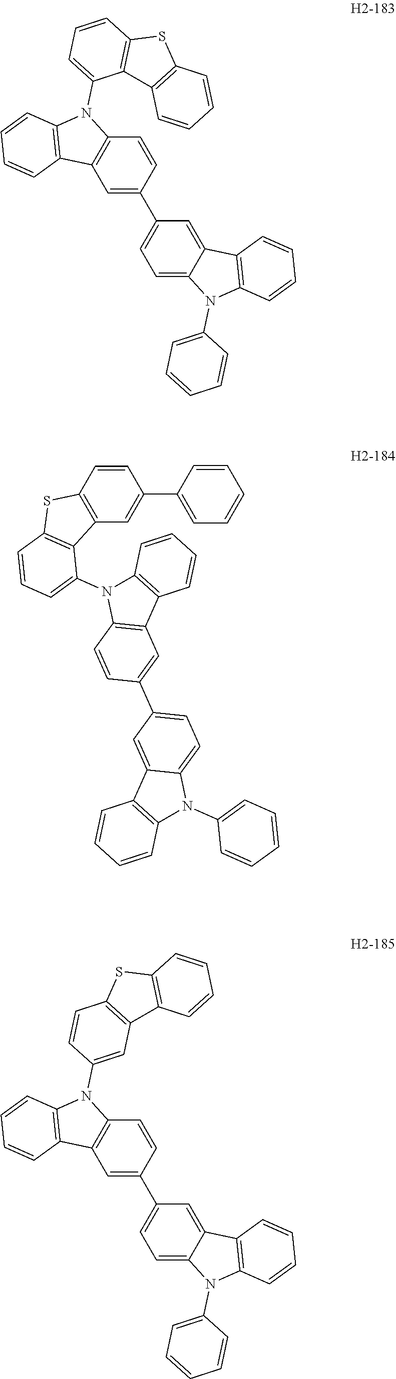 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc3sc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5sc6ccccc6c5c4)ccc32)cc1]

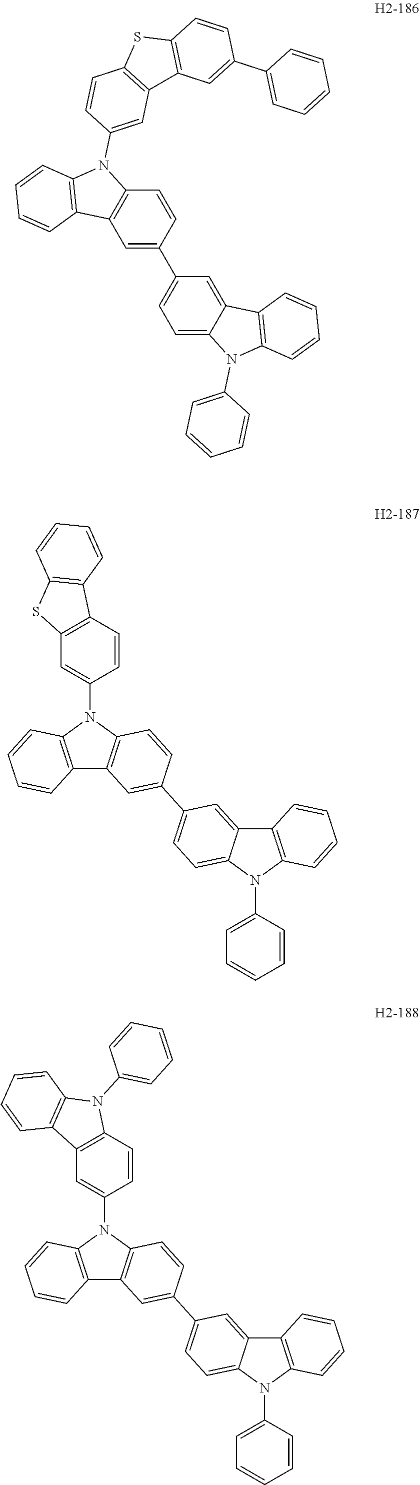 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

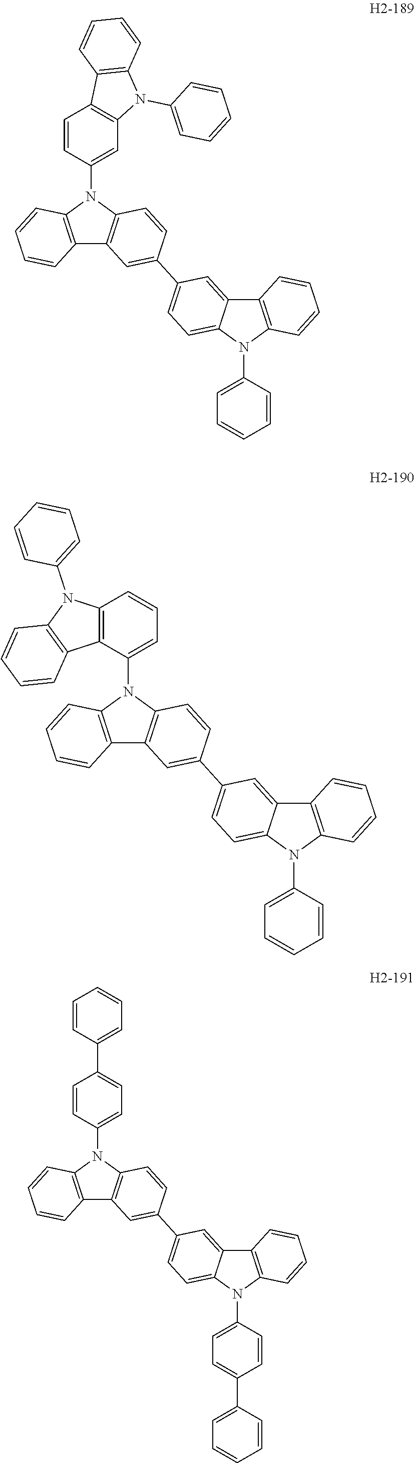 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

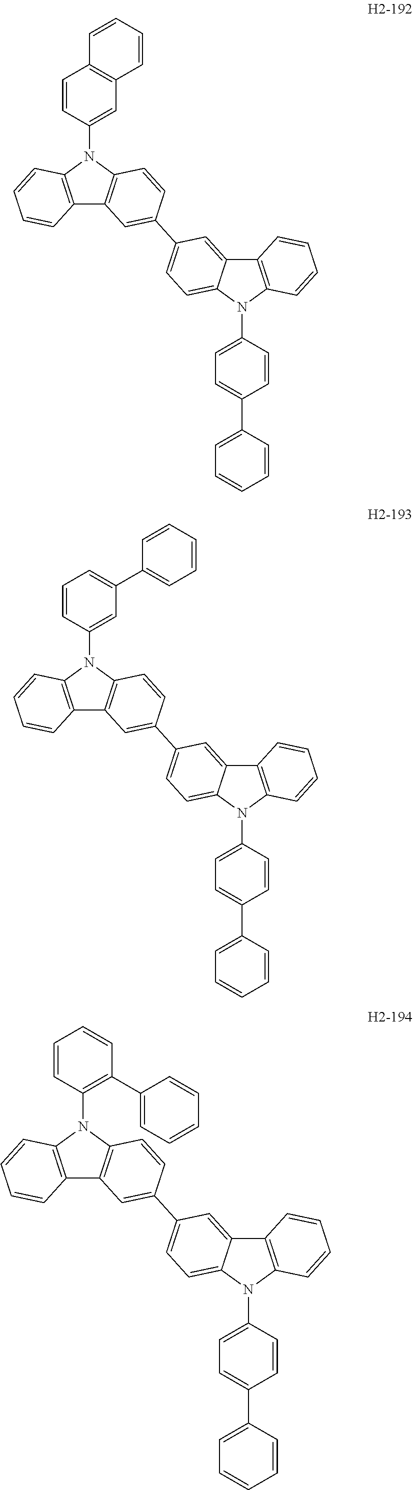 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1]

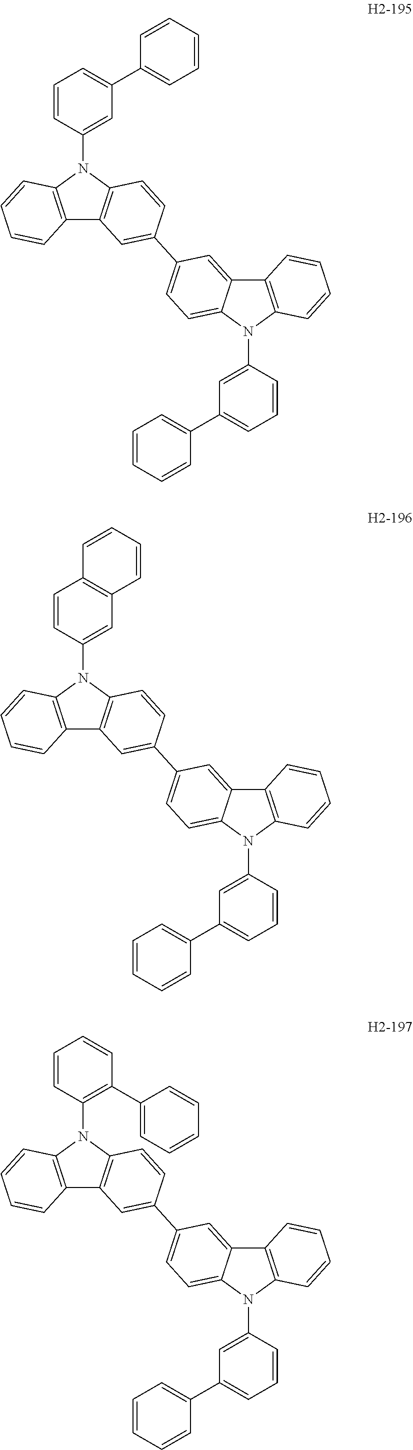 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1]

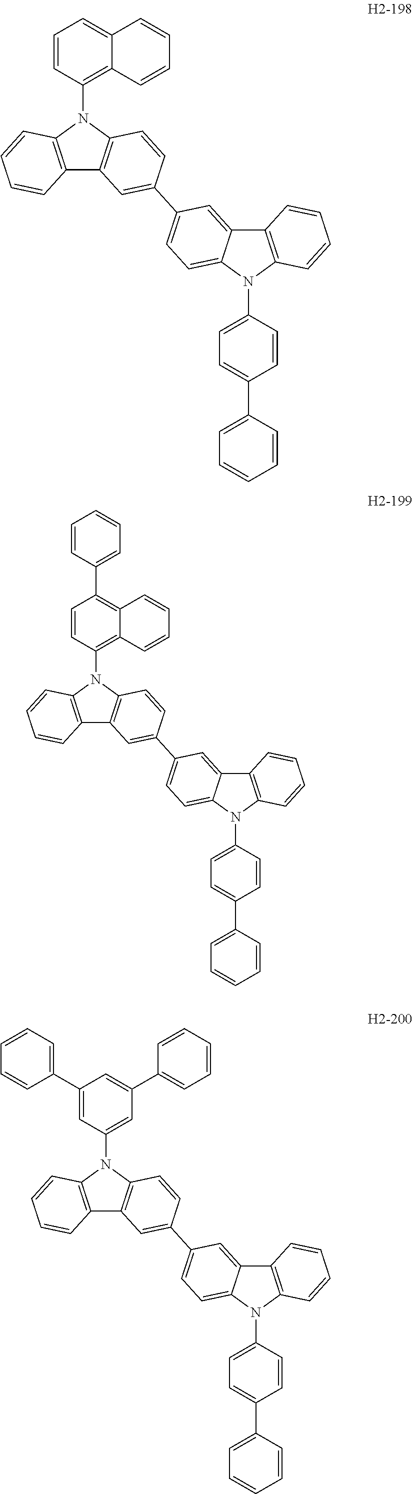 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

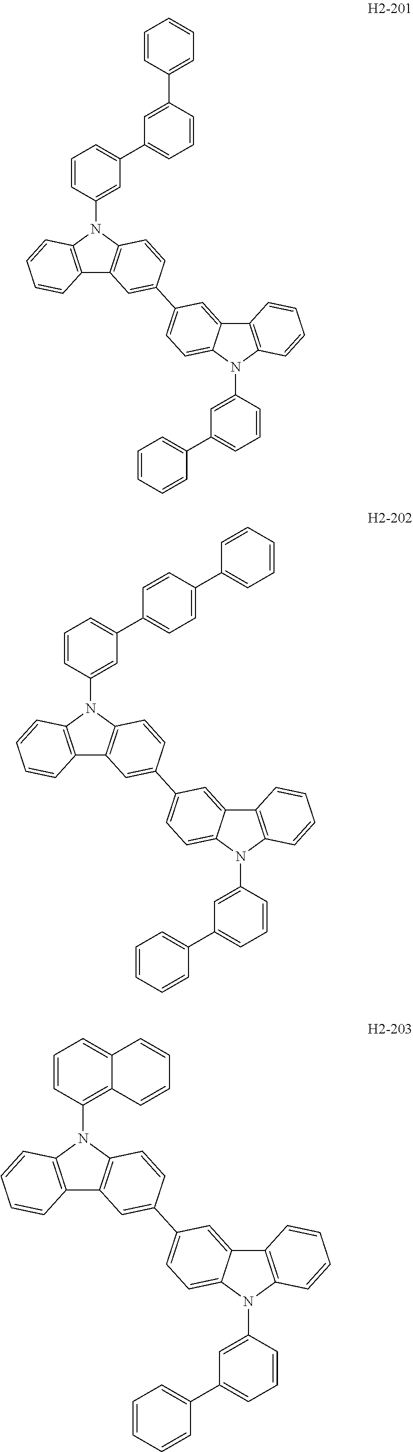 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1]

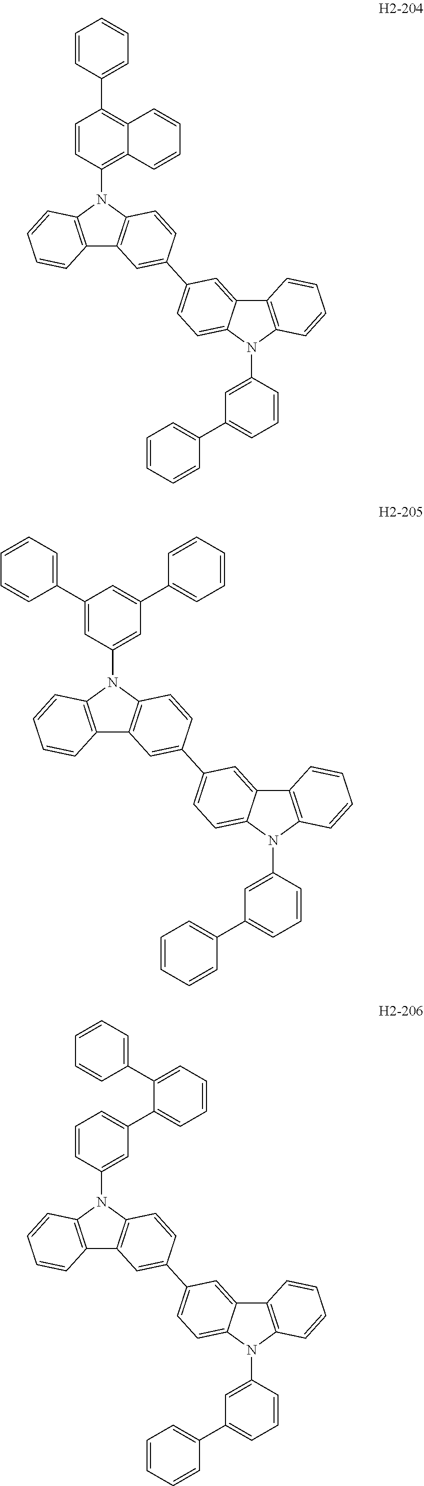 [SMILES: c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5cccc(-c6ccccc6-c6ccccc6)c5)ccc43)c2)cc1]

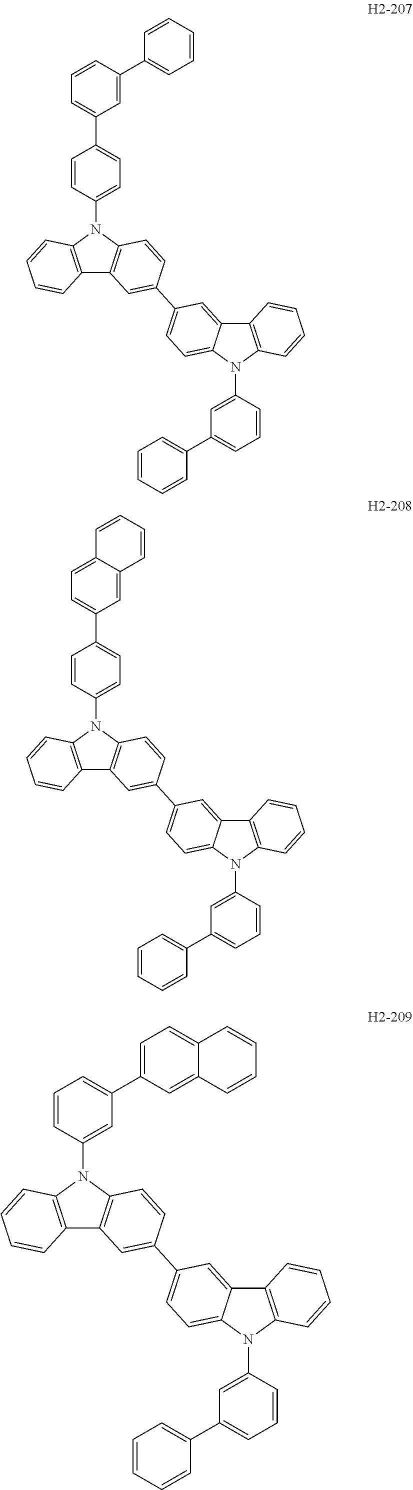

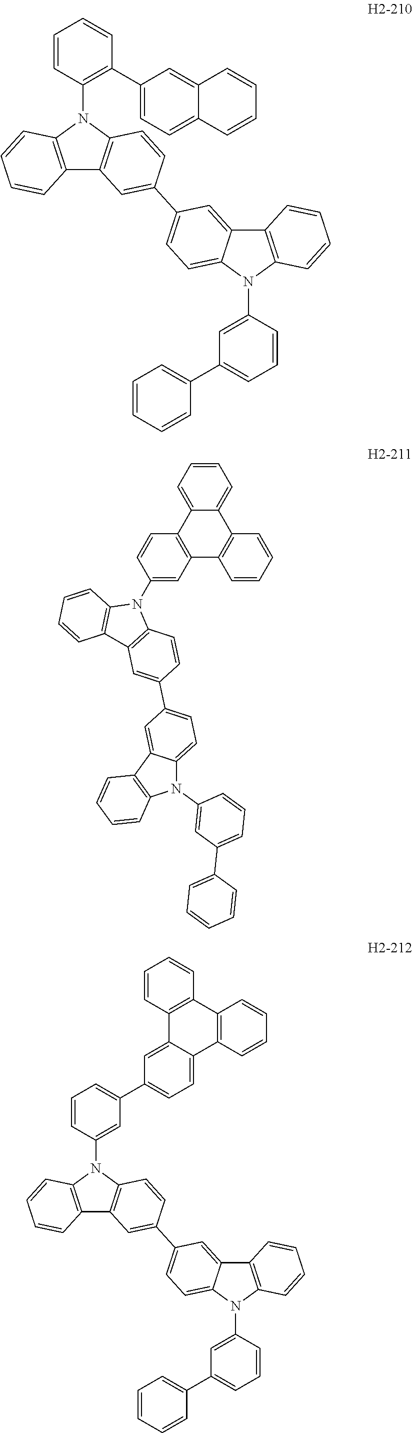 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1]

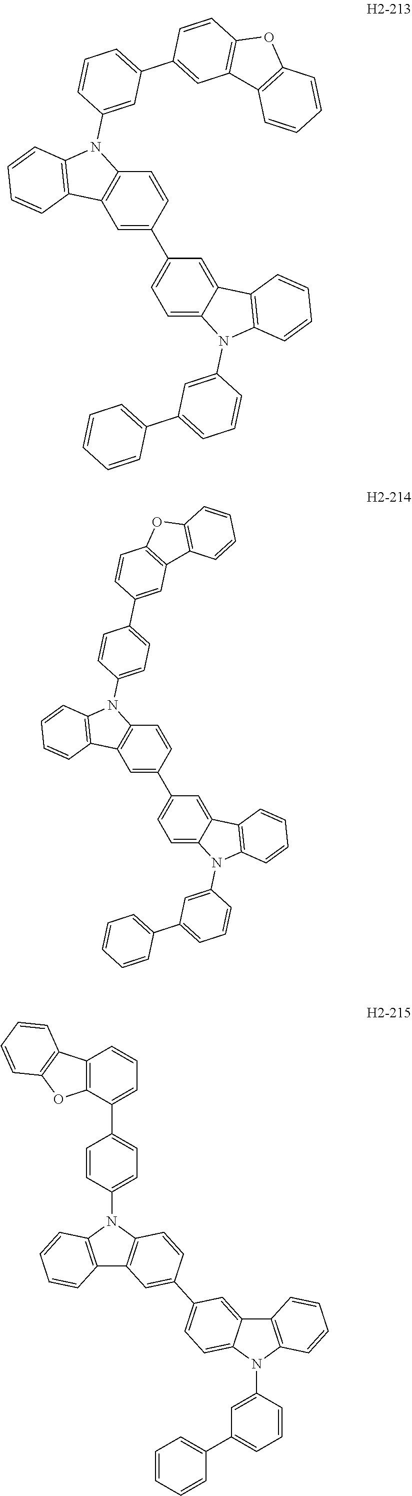 [SMILES: c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

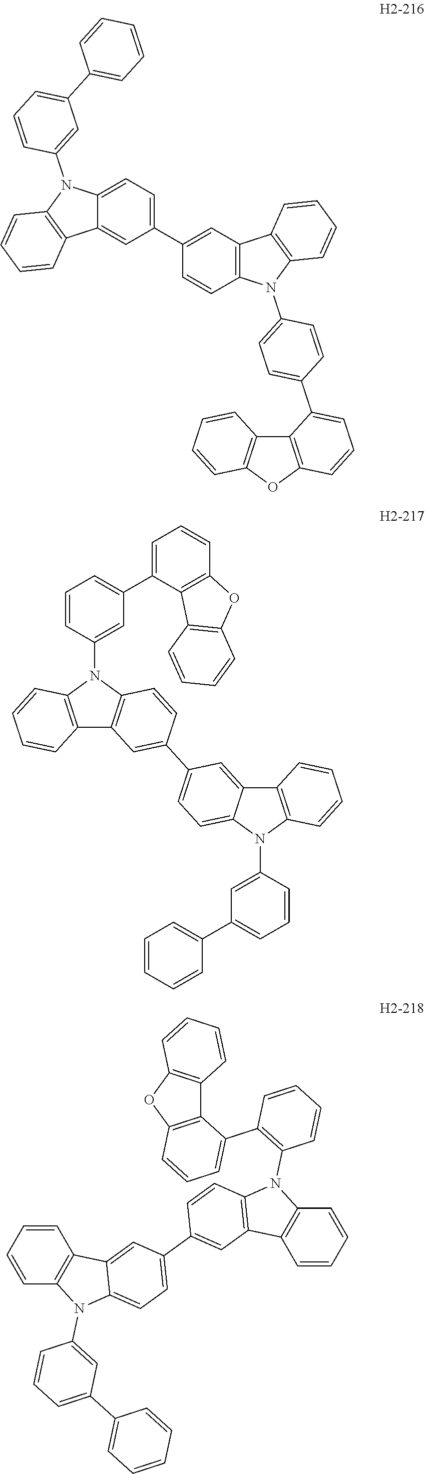

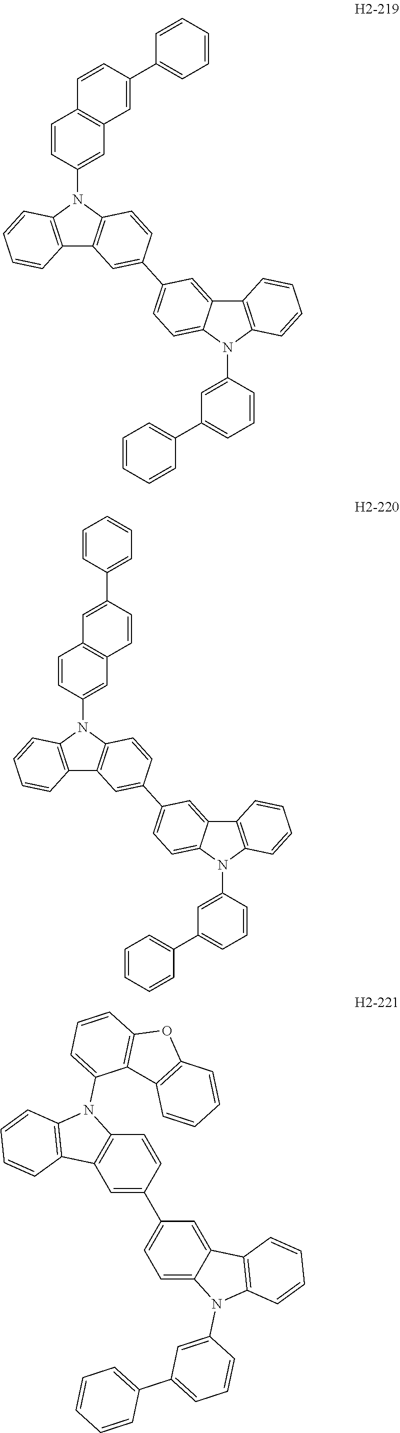

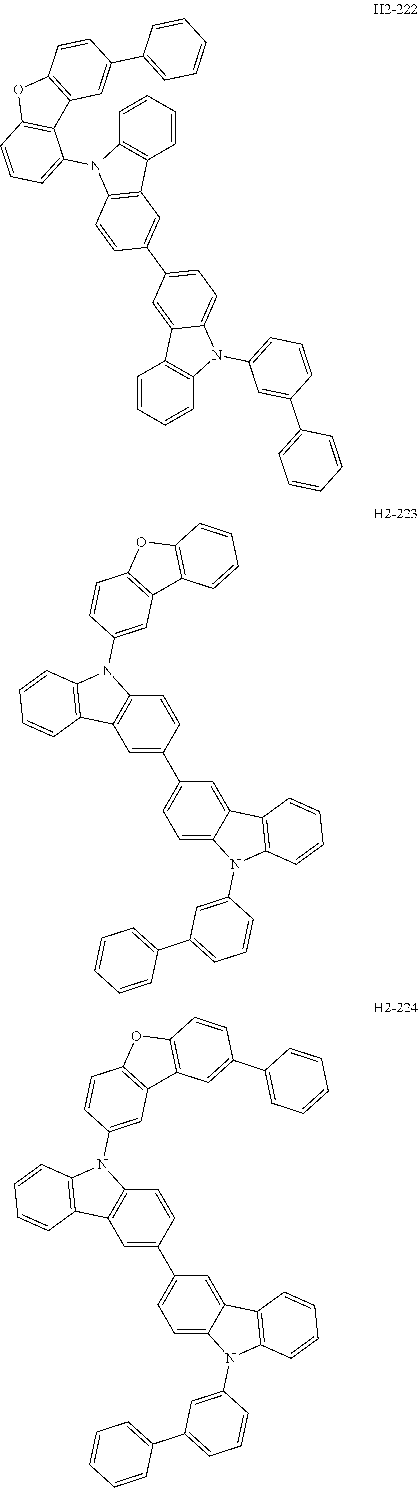

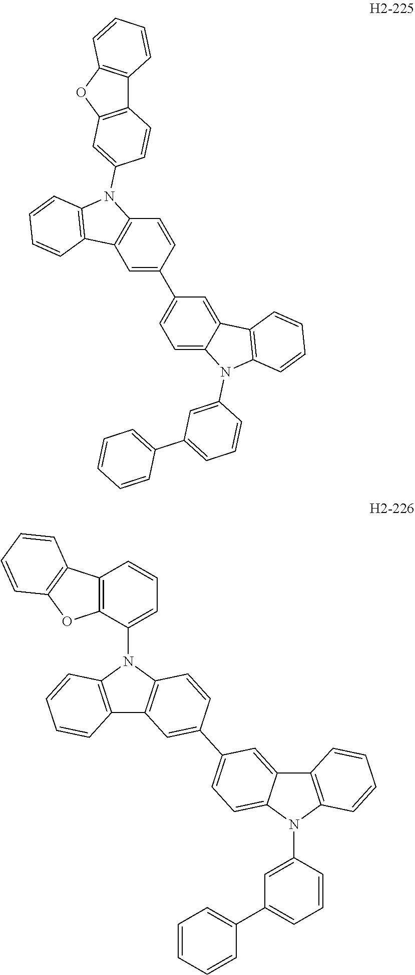 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cccc6c5oc5ccccc56)ccc43)c2)cc1]

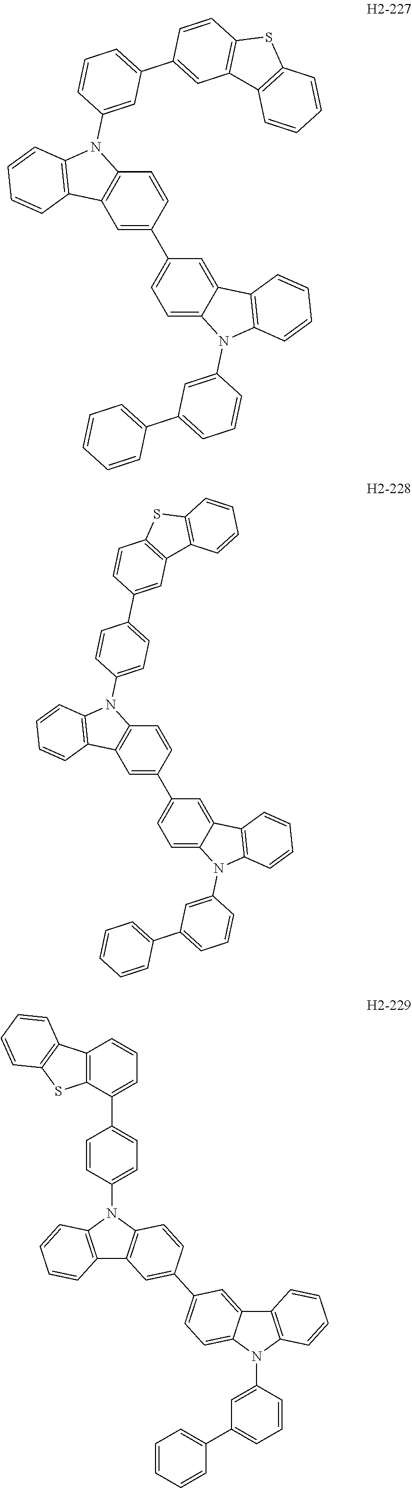 [SMILES: c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1]

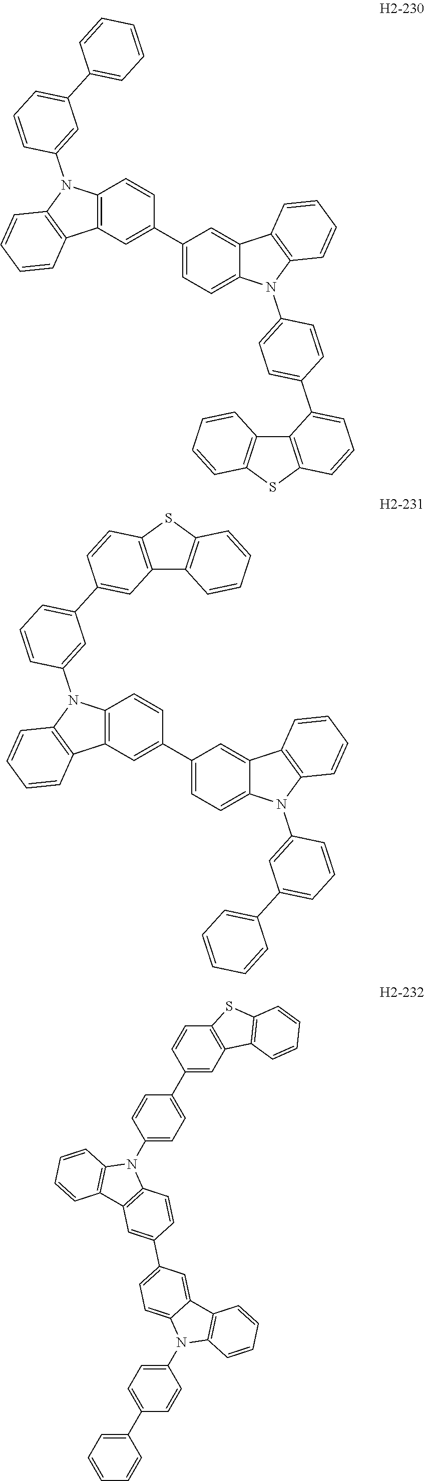

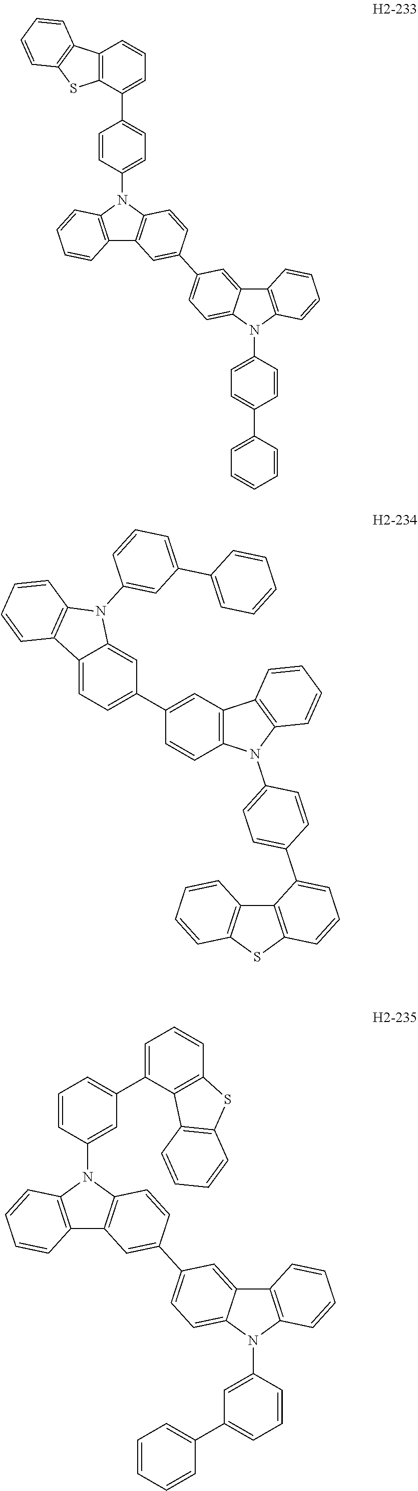 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4ccc(-c5ccc6c(c5)c5ccccc5n6-c5ccc(-c6cccc7sc8ccccc8c67)cc5)cc43)c2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1]

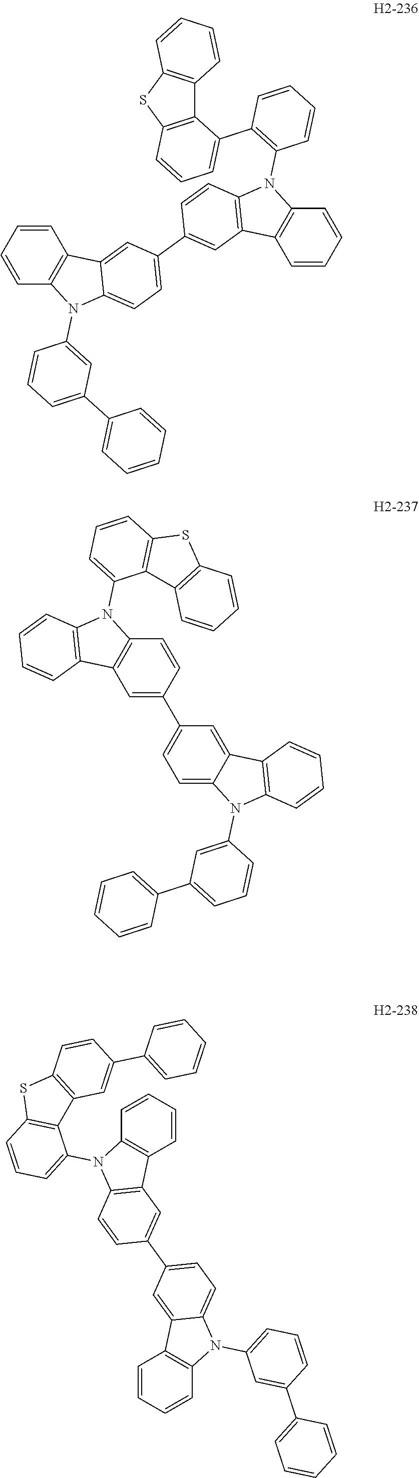

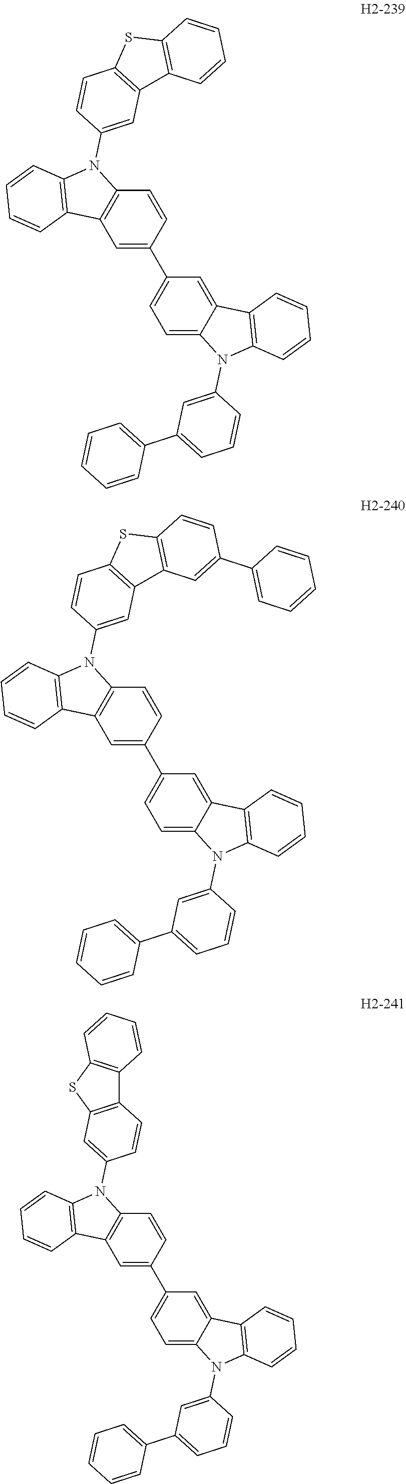

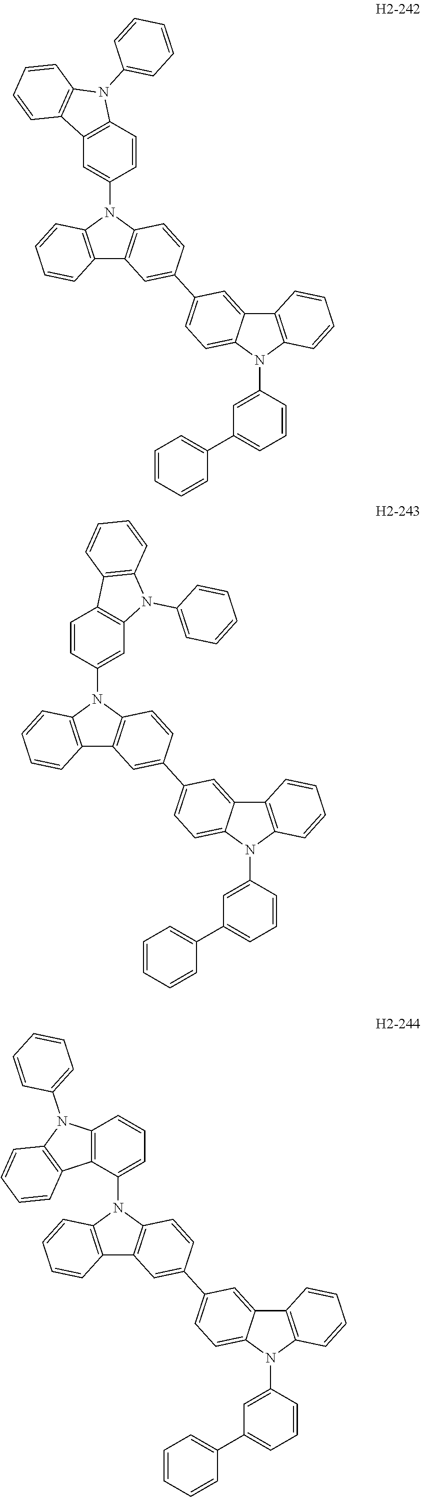 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1 ; c1ccc(-c2cccc(-n3c4ccccc4c4cc(-c5ccc6c(c5)c5ccccc5n6-c5cccc6c5c5ccccc5n6-c5ccccc5)ccc43)c2)cc1]

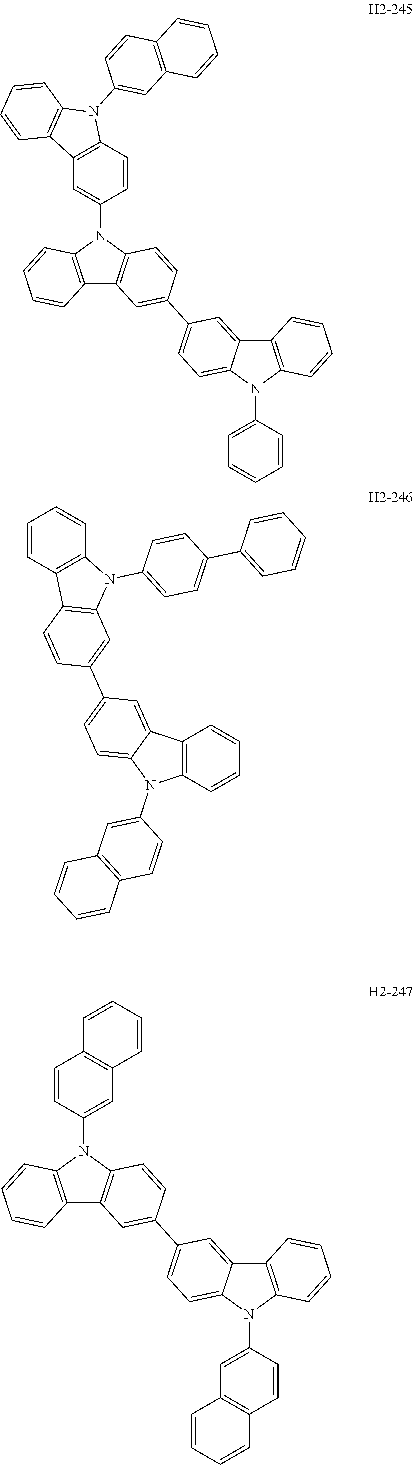

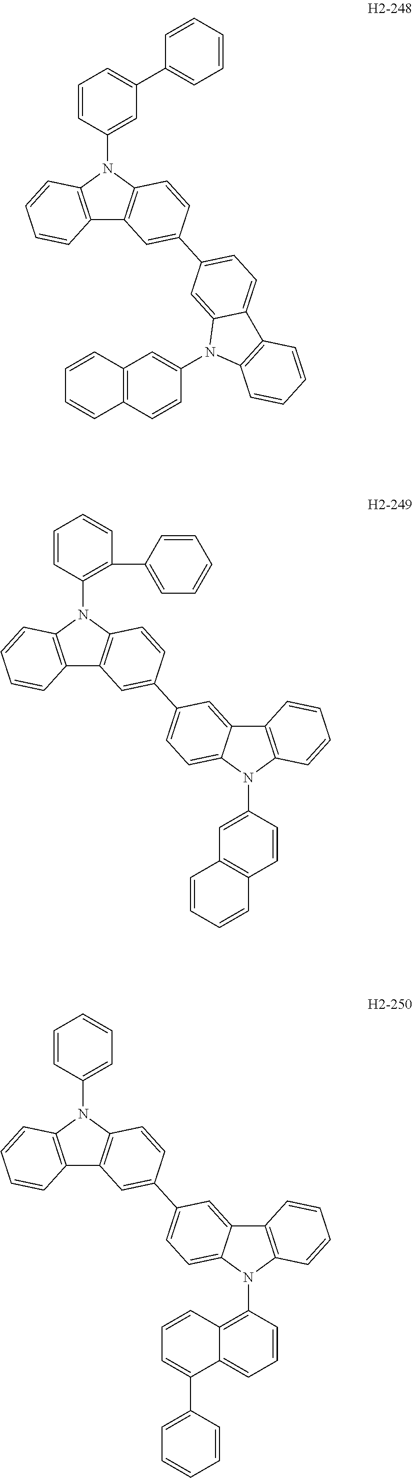 [SMILES: c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1]

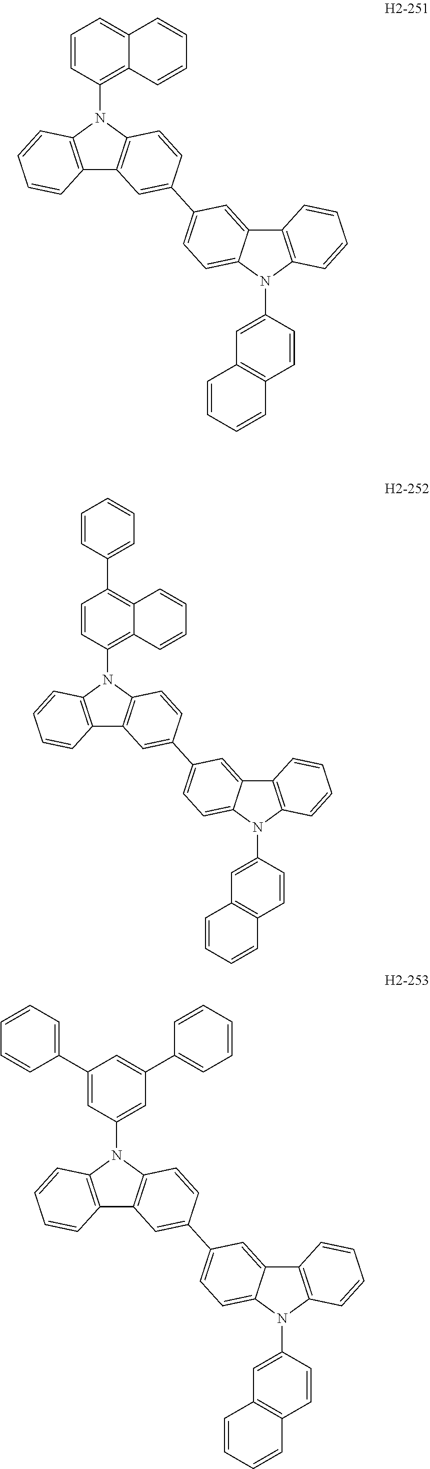 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

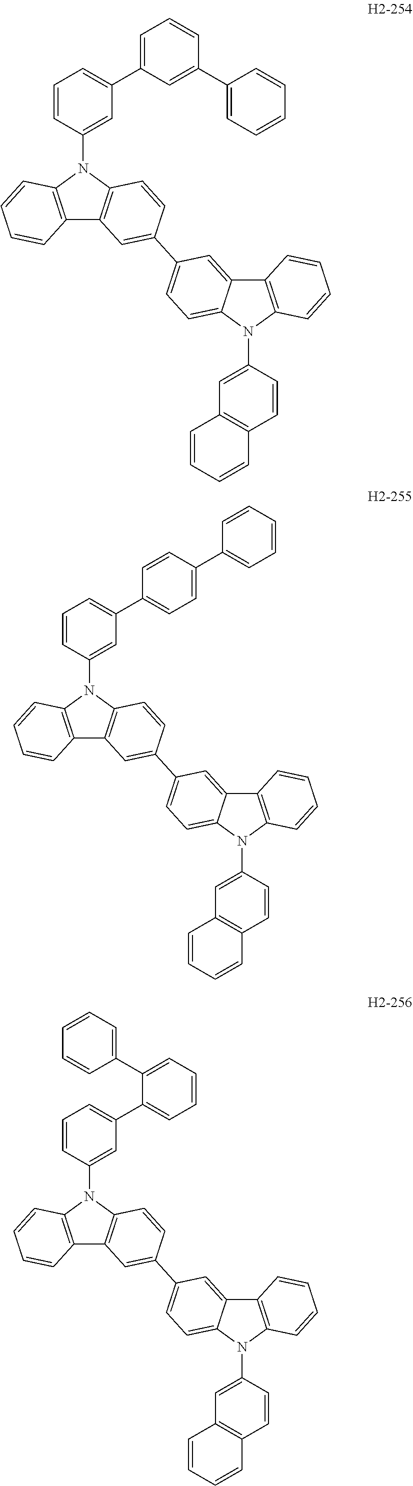

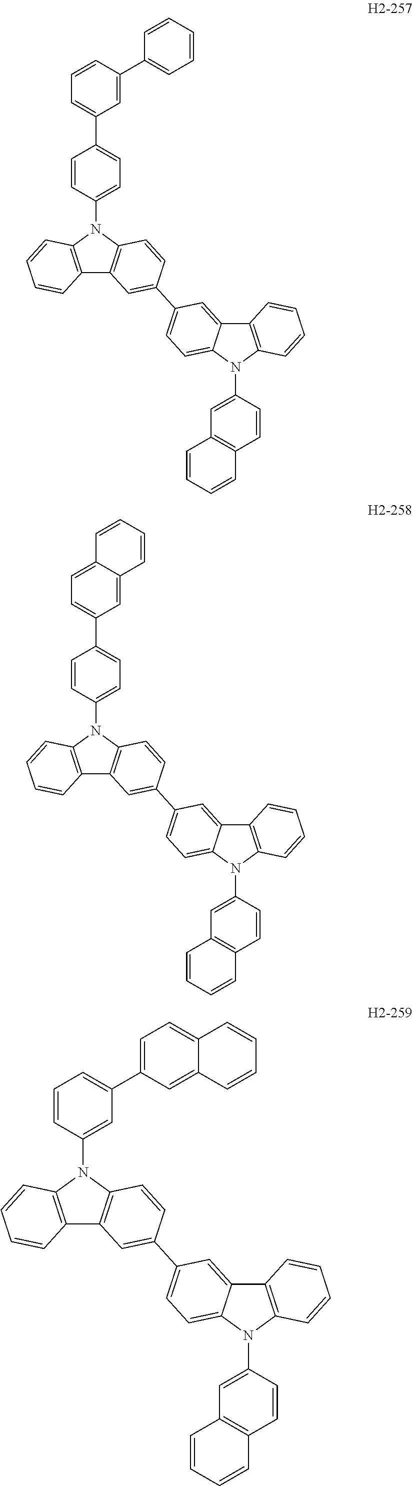

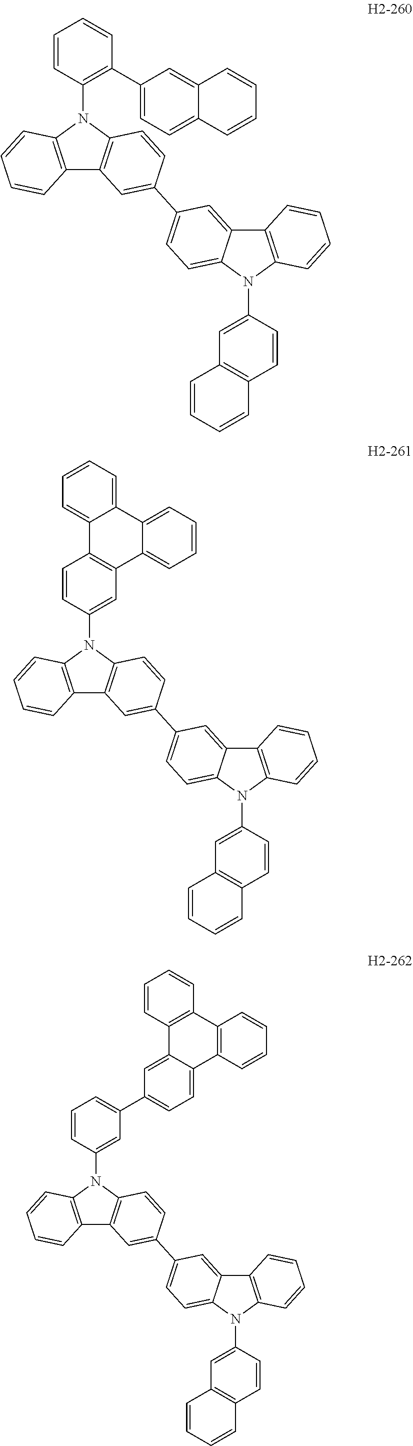 [SMILES: c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1]

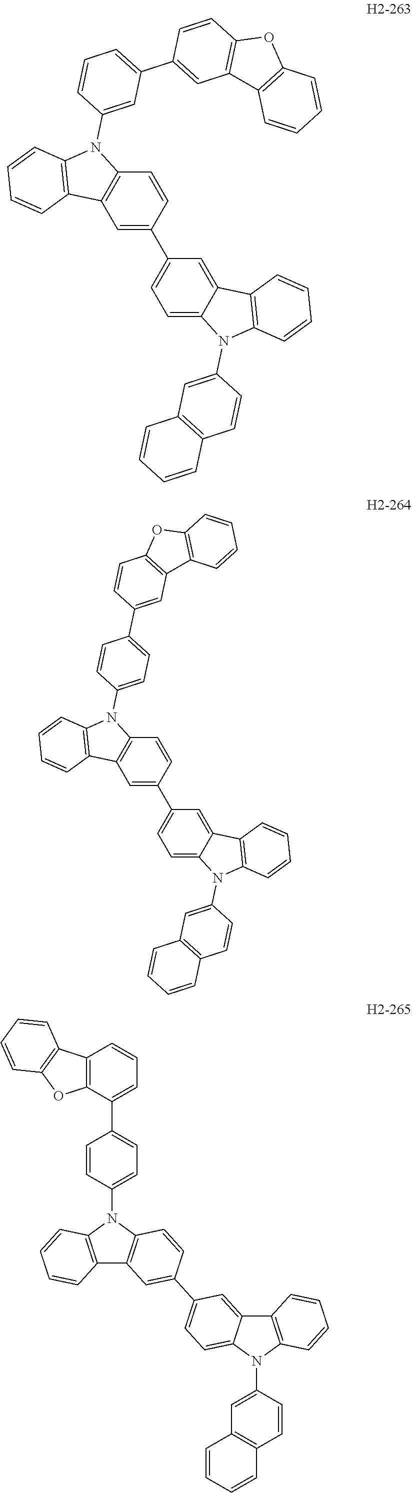 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6oc6ccccc67)cc5)ccc43)ccc2c1]

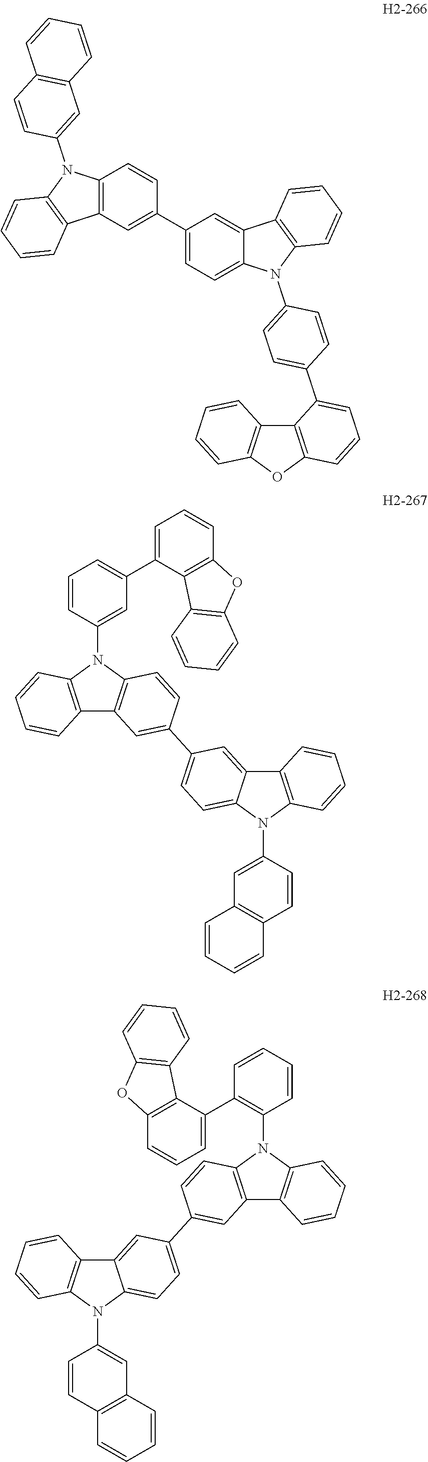 [SMILES: c1ccc2cc(-n3c4ccccc4c4cc(-c5ccc6c(c5)c5ccccc5n6-c5ccc(-c6cccc7oc8ccccc8c67)cc5)ccc43)ccc2c1 ; c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1]

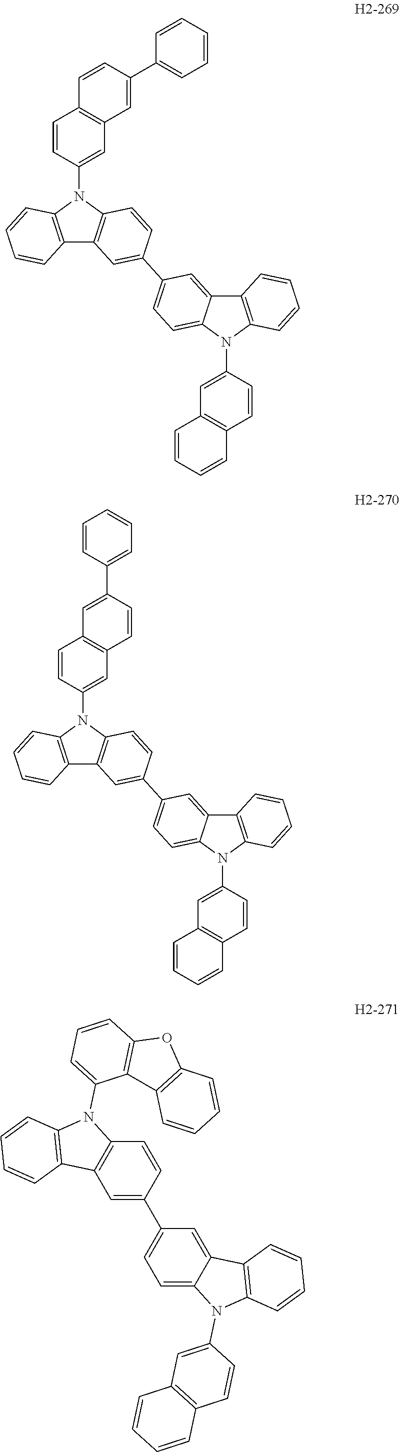 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1 ; c1ccc2cc(-n3c4ccccc4c4cc(-c5ccc6c(c5)c5ccccc5n6-c5cccc6oc7ccccc7c56)ccc43)ccc2c1]

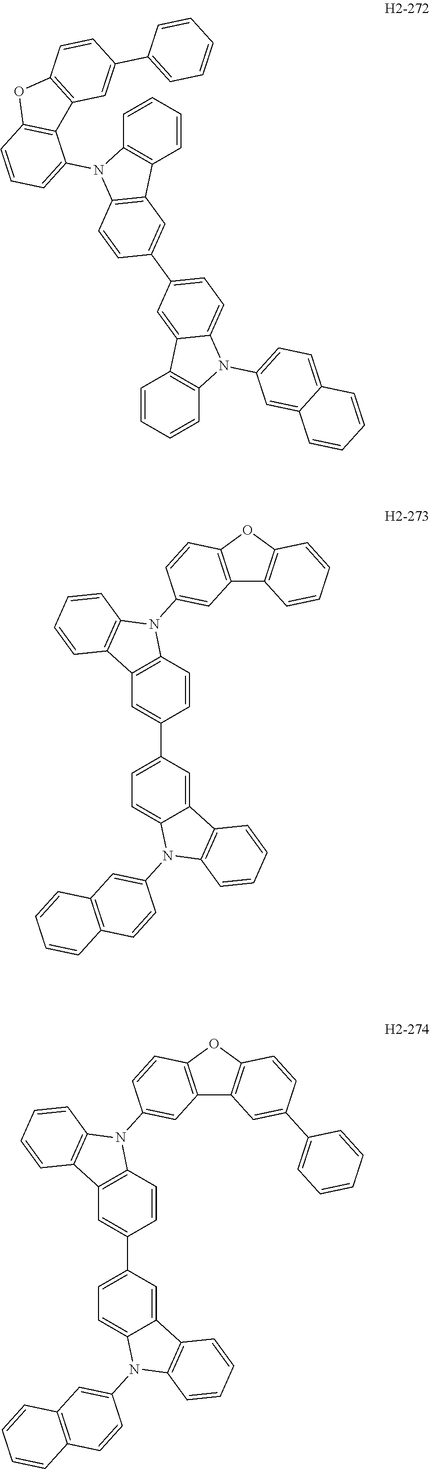 [SMILES: c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1]

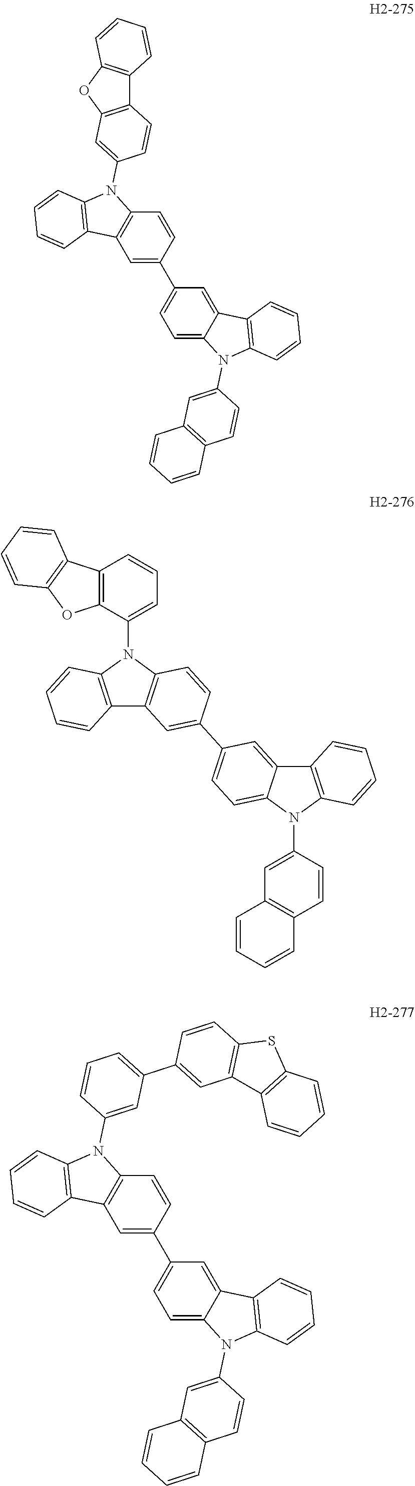 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1 ; c1cc(-c2ccc3sc4ccccc4c3c2)cc(-n2c3ccccc3c3cc(-c4ccc5c(c4)c4ccccc4n5-c4ccc5ccccc5c4)ccc32)c1]

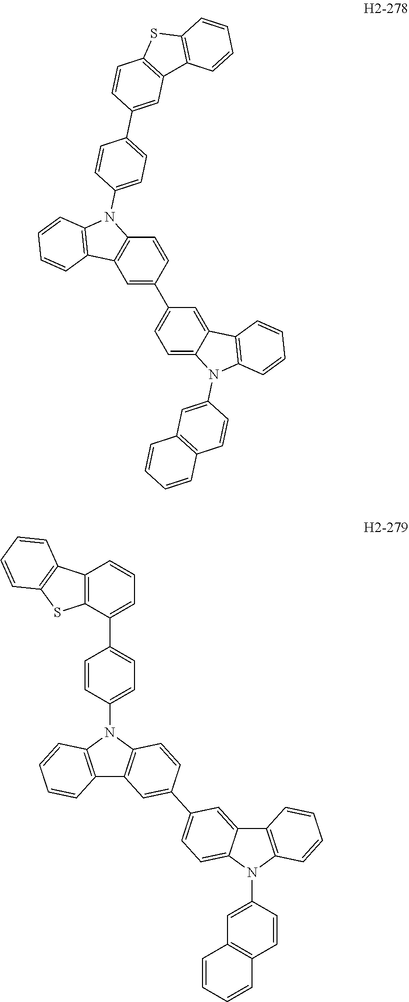 [SMILES: c1ccc2cc(-n3c4ccccc4c4cc(-c5ccc6c(c5)c5ccccc5n6-c5ccc(-c6ccc7sc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6sc6ccccc67)cc5)ccc43)ccc2c1]

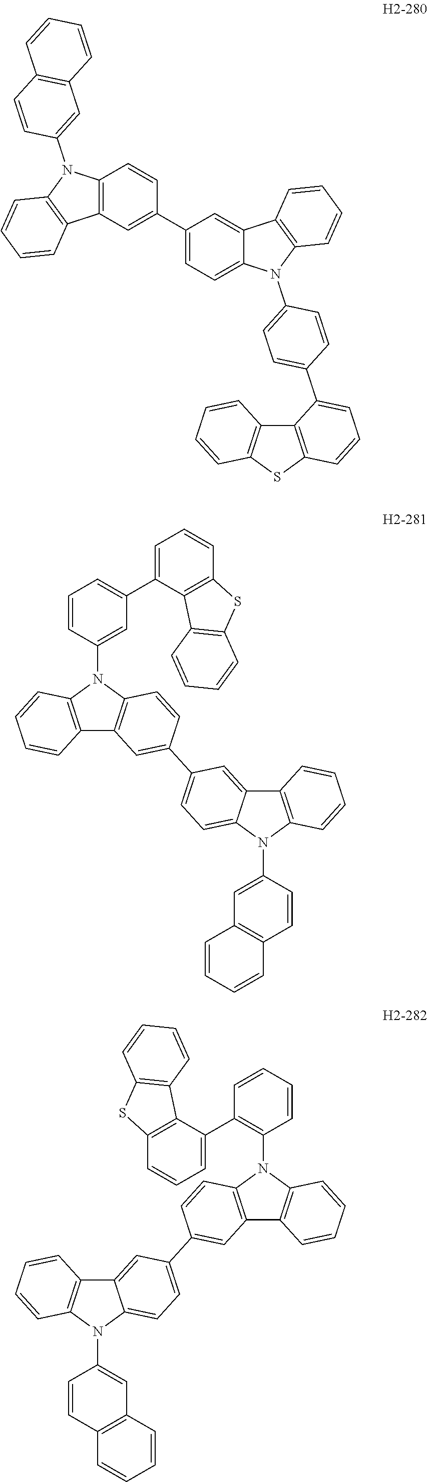 [SMILES: c1ccc2cc(-n3c4ccccc4c4cc(-c5ccc6c(c5)c5ccccc5n6-c5ccc(-c6cccc7sc8ccccc8c67)cc5)ccc43)ccc2c1 ; c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1]

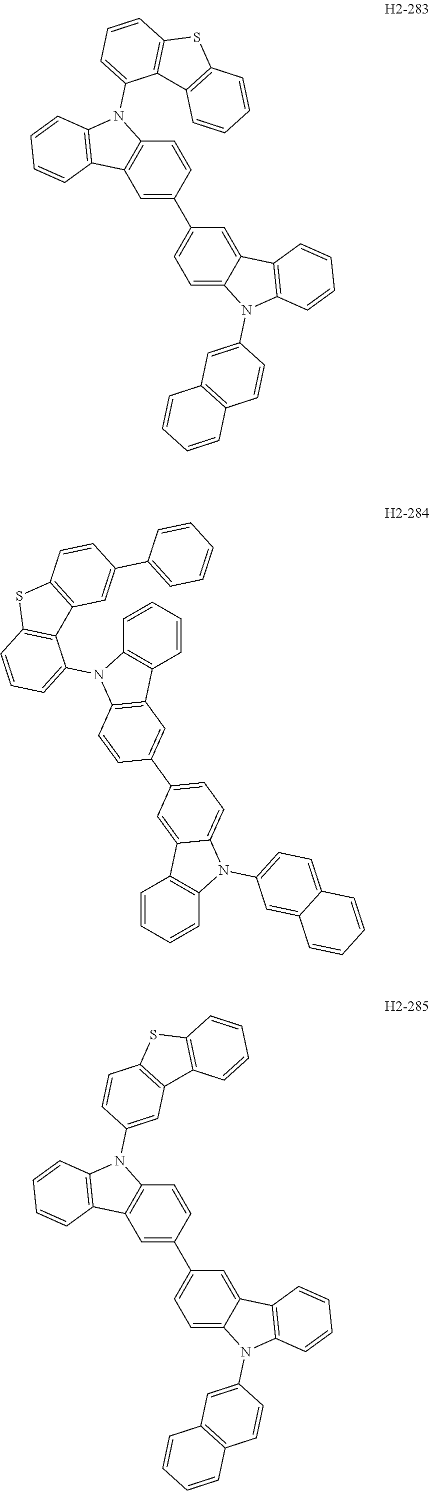 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6sc7ccccc7c6c5)ccc43)ccc2c1]

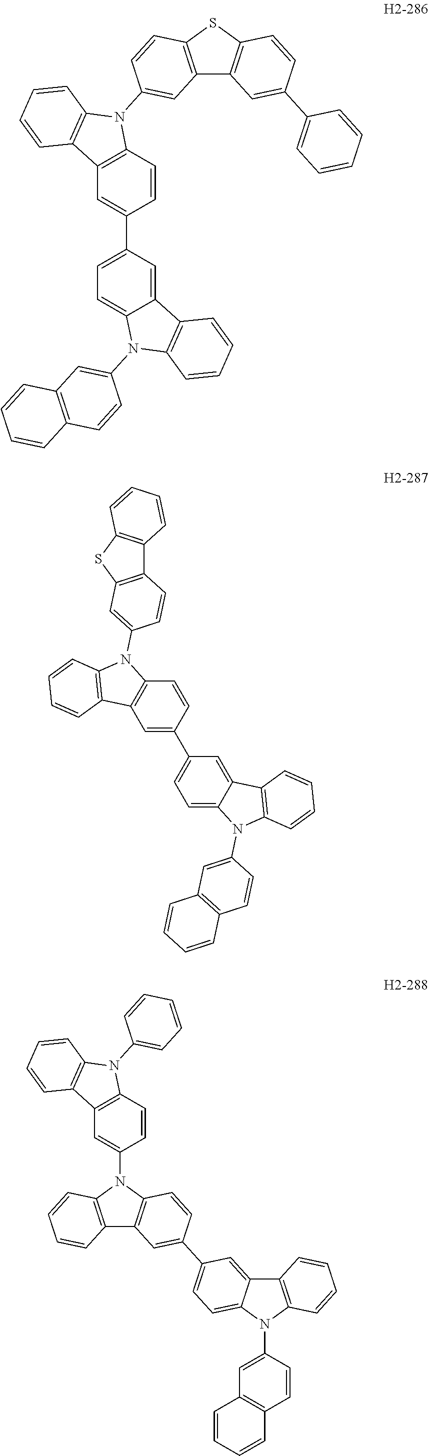 [SMILES: c1ccc(-c2ccc3sc4ccc(-n5c6ccccc6c6cc(-c7ccc8c(c7)c7ccccc7n8-c7ccc8ccccc8c7)ccc65)cc4c3c2)cc1 ; c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1]

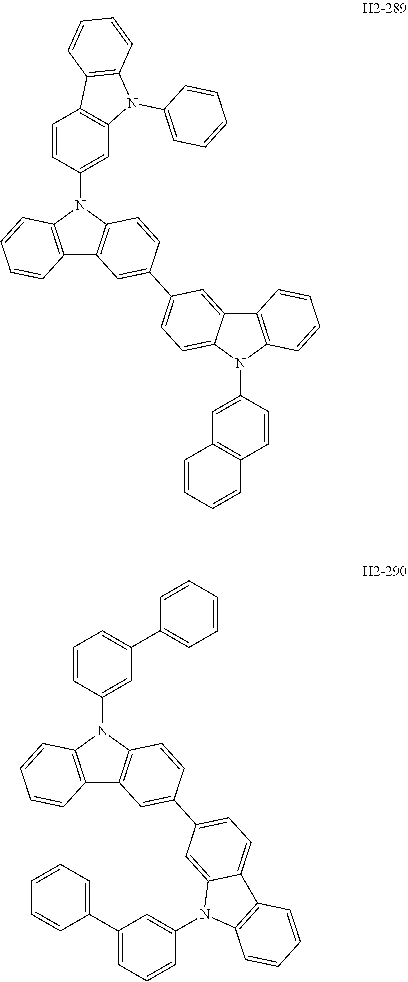 [SMILES: c1ccc(-n2c3ccccc3c3ccc(-n4c5ccccc5c5cc(-c6ccc7c(c6)c6ccccc6n7-c6ccc7ccccc7c6)ccc54)cc32)cc1 ; c1ccc(-c2cccc(-n3c4ccccc4c4cc(-c5ccc6c7ccccc7n(-c7cccc(-c8ccccc8)c7)c6c5)ccc43)c2)cc1]

In the compounds above, Dₙ means that n number of hydrogens is replaced with deuterium.

The compound represented by Formula 2 according to the present disclosure may be prepared by referring to Japanese Patent No. 3139321, etc. In addition, some of the compound represented by Formula 2 according to the present disclosure may be prepared as shown in the following Reaction Scheme 1, but is not limited thereto.

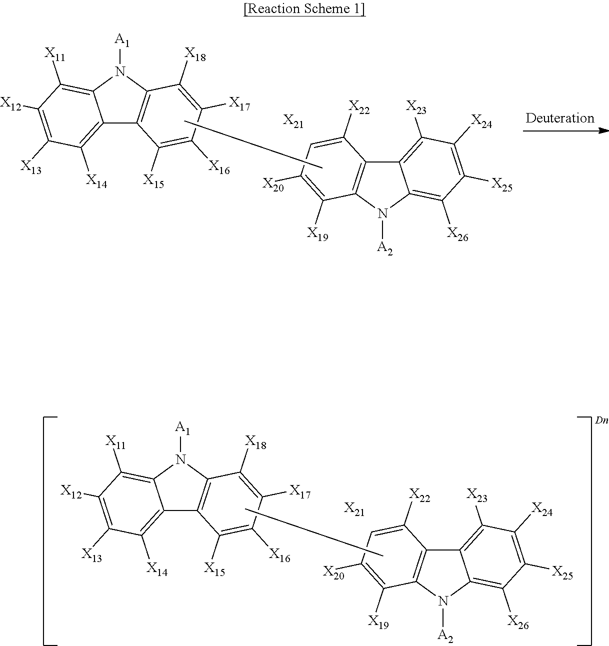

In reaction scheme 1, A₁, A₂, and X₁₁ to X₂₆ are as defined in Formula 2, and Dₙ means that n number of hydrogens is replaced with deuterium.

As described above, exemplary synthesis examples of the compounds represented by Formula 2 according to the present disclosure are described, but they are based on Buchwald-Hartwig cross coupling reaction, N-arylation reaction, H-mont-mediated etherification reaction, Miyaura borylation reaction, Suzuki cross-coupling reaction, intramolecular acid-induced cyclization reaction, Pd(II)-catalyzed oxidative cyclization reaction, Grignard reaction, Heck reaction, cyclic dehydration reaction, S_{N}1 substitution reaction, S_{N}2 substitution reaction, phosphine-mediated reductive cyclization reaction, etc. It will be understood by one skilled in the art that the above reaction proceeds even if other substituents defined in Formula 2 other than the substituents described in the specific synthesis examples are bonded.

According to another embodiment, the present disclosure provides an organic electroluminescent material comprising the compound represented by Formula 1 above and a compound represented by the following Formula 3.

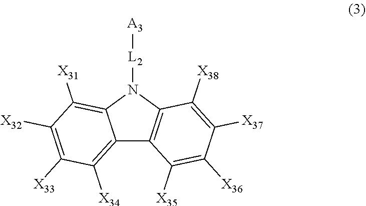

In Formula 3,
A₃ represents a substituted or unsubstituted (C₆-C₃₀)aryl, a substituted or unsubstituted dibenzofuranyl, a substituted or unsubstituted dibenzothiophenyl, a substituted or unsubstituted dibenzoselenophenyl, or a substituted or unsubstituted carbazolyl;
L₂ represents a single bond or a substituted or unsubstituted (C₆-C₃₀)arylene;
a pair of adjacent X₃₅ to X₃₈ are linked to the following Formula 3-A to form a ring(s), and X₃₅ to X₃₈ which do not form a ring, and X₃₁ to X₃₄ each independently represent hydrogen, deuterium, a substituted or unsubstituted (C₆-C₃₀)aryl, or a substituted or unsubstituted (3- to 30-membered)heteroaryl;

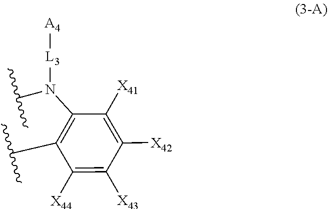

In Formula 3-A,
A₄ represents a substituted or unsubstituted (C₆-C₃₀)aryl, a substituted or unsubstituted dibenzofuranyl, a substituted or unsubstituted dibenzothiophenyl, a substituted or unsubstituted dibenzoselenophenyl, or a substituted or unsubstituted carbazolyl;
L₃ represents a single bond or a substituted or unsubstituted (C₆-C₃₀)arylene; and
X₄₁ to X₄₄ each independently represent hydrogen, deuterium, a substituted or unsubstituted (C₆-C₃₀)aryl, or a substituted or unsubstituted (3- to 30-membered)heteroaryl.

In one embodiment, L₂ and L₃ each independently may be a single bond or a substituted or unsubstituted (C₆-C₃₀)arylene, preferably a single bond or a substituted or unsubstituted (C₆-C₂₅)arylene, more preferably a single bond or a substituted or unsubstituted (C₆-C₁₈)arylene. For example, L₂ and L₃ each independently may be a single bond, a substituted or unsubstituted phenylene, or a substituted or unsubstituted naphthylene.

In one embodiment, A₃ and A₄ each independently may be a substituted or unsubstituted (C₆-C₂₅)aryl, a substituted or unsubstituted dibenzofuranyl, a substituted or unsubstituted dibenzothiophenyl, a substituted or unsubstituted dibenzoselenophenyl, or a substituted or unsubstituted carbazolyl. For example, A₃ and A₄ each independently may be phenyl unsubstituted or substituted with naphthyl or triphenylenyl, naphthyl unsubstituted or substituted with phenyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted o-terphenyl, a substituted or unsubstituted phenanthrenyl, a substituted or unsubstituted triphenylenyl, a substituted or unsubstituted dimethylfluorenyl, a substituted or unsubstituted diphenylfluorenyl, a substituted or unsubstituted spirobifluorenyl, a substituted or unsubstituted chrysenyl, a substituted or unsubstituted dibenzofuranyl, a substituted or unsubstituted dibenzothiophenyl, or a substituted or unsubstituted dibenzoselenophenyl.

In one embodiment, a pair of adjacent X₃₅ to X₃₈ are linked to Formula 3-A above to form a ring, which may be represented by any one of the following Formulas 3-1 to 3-6.

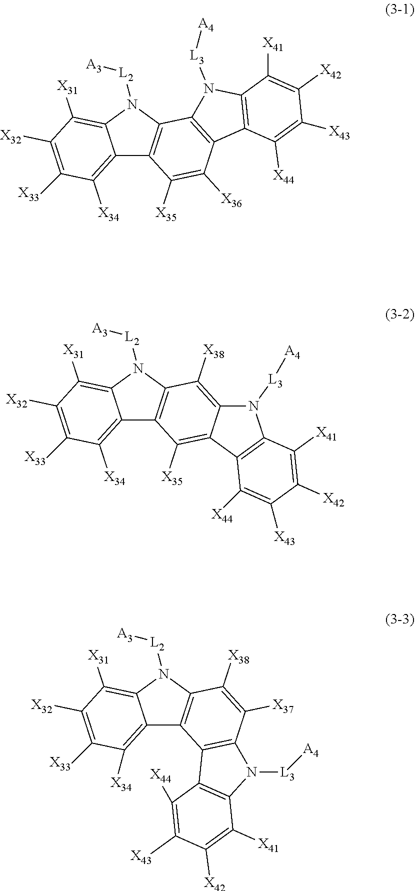

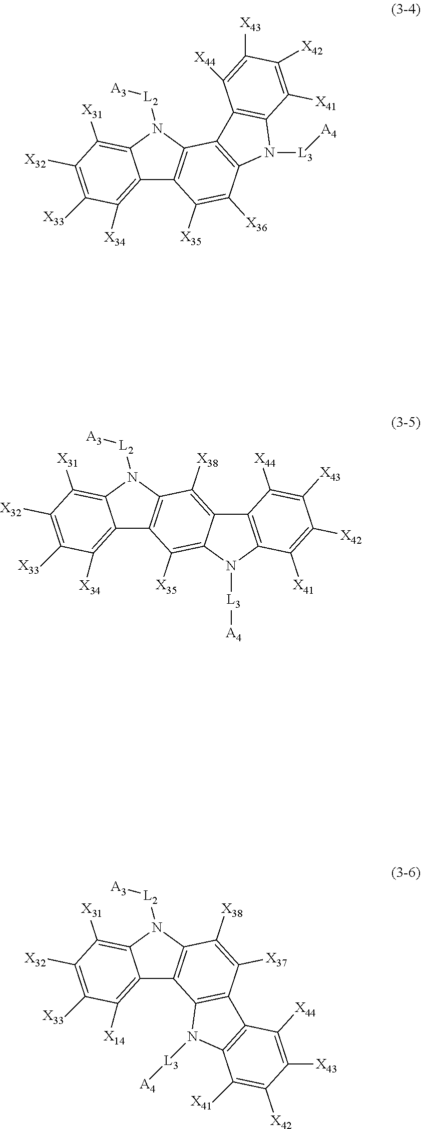

In Formulas 3-1 to 3-6,
A₃, A₄, L₂, L₃, X₃₁ to X₃₈, and X₄₁ to X₄₄ are as defined in Formula 3.

In one embodiment, X₃₅ to X₃₈ which do not form a ring, X₃₁ to X₃₄, and X₄₁ to X₄₄ each independently may be hydrogen or deuterium.

According to one embodiment, in the compound represented by Formula 3, the deuterium substitution rate may be 40% to 100%, for example, 50% to 100%, for example, 60% to 100%, or, for example, 70% to 100%. The upper limit of the deuterium substitution rate may be 100%, but may also be less than 100%, for example, about 99%. That is, the compound represented by Formula 3 can be a compound in which all hydrogens at substitutable positions are substituted with deuterium, or a compound in which hydrogens at substitutable positions are partially substituted with deuterium.

When the compound represented by Formula 3 contains deuterium substituted according to the above-mentioned number or ratio, the bond dissociation energy according to deuteration increases, thereby increasing the stability of the compound represented by Formula 3. When such a compound represented by Formula 3 is used in an organic electroluminescent device, improved lifespan characteristics may be exhibited.

According to one embodiment, the compound represented by Formula 3 may be more specifically illustrated by the following compounds, but is not limited thereto.

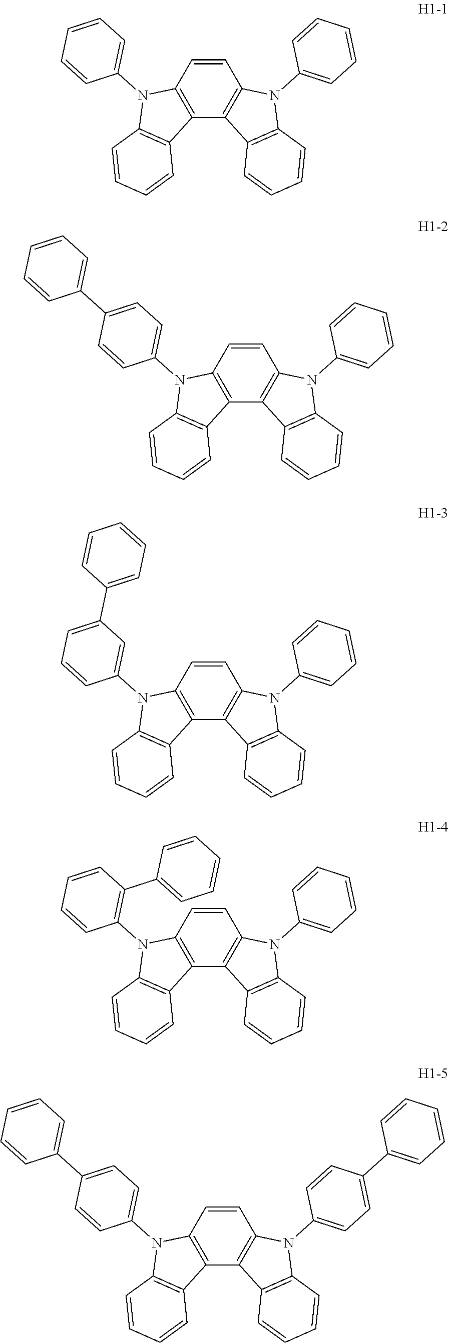 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccccc2-n2c3ccccc3c3c4c5ccccc5n(-c5ccccc5)c4ccc32)cc1 ; c1ccc(-c2ccc(-n3c4ccccc4c4c5c6ccccc6n(-c6ccc(-c7ccccc7)cc6)c5ccc43)cc2)cc1]

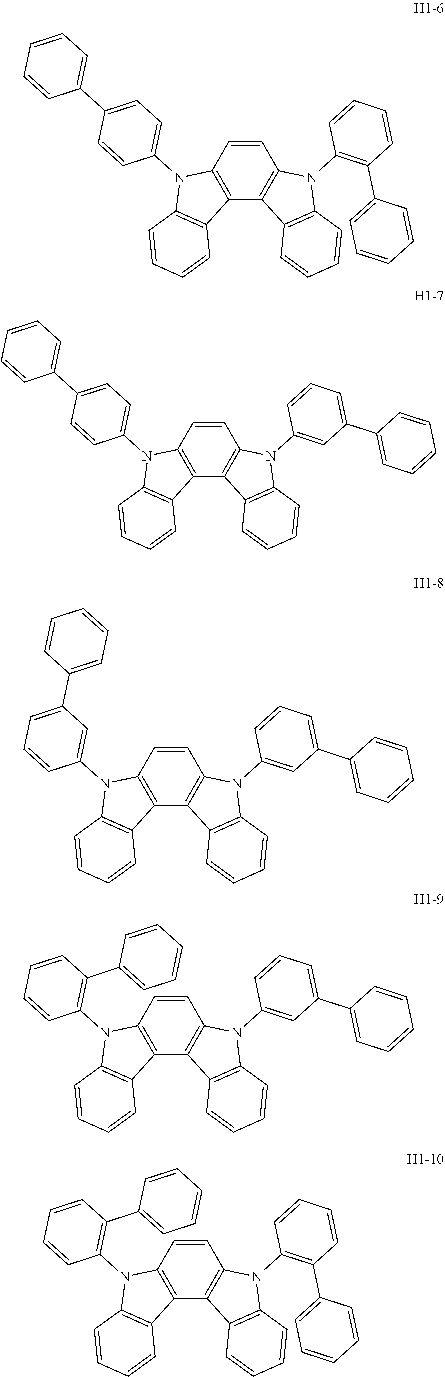 [SMILES: c1ccc(-c2ccc(-n3c4ccccc4c4c5c6ccccc6n(-c6ccccc6-c6ccccc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2cccc(-n3c4ccccc4c4c5c6ccccc6n(-c6cccc(-c7ccccc7)c6)c5ccc43)c2)cc1 ; c1ccc(-c2cccc(-n3c4ccccc4c4c5c6ccccc6n(-c6ccccc6-c6ccccc6)c5ccc43)c2)cc1 ; c1ccc(-c2ccccc2-n2c3ccccc3c3c4c5ccccc5n(-c5ccccc5-c5ccccc5)c4ccc32)cc1]

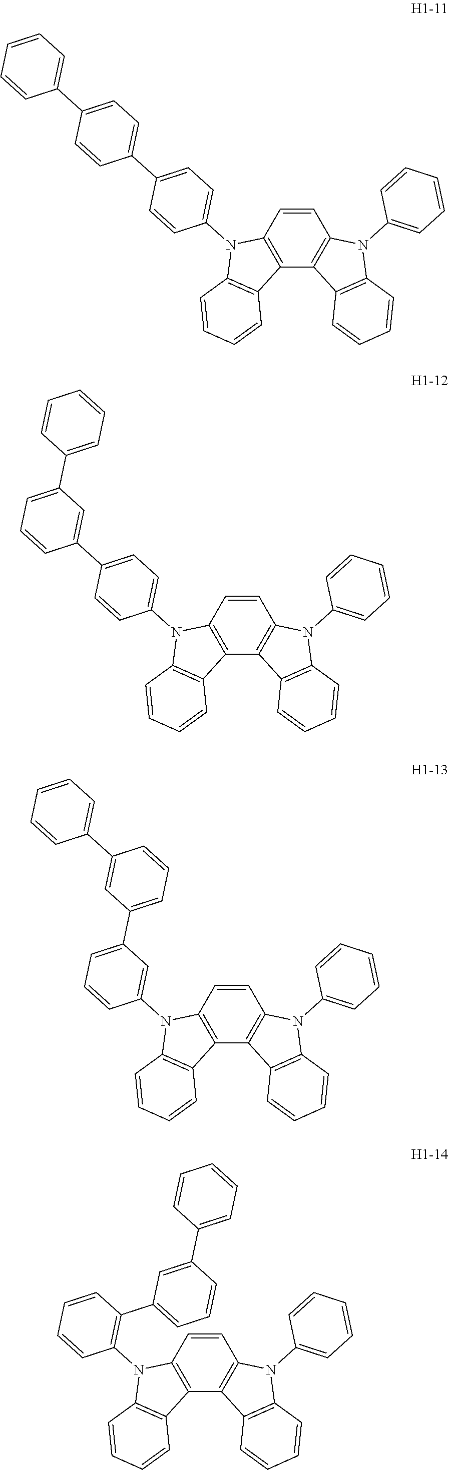 [SMILES: c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2cccc(-c3ccccc3-n3c4ccccc4c4c5c6ccccc6n(-c6ccccc6)c5ccc43)c2)cc1]

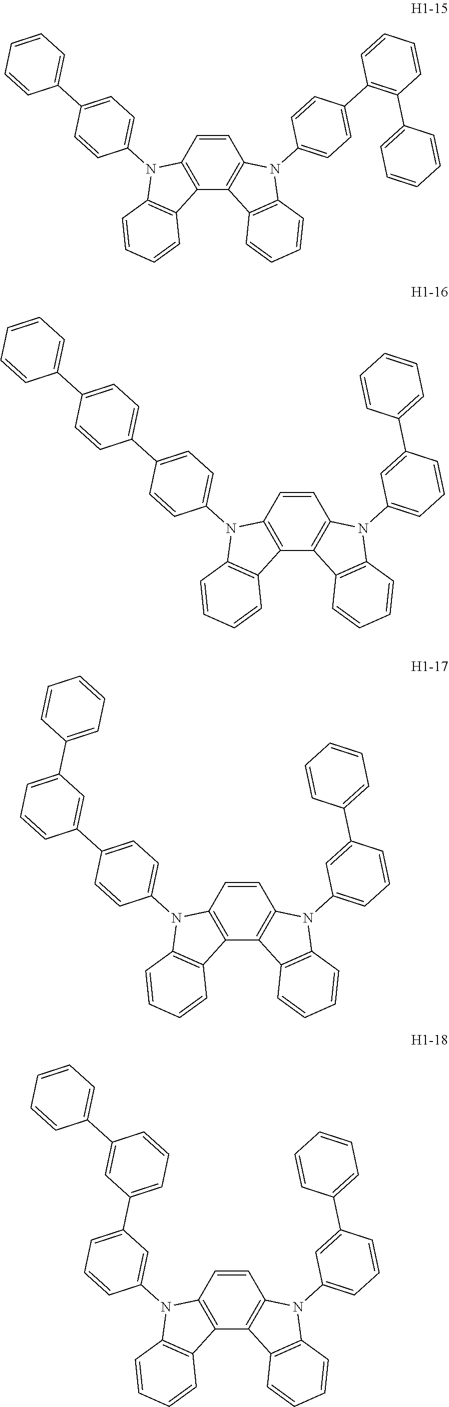 [SMILES: c1ccc(-c2ccc(-n3c4ccccc4c4c5c6ccccc6n(-c6ccc(-c7ccccc7-c7ccccc7)cc6)c5ccc43)cc2)cc1 ; c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2cccc(-c3cccc(-n4c5ccccc5c5c6c7ccccc7n(-c7cccc(-c8ccccc8)c7)c6ccc54)c3)c2)cc1]

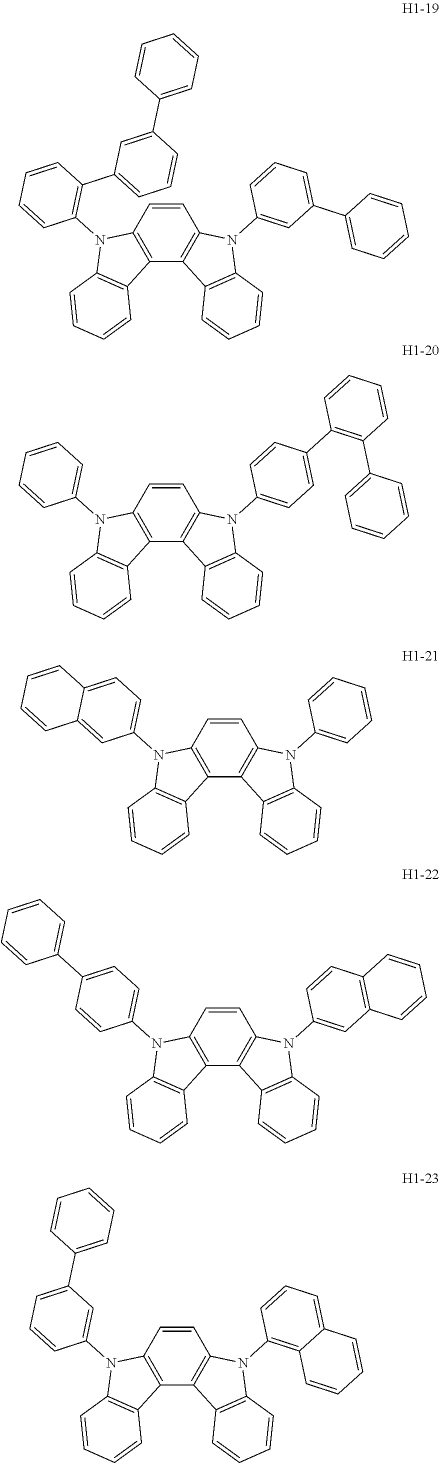 [SMILES: c1ccc(-c2cccc(-c3ccccc3-n3c4ccccc4c4c5c6ccccc6n(-c6cccc(-c7ccccc7)c6)c5ccc43)c2)cc1 ; c1ccc(-c2ccccc2-c2ccc(-n3c4ccccc4c4c5c6ccccc6n(-c6ccccc6)c5ccc43)cc2)cc1 ; c1ccc(-n2c3ccccc3c3c4c5ccccc5n(-c5ccc6ccccc6c5)c4ccc32)cc1 ; c1ccc(-c2ccc(-n3c4ccccc4c4c5c6ccccc6n(-c6ccc7ccccc7c6)c5ccc43)cc2)cc1 ; c1ccc(-c2cccc(-n3c4ccccc4c4c5c6ccccc6n(-c6cccc7ccccc67)c5ccc43)c2)cc1]

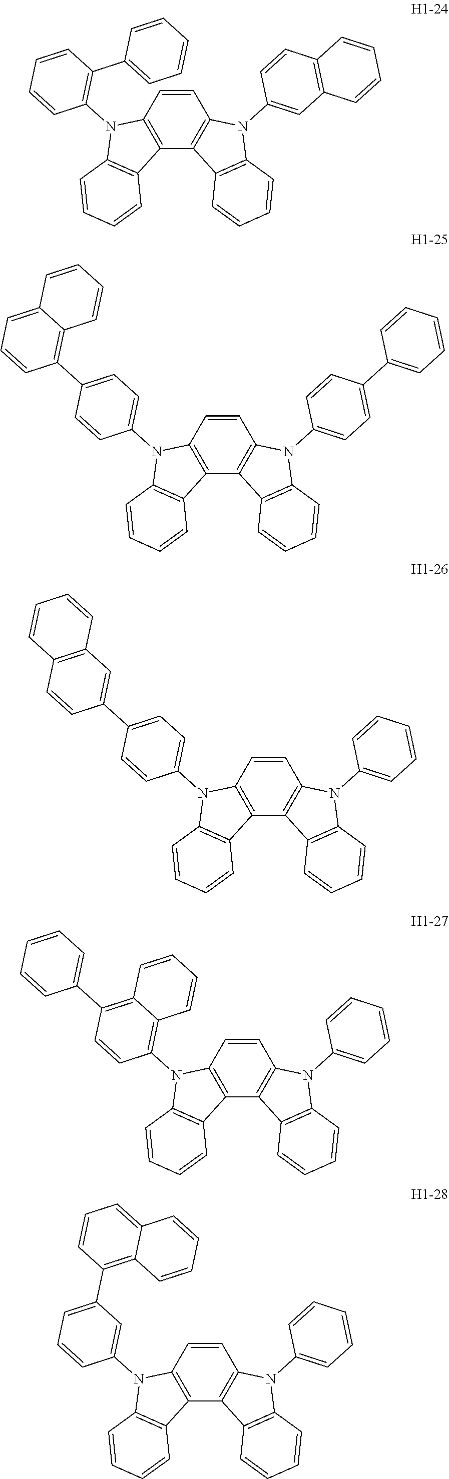

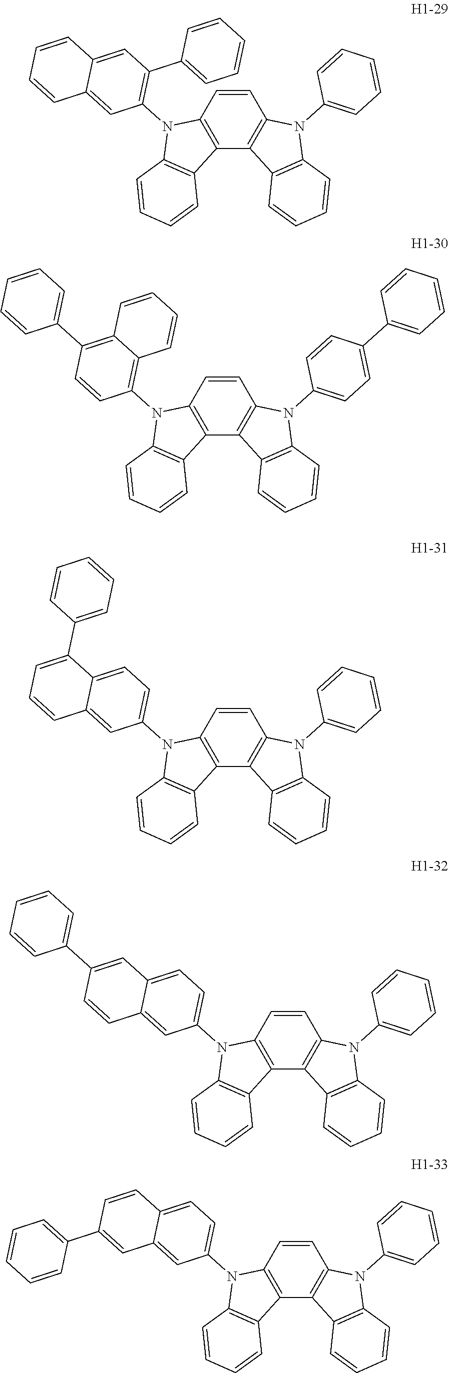 [SMILES: c1ccc(-c2cc3ccccc3cc2-n2c3ccccc3c3c4c5ccccc5n(-c5ccccc5)c4ccc32)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc3cc(-n4c5ccccc5c5c6c7ccccc7n(-c7ccccc7)c6ccc54)ccc23)cc1 ; c1ccc(-c2ccc3cc(-n4c5ccccc5c5c6c7ccccc7n(-c7ccccc7)c6ccc54)ccc3c2)cc1 ; c1ccc(-c2ccc3ccc(-n4c5ccccc5c5c6c7ccccc7n(-c7ccccc7)c6ccc54)cc3c2)cc1]

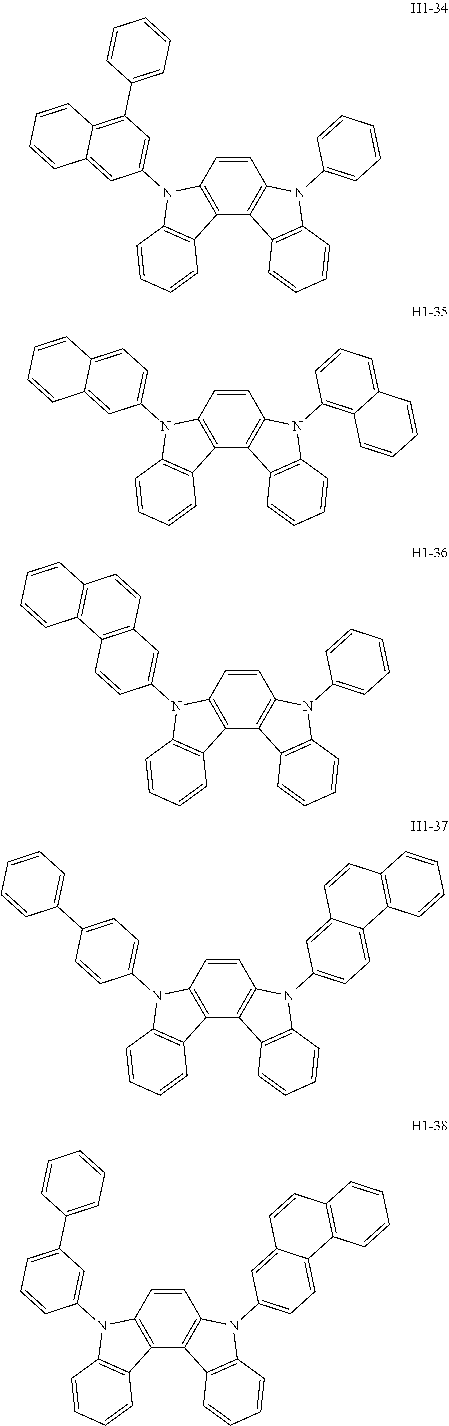 [SMILES: c1ccc(-c2cc(-n3c4ccccc4c4c5c6ccccc6n(-c6ccccc6)c5ccc43)cc3ccccc23)cc1 ; c1ccc2cc(-n3c4ccccc4c4c5c6ccccc6n(-c6cccc7ccccc67)c5ccc43)ccc2c1 ; c1ccc(-n2c3ccccc3c3c4c5ccccc5n(-c5ccc6c(ccc7ccccc76)c5)c4ccc32)cc1 ; c1ccc(-c2ccc(-n3c4ccccc4c4c5c6ccccc6n(-c6ccc7c(ccc8ccccc87)c6)c5ccc43)cc2)cc1 ; c1ccc(-c2cccc(-n3c4ccccc4c4c5c6ccccc6n(-c6ccc7c(ccc8ccccc87)c6)c5ccc43)c2)cc1]

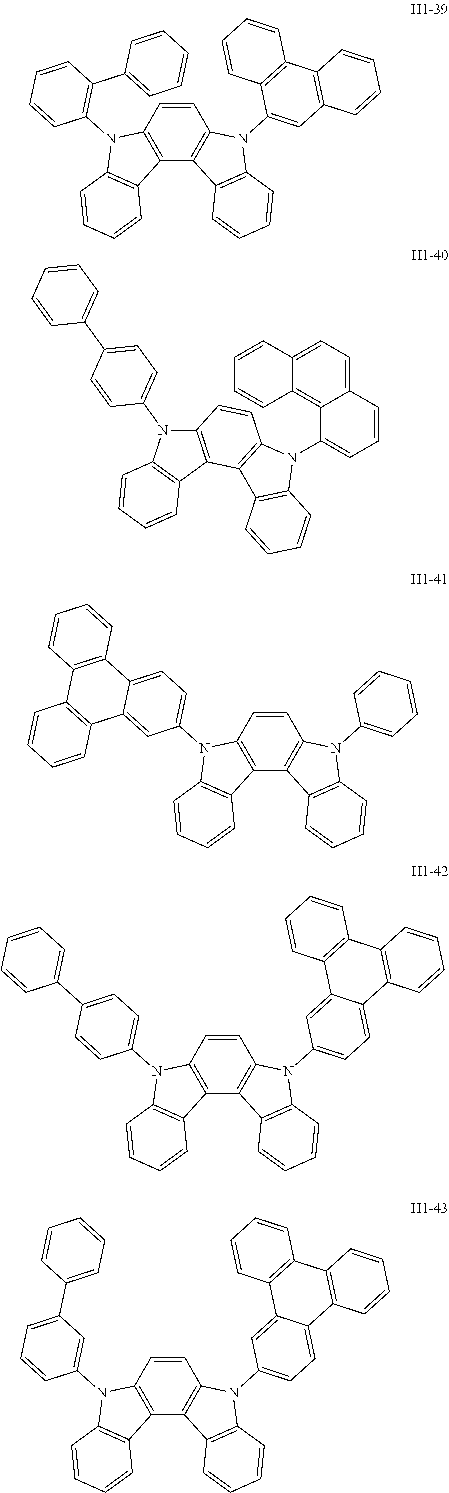

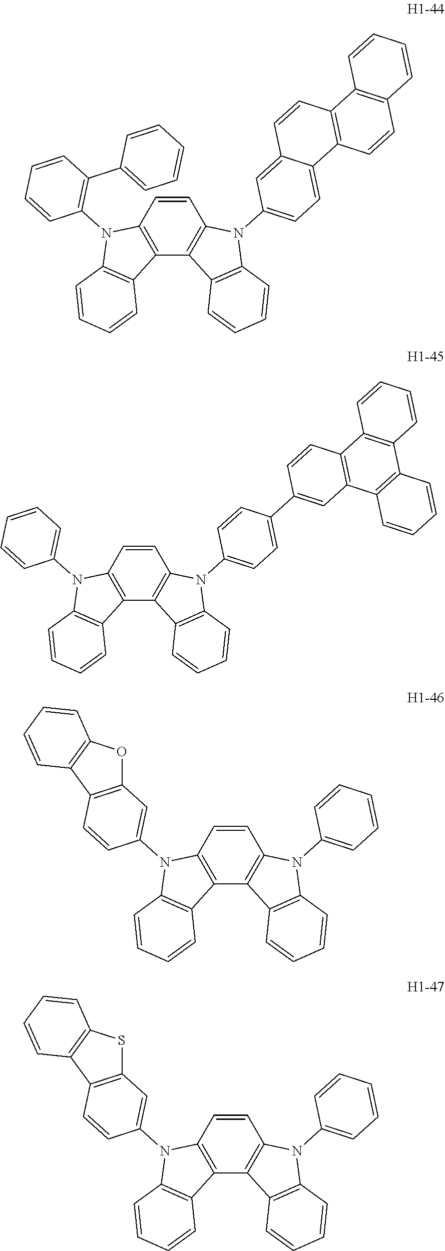

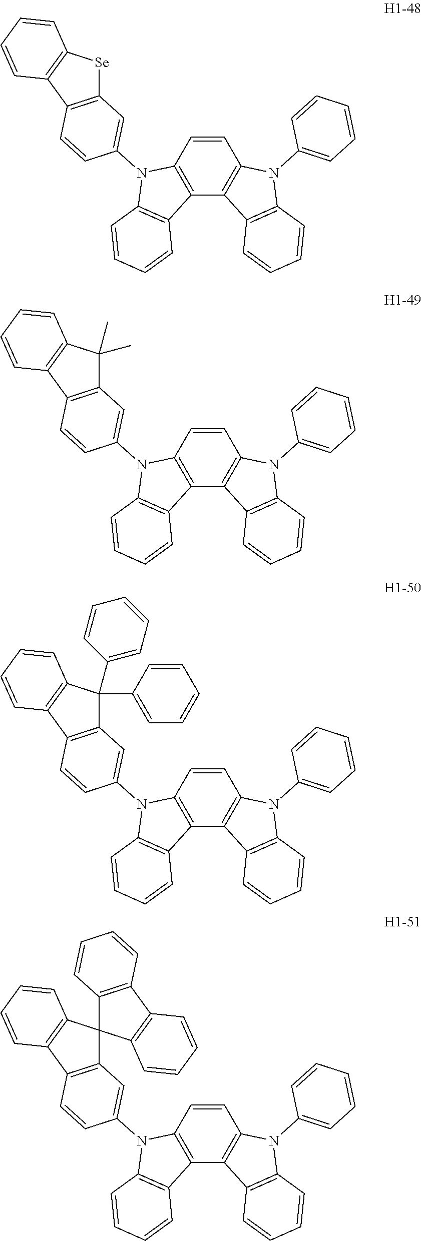

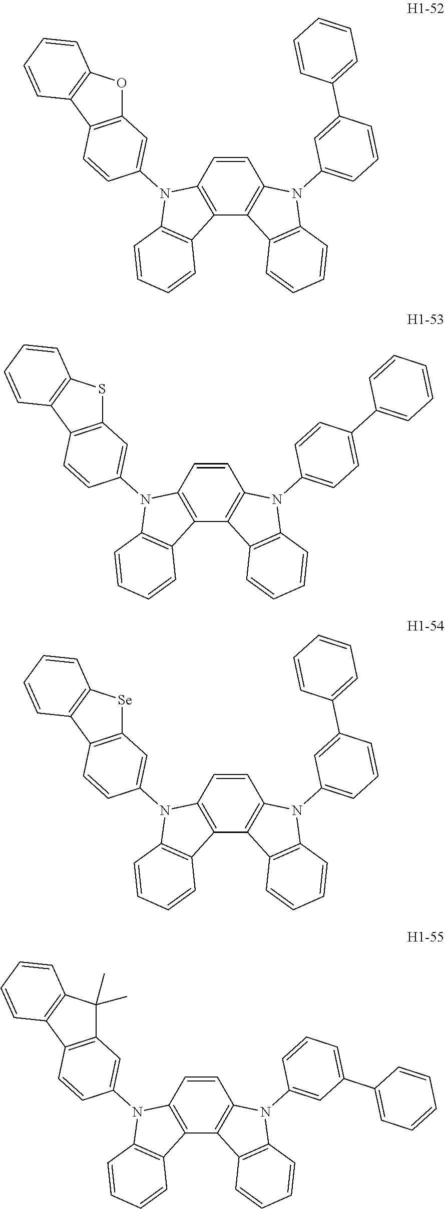 [SMILES: c1ccc(-c2cccc(-n3c4ccccc4c4c5c6ccccc6n(-c6ccc7c(c6)oc6ccccc67)c5ccc43)c2)cc1 ; c1ccc(-c2ccc(-n3c4ccccc4c4c5c6ccccc6n(-c6ccc7c(c6)sc6ccccc67)c5ccc43)cc2)cc1 ; c1ccc(-c2cccc(-n3c4ccccc4c4c5c6ccccc6n(-c6ccc7c(c6)[se]c6ccccc67)c5ccc43)c2)cc1 ; CC1(C)c2ccccc2-c2ccc(-n3c4ccccc4c4c5c6ccccc6n(-c6cccc(-c7ccccc7)c6)c5ccc43)cc21]

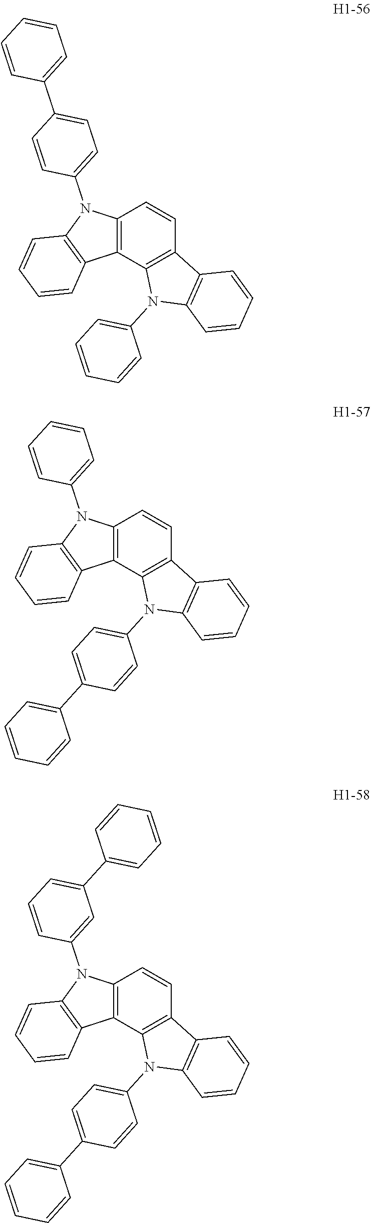 [SMILES: c1ccc(-c2ccc(-n3c4ccccc4c4c3ccc3c5ccccc5n(-c5ccccc5)c34)cc2)cc1 ; c1ccc(-c2ccc(-n3c4ccccc4c4ccc5c(c6ccccc6n5-c5ccccc5)c43)cc2)cc1 ; c1ccc(-c2ccc(-n3c4ccccc4c4ccc5c(c6ccccc6n5-c5cccc(-c6ccccc6)c5)c43)cc2)cc1]

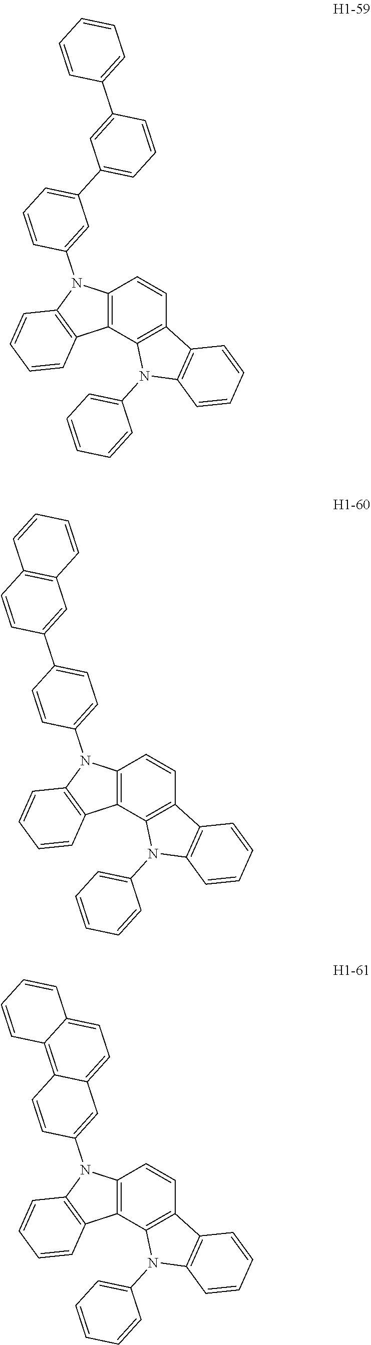 [SMILES: c1ccc(-c2cccc(-c3cccc(-n4c5ccccc5c5c4ccc4c6ccccc6n(-c6ccccc6)c45)c3)c2)cc1 ; c1ccc(-n2c3ccccc3c3ccc4c(c5ccccc5n4-c4ccc(-c5ccc6ccccc6c5)cc4)c32)cc1 ; c1ccc(-n2c3ccccc3c3ccc4c(c5ccccc5n4-c4ccc5c(ccc6ccccc65)c4)c32)cc1]

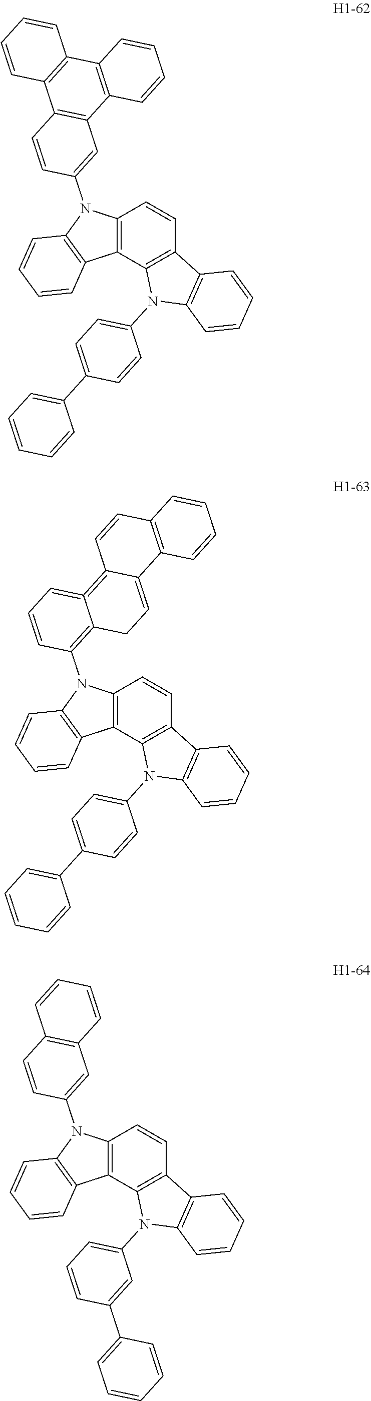

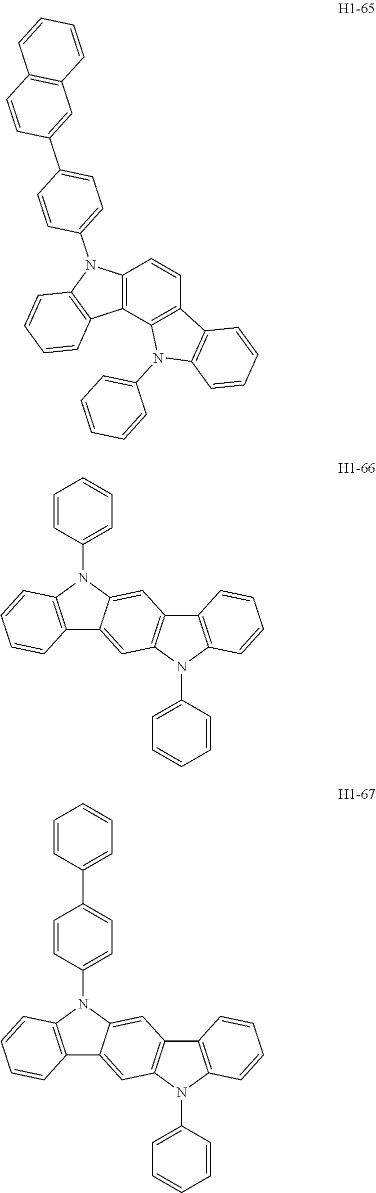

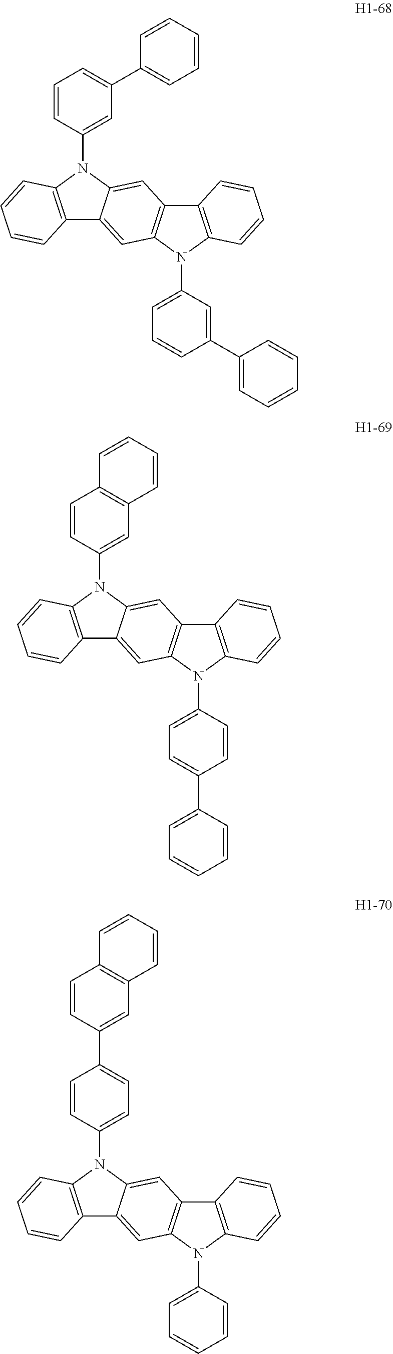 [SMILES: c1ccc(-c2cccc(-n3c4ccccc4c4cc5c(cc43)c3ccccc3n5-c3cccc(-c4ccccc4)c3)c2)cc1 ; c1ccc(-c2ccc(-n3c4ccccc4c4cc5c(cc43)c3ccccc3n5-c3ccc4ccccc4c3)cc2)cc1 ; c1ccc(-n2c3ccccc3c3cc4c(cc32)c2ccccc2n4-c2ccc(-c3ccc4ccccc4c3)cc2)cc1]

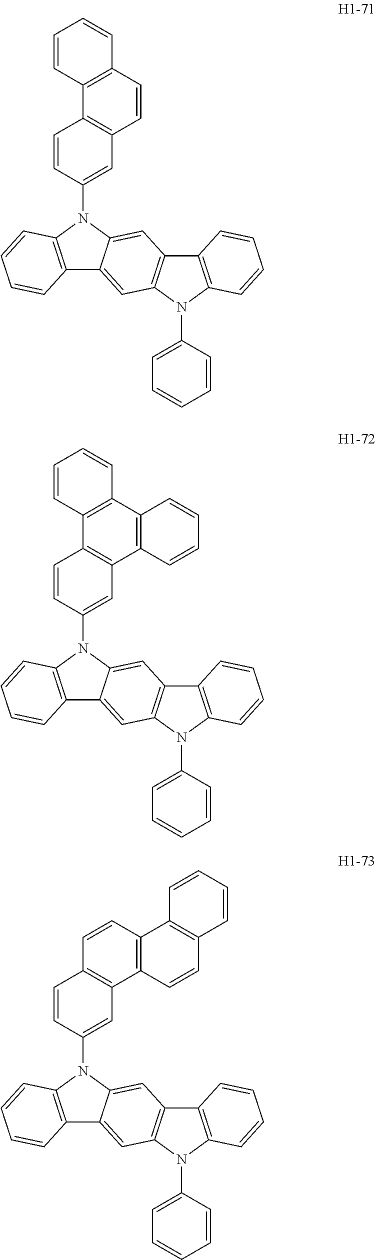

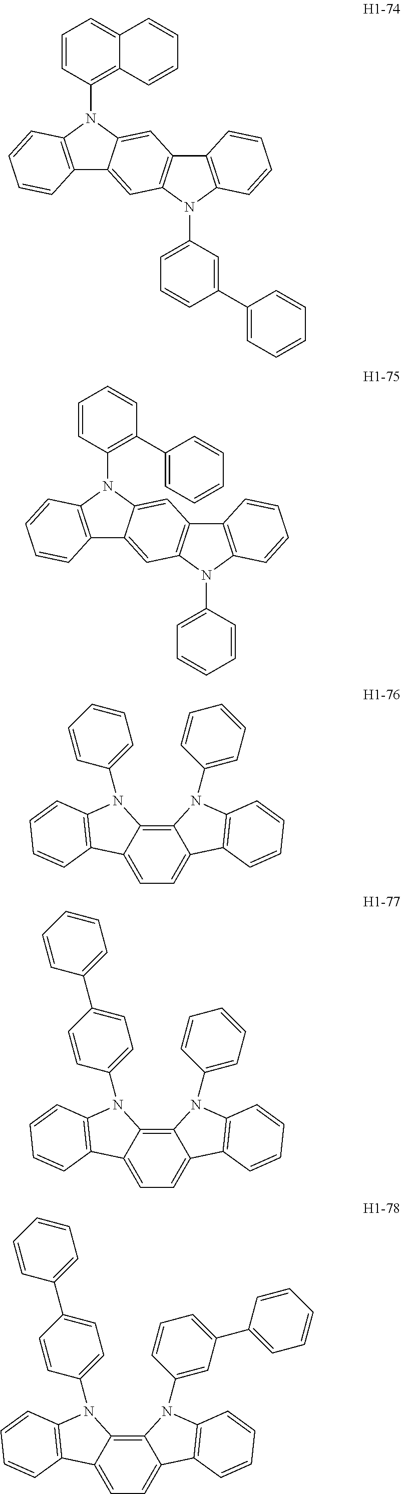

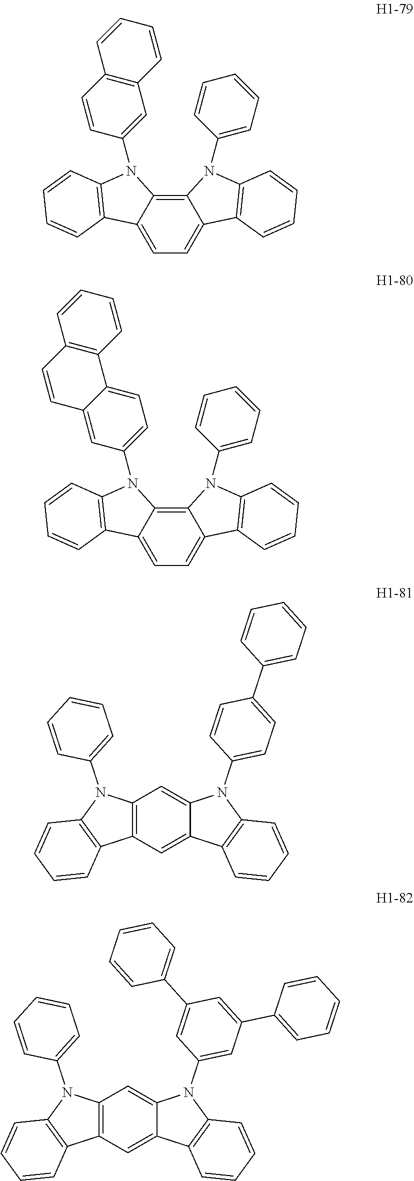 [SMILES: c1ccc(-n2c3ccccc3c3ccc4c5ccccc5n(-c5ccc6ccccc6c5)c4c32)cc1 ; c1ccc(-n2c3ccccc3c3ccc4c5ccccc5n(-c5ccc6c(ccc7ccccc76)c5)c4c32)cc1 ; c1ccc(-c2ccc(-n3c4ccccc4c4cc5c6ccccc6n(-c6ccccc6)c5cc43)cc2)cc1 ; c1ccc(-c2cc(-c3ccccc3)cc(-n3c4ccccc4c4cc5c6ccccc6n(-c6ccccc6)c5cc43)c2)cc1]

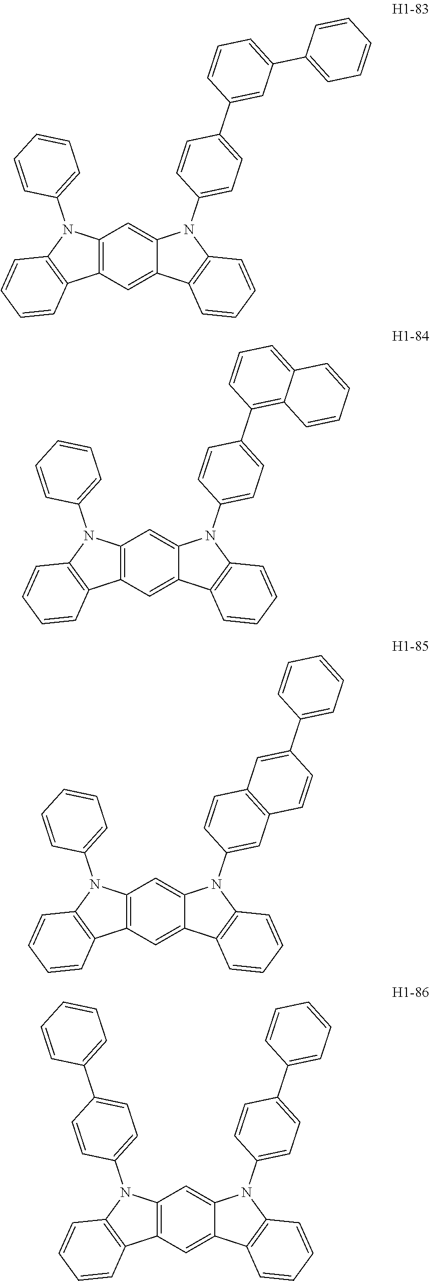

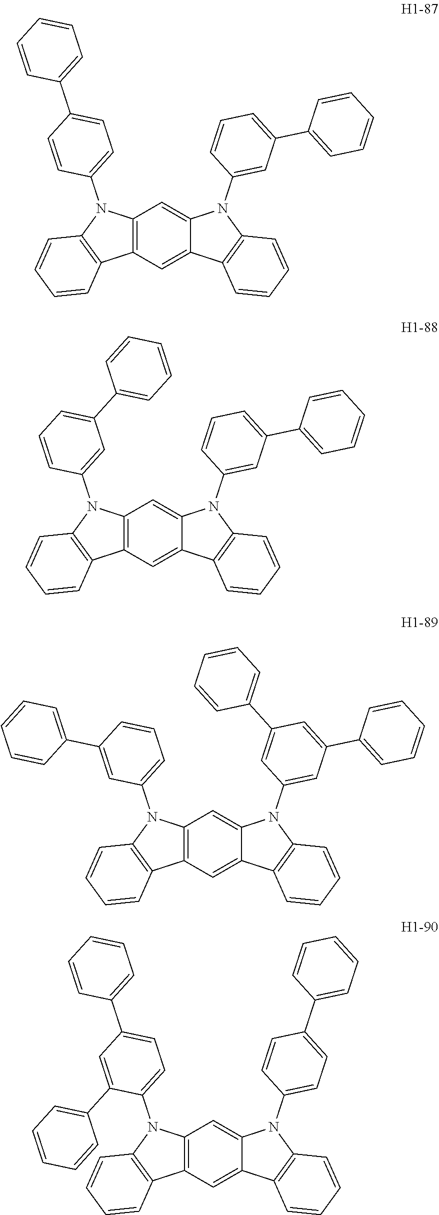

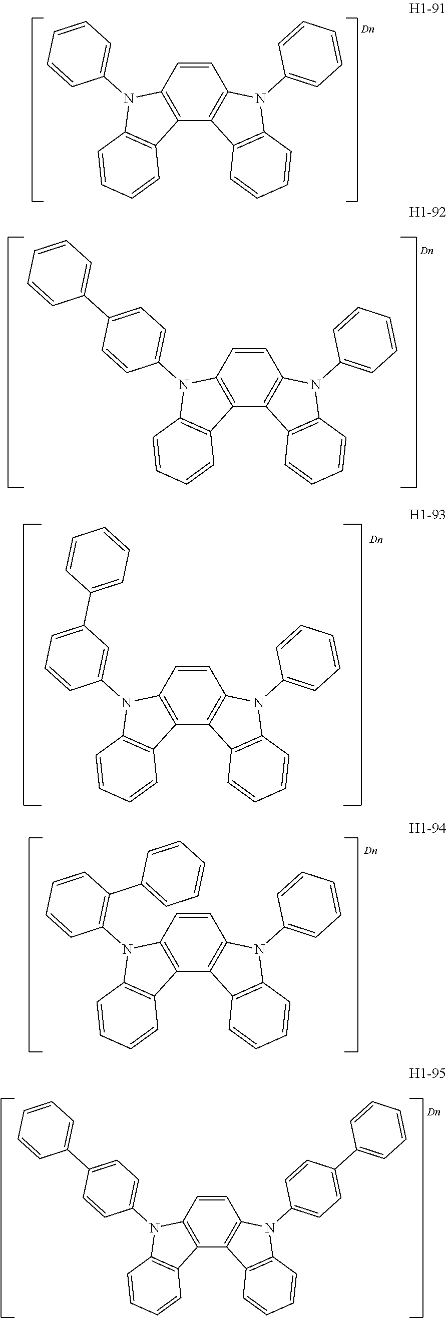

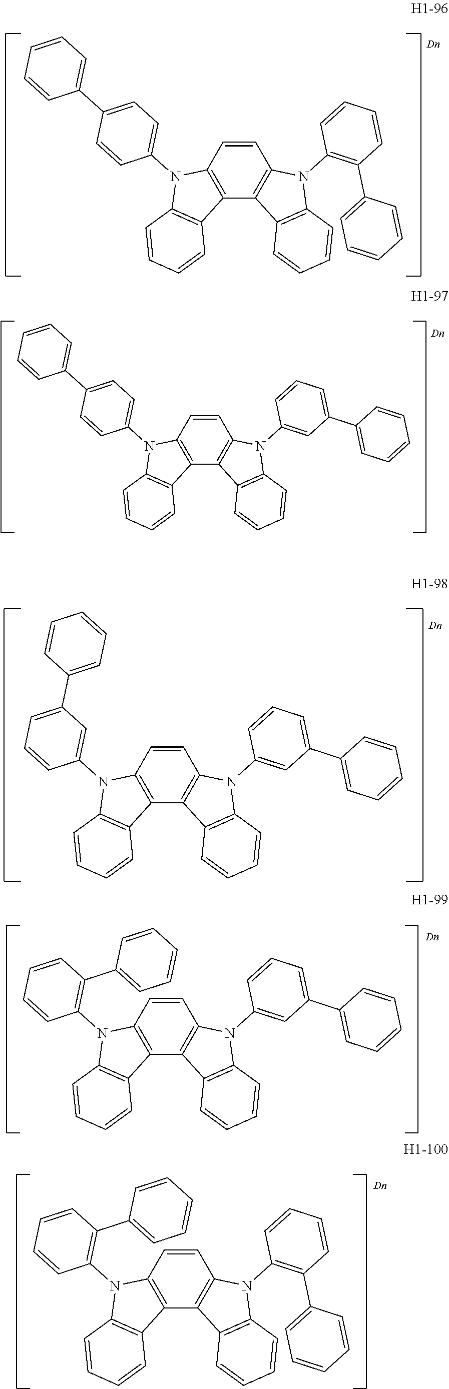

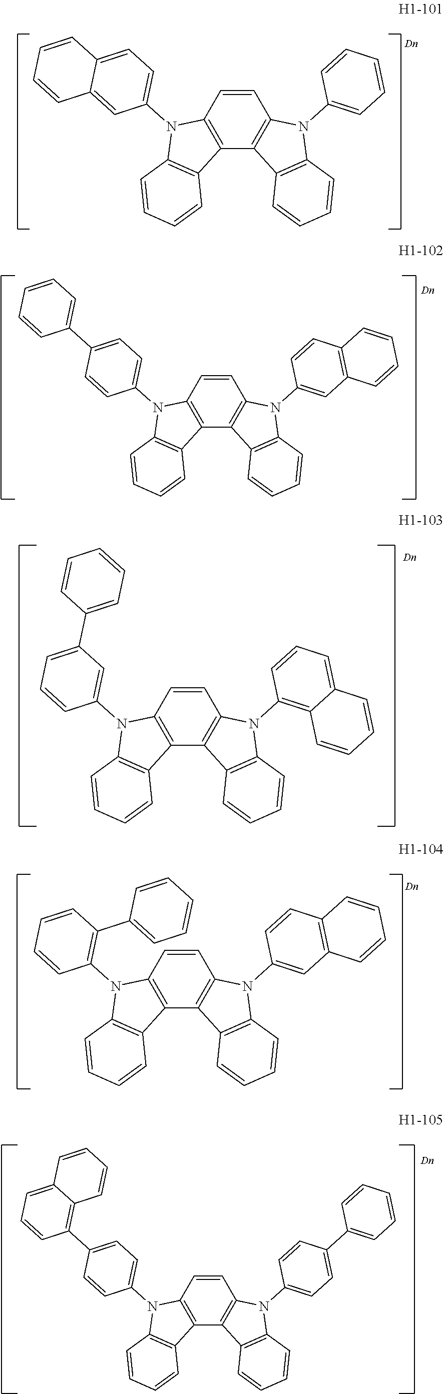

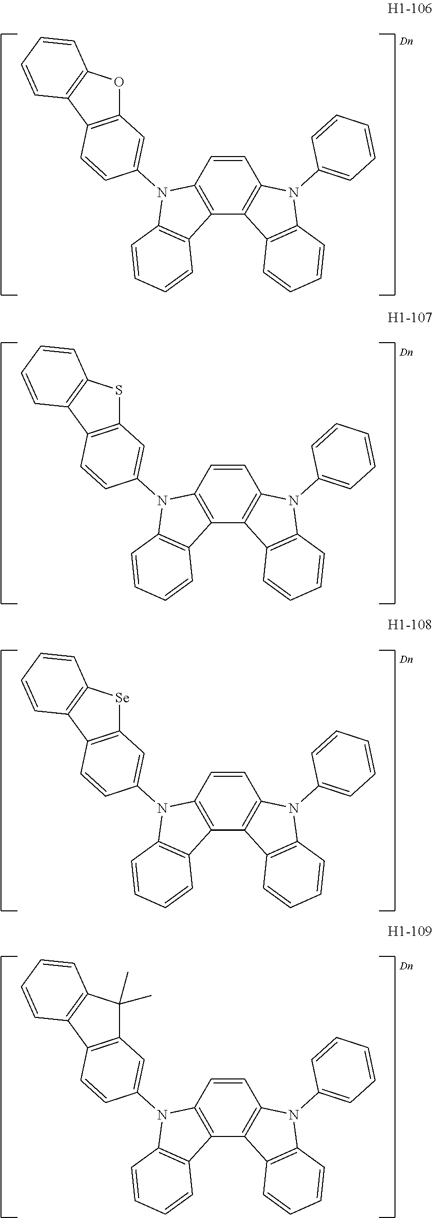

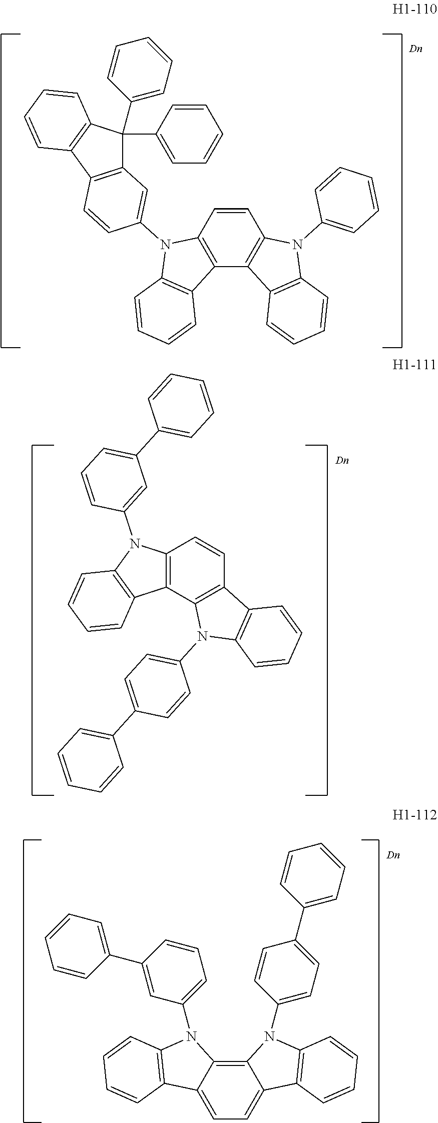

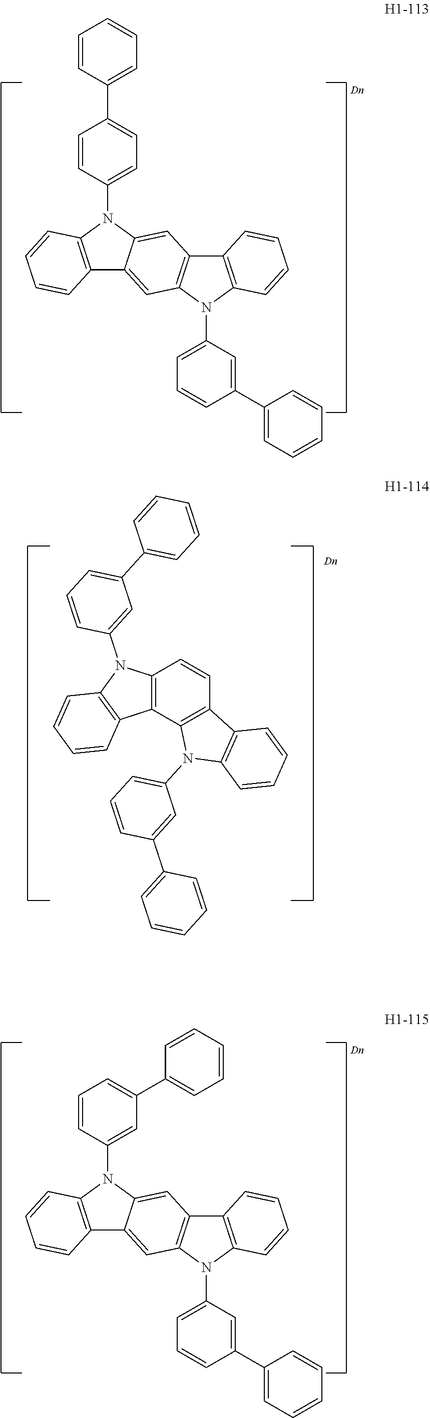

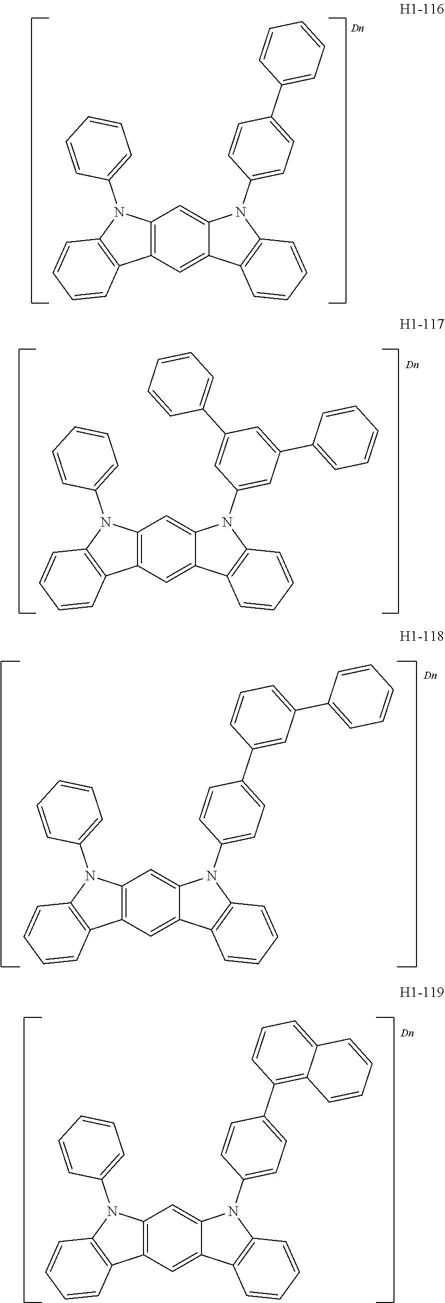

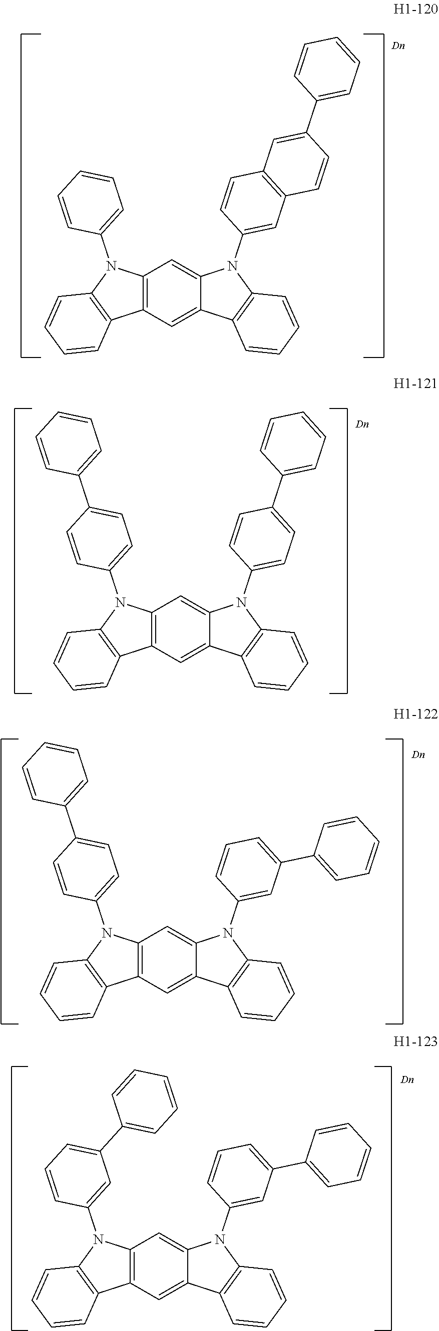

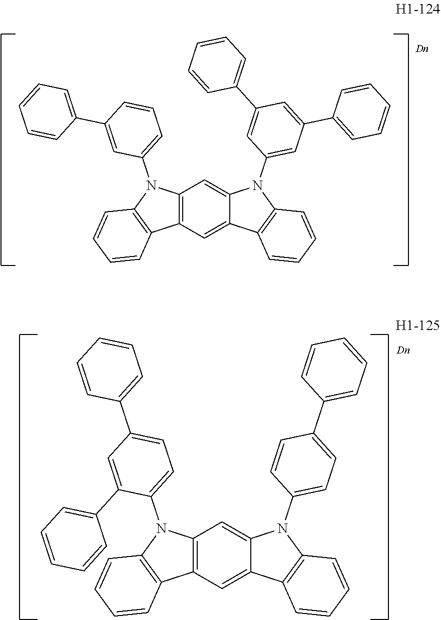

In the compounds above, Dₙ means that n number of hydrogens is replaced with deuterium, wherein n is an integer of 1 or more, and the upper limit of n is determined according to the number of hydrogens that may be substituted for each compound.

Hereinafter, an organic electroluminescent device will be described to which the aforementioned compound and/or an organic electroluminescent compound comprising the same is (are) applied.

The organic electroluminescent device according to one embodiment includes a first electrode, a second electrode, and at least one organic layer(s) interposed between the first electrode and the second electrode. The organic layer includes at least one light-emitting layer(s).

According to one embodiment, the at least one light-emitting layer may comprise a compound represented by Formula 1. According to another embodiment, the at least one light-emitting layer may comprise a compound represented by Formula 1 as a first host compound and a compound represented by Formula 2 or 3 as a second host compound, respectively, wherein the weight ratio of the first host compound to the second host compound may be in the range of about 1:99 to about 99:1, preferably about 10:90 to about 90:10, more preferably about 30:70 to about 70:30, more preferably about 40:60 to about 60:40, and even more preferably about 50:50 in the light-emitting layer.

According to one embodiment, the organic electroluminescent material of the present disclosure comprises at least one compound of Compounds C-1 to C-100, and at least one compound of Compounds H2-1 to H2-290, or at least one compound of Compounds H1-1 to H1-125. The organic electroluminescent material may be included in the same organic layer, for example, the same light-emitting layer, or may each be included in different light-emitting layers.

The organic layer may further comprise at least one layer selected from a hole injection layer, a hole transport layer, a hole auxiliary layer, a light-emitting auxiliary layer, an electron transport layer, an electron injection layer, an interlayer, a hole-blocking layer, an electron-blocking layer and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron-blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron-blocking material. Also, the electron transport layer, the electron injection layer, the electron buffer layer, or the hole-blocking layer may contain the azine-based compound as an electron transport material, an electron injection material, an electron buffer material, or a hole-blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent material according to one embodiment may be used as light-emitting materials for a white organic light-emitting device. The white organic light-emitting device has various suggested structures such as a parallel side-by-side arrangement method, a stacking arrangement method, or a CCM (color conversion material) method, etc., depending on the arrangement of R (red), G (green), YG (yellowish green), or B (blue) light-emitting units. In addition, the organic electroluminescent material according to one embodiment may also be applied to the organic electroluminescent device comprising a QD (quantum dot).

One of the first electrode and the second electrode may be an anode, and the other may be a cathode, wherein the first electrode and the second electrode may each be formed as a transmissive conductive material, a transflective conductive material, or a reflective conductive material. The organic electroluminescent device may be a top emission type, a bottom emission type, or a both-sides emission type depending on the kinds of the material forming the first electrode and the second electrode.

A hole injection layer, a hole transport layer, an electron-blocking layer, or a combination thereof can be used between the anode and the light-emitting layer. The hole injection layer may be multi-layers in order to lower the hole injection barrier (or hole injection voltage) from the anode to the hole transport layer or the electron-blocking layer, wherein each of the multi-layers may use two compounds simultaneously. Also, the hole injection layer may be doped as a p-dopant. Also, the electron-blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron-blocking layer may be multi-layers, wherein each layer may use a plurality of compounds.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ (1≤X≤2), AlOₓ (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation; thus, it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare earth metals, and mixtures thereof. Also, a reductive dopant layer may be employed as a charge generating layer to prepare an organic electroluminescent device having two or more light-emitting layers and emitting white light.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. According to one embodiment, the organic electroluminescent device may include a plurality of light-emitting units, and each of the light-emitting units may include a hole transport band, a light-emitting layer, and an electron transport band, and the hole transport band may include a hole injection layer and a hole transport layer, and the electron transfer zone may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. This may include one or more charge generation layers located between each light-emitting unit. The charge generation layer refers to the layer in which holes and electrons are generated when voltage is applied. When there are three or more light-emitting units, a charge generation layer may be located between each light-emitting unit. Here, the plurality of charge generation layers may be the same as or different from each other. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an n-type charge generation layer and a p-type charge generation layer, and the n-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The p-type charge generation layer may be made of a metal or an organic material doped with a p-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the p-type dopant and host materials used in the p-type doped organic material.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may preferably be a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

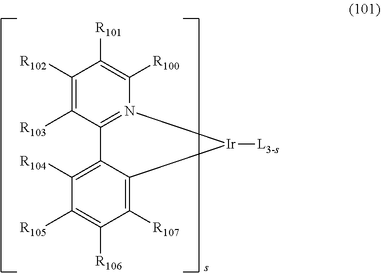

In Formula 101,
L is any one selected from the following structures 1 to 3:

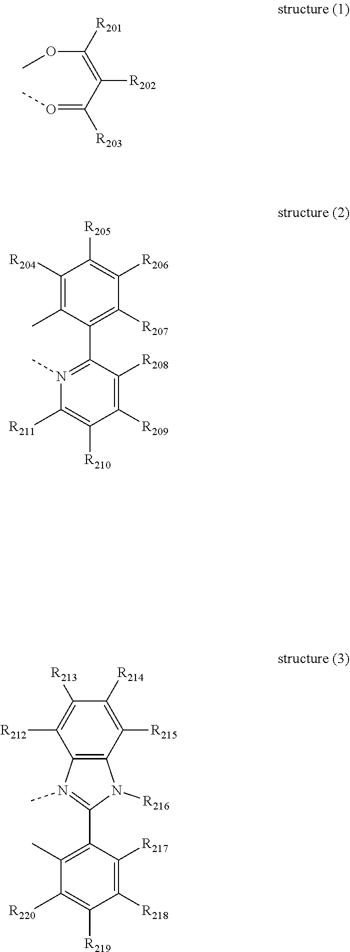

in structures 1 to 3,
R₁₀₀ to R₁₀₃ each independently represent hydrogen, deuterium, halogen, (C₁-C₃₀)alkyl unsubstituted or substituted with deuterium and/or halogen, a substituted or unsubstituted (C₃-C₃₀)cycloalkyl, a substituted or unsubstituted (C₆-C₃₀)aryl, cyano, a substituted or unsubstituted (3- to 30-membered)heteroaryl, or a substituted or unsubstituted (C₁-C₃₀)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent hydrogen, deuterium, halogen, (C₁-C₃₀)alkyl unsubstituted or substituted with deuterium and/or halogen, a substituted or unsubstituted (C₃-C₃₀)cycloalkyl, a substituted or unsubstituted (C₆-C₃₀)aryl, a substituted or unsubstituted (3- to 30-membered)heteroaryl, cyano, or a substituted or unsubstituted (C₁-C₃₀)alkoxy; or may be linked to an adjacent substituent(s) to form a substituted or unsubstituted ring(s), for example, to form a substituted or unsubstituted ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent hydrogen, deuterium, halogen, (C₁-C₃₀)alkyl unsubstituted or substituted with deuterium and/or halogen, a substituted or unsubstituted (C₃-C₃₀)cycloalkyl, or a substituted or unsubstituted (C₆-C₃₀)aryl; or may be linked to an adjacent substituent(s) to form a substituted or unsubstituted ring(s); and
s represents an integer of 1 to 3.

Specifically, the specific examples of the dopant compound include the following, but are not limited thereto.

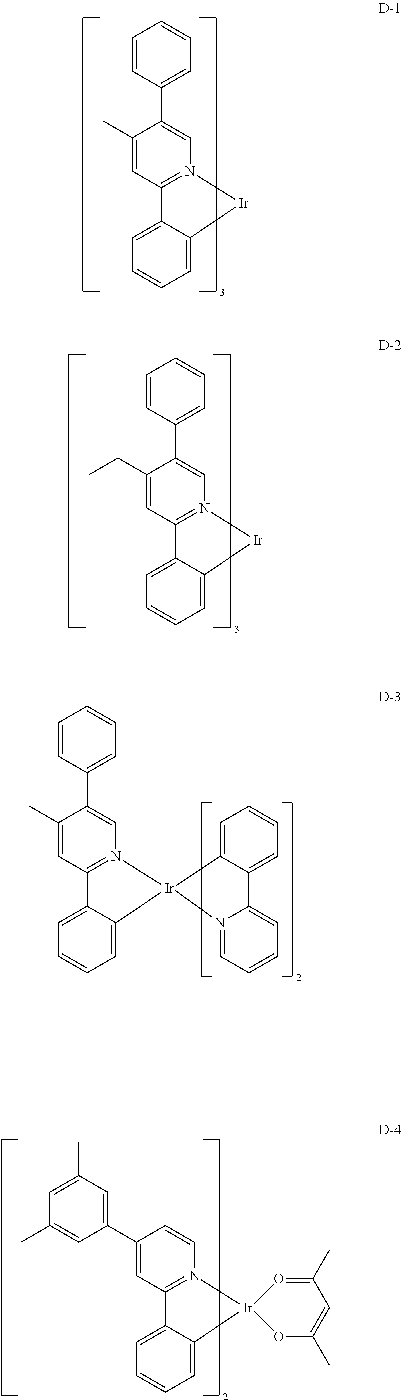

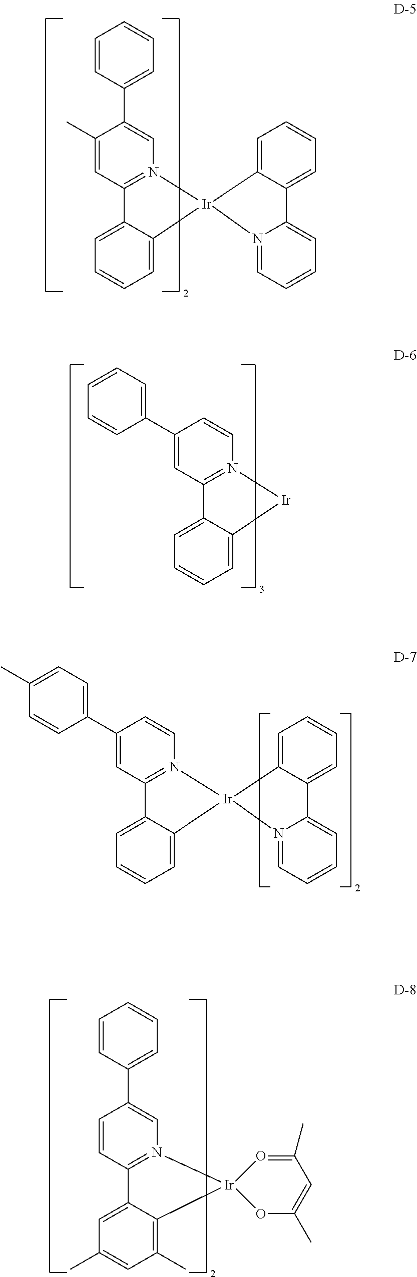

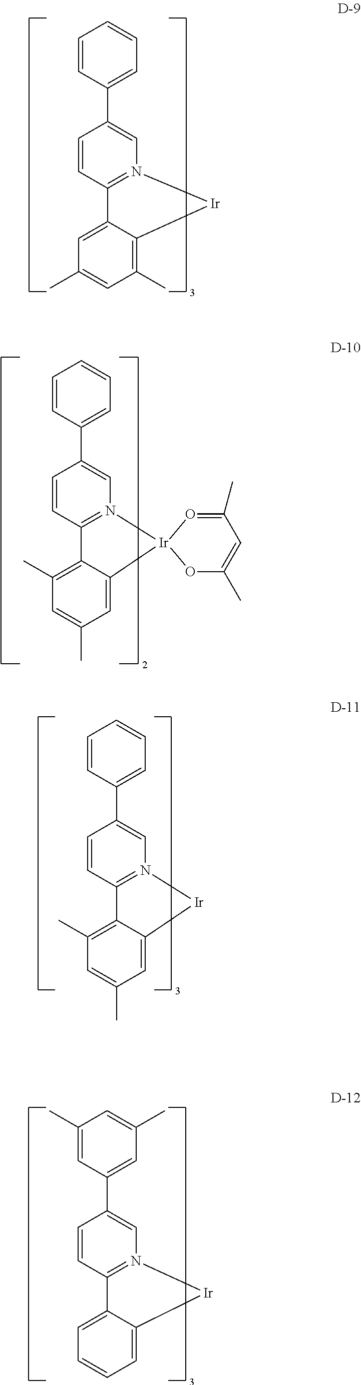

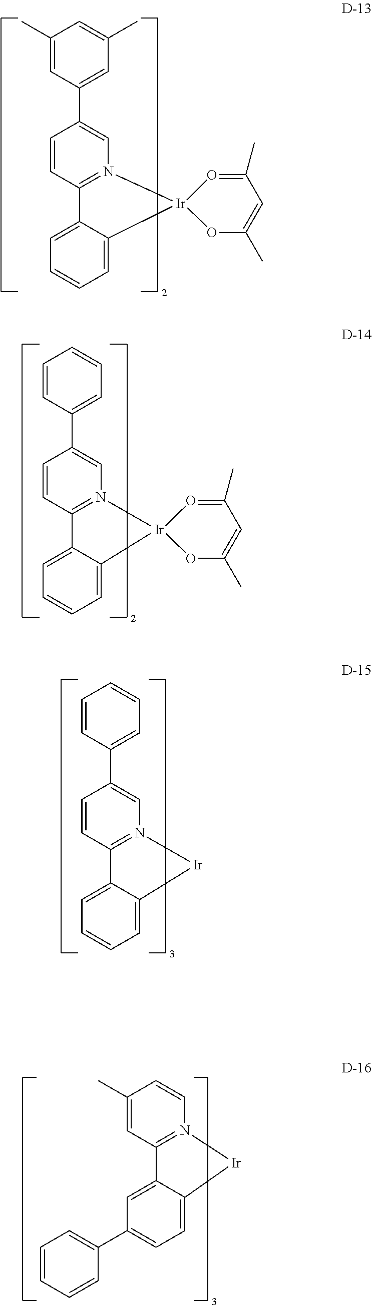

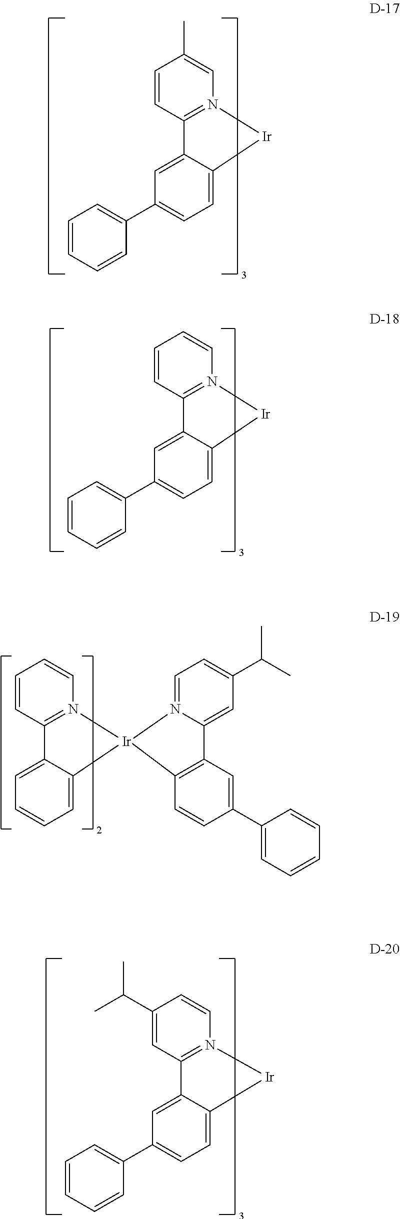

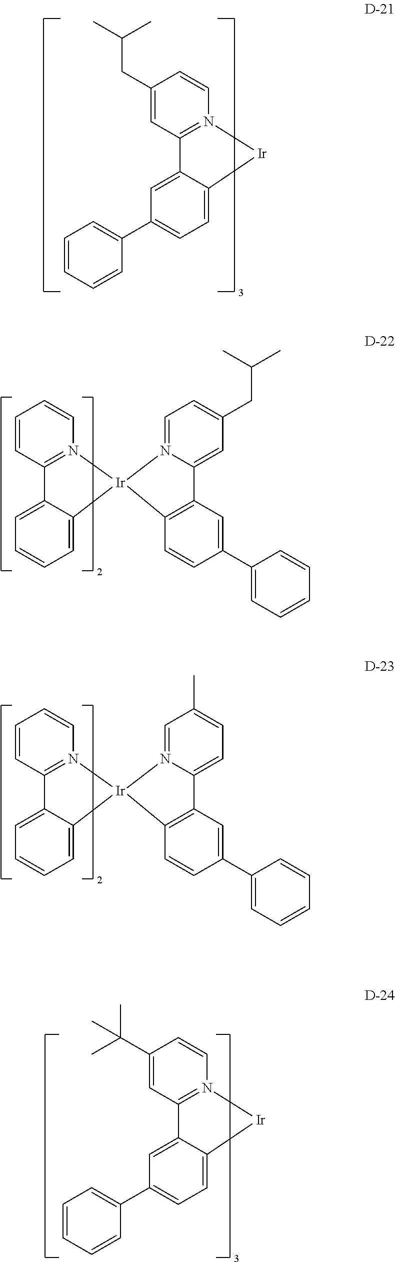

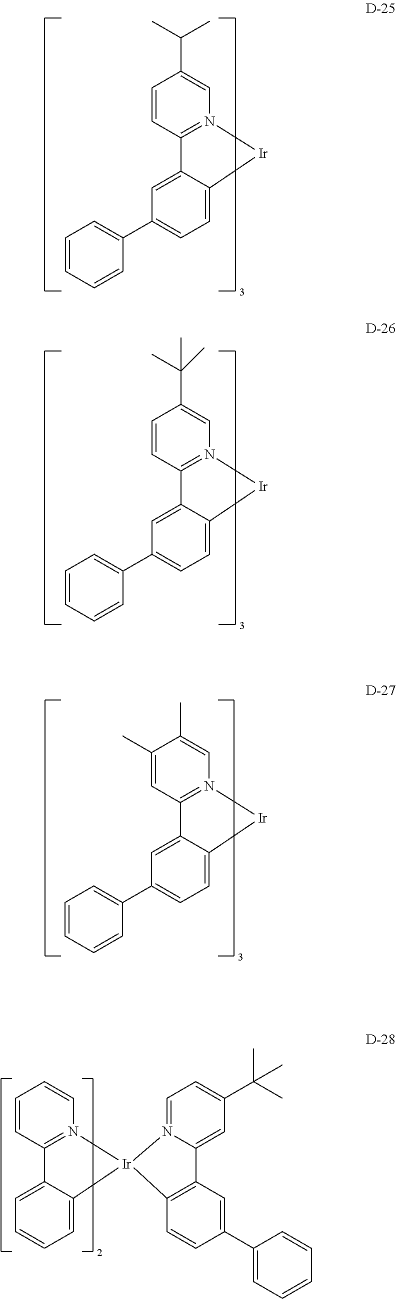

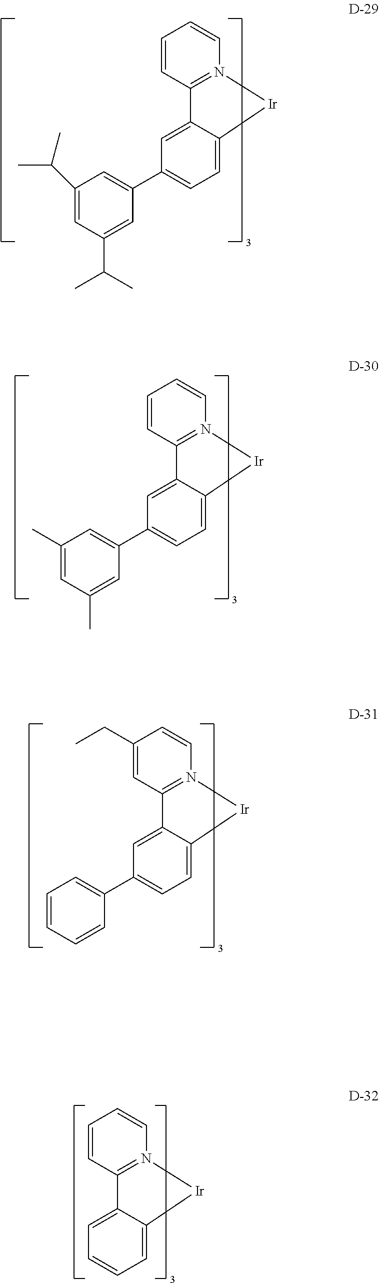

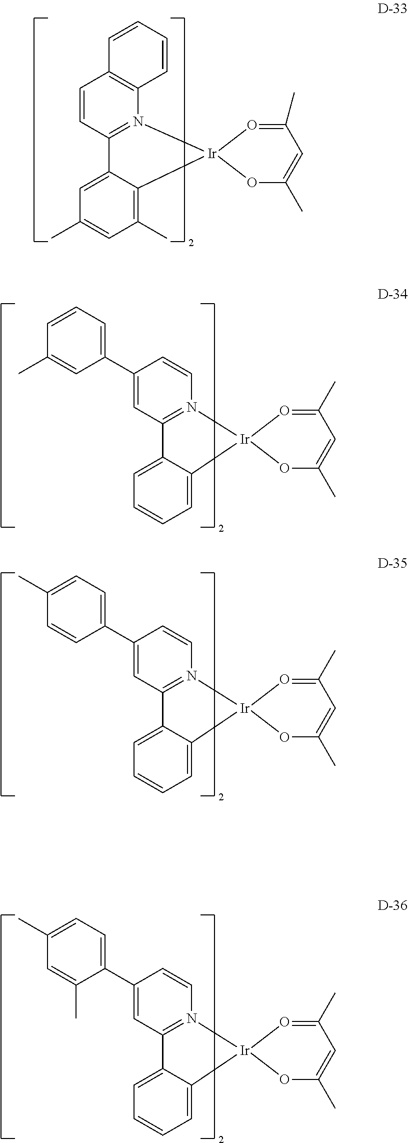

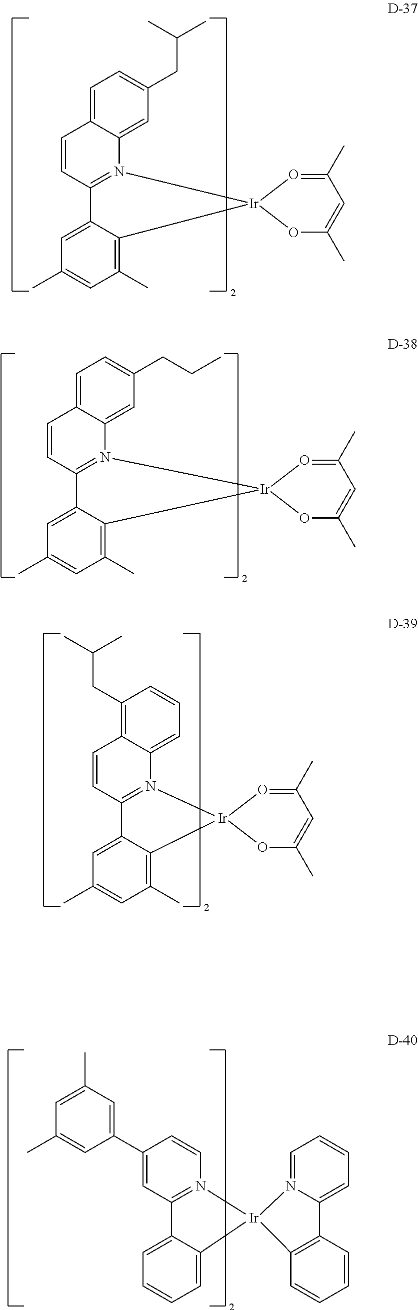

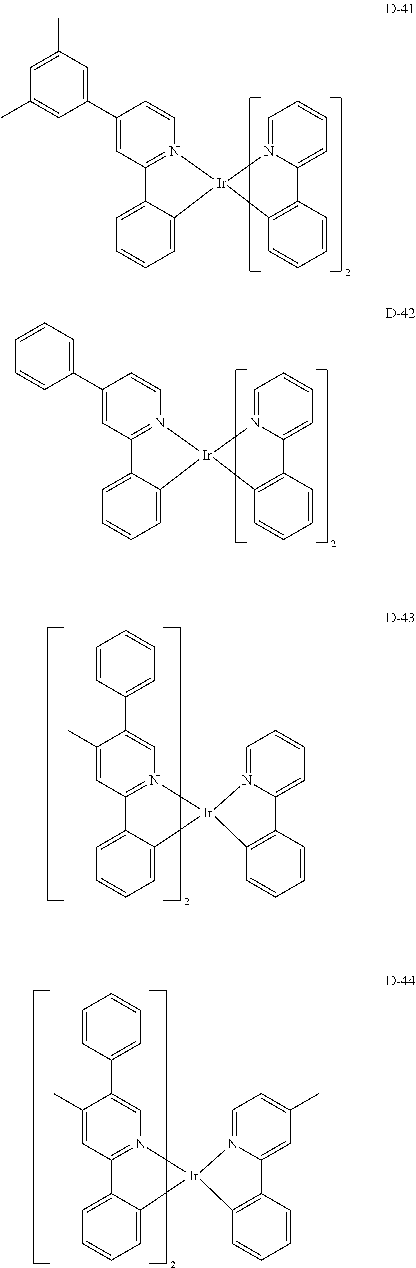

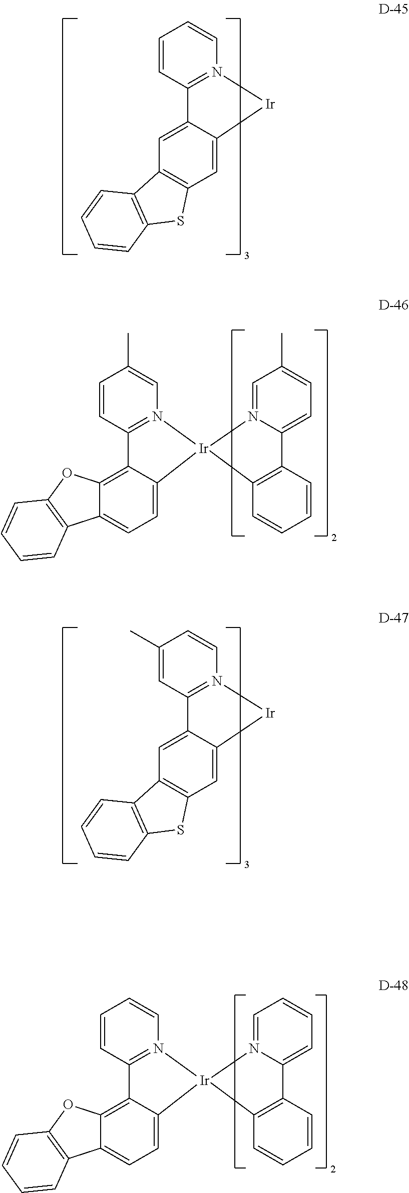

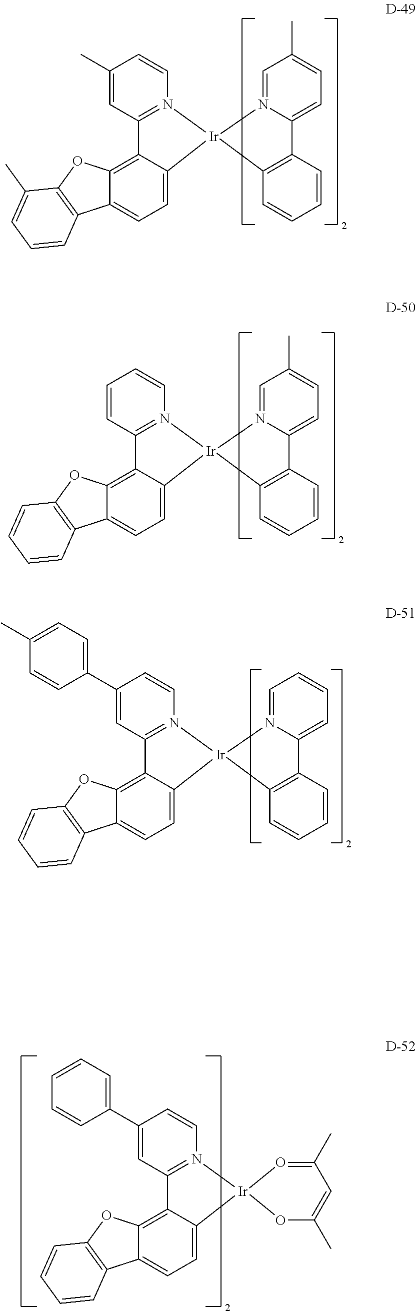

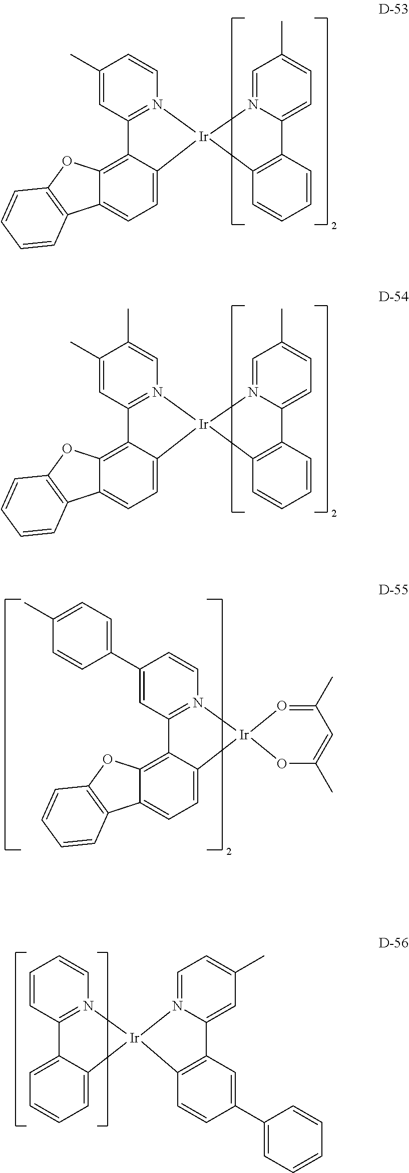

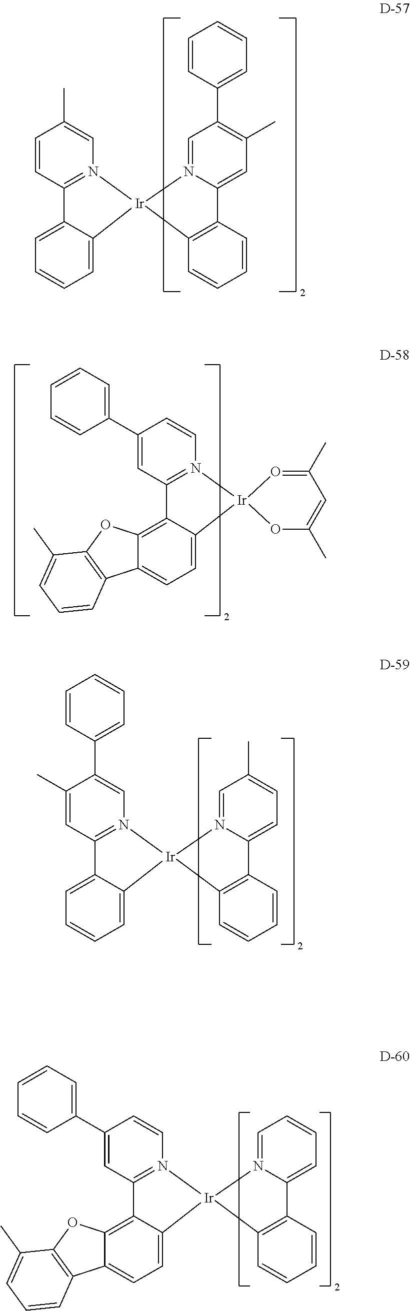

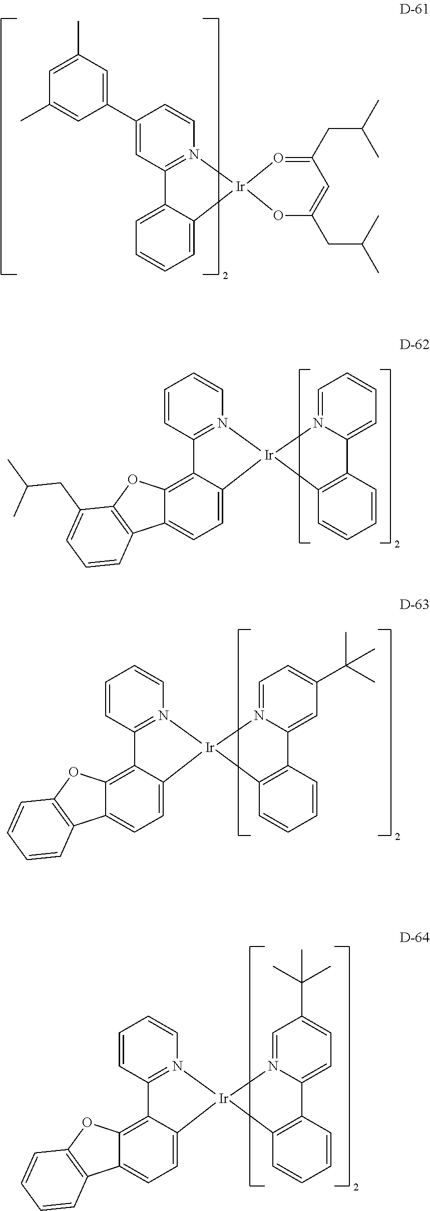

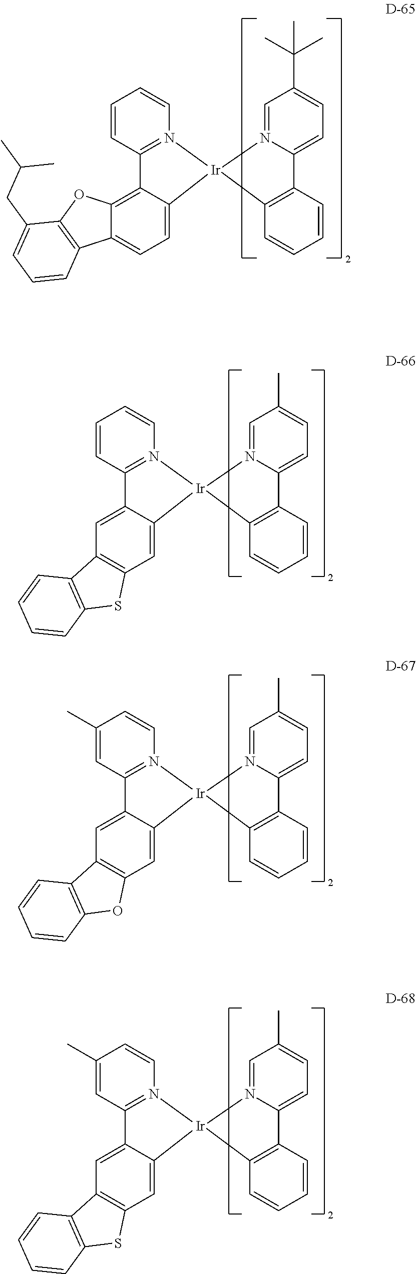

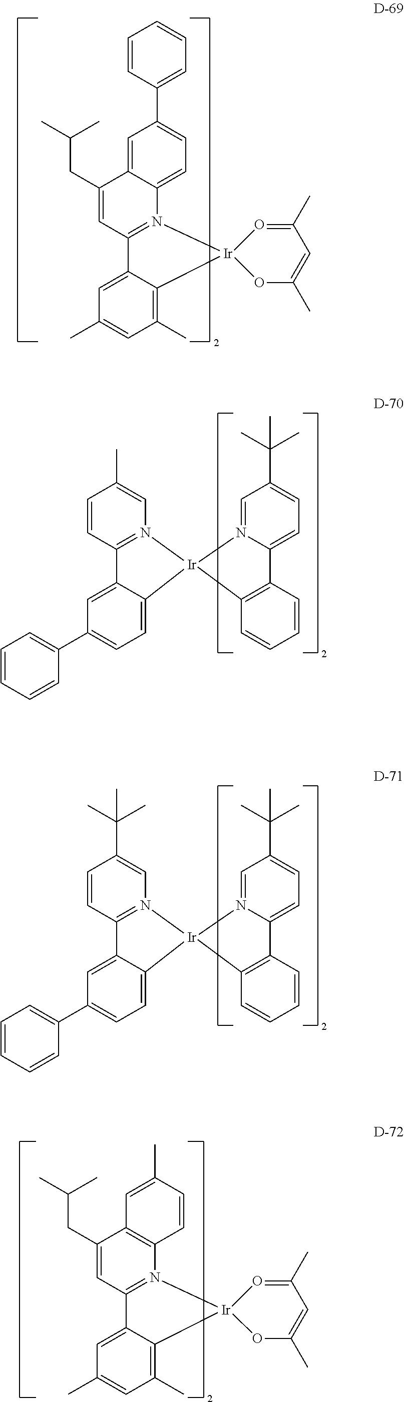

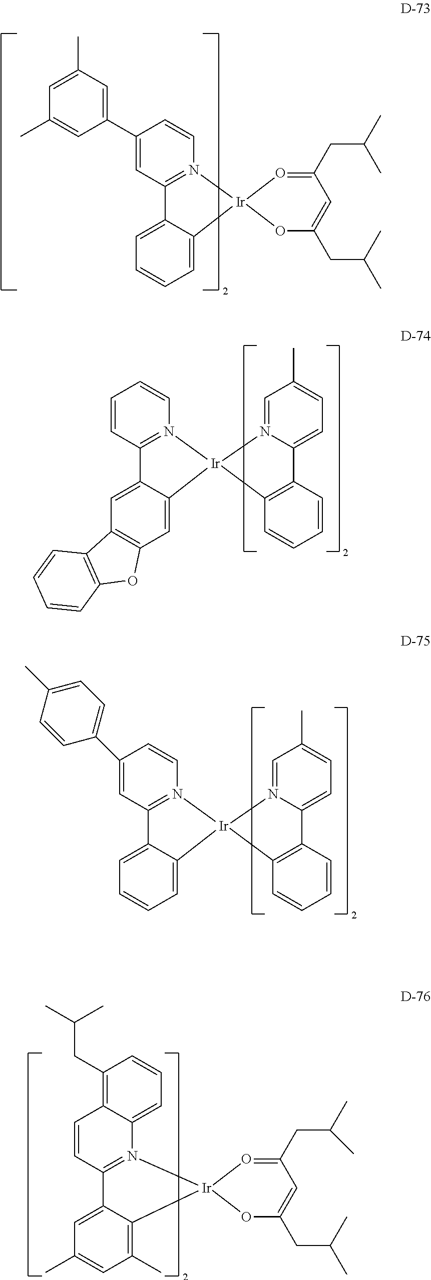

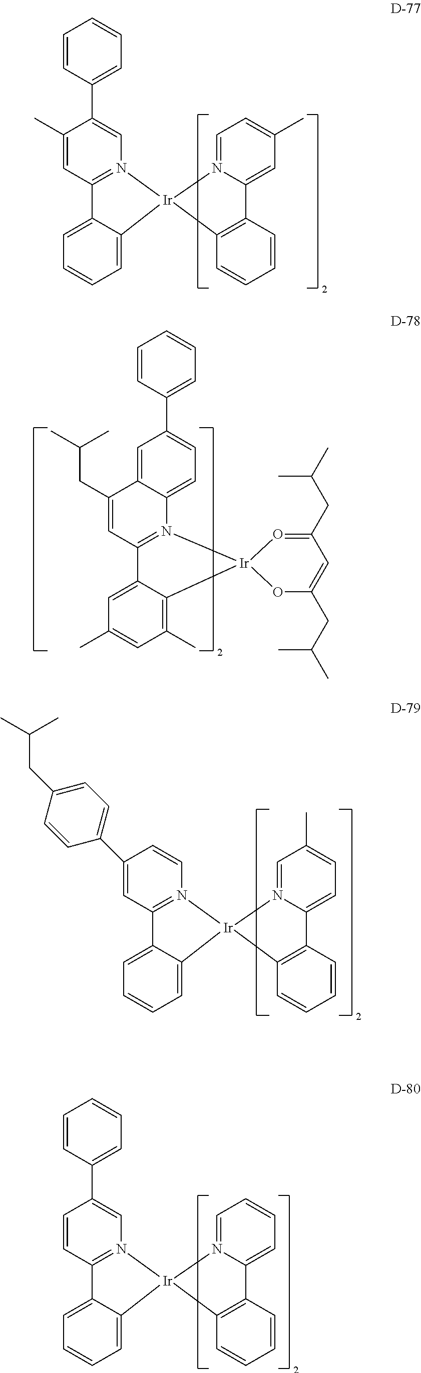

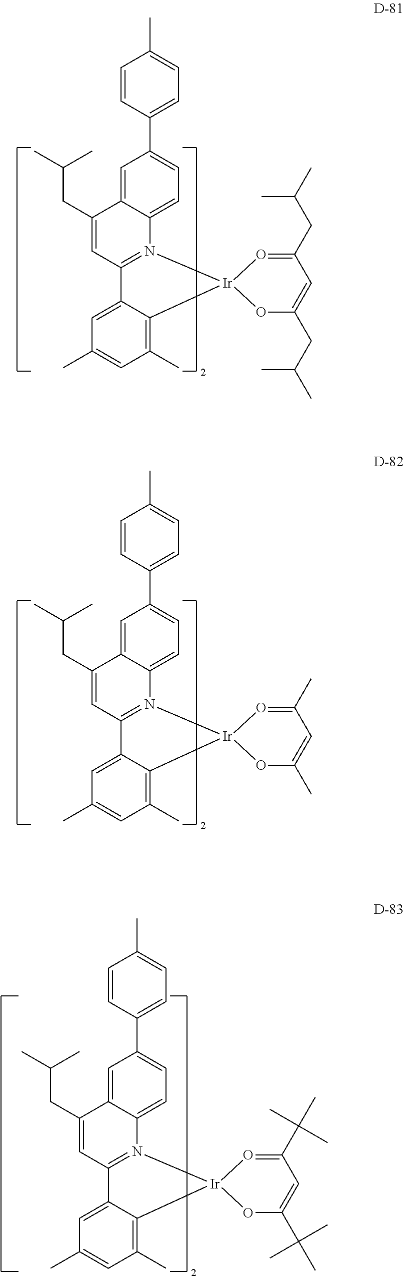

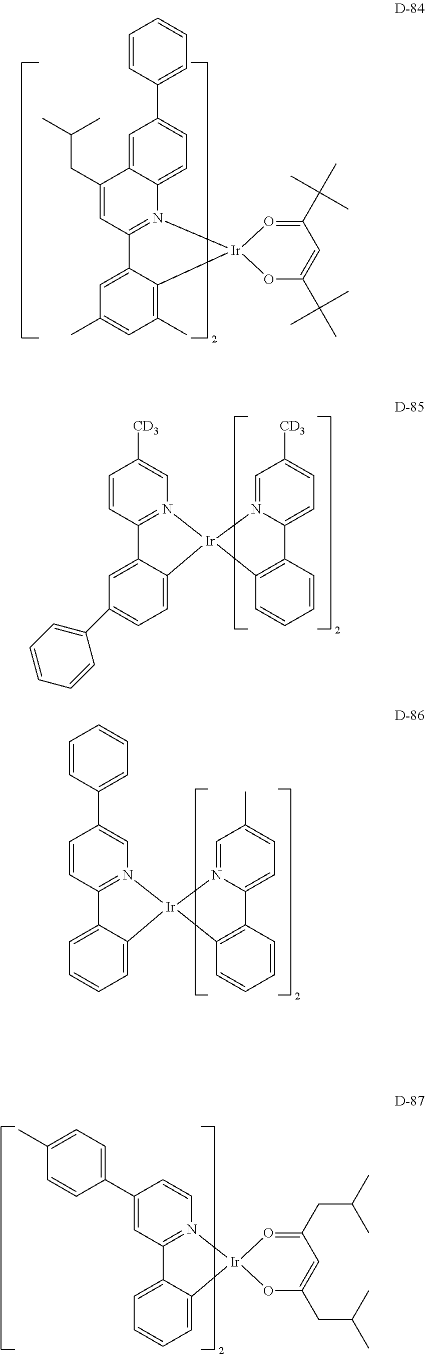

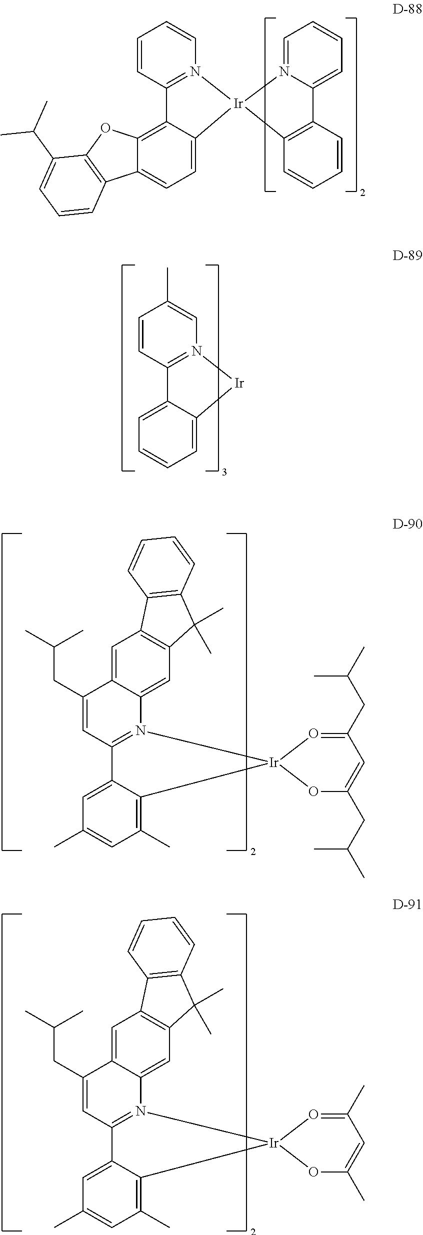

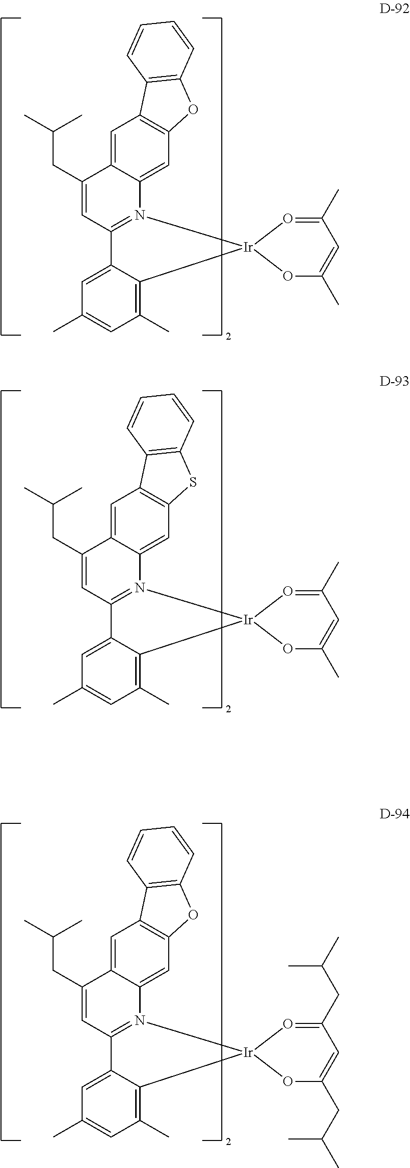

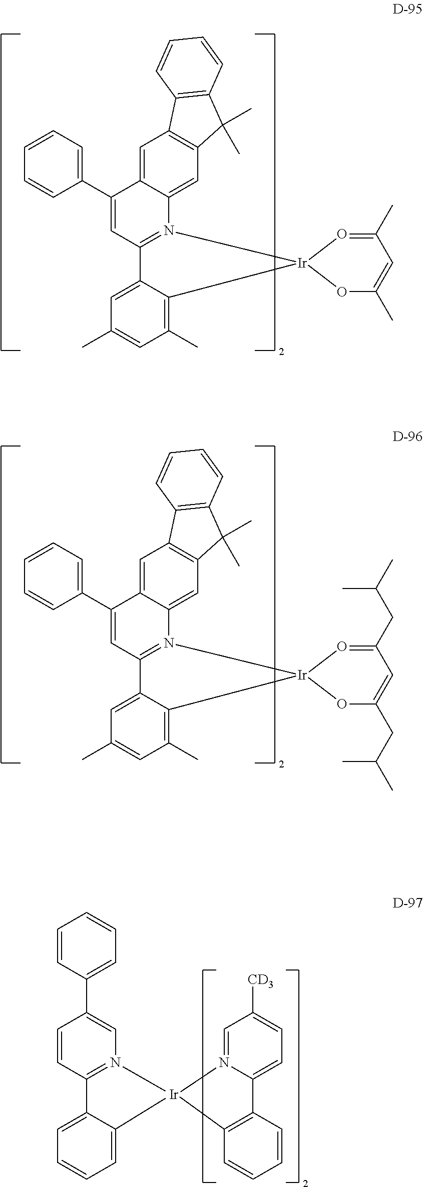

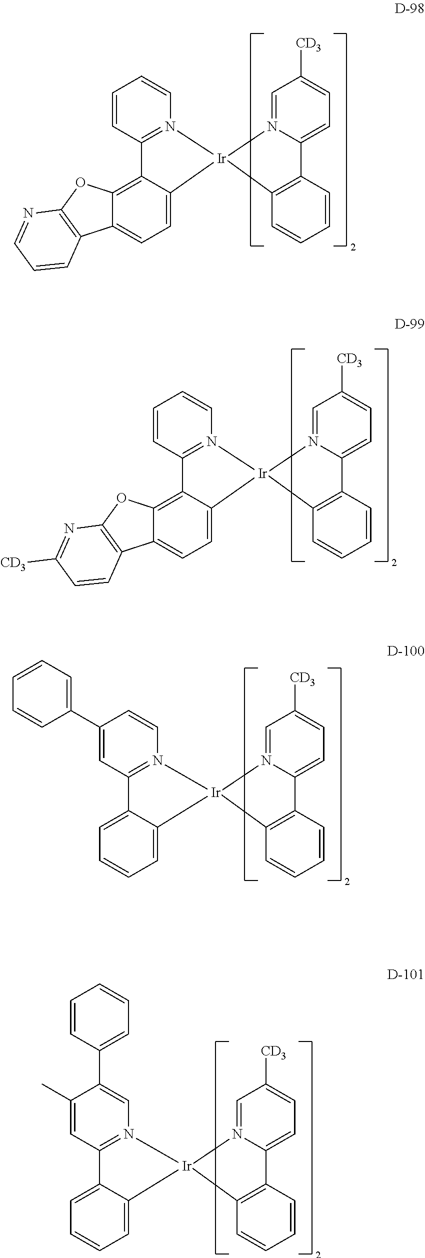

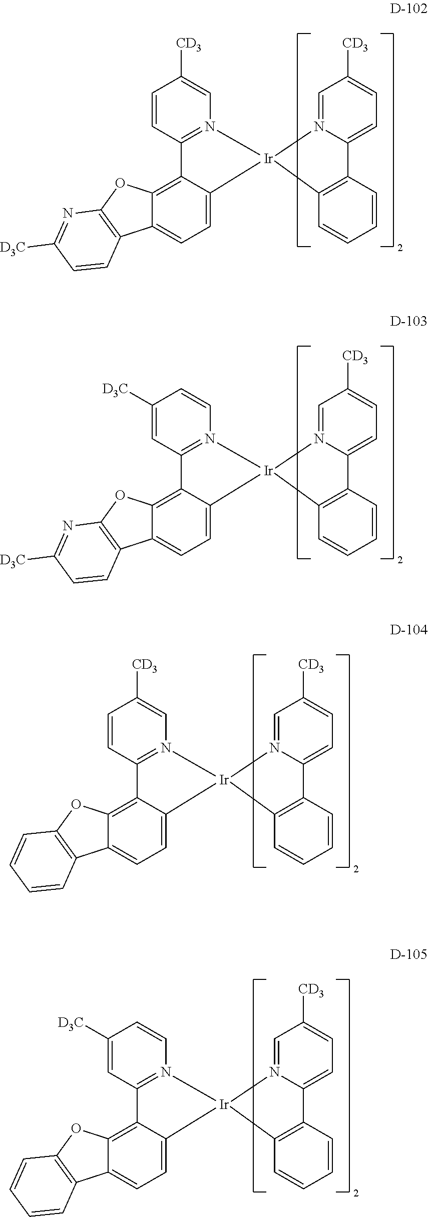

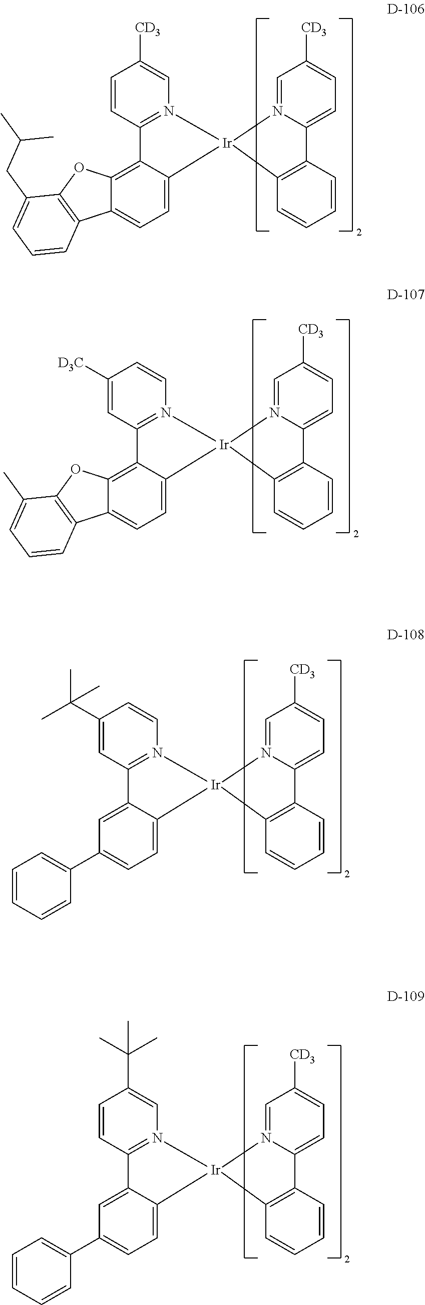

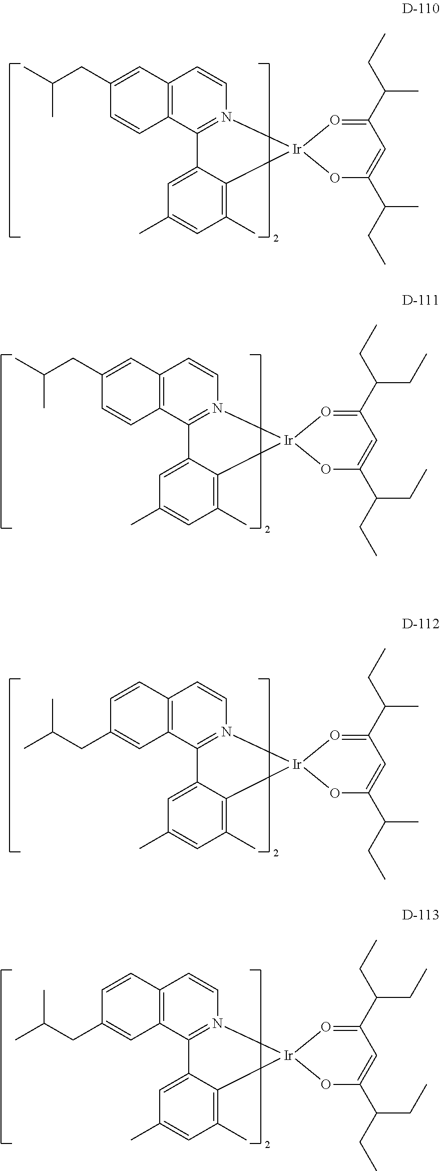

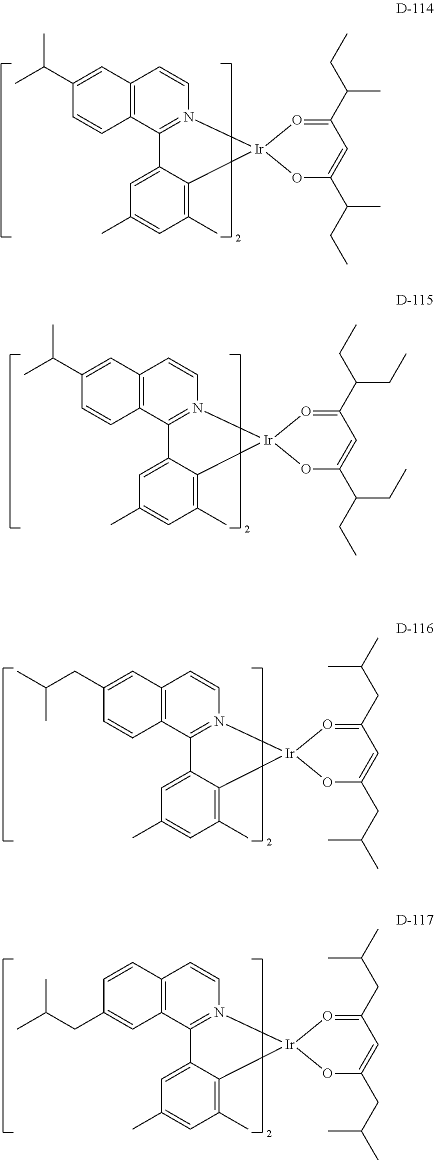

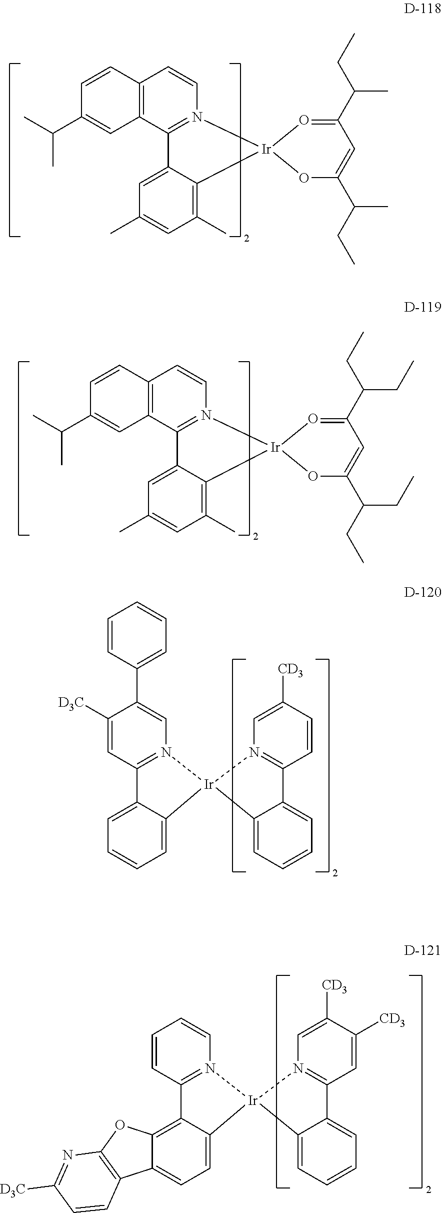

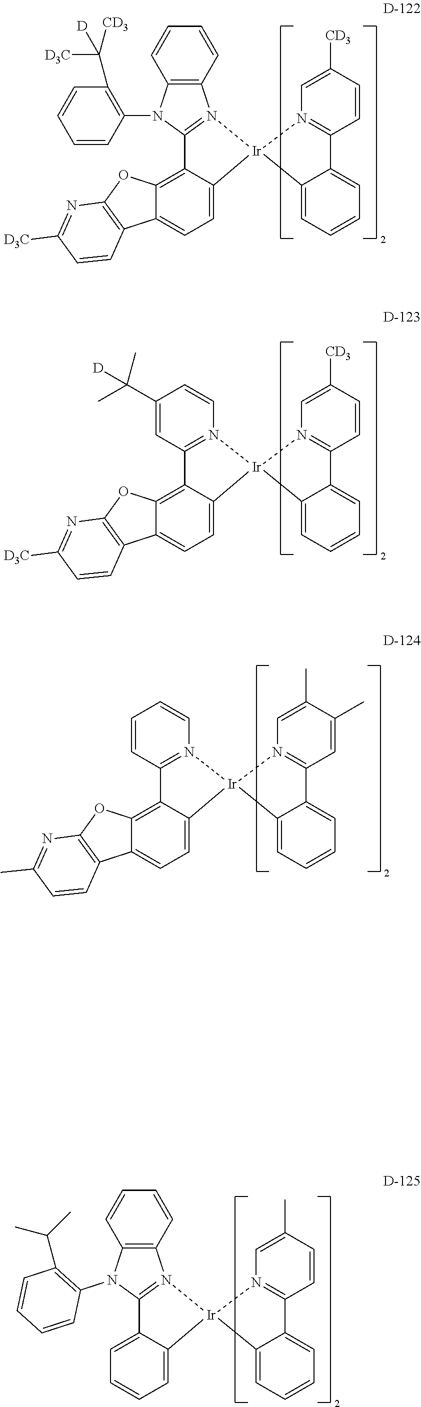

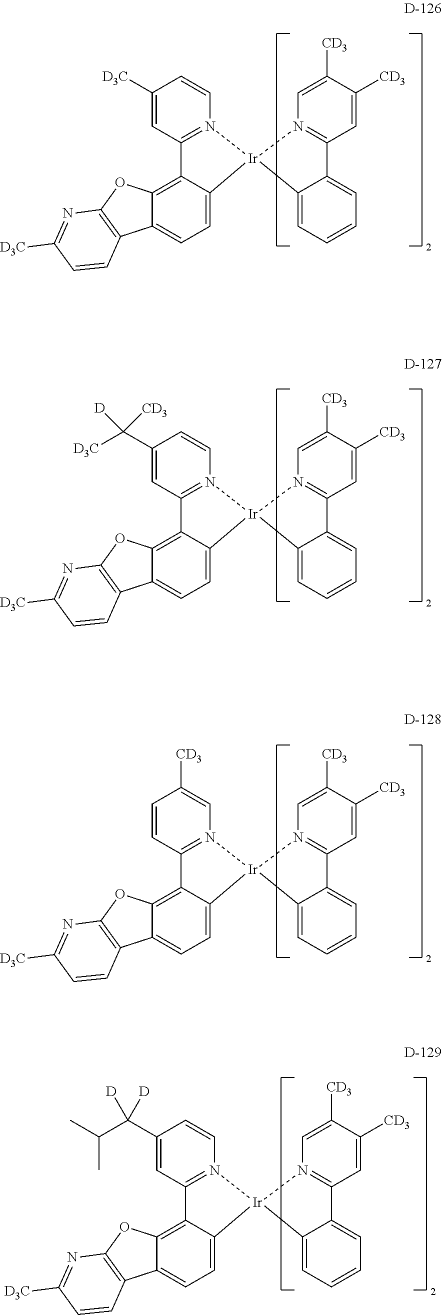

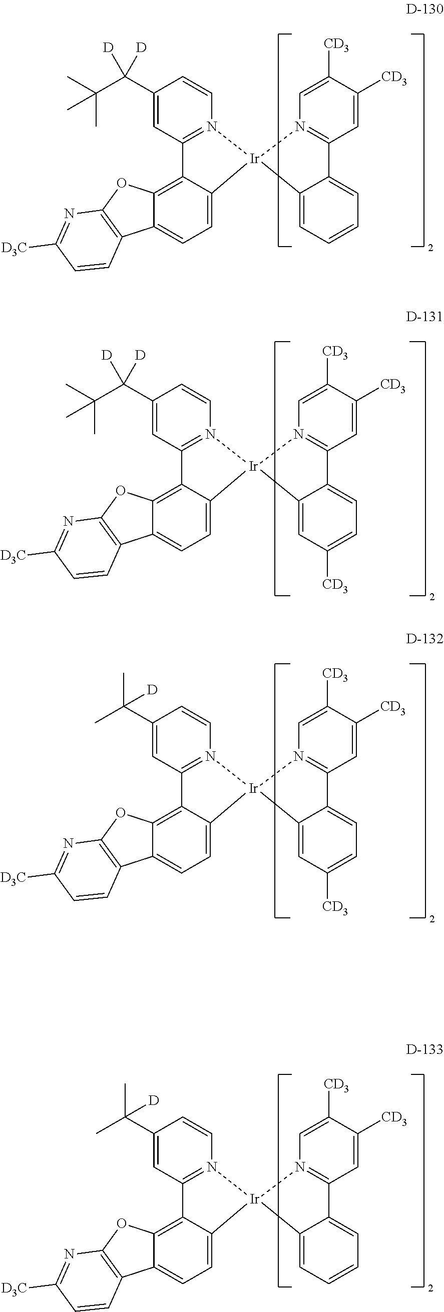

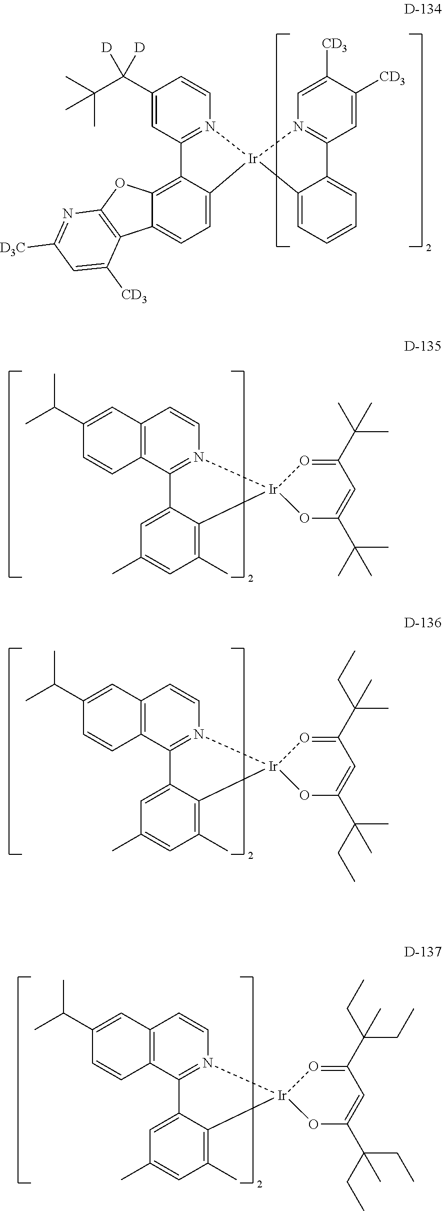

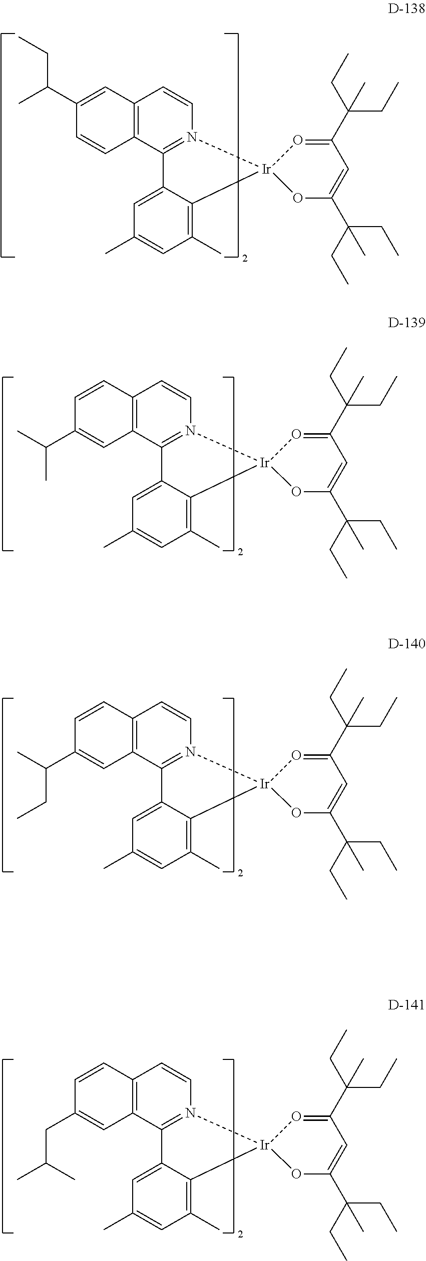

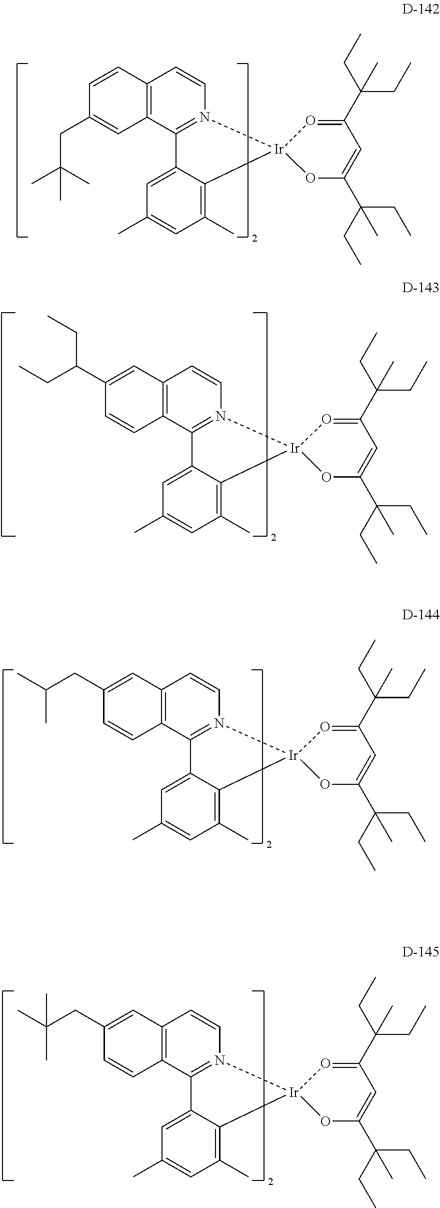

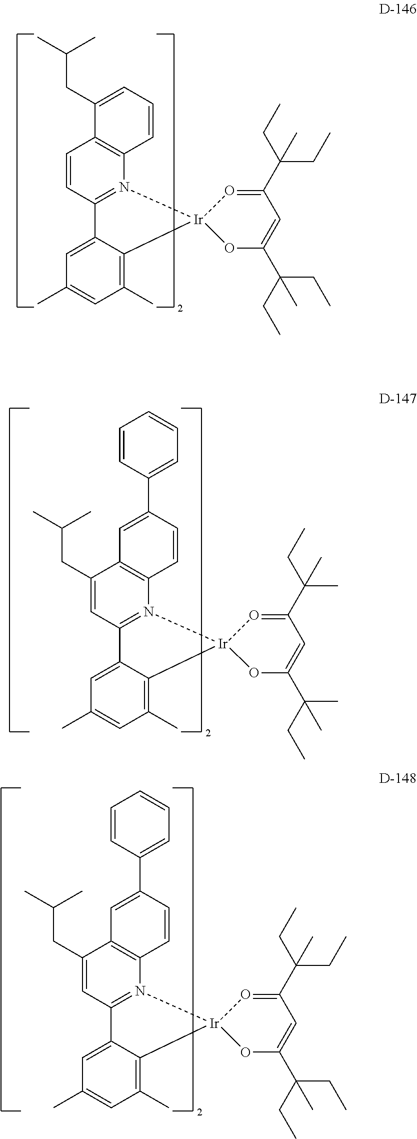

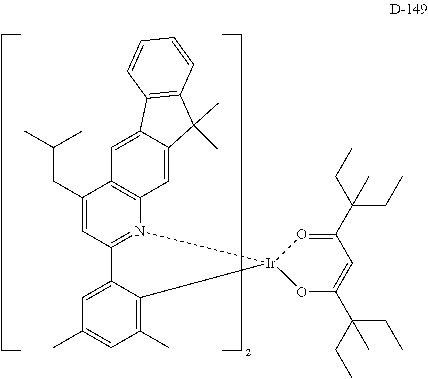

In order to form each layer of the organic electroluminescent device of the present disclosure, dry film-forming methods such as vacuum evaporation, sputtering, plasma, ion plating methods, etc. or wet film-forming methods such as spin coating, dip coating, flow coating methods, etc. can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the organic electroluminescent material according to one embodiment, the layer can be formed by the above-listed methods, and can often be formed by co-deposition or mixture-deposition. The co-deposition is a mixed deposition method in which two or more materials are put into respective individual crucible sources, and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, the present disclosure can provide a display device comprising a compound represented by Formula 1, and/or a display device comprising an organic electroluminescent material comprising a first host compound represented by Formula 1, and a second host compound represented by Formula 2 or 3. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the compound according to the present disclosure will be explained with reference to the synthesis method of a representative compound or intermediate compound in order to understand the present disclosure in detail.

### [Example 1] Synthesis of Compound C-32

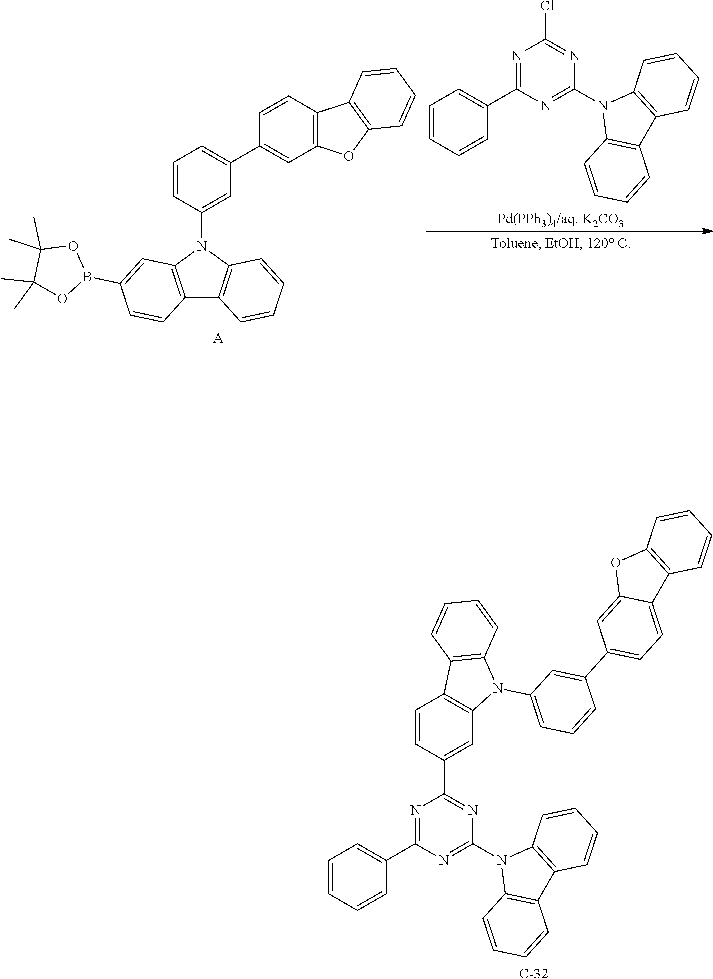

Compound A (6.0 g, 11.21 mmol), 9-(4-chloro-6-phenyl-1,3,5-triazin-2-yl)-9H-carbazole (4.4 g, 12.33 mmol), tetrakis(triphenylphosphine)palladium (Pd(PPh₃)₄) (0.4 g, 0.34 mmol), potassium carbonate (K₂CO₃) (3.8 g, 28.01 mmol), 56 mL of toluene, 14 mL of ethanol, and 14 mL of distilled water were added to a flask and dissolved, and this was then stirred under reflux for 4 hours. When the reaction was completed, the organic layer was extracted with ethyl acetate and was separated by column chromatography to obtain Compound C-32 (4.8 g, yield: 58%).

| | MW | M.P |
|---|---|---|
| C-32 | 729.84 | 273° C. |

Hereinafter, the preparation method of an organic electroluminescent device comprising the compound according to the present disclosure and an organic electroluminescent material comprising the same and the device properties thereof will be explained in order to understand the present disclosure in detail.

### [Device Example 1] Preparation of a Green Light-Emitting OLED Deposited with an Organic Electroluminescent Material According to the Present Disclosure

An OLED according to the present disclosure was prepared. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-1 was introduced into another cell. The two materials were evaporated at different rates, and Compound HI-1 was deposited in a doping amount of 3 wt % based on the total amount of Compounds HI-1 and HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: each of the first host compound and the second host compound described in the following Table 1 were introduced into two cells of the vacuum vapor deposition apparatus as hosts, respectively, and Compound D-130 was introduced into another cell as a dopant. The two host materials were evaporated at a ratio of 1:2 (the first host:the second host), and the dopant material was evaporated at a different ratio, simultaneously, and was deposited in a doping amount of 10 wt % based on the total amount of the hosts and dopant to form a light-emitting layer having a thickness of 40 nm on the second hole transport layer. Next, Compounds ETL-1 and EIL-1 as electron transport materials were deposited at a weight ratio of 40:60 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. Each compound used for all of the materials were purified by vacuum sublimation at 10⁻⁶ Torr.

### [Comparative Example 1] Preparation of an OLED Comprising the Comparative Compound as a Host

An OLED was manufactured in the same manner as in Device Example 1, except that Compound A-1 was used as the first host of the light-emitting layer.

### [Comparative Example 2] Preparation of an OLED Comprising the Comparative Compound as a Host

An OLED was manufactured in the same manner as in Device Example 1, except that Compound A-2 was used as the first host of the light-emitting layer.

The driving voltage and the luminous color at a luminance of 1,000 nit and the time taken for luminance to decrease from 100% to 80% at a luminance of 60,000 nit (lifespan: T₈₀) of the OLEDs of Device Example 1 and Comparative Examples 1 and 2 produced as described above were measured, and the results thereof are shown in Table 1 below.

TABLE 1

| | | | Driving | | |
|---|---|---|---|---|---|
| | | | Voltage | Luminous | Lifespan |
| | First Host | Second Host | (V) | Color | (T₈₀, hr) |
| Device | C-32 | H2-147 | 3.1 | Green | 97.0 |
| Example 1 | | | | | |
| Comparative | A-1 | H2-147 | 2.8 | Green | 67.3 |
| Example 1 | | | | | |
| Comparative | A-2 | H2-147 | 3.2 | Green | 79.4 |
| Example 2 | | | | | |

### [Device Example 2] Preparation of a Green Light-Emitting OILED Deposited with an Organic Electroluminescent Material According to the Present Disclosure

An OLED was manufactured in the same manner as in Device Example 1, except that Compound H2-2-D23 was used as the second host of the light-emitting layer.

### [Comparative Example 3] Preparation of an OILED Comprising the Comparative Compound as a Host

An OLED was manufactured in the same manner as in Device Example 2, except that Compound A-1 was used as the first host of the light-emitting layer.

### [Comparative Example 4] Preparation of an OILED Comprising the Comparative Compound as a Host

An OLED was manufactured in the same manner as in Device Example 2, except that Compound A-2 was used as the first host of the light-emitting layer.

The driving voltage and the luminous color at a luminance of 1,000 nit and the time taken for luminance to decrease from 100% to 80% at a luminance of 60,000 nit (Lifespan: T₈₀) of the OLEDs of Device Example 2 and Comparative Examples 3 and 4 produced as described above were measured, and the results thereof are shown in Table 2 below.

TABLE 2

| | | | Driving | | |
|---|---|---|---|---|---|
| | | | Voltage | Luminous | Lifespan |
| | First Host | Second Host | (V) | Color | (T₈₀, hr) |
| Device | C-32 | H2-2-D23 | 3.1 | Green | 172.8 |
| Example 2 | | | | | |
| Comparative | A-1 | H2-2-D23 | 2.8 | Green | 83.9 |
| Example 3 | | | | | |
| Comparative | A-2 | H2-2-D23 | 3.2 | Green | 101.0 |
| Example 4 | | | | | |

As shown in Tables 1 and 2 above, it was confirmed that the organic electroluminescent devices (Device Examples 1 and 2) including organic electroluminescent materials according to the present disclosure exhibited superior lifespan characteristics compared to organic electroluminescent devices (Comparative Examples 1 to 4) including the conventional hosts combination.

The lifespan of a green light-emitting organic electroluminescent device is generally shorter than that of a red light-emitting organic electroluminescent device. In order to improve the lifespan characteristics of the green light-emitting organic electroluminescent device, a compound having a novel structure and a compound introducing a deuterium-modified moiety are used in the present disclosure. Without being limited by theory, when an organic electroluminescent compound is substituted with deuterium, the zero-point vibration energy of the compound is lowered, thereby increasing the bond dissociation energy (BDE) in the compound. As a result, the stability of the compound can be increased.

### [Device Example 3] Preparation of an OLED Comprising a Compound According to the Present Disclosure as a Single Host Material

An OLED according to the present disclosure was prepared. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-1 was introduced into another cell. The two materials were evaporated at different rates, and Compound HI-1 was deposited in a doping amount of 3 wt % based on the total amount of Compounds HI-1 and HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: Compound C-32 was introduced into a cell of the vacuum vapor deposition apparatus as a host, and Compound D-130 was introduced into another cell as a dopant. The dopant material was evaporated at a different rate, simultaneously, and was deposited in a doping amount of 10 wt % based on the total amount of the host and dopant to form a light-emitting layer having a thickness of 40 nm on the second hole transport layer. Next, Compounds ETL-1 and EIL-1 as electron transport materials were deposited at a weight ratio of 40:60 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. Each compound used for all the materials were purified by vacuum sublimation at 10⁻⁶ Torr.

### [Comparative Example 5] Preparation of an OLED Comprising the Conventional Compound as a Host

An OLED was manufactured in the same manner as in Device Example 3, except that Compound A-1 was used as the host of the light-emitting layer.

### [Comparative Example 6] Preparation of an OLED Comprising the Conventional Compound as a Host

An OLED was manufactured in the same manner as in Device Example 3, except that Compound A-2 was used as the host of the light-emitting layer.

The driving voltage and the luminous color at a luminance of 1,000 nit and the time taken for luminance to decrease from 100% to 80% at a luminance of 20,000 nit (lifespan: T₈₀) of the OLEDs of Device Example 3 and Comparative Examples 5 and 6 produced as described above were measured, and the results thereof are shown in Table 3 below.

TABLE 3

| | | Driving | | |
|---|---|---|---|---|
| | | Voltage | Luminous | Lifespan |
| | Host | (V) | Color | (T₈₀, hr) |
| Device Example 3 | C-32 | 2.7 | Green | 301.0 |
| Comparative | A-1 | 2.6 | Green | 69.4 |
| Example 5 | | | | |
| Comparative | A-2 | 3.6 | Green | 68.6 |
| Example 6 | | | | |

From Table 3 above, it can be confirmed that the organic electroluminescent device (Device Example 3) including the compound having the novel structure according to the present disclosure as a host material of the light-emitting layer exhibits lower driving voltage and/or superior lifespan characteristics compared to the organic electroluminescent devices (Comparative Examples 5 and 6) including conventional host compounds.

The compounds used in Device Examples and Comparative Examples are specifically shown in Table 4 below.

TABLE 4

| | | |
|---|---|---|
| Hole Injection Layer/Hole Transport Layer | 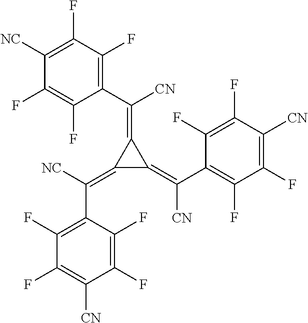 HI-1 | 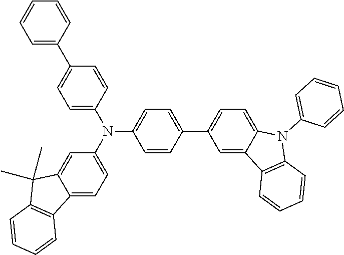 HT-1 |
| | 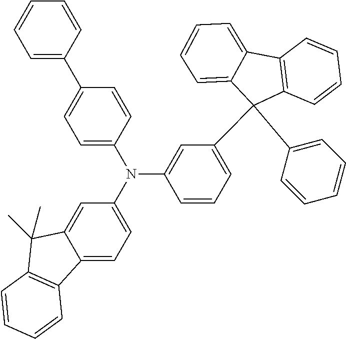 HT-2 | |
| Light- Emitting Layer | 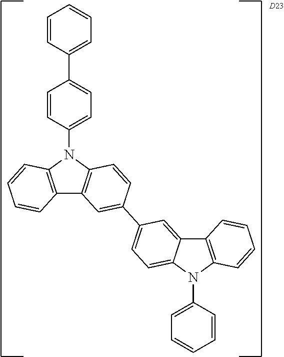 H2-2-D23 | 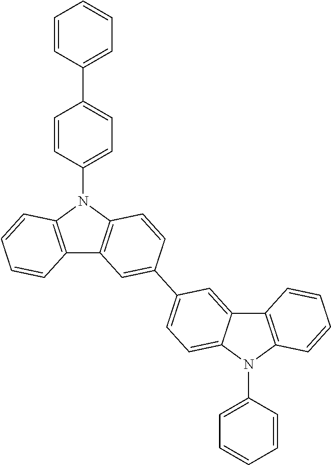 H2-147 |
| | 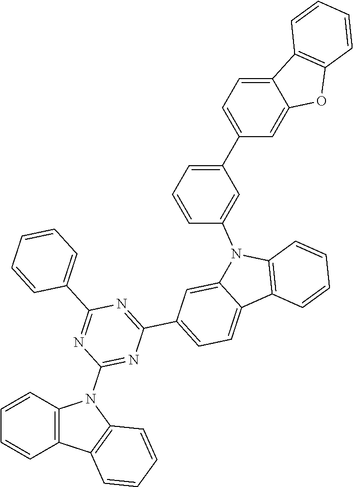 C-32 | 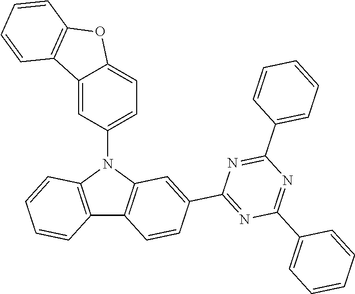 A-1 |
| | 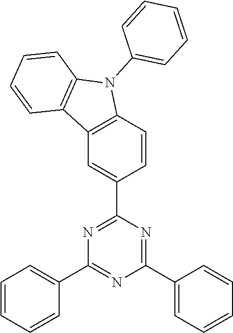 A-2 | 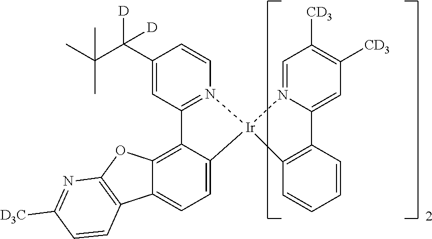 D-130 |
| Electron Transport Layer/ Electron Injection Layer | 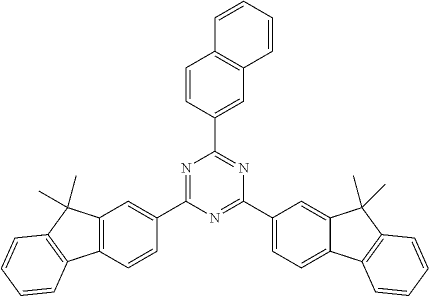 ETL-1 | 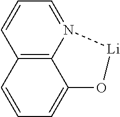 EIL-1 |

### [Device Example 4] Preparation of a Green Light-Emitting OLED Deposited with an Organic Electroluminescent Material According to the Present Disclosure

An OLED was manufactured in the same manner as in Device Example 1, except that Compound H1-7 was used as the second host of the light-emitting layer.

### [Device Example 5] Preparation of a Green Light-Emitting OLED Deposited with an Organic Electroluminescent Material According to the Present Disclosure

An OLED was manufactured in the same manner as in Device Example 1, except that Compound H1-97-D20 was used as the second host of the light-emitting layer.

### [Comparative Example 7] Preparation of an OLED Comprising the Comparative Compound as a Host

An OLED was manufactured in the same manner as in Device Example 4, except that Compound A-2 was used as the first host of the light-emitting layer.

### [Comparative Example 8] Preparation of an OLED Comprising the Comparative Compound as a Host

An OLED was manufactured in the same manner as in Device Example 5, except that Compound A-2 was used as the first host of the light-emitting layer.

The driving voltage, the luminous efficiency, and the luminous color at a luminance of 1,000 nit of the OLEDs of Device Examples 4 and 5 and Comparative Examples 7 and 8 produced as described above were measured, and the results thereof are shown in Table 5 below.

TABLE 5

| | | | Driving | Luminous | |
|---|---|---|---|---|---|
| | | | Voltage | Efficiency | Luminous |
| | First Host | Second Host | (V) | (cd/A) | Color |
| Device | C-32 | H1-7 | 2.8 | 95.2 | Green |
| Example 4 | | | | | |
| Device | C-32 | H1-97-D20 | 2.8 | 95.9 | Green |
| Example 5 | | | | | |
| Comparative | A-2 | H1-7 | 3.0 | 94.0 | Green |
| Example 7 | | | | | |
| Comparative | A-2 | H1-97-D20 | 3.0 | 92.6 | Green |
| Example 8 | | | | | |

As shown in Table 5 above, it was confirmed that the organic electroluminescent devices (Device Examples 4 and 5) including the organic electroluminescent material according to the present disclosure exhibited lower driving voltage and/or superior efficiency characteristics compared to the organic electroluminescent devices (Device Comparative Examples 7 and 8) including the conventional hosts combination.

The compounds used in Device Examples and Device Comparative Examples are specifically shown in Table 6 below.

TABLE 6

| | | |
|---|---|---|
| Hole Injection Layer/Hole Transport Layer | 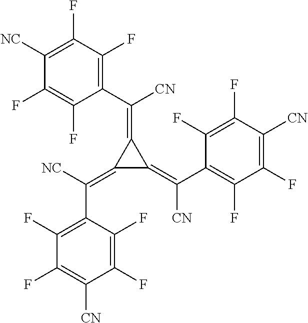 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] HI-1 | 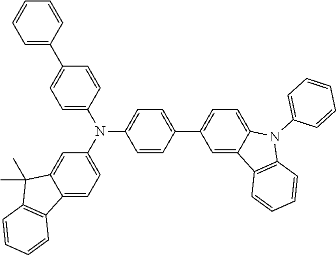 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] HT-1 |
| | 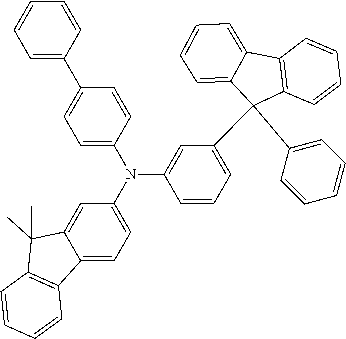 [SMILES: CC1(C)c2ccccc2-c2ccc(N(c3ccc(-c4ccccc4)cc3)c3cccc(C4(c5ccccc5)c5ccccc5-c5ccccc54)c3)cc21] HT-2 | |
| Light- Emitting Layer | 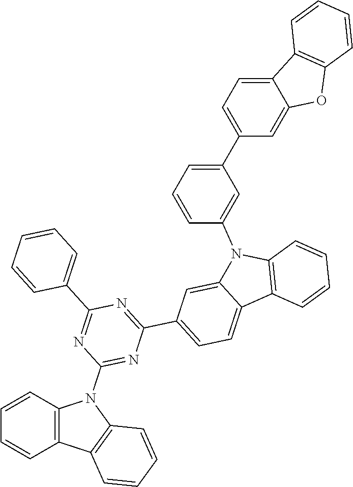 [SMILES: c1ccc(-c2nc(-c3ccc4c5ccccc5n(-c5cccc(-c6ccc7c(c6)oc6ccccc67)c5)c4c3)nc(-n3c4ccccc4c4ccccc43)n2)cc1] C-32 | 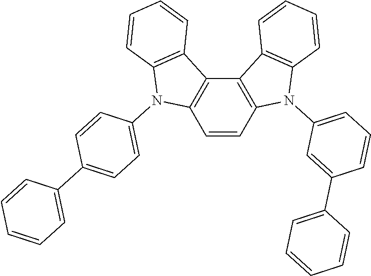 [SMILES: c1ccc(-c2ccc(-n3c4ccccc4c4c5c6ccccc6n(-c6cccc(-c7ccccc7)c6)c5ccc43)cc2)cc1] HI-7 |
| | 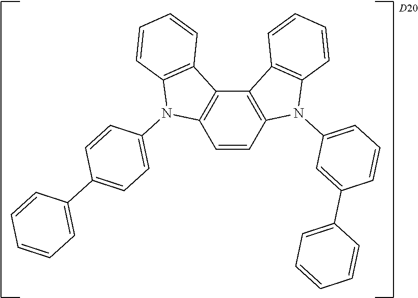 H1-97-D20 | 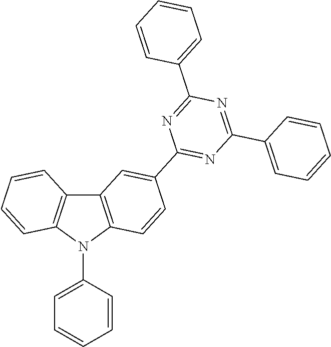 [SMILES: c1ccc(-c2nc(-c3ccccc3)nc(-c3ccc4c(c3)c3ccccc3n4-c3ccccc3)n2)cc1] A-2 |
| | 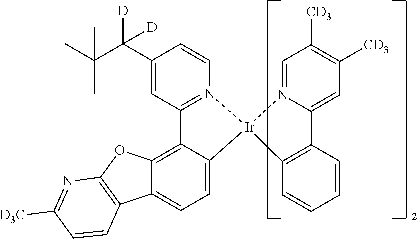 D-130 | |
| Electron Transport Layer / Electron Injection Layer | 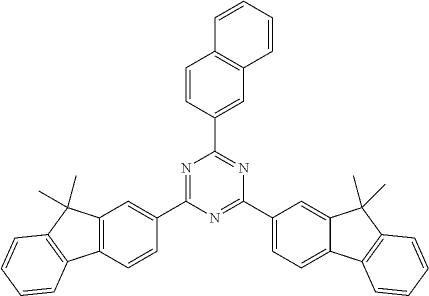 [SMILES: CC1(C)c2ccccc2-c2ccc(-c3nc(-c4ccc5c(c4)C(C)(C)c4ccccc4-5)nc(-c4ccc5ccccc5c4)n3)cc21] ETL-1 | 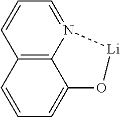 EIL-1 |

1. A compound represented by the following Formula 1: 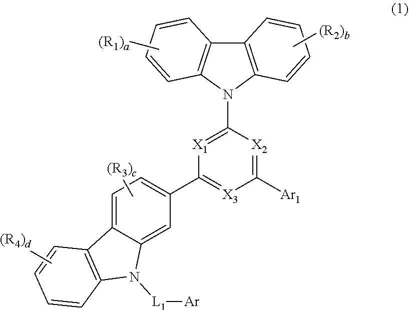
wherein,
X₁ to X₃ each independently represent CR₅ or N, provided that at least one of X₁ to X₃ is N;
Ar₁ represents hydrogen, deuterium, halogen, cyano, amino, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, —Si(R₁₁)(R₁₂)(R₁₃), or —N(R₁₄)(R₁₅);
R₁₁ to R₁₅ each independently represent a substituted or unsubstituted (C₁-C₃₀)alkyl, a substituted or unsubstituted (C₂-C₃₀)alkenyl, a substituted or unsubstituted (C₆-C₃₀)aryl, or a substituted or unsubstituted (3- to 30-membered)heteroaryl;
L₁ represents a single bond, a substituted or unsubstituted (C₆-C₃₀)arylene, or a substituted or unsubstituted (3- to 30-membered)heteroarylene;
Ar represents a substituted or unsubstituted (C₆-C₃₀)aryl or a substituted or unsubstituted (3- to 30-membered)heteroaryl;
R₁ to R₅ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s); and
a, b, and d each independently represent an integer of 1 to 4, c represents an integer of 1 to 3, and when a to d are an integer of 2 or more, each of R₁ to R₄ may be the same as or different from each other.

2. The compound according to claim 1, wherein the substituted alkyl, the substituted alkenyl, the substituted cycloalkyl, the substituted aryl(ene), the substituted heteroaryl(ene), the substituted alkoxy, the substituted trialkylsilyl, the substituted dialkylarylsilyl, the substituted alkyldiarylsilyl, and the substituted triarylsilyl, each independently are substituted with at least one selected from the group consisting of deuterium; halogen; cyano; carboxyl; nitro; hydroxyl; phosphine oxide; (C₁-C₃₀)alkyl; halo(C₁-C₃₀)alkyl; (C₂-C₃₀)alkenyl; (C₂-C₃₀)alkynyl; (C₁-C₃₀)alkoxy; (C₁-C₃₀)alkylthio; (C₃-C₃₀)cycloalkyl; (C₃-C₃₀)cycloalkenyl; (3- to 7-membered)heterocycloalkyl; (C₆-C₃₀)aryloxy; (C₆-C₃₀)arylthio; (C₆-C₃₀)aryl unsubstituted or substituted with at least one of (C₁-C₃₀)alkyl and di(C₆-C₃₀)arylamino; (3- to 30-membered)heteroaryl unsubstituted or substituted with at least one (C₆-C₃₀)aryl; tri(C₁-C₃₀)alkylsilyl; tri(C₆-C₃₀)arylsilyl; di(C₁-C₃₀)alkyl(C₆-C₃₀)arylsilyl; (C₁-C₃₀)alkyldi(C₆-C₃₀)arylsilyl; a fused ring of (C₃-C₃₀) aliphatic ring and (C₆-C₃₀) aromatic ring; amino; mono- or di(C₁-C₃₀)alkylamino; mono- or di(C₂-C₃₀)alkenylamino; mono- or di(C₆-C₃₀)arylamino unsubstituted or substituted with (C₁-C₃₀)alkyl; mono- or di(3- to 30-membered)heteroarylamino; (C₁-C₃₀)alkyl(C₂-C₃₀)alkenylamino; (C₁-C₃₀)alkyl(C₆-C₃₀)arylamino; (C₁-C₃₀)alkyl(3- to 30-membered)heteroarylamino; (C₂-C₃₀)alkenyl(C₆-C₃₀)arylamino; (C₂-C₃₀)alkenyl(3- to 30-membered)heteroarylamino; (C₆-C₃₀)aryl(3- to 30-membered)heteroarylamino; (C₁-C₃₀)alkylcarbonyl; (C₁-C₃₀)alkoxycarbonyl; (C₆-C₃₀)arylcarbonyl; di(C₆-C₃₀)arylboronyl; di(C₁-C₃₀)alkylboronyl; (C₁-C₃₀)alkyl(C₆-C₃₀)arylboronyl; (C₆-C₃₀)ar(C₁-C₃₀)alkyl; and (C₁-C₃₀)alkyl(C₆-C₃₀)aryl.

3. The compound according to claim 1, wherein Formula 1 is represented by the following Formula 1-1: 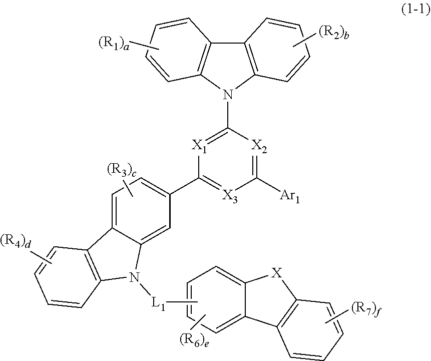
wherein,
R₁ to R₄, X₁ to X₃, Ar₁, L₁, and a to d are as defined in claim 1;
X represents O, S, or Se;
R₆ and R₇ are as defined as R₁ in claim 1; and
e represents an integer of 1 to 3, f represents an integer of 1 to 4, and when e and f are an integer of 2 or more, each of R₆ and each of R₇ may be the same as or different from each other.

4. The compound according to claim 1, wherein the compound represented by Formula 1 is selected from the following compounds: 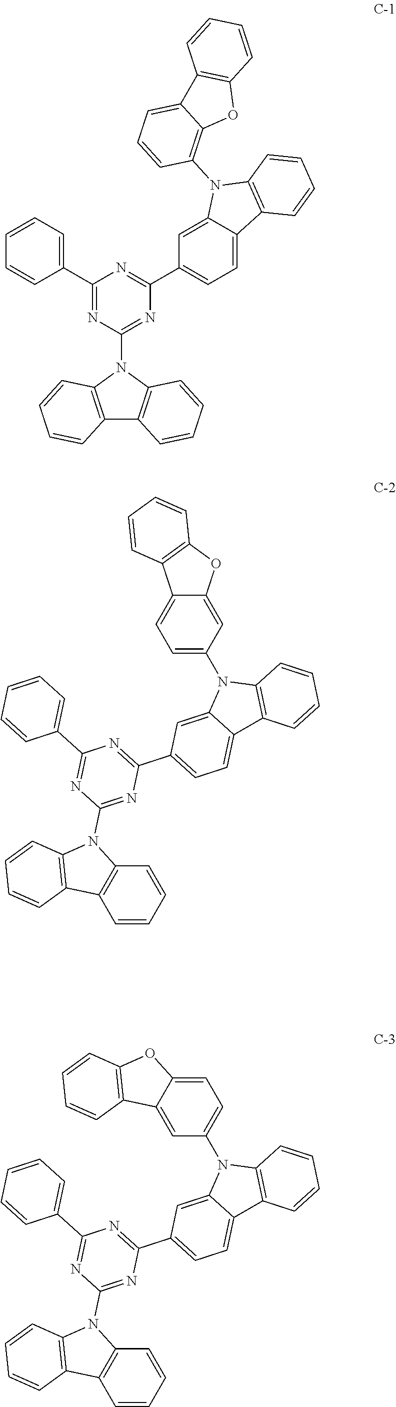 [SMILES: c1ccc(-c2nc(-c3ccc4c5ccccc5n(-c5cccc6c5oc5ccccc56)c4c3)nc(-n3c4ccccc4c4ccccc43)n2)cc1 ; c1ccc(-c2nc(-c3ccc4c5ccccc5n(-c5ccc6c(c5)oc5ccccc56)c4c3)nc(-n3c4ccccc4c4ccccc43)n2)cc1 ; c1ccc(-c2nc(-c3ccc4c5ccccc5n(-c5ccc6oc7ccccc7c6c5)c4c3)nc(-n3c4ccccc4c4ccccc43)n2)cc1] 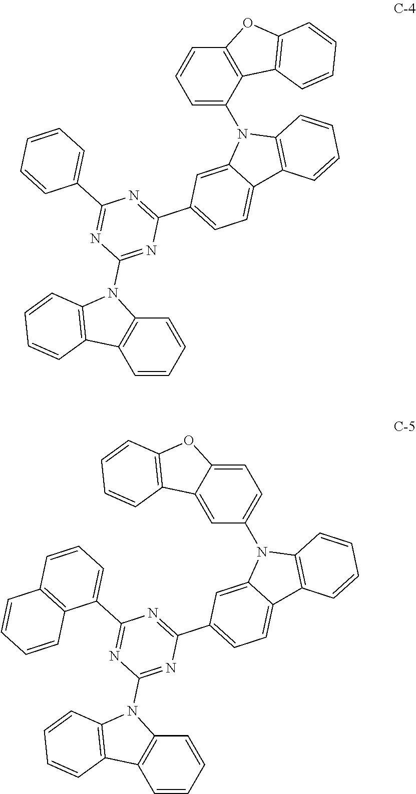 [SMILES: c1ccc(-c2nc(-c3ccc4c5ccccc5n(-c5cccc6oc7ccccc7c56)c4c3)nc(-n3c4ccccc4c4ccccc43)n2)cc1 ; c1ccc2c(-c3nc(-c4ccc5c6ccccc6n(-c6ccc7oc8ccccc8c7c6)c5c4)nc(-n4c5ccccc5c5ccccc54)n3)cccc2c1] 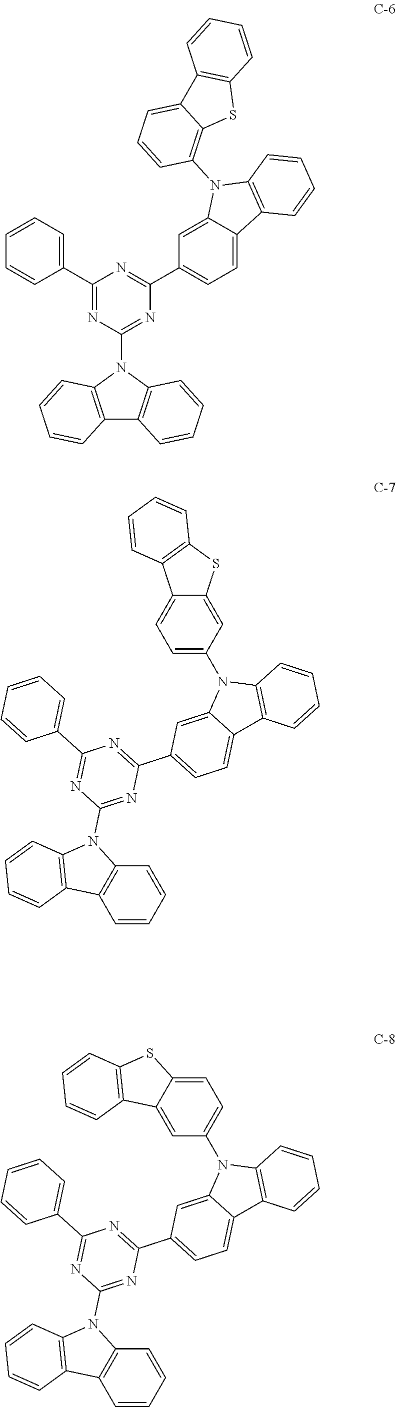 [SMILES: c1ccc(-c2nc(-c3ccc4c5ccccc5n(-c5cccc6c5sc5ccccc56)c4c3)nc(-n3c4ccccc4c4ccccc43)n2)cc1 ; c1ccc(-c2nc(-c3ccc4c5ccccc5n(-c5ccc6c(c5)sc5ccccc56)c4c3)nc(-n3c4ccccc4c4ccccc43)n2)cc1 ; c1ccc(-c2nc(-c3ccc4c5ccccc5n(-c5ccc6sc7ccccc7c6c5)c4c3)nc(-n3c4ccccc4c4ccccc43)n2)cc1] 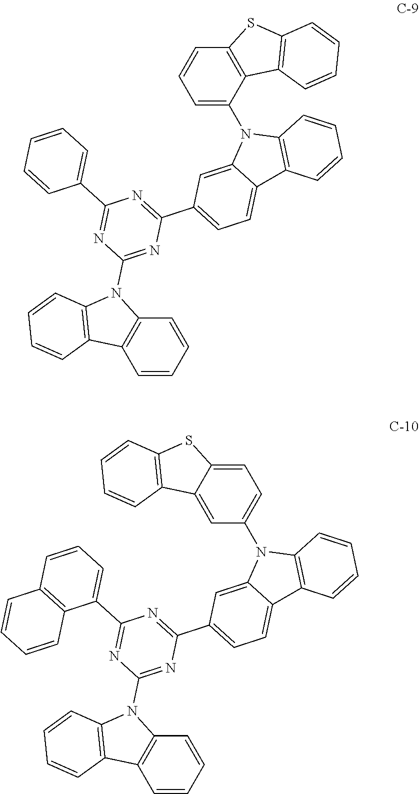 [SMILES: c1ccc(-c2nc(-c3ccc4c5ccccc5n(-c5cccc6sc7ccccc7c56)c4c3)nc(-n3c4ccccc4c4ccccc43)n2)cc1 ; c1ccc2c(-c3nc(-c4ccc5c6ccccc6n(-c6ccc7sc8ccccc8c7c6)c5c4)nc(-n4c5ccccc5c5ccccc54)n3)cccc2c1] 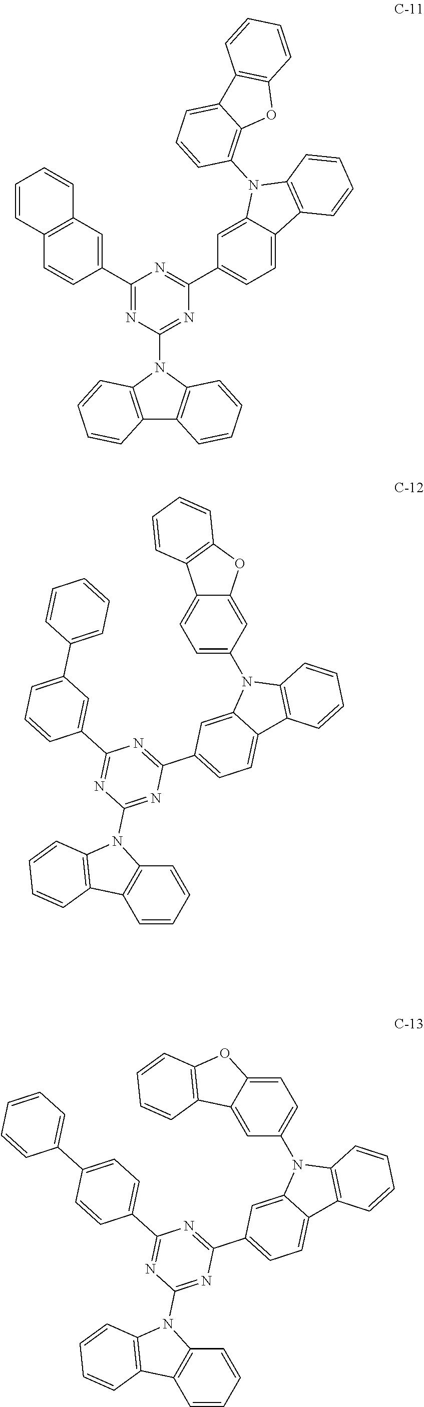 [SMILES: c1ccc2cc(-c3nc(-c4ccc5c6ccccc6n(-c6cccc7c6oc6ccccc67)c5c4)nc(-n4c5ccccc5c5ccccc54)n3)ccc2c1 ; c1ccc(-c2cccc(-c3nc(-c4ccc5c6ccccc6n(-c6ccc7c(c6)oc6ccccc67)c5c4)nc(-n4c5ccccc5c5ccccc54)n3)c2)cc1 ; c1ccc(-c2ccc(-c3nc(-c4ccc5c6ccccc6n(-c6ccc7oc8ccccc8c7c6)c5c4)nc(-n4c5ccccc5c5ccccc54)n3)cc2)cc1] 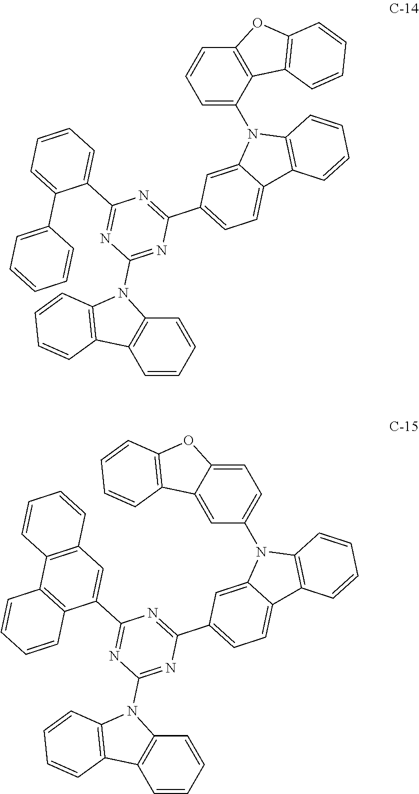 [SMILES: c1ccc(-c2ccccc2-c2nc(-c3ccc4c5ccccc5n(-c5cccc6oc7ccccc7c56)c4c3)nc(-n3c4ccccc4c4ccccc43)n2)cc1 ; c1ccc2c(c1)cc(-c1nc(-c3ccc4c5ccccc5n(-c5ccc6oc7ccccc7c6c5)c4c3)nc(-n3c4ccccc4c4ccccc43)n1)c1ccccc12] 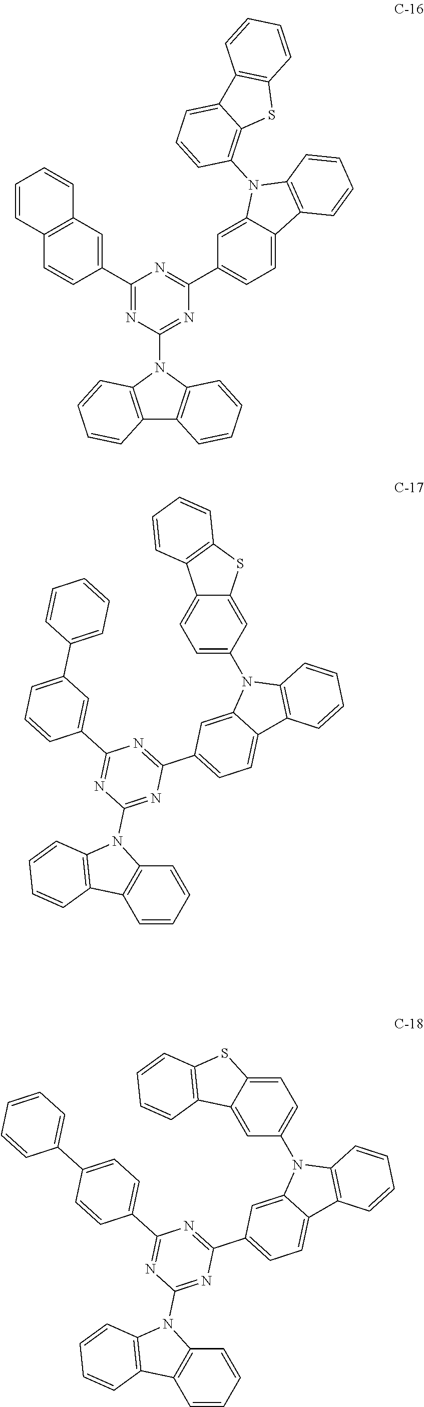 [SMILES: c1ccc2cc(-c3nc(-c4ccc5c6ccccc6n(-c6cccc7c6sc6ccccc67)c5c4)nc(-n4c5ccccc5c5ccccc54)n3)ccc2c1 ; c1ccc(-c2cccc(-c3nc(-c4ccc5c6ccccc6n(-c6ccc7c(c6)sc6ccccc67)c5c4)nc(-n4c5ccccc5c5ccccc54)n3)c2)cc1 ; c1ccc(-c2ccc(-c3nc(-c4ccc5c6ccccc6n(-c6ccc7sc8ccccc8c7c6)c5c4)nc(-n4c5ccccc5c5ccccc54)n3)cc2)cc1] 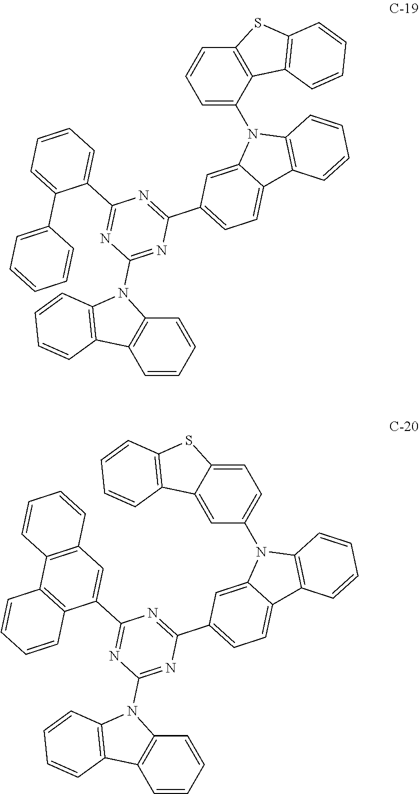 [SMILES: c1ccc(-c2ccccc2-c2nc(-c3ccc4c5ccccc5n(-c5cccc6sc7ccccc7c56)c4c3)nc(-n3c4ccccc4c4ccccc43)n2)cc1 ; c1ccc2c(c1)cc(-c1nc(-c3ccc4c5ccccc5n(-c5ccc6sc7ccccc7c6c5)c4c3)nc(-n3c4ccccc4c4ccccc43)n1)c1ccccc12] 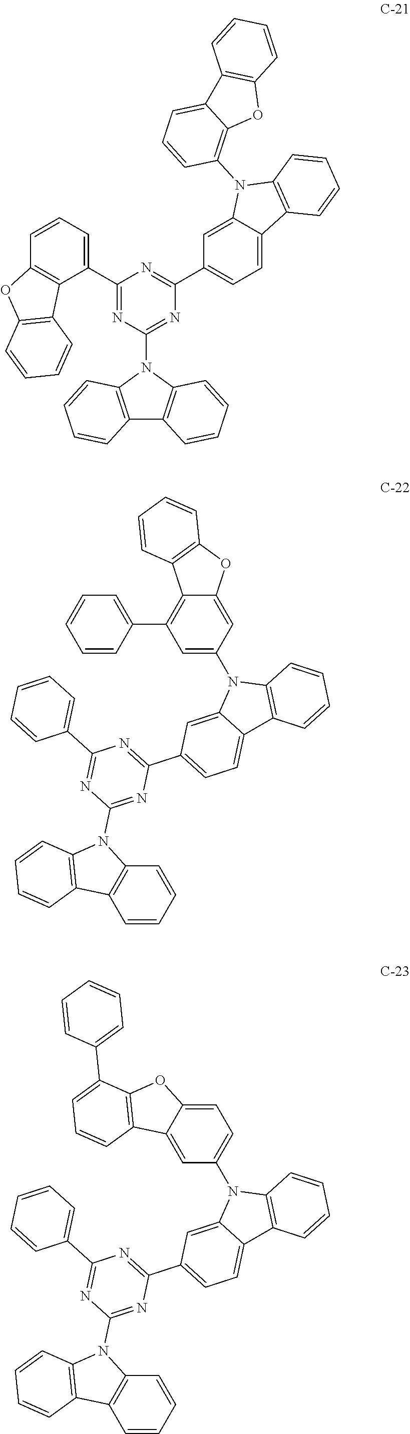 [SMILES: c1ccc2c(c1)oc1c(-n3c4ccccc4c4ccc(-c5nc(-c6cccc7oc8ccccc8c67)nc(-n6c7ccccc7c7ccccc76)n5)cc43)cccc12 ; c1ccc(-c2nc(-c3ccc4c5ccccc5n(-c5cc(-c6ccccc6)c6c(c5)oc5ccccc56)c4c3)nc(-n3c4ccccc4c4ccccc43)n2)cc1 ; c1ccc(-c2nc(-c3ccc4c5ccccc5n(-c5ccc6oc7c(-c8ccccc8)cccc7c6c5)c4c3)nc(-n3c4ccccc4c4ccccc43)n2)cc1] 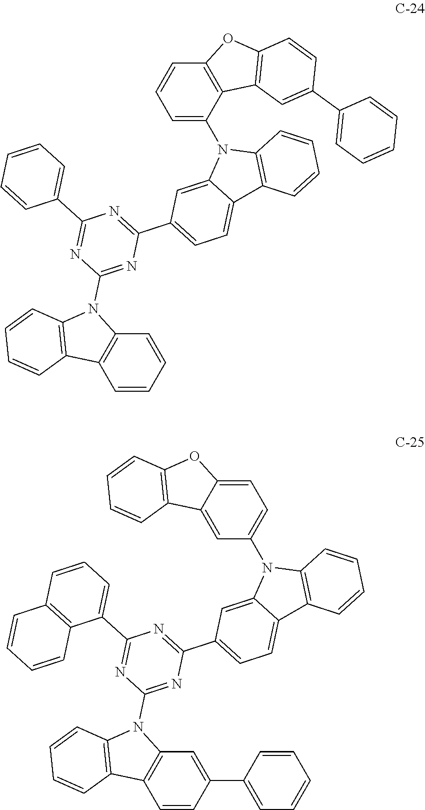 [SMILES: c1ccc(-c2ccc3oc4cccc(-n5c6ccccc6c6ccc(-c7nc(-c8ccccc8)nc(-n8c9ccccc9c9ccccc98)n7)cc65)c4c3c2)cc1 ; c1ccc(-c2ccc3c4ccccc4n(-c4nc(-c5ccc6c7ccccc7n(-c7ccc8oc9ccccc9c8c7)c6c5)nc(-c5cccc6ccccc56)n4)c3c2)cc1] 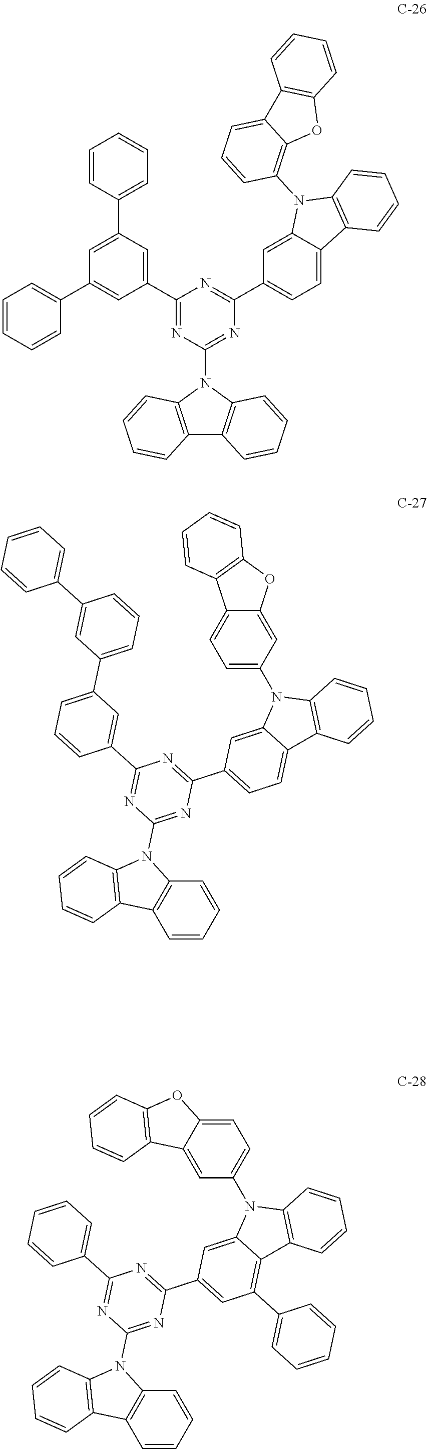 [SMILES: c1ccc(-c2cc(-c3ccccc3)cc(-c3nc(-c4ccc5c6ccccc6n(-c6cccc7c6oc6ccccc67)c5c4)nc(-n4c5ccccc5c5ccccc54)n3)c2)cc1 ; c1ccc(-c2cccc(-c3cccc(-c4nc(-c5ccc6c7ccccc7n(-c7ccc8c(c7)oc7ccccc78)c6c5)nc(-n5c6ccccc6c6ccccc65)n4)c3)c2)cc1 ; c1ccc(-c2nc(-c3cc(-c4ccccc4)c4c5ccccc5n(-c5ccc6oc7ccccc7c6c5)c4c3)nc(-n3c4ccccc4c4ccccc43)n2)cc1] 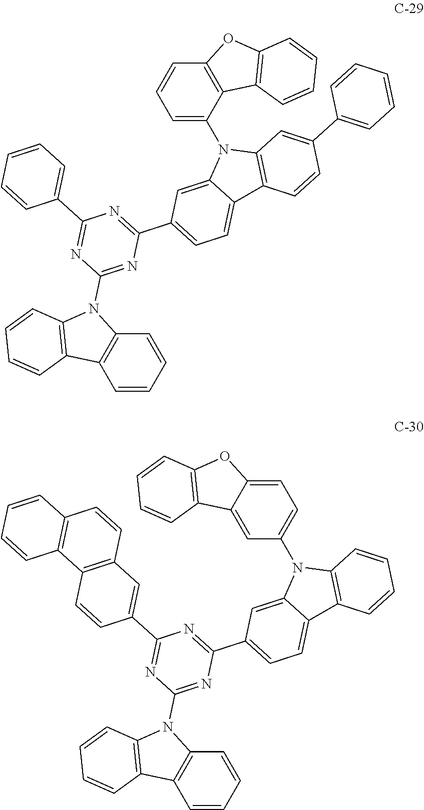 [SMILES: c1ccc(-c2ccc3c4ccc(-c5nc(-c6ccccc6)nc(-n6c7ccccc7c7ccccc76)n5)cc4n(-c4cccc5oc6ccccc6c45)c3c2)cc1 ; c1ccc2c(c1)ccc1cc(-c3nc(-c4ccc5c6ccccc6n(-c6ccc7oc8ccccc8c7c6)c5c4)nc(-n4c5ccccc5c5ccccc54)n3)ccc12] 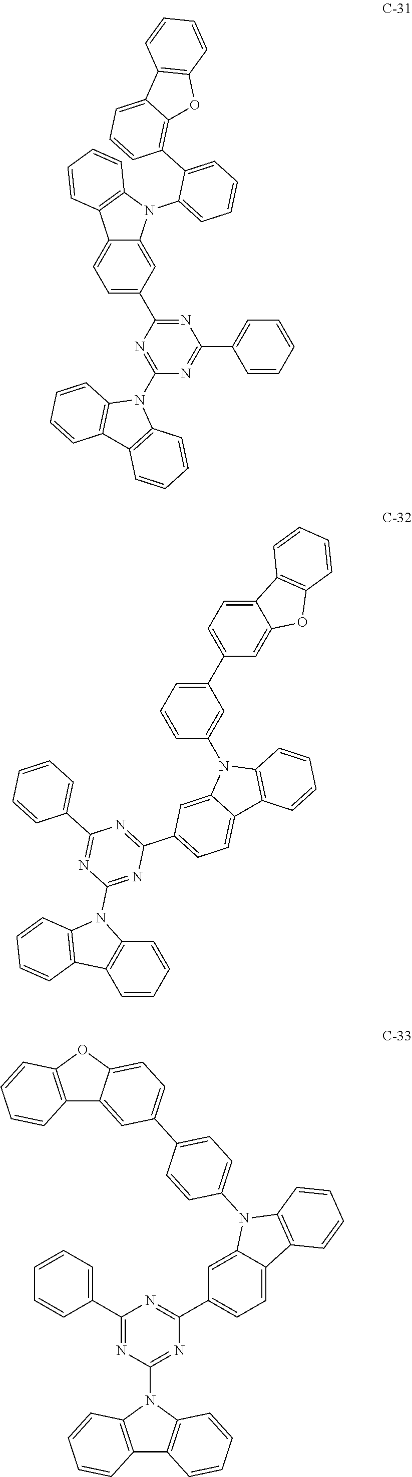 [SMILES: c1ccc(-c2nc(-c3ccc4c5ccccc5n(-c5ccccc5-c5cccc6c5oc5ccccc56)c4c3)nc(-n3c4ccccc4c4ccccc43)n2)cc1 ; c1ccc(-c2nc(-c3ccc4c5ccccc5n(-c5cccc(-c6ccc7c(c6)oc6ccccc67)c5)c4c3)nc(-n3c4ccccc4c4ccccc43)n2)cc1 ; c1ccc(-c2nc(-c3ccc4c5ccccc5n(-c5ccc(-c6ccc7oc8ccccc8c7c6)cc5)c4c3)nc(-n3c4ccccc4c4ccccc43)n2)cc1] 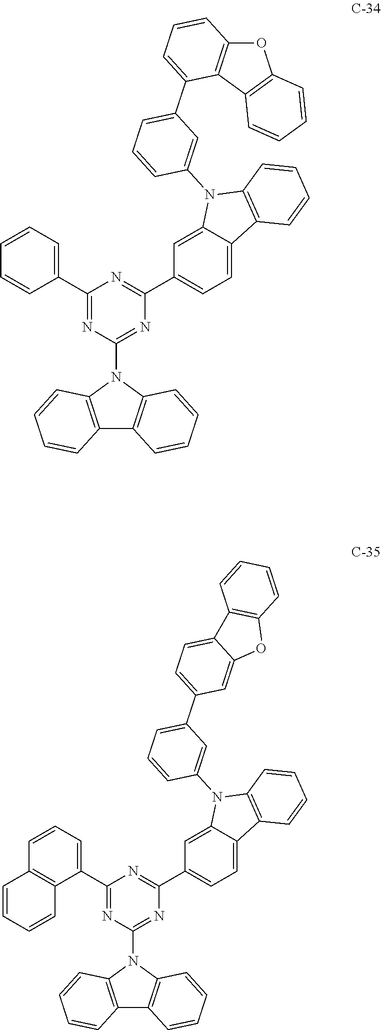 [SMILES: c1ccc(-c2nc(-c3ccc4c5ccccc5n(-c5cccc(-c6cccc7oc8ccccc8c67)c5)c4c3)nc(-n3c4ccccc4c4ccccc43)n2)cc1 ; c1cc(-c2ccc3c(c2)oc2ccccc23)cc(-n2c3ccccc3c3ccc(-c4nc(-c5cccc6ccccc56)nc(-n5c6ccccc6c6ccccc65)n4)cc32)c1] 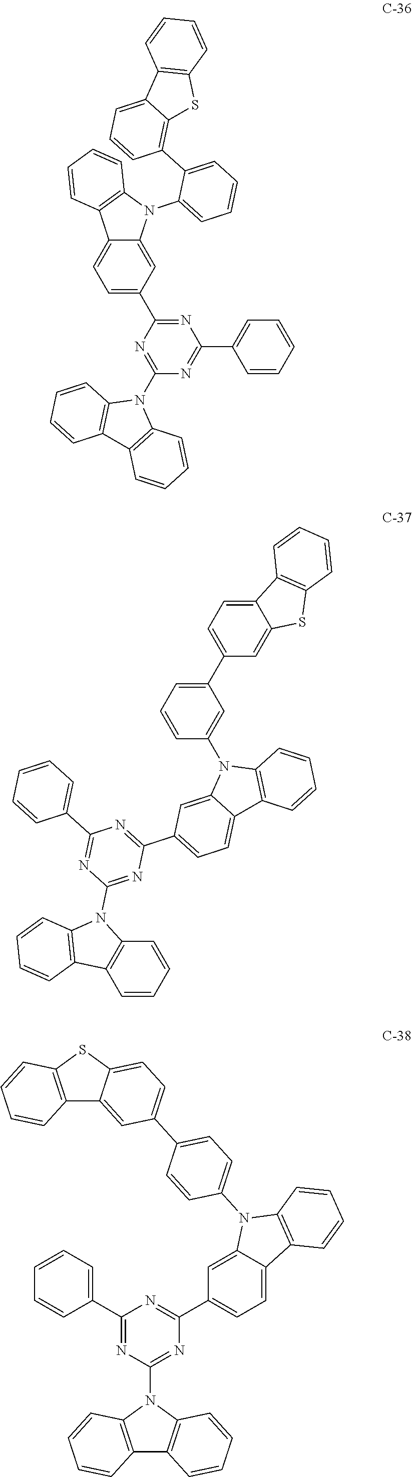 [SMILES: c1ccc(-c2nc(-c3ccc4c5ccccc5n(-c5ccccc5-c5cccc6c5sc5ccccc56)c4c3)nc(-n3c4ccccc4c4ccccc43)n2)cc1 ; c1ccc(-c2nc(-c3ccc4c5ccccc5n(-c5cccc(-c6ccc7c(c6)sc6ccccc67)c5)c4c3)nc(-n3c4ccccc4c4ccccc43)n2)cc1 ; c1ccc(-c2nc(-c3ccc4c5ccccc5n(-c5ccc(-c6ccc7sc8ccccc8c7c6)cc5)c4c3)nc(-n3c4ccccc4c4ccccc43)n2)cc1] 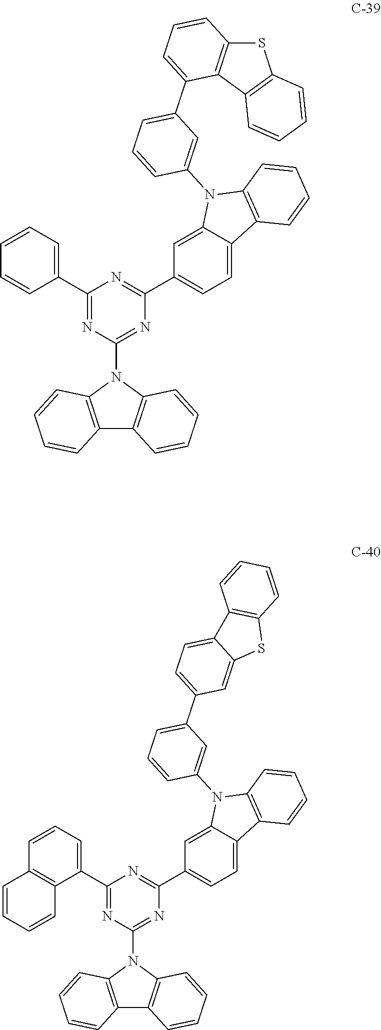 [SMILES: c1ccc(-c2nc(-c3ccc4c5ccccc5n(-c5cccc(-c6cccc7sc8ccccc8c67)c5)c4c3)nc(-n3c4ccccc4c4ccccc43)n2)cc1 ; c1cc(-c2ccc3c(c2)sc2ccccc23)cc(-n2c3ccccc3c3ccc(-c4nc(-c5cccc6ccccc56)nc(-n5c6ccccc6c6ccccc65)n4)cc32)c1] 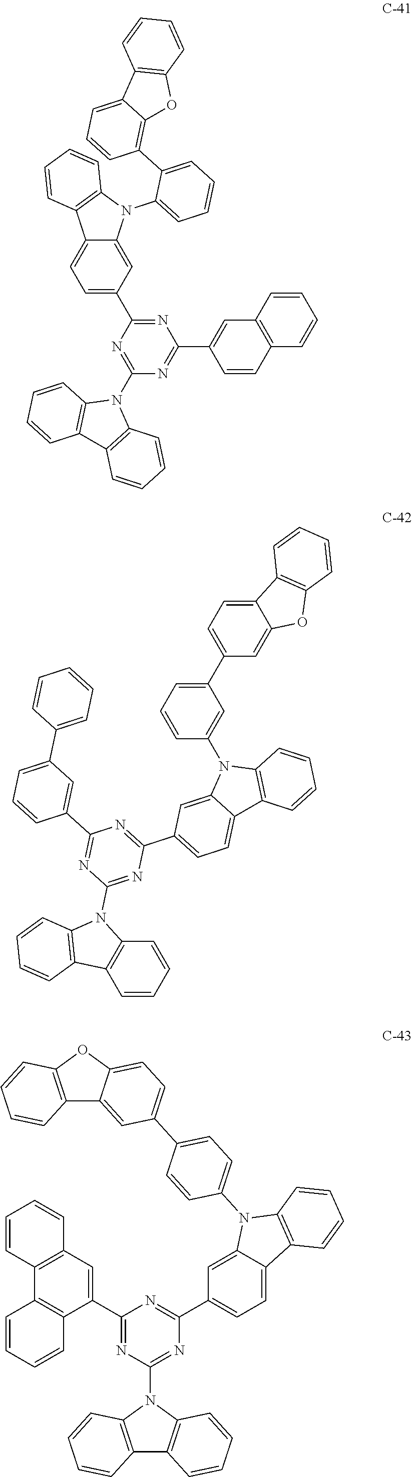 [SMILES: c1ccc(-n2c3ccccc3c3ccc(-c4nc(-c5ccc6ccccc6c5)nc(-n5c6ccccc6c6ccccc65)n4)cc32)c(-c2cccc3c2oc2ccccc23)c1 ; c1ccc(-c2cccc(-c3nc(-c4ccc5c6ccccc6n(-c6cccc(-c7ccc8c(c7)oc7ccccc78)c6)c5c4)nc(-n4c5ccccc5c5ccccc54)n3)c2)cc1 ; c1ccc2c(c1)cc(-c1nc(-c3ccc4c5ccccc5n(-c5ccc(-c6ccc7oc8ccccc8c7c6)cc5)c4c3)nc(-n3c4ccccc4c4ccccc43)n1)c1ccccc12] 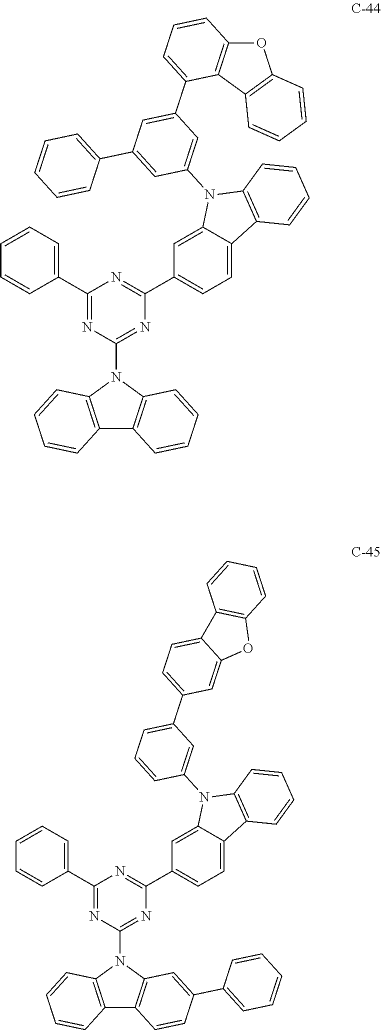 [SMILES: c1ccc(-c2cc(-c3cccc4oc5ccccc5c34)cc(-n3c4ccccc4c4ccc(-c5nc(-c6ccccc6)nc(-n6c7ccccc7c7ccccc76)n5)cc43)c2)cc1 ; c1ccc(-c2ccc3c4ccccc4n(-c4nc(-c5ccccc5)nc(-c5ccc6c7ccccc7n(-c7cccc(-c8ccc9c(c8)oc8ccccc89)c7)c6c5)n4)c3c2)cc1] 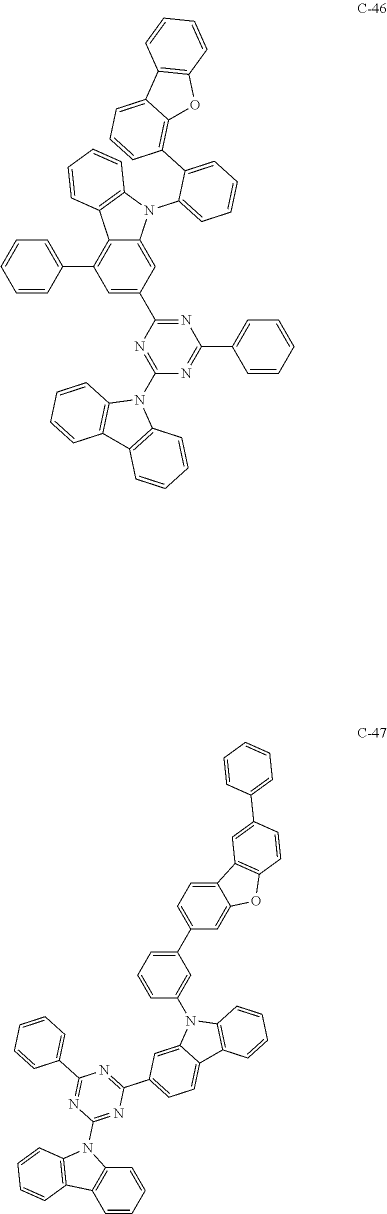 [SMILES: c1ccc(-c2nc(-c3cc(-c4ccccc4)c4c5ccccc5n(-c5ccccc5-c5cccc6c5oc5ccccc56)c4c3)nc(-n3c4ccccc4c4ccccc43)n2)cc1 ; c1ccc(-c2ccc3oc4cc(-c5cccc(-n6c7ccccc7c7ccc(-c8nc(-c9ccccc9)nc(-n9c%10ccccc%10c%10ccccc%109)n8)cc76)c5)ccc4c3c2)cc1] 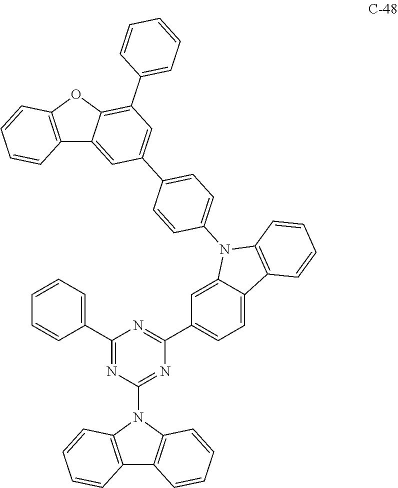 [SMILES: c1ccc(-c2nc(-c3ccc4c5ccccc5n(-c5ccc(-c6cc(-c7ccccc7)c7oc8ccccc8c7c6)cc5)c4c3)nc(-n3c4ccccc4c4ccccc43)n2)cc1] 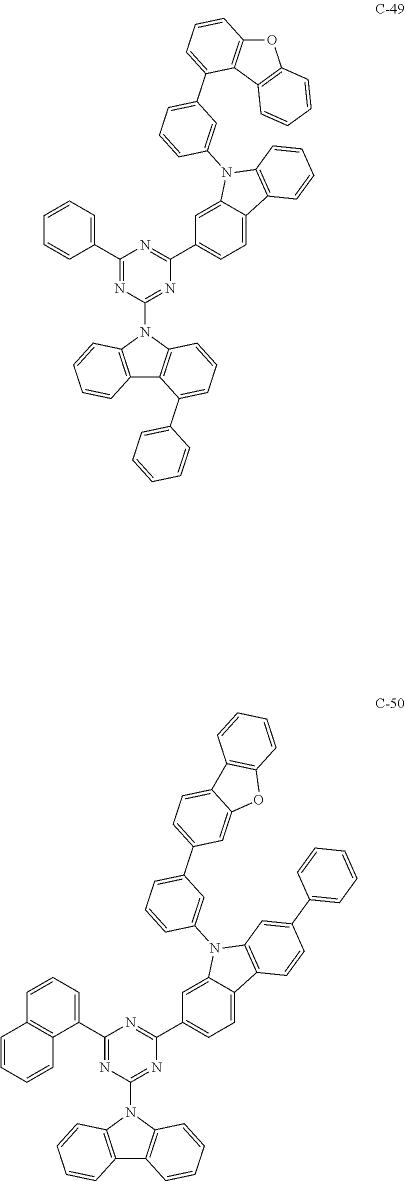 [SMILES: c1ccc(-c2nc(-c3ccc4c5ccccc5n(-c5cccc(-c6cccc7oc8ccccc8c67)c5)c4c3)nc(-n3c4ccccc4c4c(-c5ccccc5)cccc43)n2)cc1 ; c1ccc(-c2ccc3c4ccc(-c5nc(-c6cccc7ccccc67)nc(-n6c7ccccc7c7ccccc76)n5)cc4n(-c4cccc(-c5ccc6c(c5)oc5ccccc56)c4)c3c2)cc1] 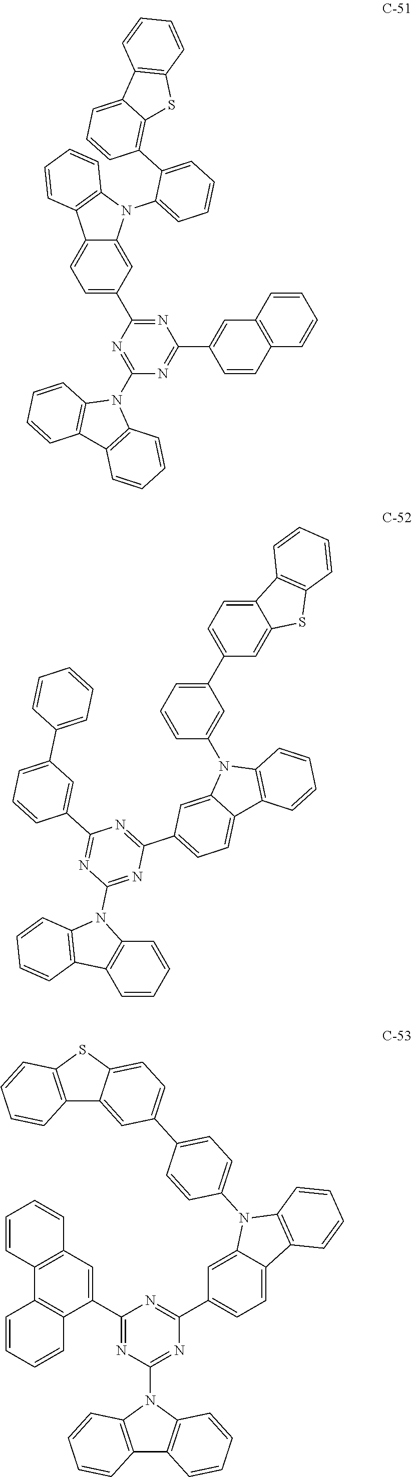 [SMILES: c1ccc(-n2c3ccccc3c3ccc(-c4nc(-c5ccc6ccccc6c5)nc(-n5c6ccccc6c6ccccc65)n4)cc32)c(-c2cccc3c2sc2ccccc23)c1 ; c1ccc(-c2cccc(-c3nc(-c4ccc5c6ccccc6n(-c6cccc(-c7ccc8c(c7)sc7ccccc78)c6)c5c4)nc(-n4c5ccccc5c5ccccc54)n3)c2)cc1 ; c1ccc2c(c1)cc(-c1nc(-c3ccc4c5ccccc5n(-c5ccc(-c6ccc7sc8ccccc8c7c6)cc5)c4c3)nc(-n3c4ccccc4c4ccccc43)n1)c1ccccc12] 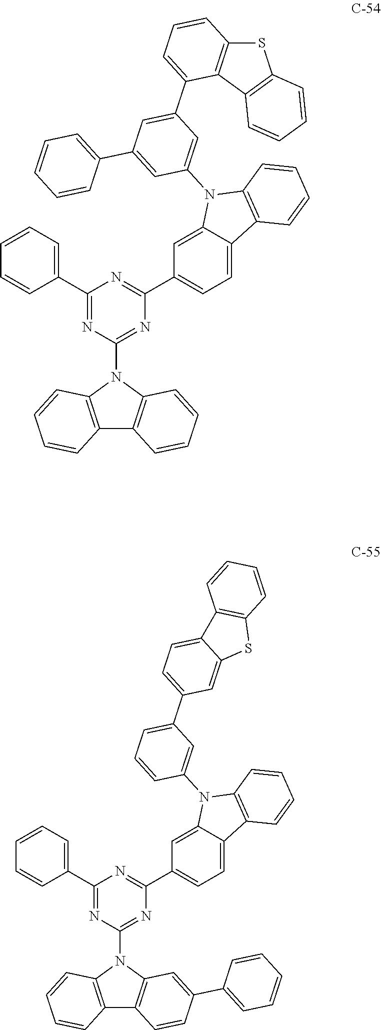 [SMILES: c1ccc(-c2cc(-c3cccc4sc5ccccc5c34)cc(-n3c4ccccc4c4ccc(-c5nc(-c6ccccc6)nc(-n6c7ccccc7c7ccccc76)n5)cc43)c2)cc1 ; c1ccc(-c2ccc3c4ccccc4n(-c4nc(-c5ccccc5)nc(-c5ccc6c7ccccc7n(-c7cccc(-c8ccc9c(c8)sc8ccccc89)c7)c6c5)n4)c3c2)cc1] 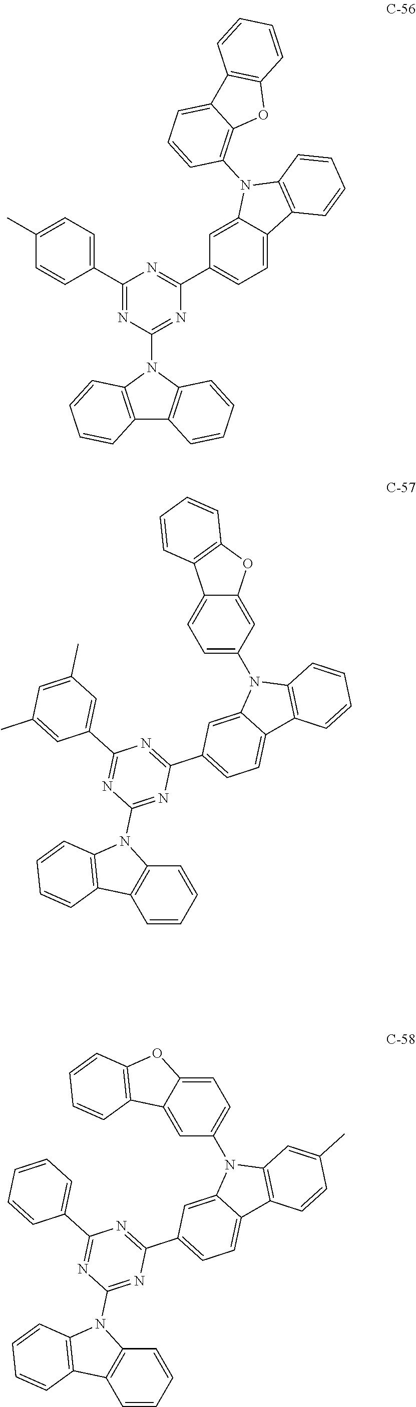 [SMILES: Cc1ccc(-c2nc(-c3ccc4c5ccccc5n(-c5cccc6c5oc5ccccc56)c4c3)nc(-n3c4ccccc4c4ccccc43)n2)cc1 ; Cc1cc(C)cc(-c2nc(-c3ccc4c5ccccc5n(-c5ccc6c(c5)oc5ccccc56)c4c3)nc(-n3c4ccccc4c4ccccc43)n2)c1 ; Cc1ccc2c3ccc(-c4nc(-c5ccccc5)nc(-n5c6ccccc6c6ccccc65)n4)cc3n(-c3ccc4oc5ccccc5c4c3)c2c1] 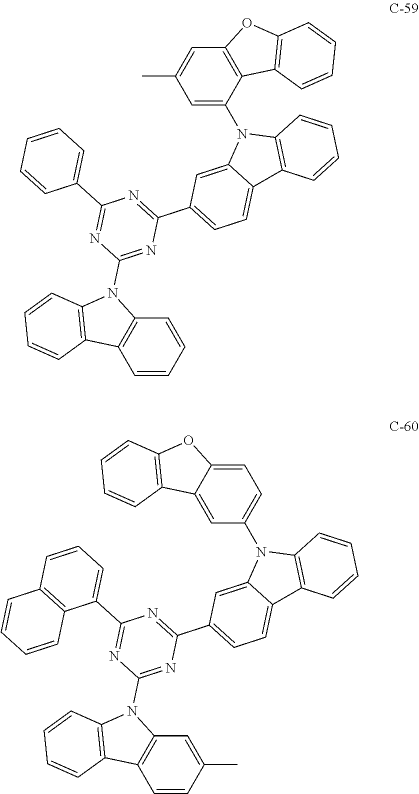 [SMILES: Cc1cc(-n2c3ccccc3c3ccc(-c4nc(-c5ccccc5)nc(-n5c6ccccc6c6ccccc65)n4)cc32)c2c(c1)oc1ccccc12 ; Cc1ccc2c3ccccc3n(-c3nc(-c4ccc5c6ccccc6n(-c6ccc7oc8ccccc8c7c6)c5c4)nc(-c4cccc5ccccc45)n3)c2c1] 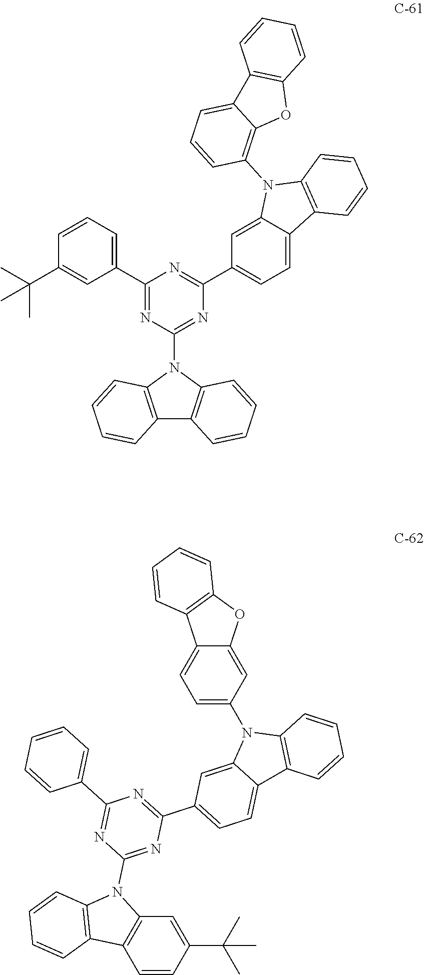 [SMILES: CC(C)(C)c1cccc(-c2nc(-c3ccc4c5ccccc5n(-c5cccc6c5oc5ccccc56)c4c3)nc(-n3c4ccccc4c4ccccc43)n2)c1 ; CC(C)(C)c1ccc2c3ccccc3n(-c3nc(-c4ccccc4)nc(-c4ccc5c6ccccc6n(-c6ccc7c(c6)oc6ccccc67)c5c4)n3)c2c1] 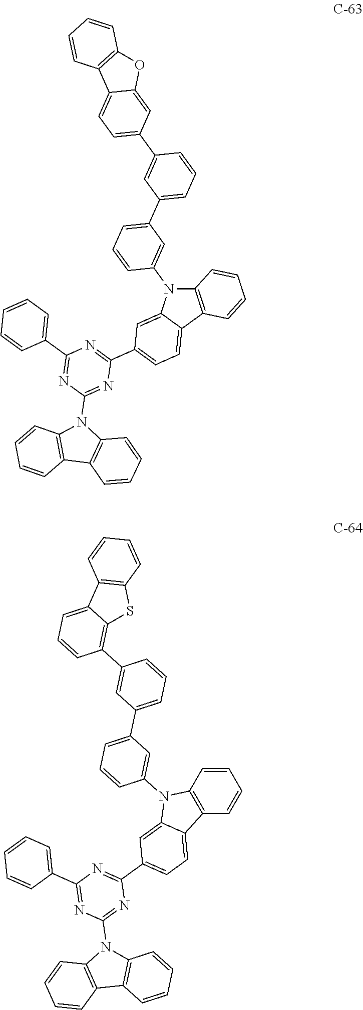 [SMILES: c1ccc(-c2nc(-c3ccc4c5ccccc5n(-c5cccc(-c6cccc(-c7ccc8c(c7)oc7ccccc78)c6)c5)c4c3)nc(-n3c4ccccc4c4ccccc43)n2)cc1 ; c1ccc(-c2nc(-c3ccc4c5ccccc5n(-c5cccc(-c6cccc(-c7cccc8c7sc7ccccc78)c6)c5)c4c3)nc(-n3c4ccccc4c4ccccc43)n2)cc1] 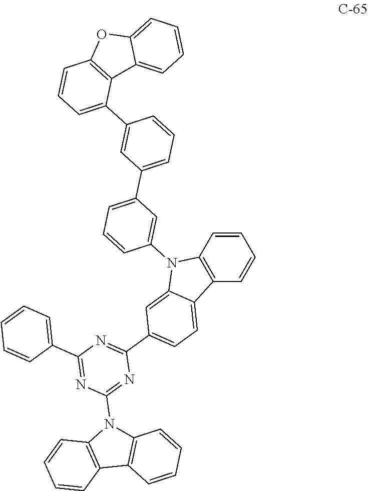 [SMILES: c1ccc(-c2nc(-c3ccc4c5ccccc5n(-c5cccc(-c6cccc(-c7cccc8oc9ccccc9c78)c6)c5)c4c3)nc(-n3c4ccccc4c4ccccc43)n2)cc1] 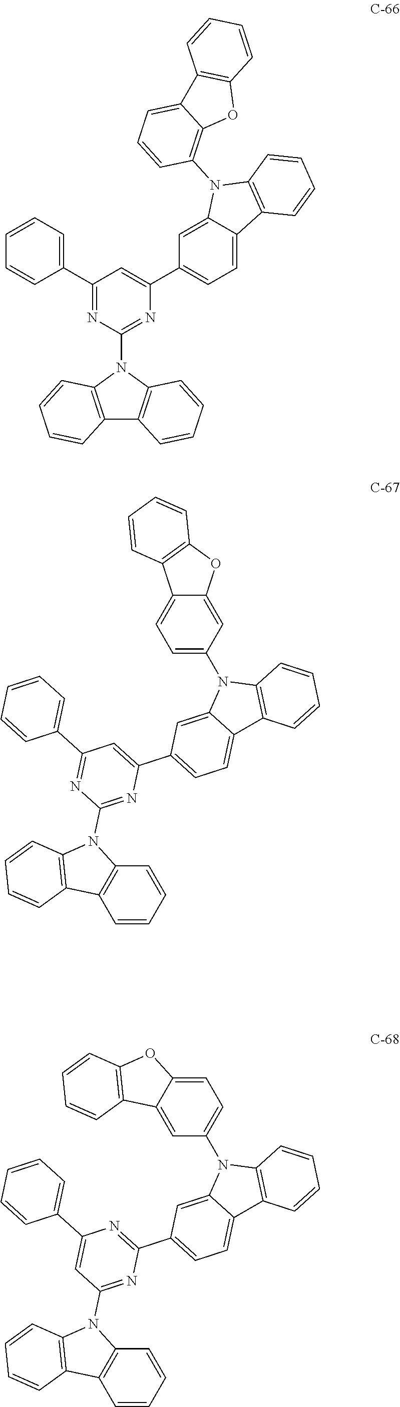 [SMILES: c1ccc(-c2cc(-c3ccc4c5ccccc5n(-c5cccc6c5oc5ccccc56)c4c3)nc(-n3c4ccccc4c4ccccc43)n2)cc1 ; c1ccc(-c2cc(-c3ccc4c5ccccc5n(-c5ccc6c(c5)oc5ccccc56)c4c3)nc(-n3c4ccccc4c4ccccc43)n2)cc1 ; c1ccc(-c2cc(-n3c4ccccc4c4ccccc43)nc(-c3ccc4c5ccccc5n(-c5ccc6oc7ccccc7c6c5)c4c3)n2)cc1] 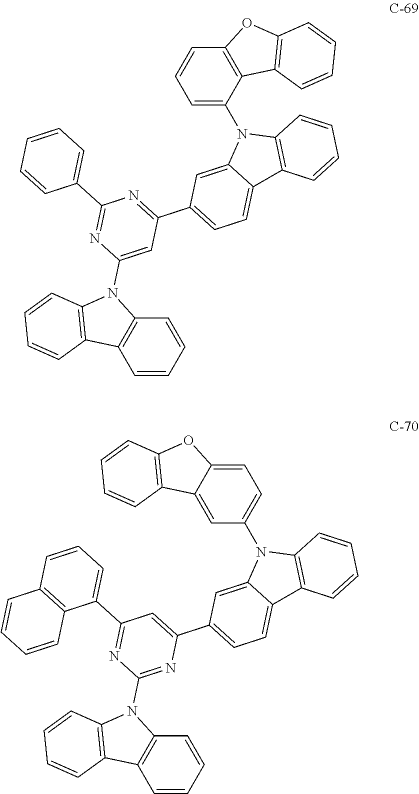 [SMILES: c1ccc(-c2nc(-c3ccc4c5ccccc5n(-c5cccc6oc7ccccc7c56)c4c3)cc(-n3c4ccccc4c4ccccc43)n2)cc1 ; c1ccc2c(-c3cc(-c4ccc5c6ccccc6n(-c6ccc7oc8ccccc8c7c6)c5c4)nc(-n4c5ccccc5c5ccccc54)n3)cccc2c1] 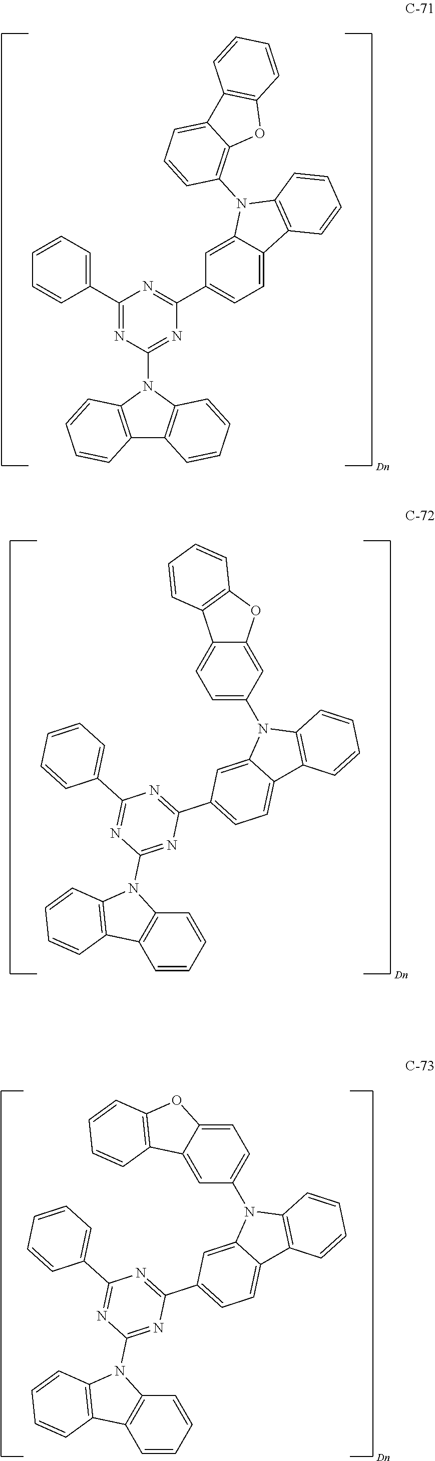 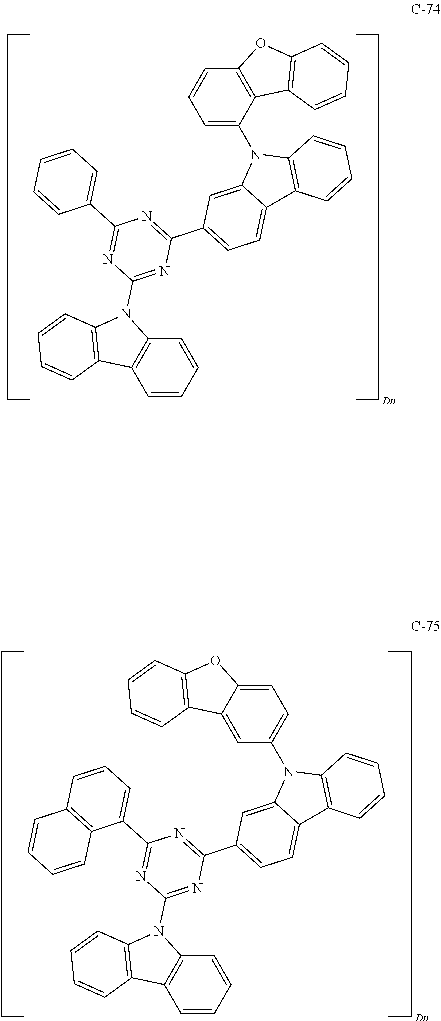 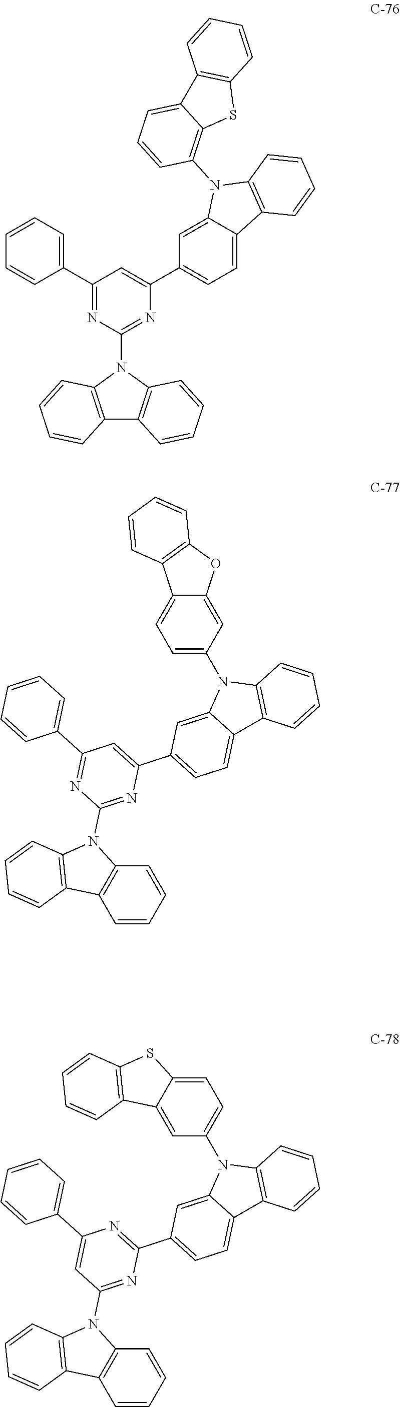 [SMILES: c1ccc(-c2cc(-c3ccc4c5ccccc5n(-c5cccc6c5sc5ccccc56)c4c3)nc(-n3c4ccccc4c4ccccc43)n2)cc1 ; c1ccc(-c2cc(-c3ccc4c5ccccc5n(-c5ccc6c(c5)oc5ccccc56)c4c3)nc(-n3c4ccccc4c4ccccc43)n2)cc1 ; c1ccc(-c2cc(-n3c4ccccc4c4ccccc43)nc(-c3ccc4c5ccccc5n(-c5ccc6sc7ccccc7c6c5)c4c3)n2)cc1] 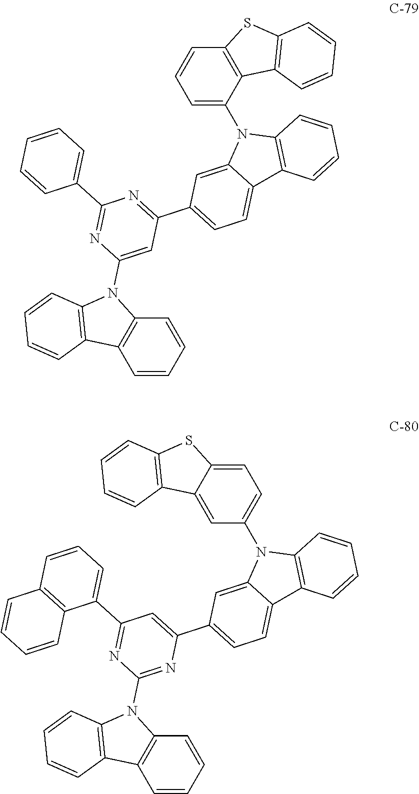 [SMILES: c1ccc(-c2nc(-c3ccc4c5ccccc5n(-c5cccc6sc7ccccc7c56)c4c3)cc(-n3c4ccccc4c4ccccc43)n2)cc1 ; c1ccc2c(-c3cc(-c4ccc5c6ccccc6n(-c6ccc7sc8ccccc8c7c6)c5c4)nc(-n4c5ccccc5c5ccccc54)n3)cccc2c1] 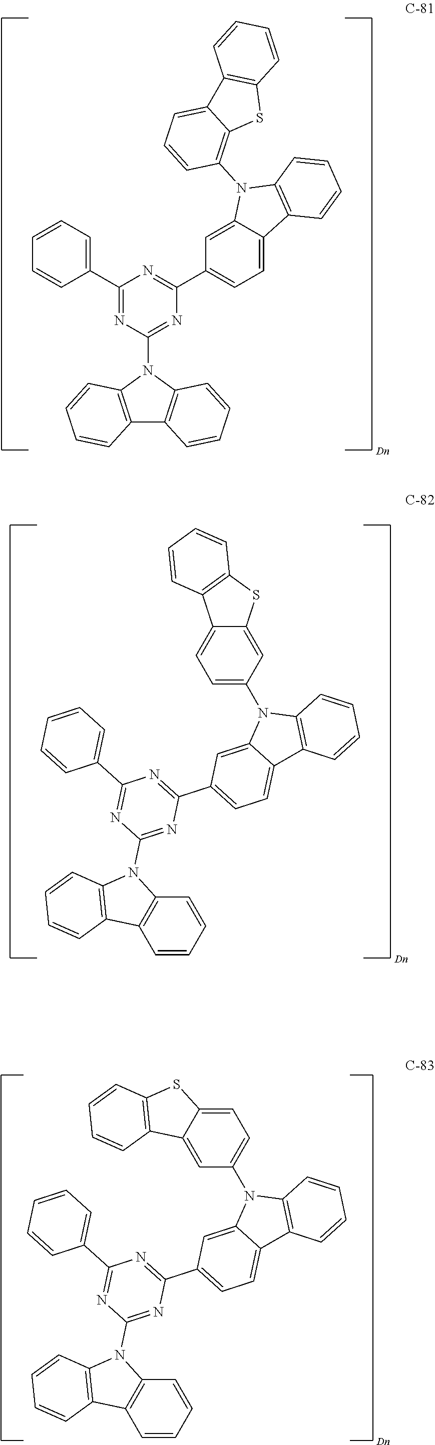 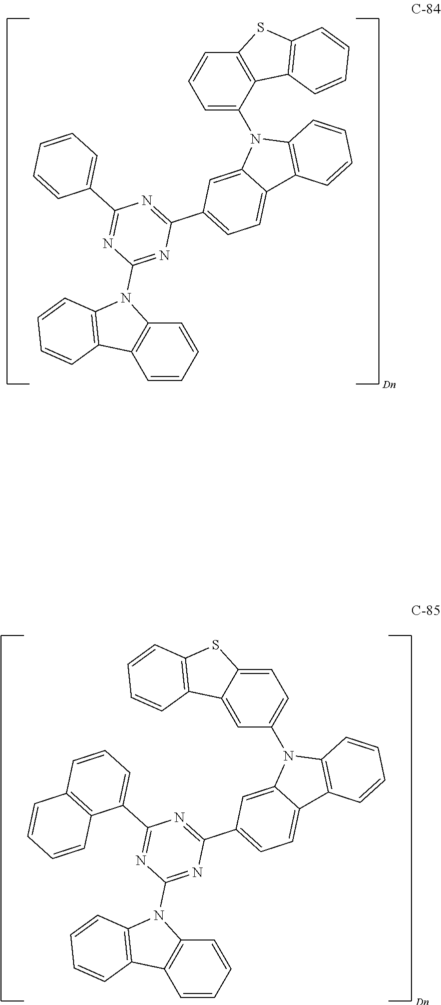 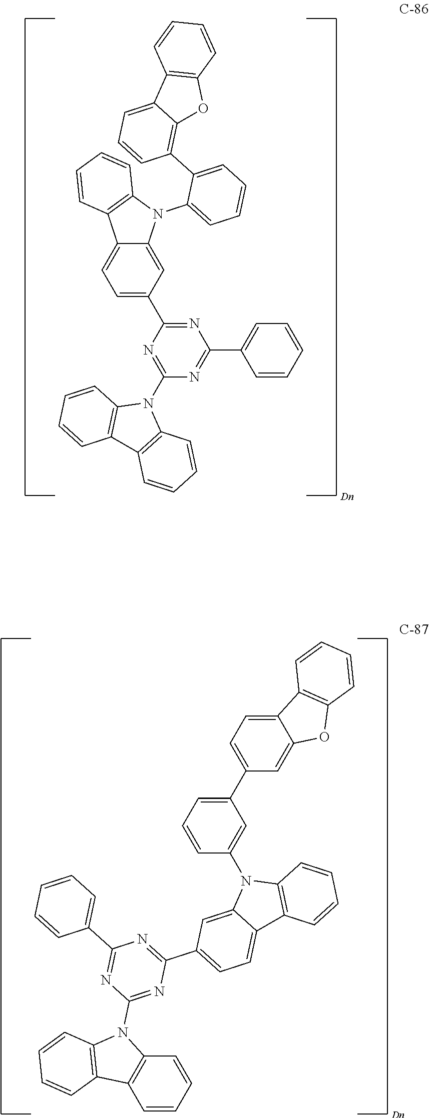 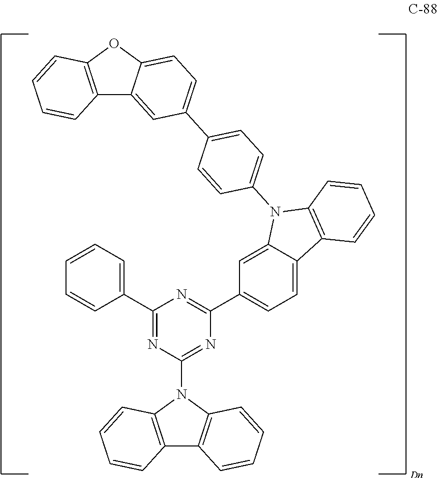 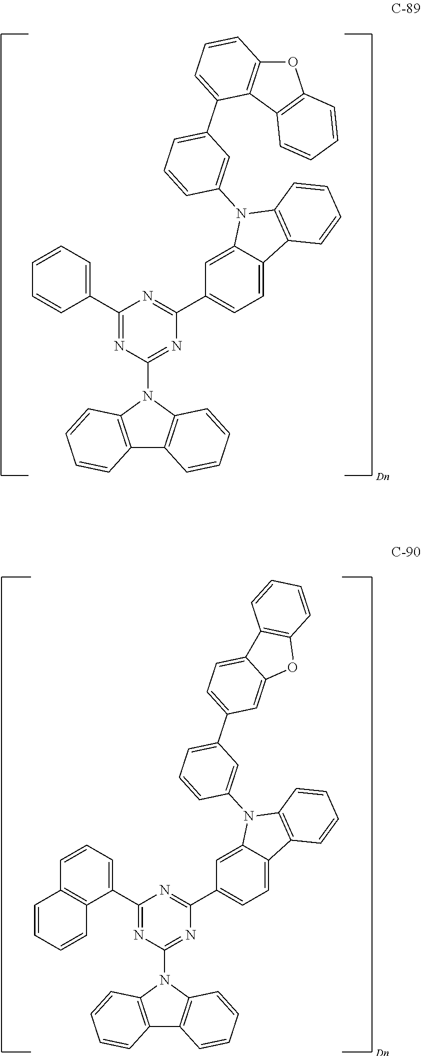 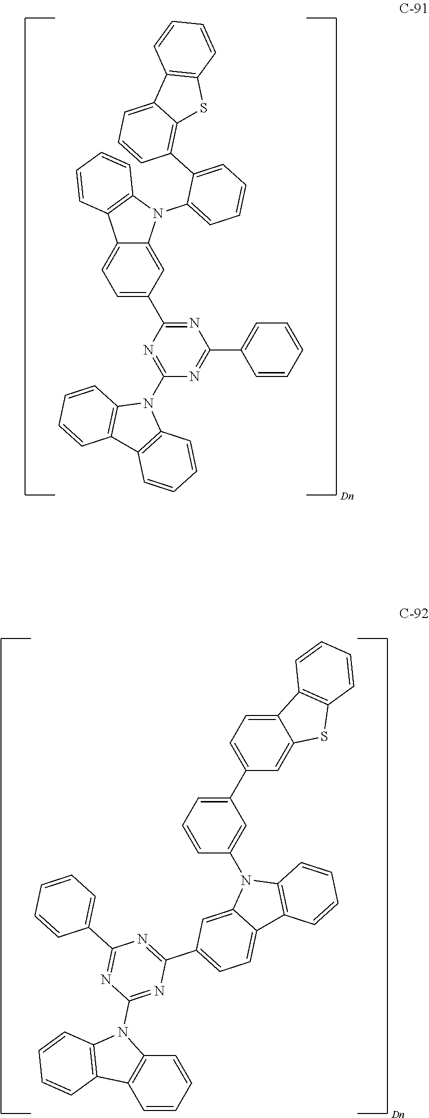 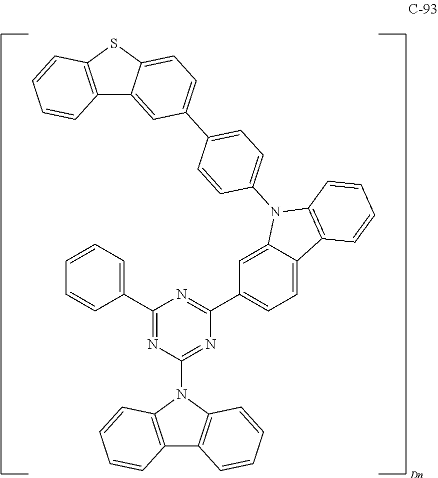 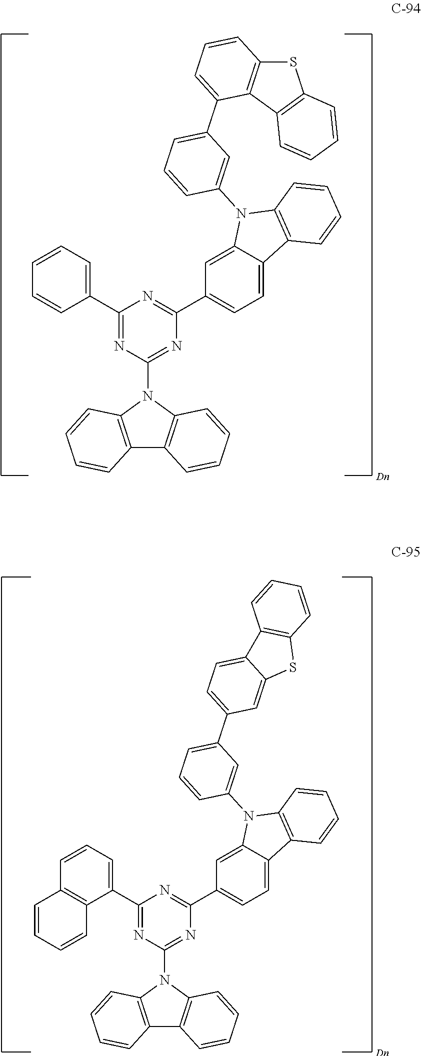 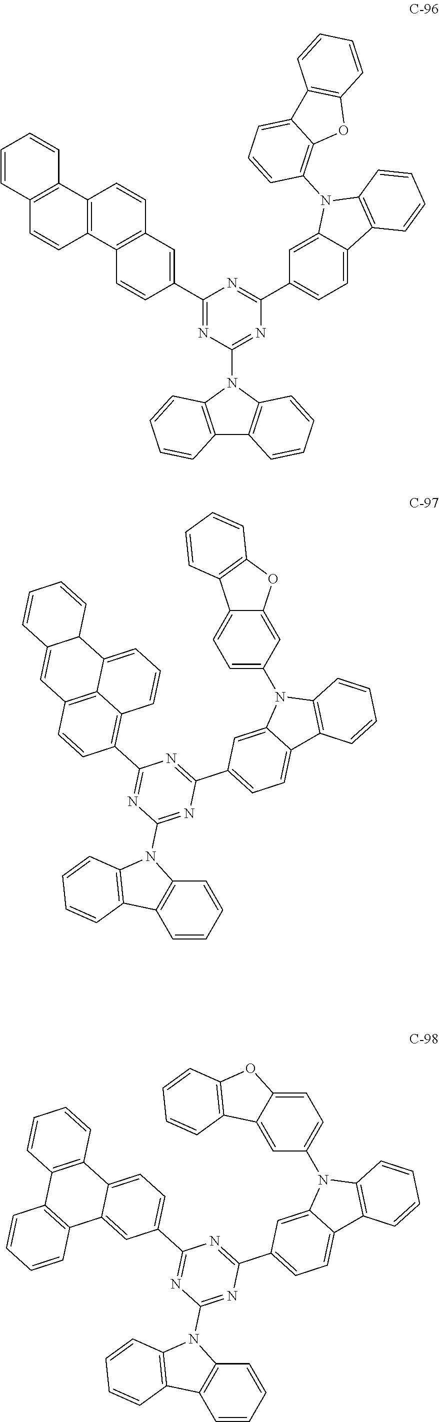 [SMILES: c1ccc2c(c1)ccc1c3ccc(-c4nc(-c5ccc6c7ccccc7n(-c7cccc8c7oc7ccccc78)c6c5)nc(-n5c6ccccc6c6ccccc65)n4)cc3ccc21 ; C1=CC2=Cc3ccc(-c4nc(-c5ccc6c7ccccc7n(-c7ccc8c(c7)oc7ccccc78)c6c5)nc(-n5c6ccccc6c6ccccc65)n4)c4cccc(c34)C2C=C1 ; c1ccc2c(c1)oc1ccc(-n3c4ccccc4c4ccc(-c5nc(-c6ccc7c8ccccc8c8ccccc8c7c6)nc(-n6c7ccccc7c7ccccc76)n5)cc43)cc12] 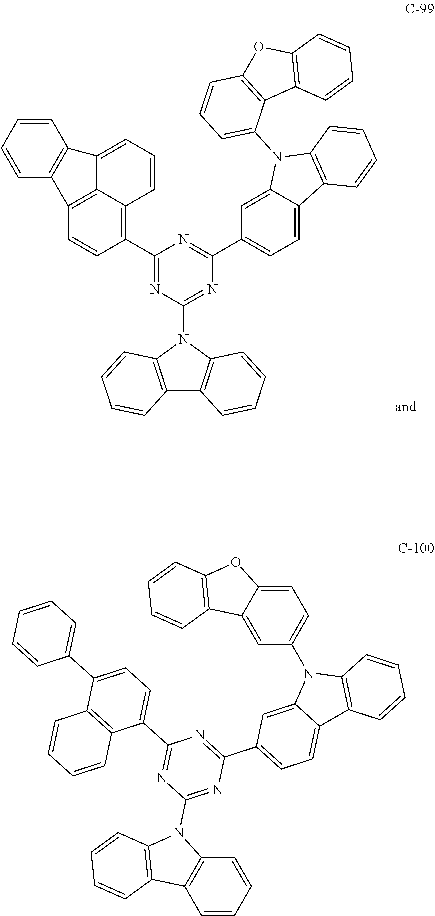 [SMILES: c1ccc2c(c1)-c1cccc3c(-c4nc(-c5ccc6c7ccccc7n(-c7cccc8oc9ccccc9c78)c6c5)nc(-n5c6ccccc6c6ccccc65)n4)ccc-2c13 ; c1ccc(-c2ccc(-c3nc(-c4ccc5c6ccccc6n(-c6ccc7oc8ccccc8c7c6)c5c4)nc(-n4c5ccccc5c5ccccc54)n3)c3ccccc23)cc1]
wherein “Dₙ” in the above compounds means that n number of hydrogens is replaced by deuterium, wherein n represents an integer of 1 or more, and the upper limit of n is determined by the number of hydrogens that can be substituted in each compound.

5. An organic electroluminescent material, comprising the compound represented by Formula 1 according to claim 1 and a compound represented by the following Formula 2 or 3: 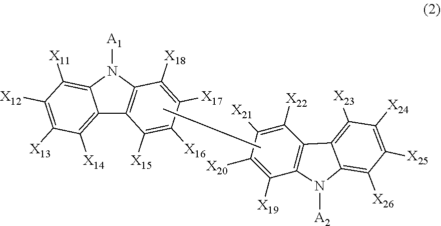
wherein,
A₁ and A₂ each independently represent a substituted or unsubstituted (C₆-C₃₀)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are linked to each other to form a single bond; and
X₁₁ to X₁₄, X₂₃ to X₂₆, and X₁₅ to X₂₂ which do not form a single bond each independently represent hydrogen, deuterium, a substituted or unsubstituted (C₆-C₃₀)aryl, or a substituted or unsubstituted (3- to 30-membered)heteroaryl; or may be linked to the adjacent substituents to form a ring(s); 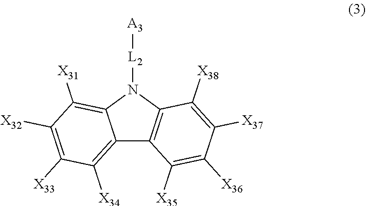
wherein,
A₃ represents a substituted or unsubstituted (C₆-C₃₀)aryl, a substituted or unsubstituted dibenzofuranyl, a substituted or unsubstituted dibenzothiophenyl, a substituted or unsubstituted dibenzoselenophenyl, or a substituted or unsubstituted carbazolyl;
L₂ represents a single bond or a substituted or unsubstituted (C₆-C₃₀)arylene;
a pair of adjacent X₃₅ to X₃₈ are linked to the following Formula 3-A to form a ring, and X₃₅ to X₃₈ which do not form a ring and X₃₁ to X₃₄ each independently represent hydrogen, deuterium, a substituted or unsubstituted (C₆-C₃₀)aryl, or a substituted or unsubstituted (3- to 30-membered)heteroaryl; and 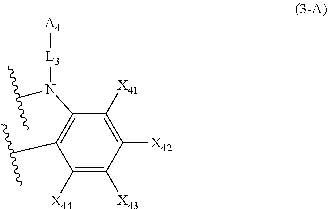
wherein,
A₄ represents a substituted or unsubstituted (C₆-C₃₀)aryl, a substituted or unsubstituted dibenzofuranyl, a substituted or unsubstituted dibenzothiophenyl, a substituted or unsubstituted dibenzoselenophenyl, or a substituted or unsubstituted carbazolyl;
L₃ represents a single bond or a substituted or unsubstituted (C₆-C₃₀)arylene; and
X₄₁ to X₄₄ each independently represent hydrogen, deuterium, a substituted or unsubstituted (C₆-C₃₀)aryl, or a substituted or unsubstituted (3- to 30-membered)heteroaryl.

6. The organic electroluminescent material according to claim 5, wherein at least one of X₁₁, X₁₈, X₁₉, and X₂₆ in Formula 2 is deuterium.

7. The organic electroluminescent material according to claim 5, wherein the deuterium substitution rate of X₁₁ to X₂₆ in Formula 2 is 25% to 100%.

8. The organic electroluminescent material according to claim 5, wherein the deuterium substitution rate of the compound represented by Formula 2 or 3 is 40% to 100%.

9. The organic electroluminescent material according to claim 5, wherein Formula 2 is represented by any one of the following Formulas 2-1 to 2-8. 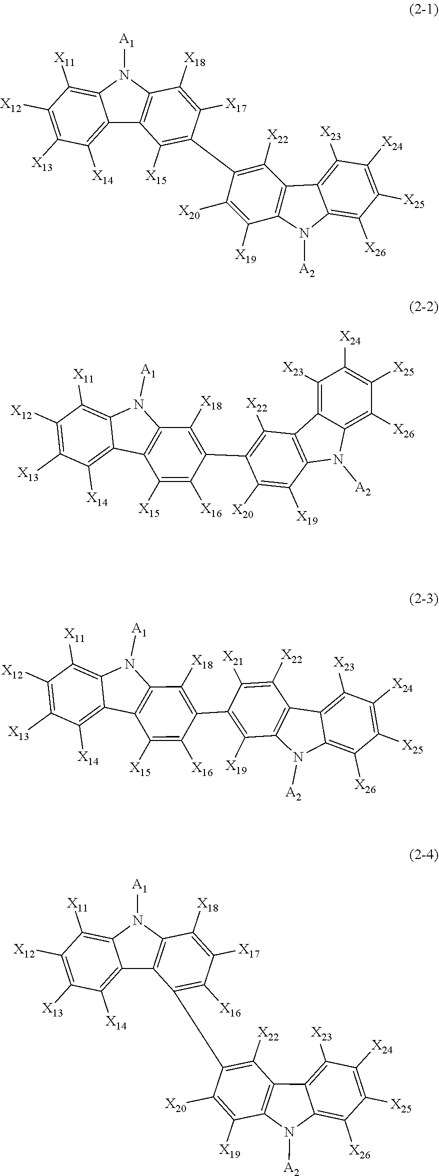 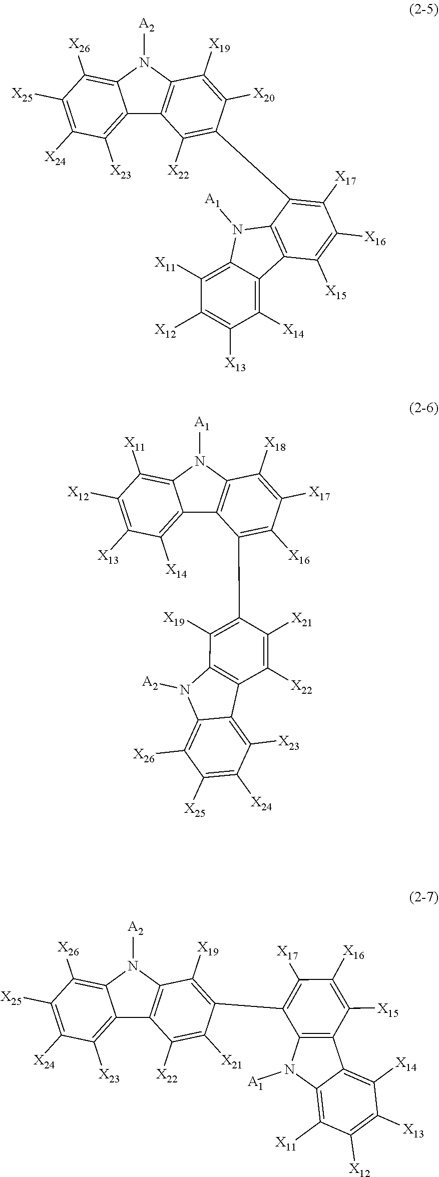 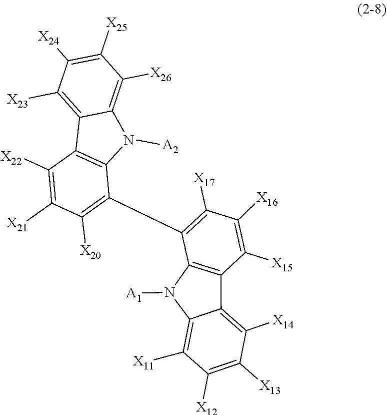
wherein,
A₁, A₂, and X₁₁ to X₂₆ each independently are as defined in claim 5.

10. The organic electroluminescent material according to claim 5, wherein A₁ and A₂ in Formula 2 each independently represent phenyl unsubstituted or substituted with deuterium; biphenyl unsubstituted or substituted with deuterium; terphenyl unsubstituted or substituted with deuterium; naphthyl unsubstituted or substituted with deuterium; fluorenyl unsubstituted or substituted with at least one of deuterium, (C₁-C₃₀)alkyl, and (C₆-C₃₀)aryl; benzofluorenyl unsubstituted or substituted with at least one of deuterium, (C₁-C₃₀)alkyl, and (C₆-C₃₀)aryl; triphenylenyl unsubstituted or substituted with deuterium; fluoranthenyl unsubstituted or substituted with deuterium; phenanthrenyl unsubstituted or substituted with deuterium; dibenzofuranyl unsubstituted or substituted with deuterium; dibenzothiophenyl unsubstituted or substituted with deuterium; carbazolyl unsubstituted or substituted with deuterium; or a combination thereof.

11. The organic electroluminescent material according to claim 5, wherein Formula 3 is represented by any one of the following Formulas 3-1 to 3-6. 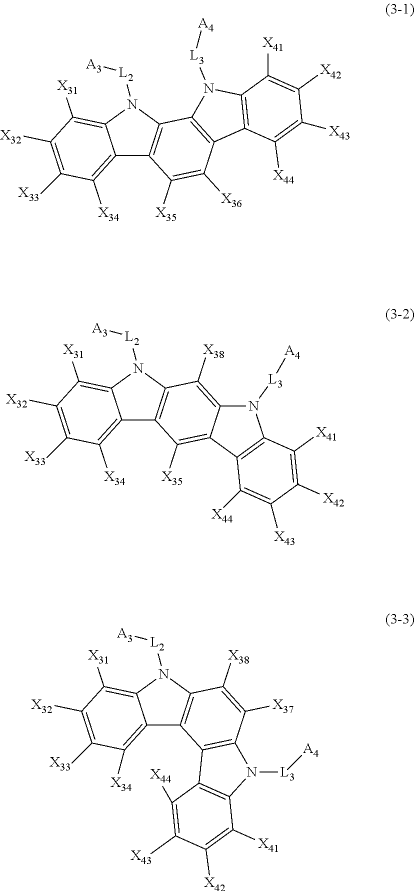 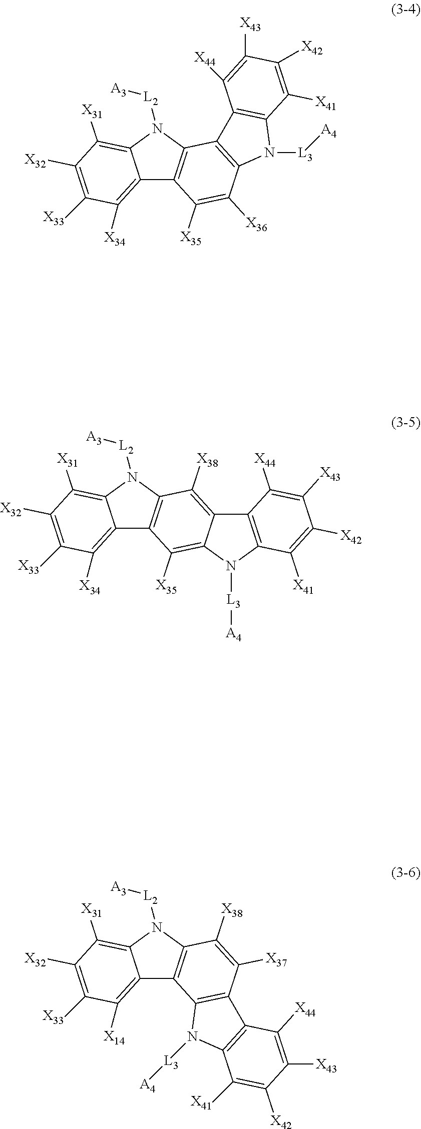
wherein,
A₃, A₄, L₂, L₃, X₃₁ to X₃₈, and X₄₁ to X₄₄ are as defined in claim 5.

12. The organic electroluminescent material according to claim 5, wherein the compound represented by Formula 2 is selected from the following compounds: 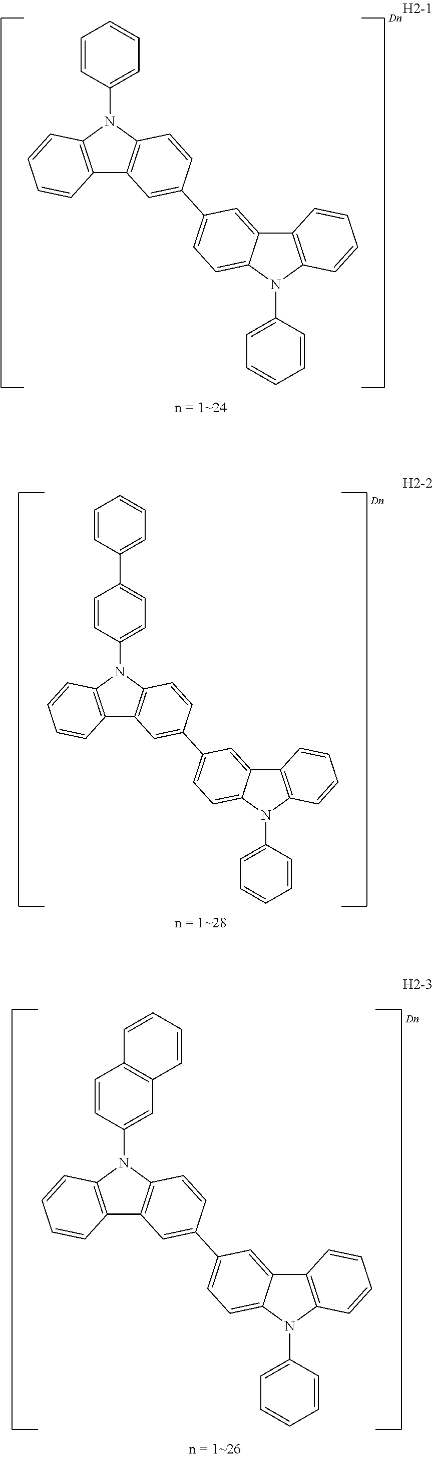 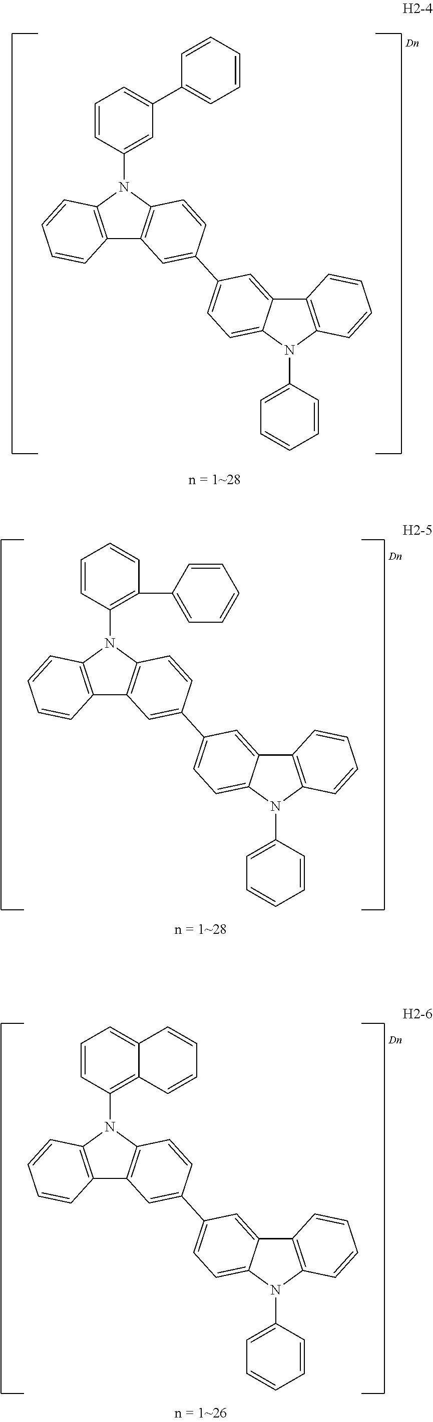 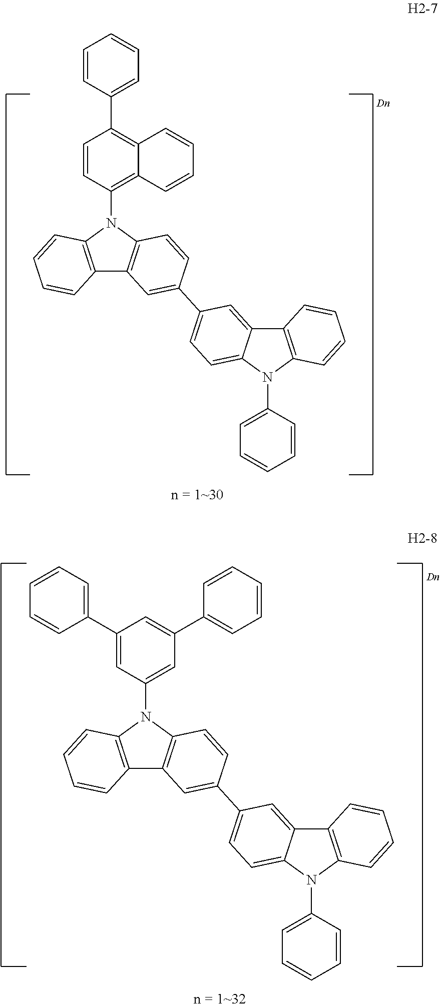 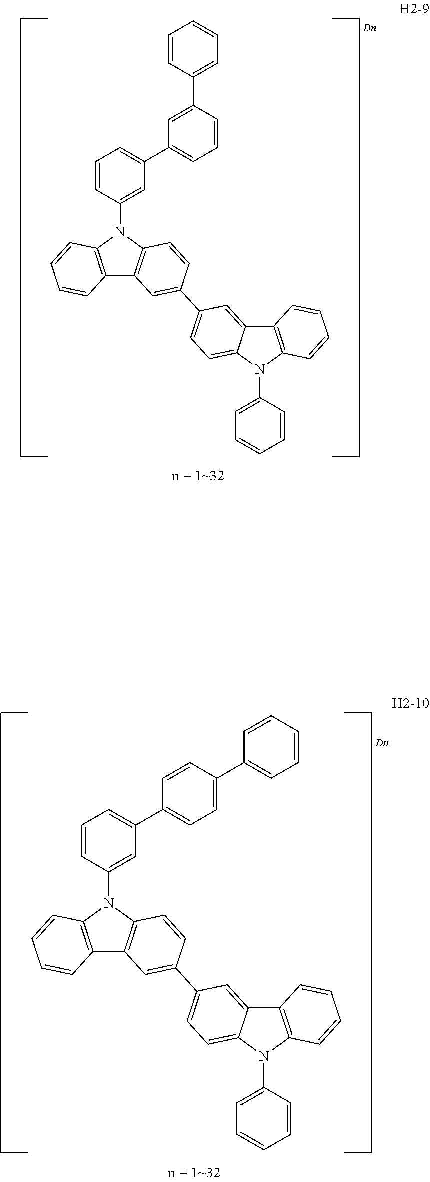 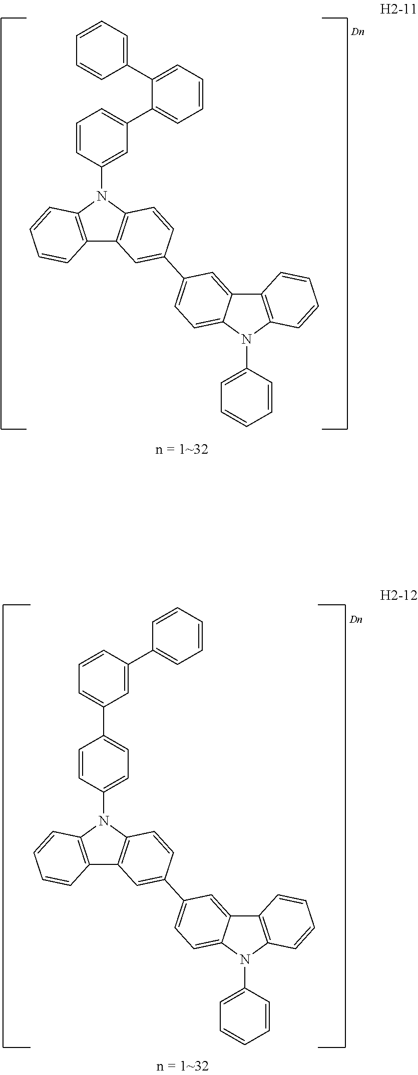 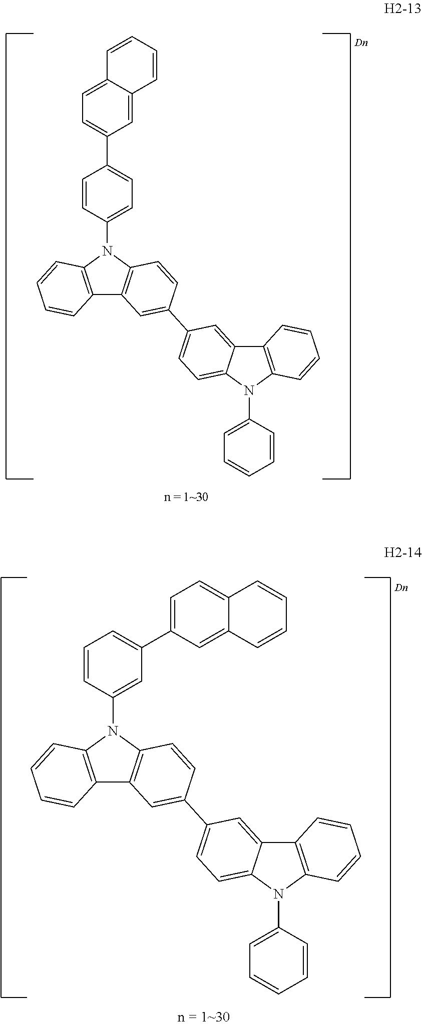 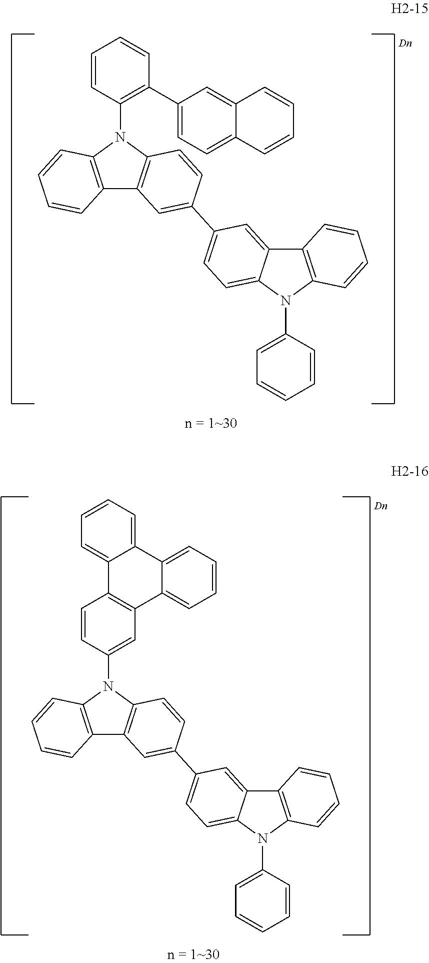 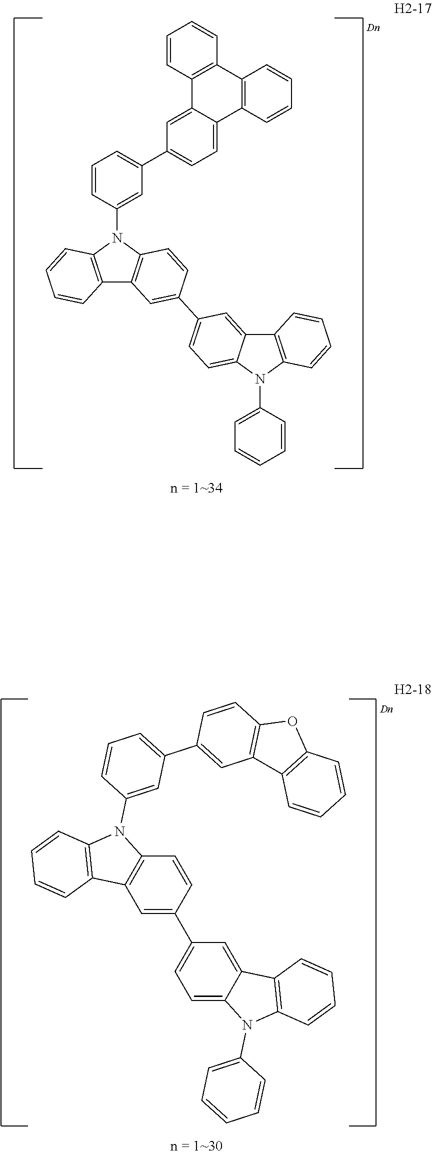 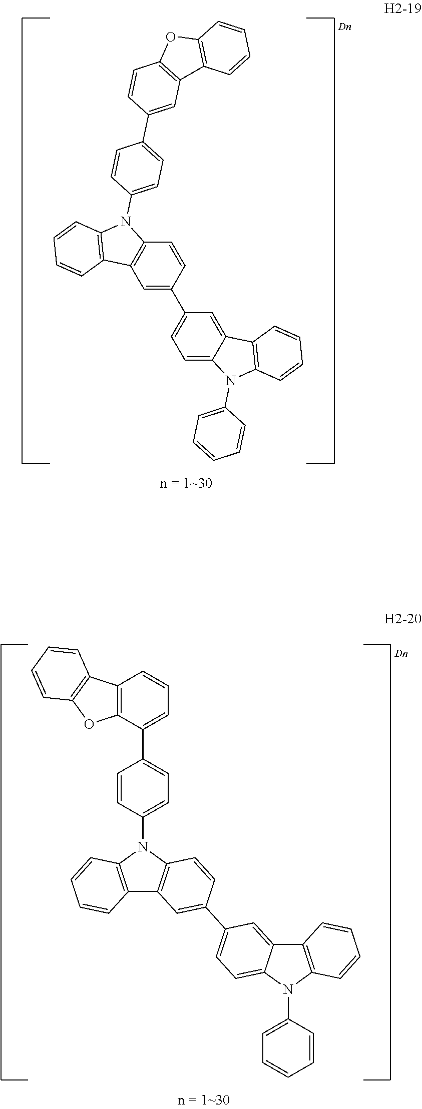 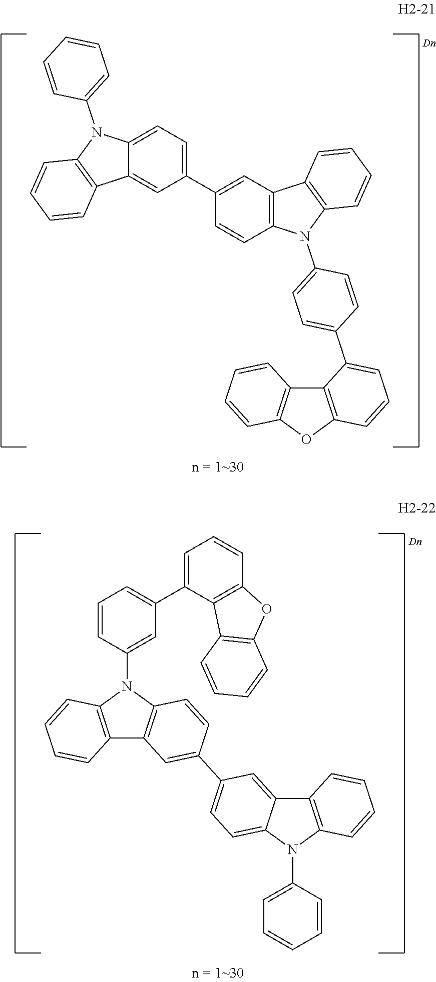 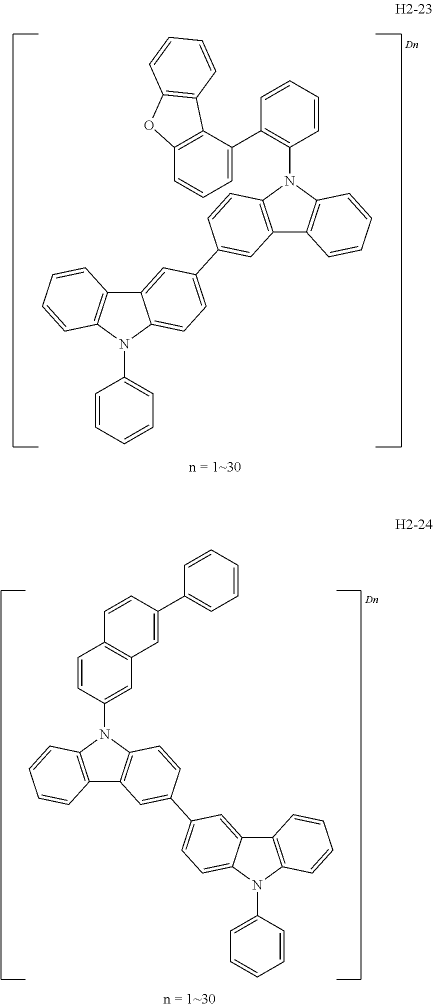 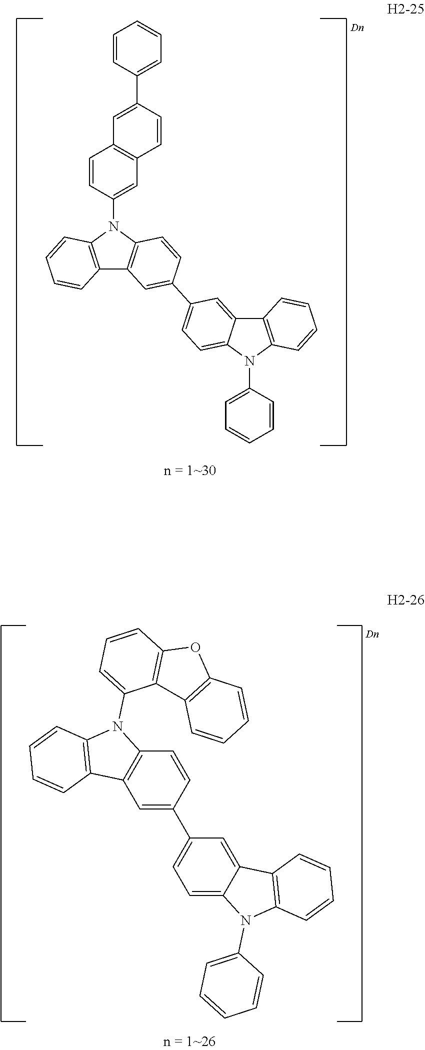 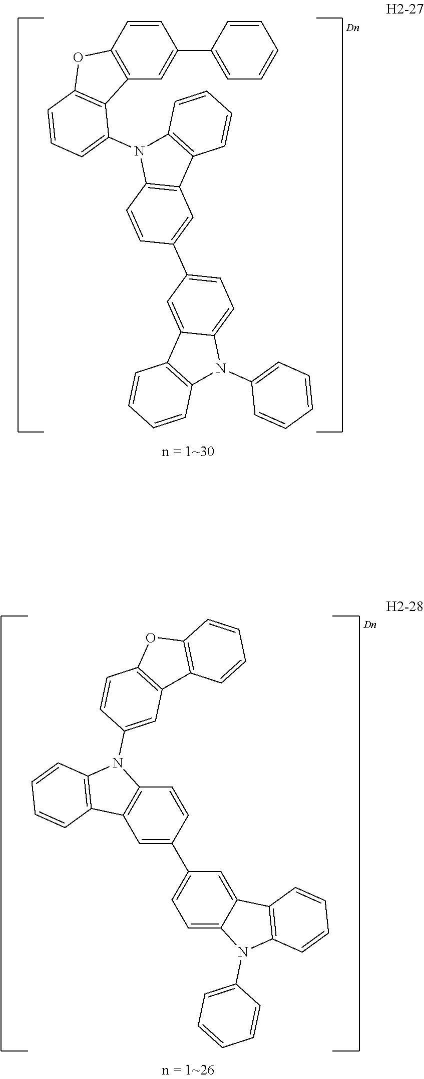 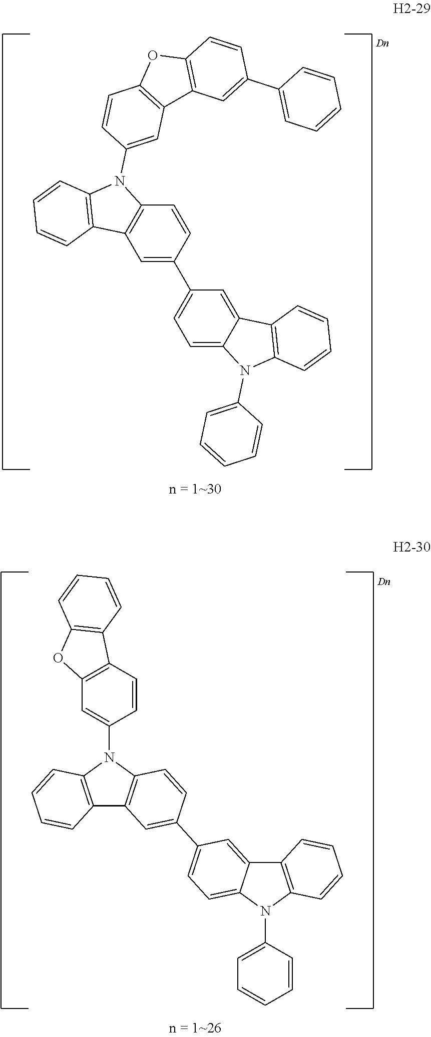 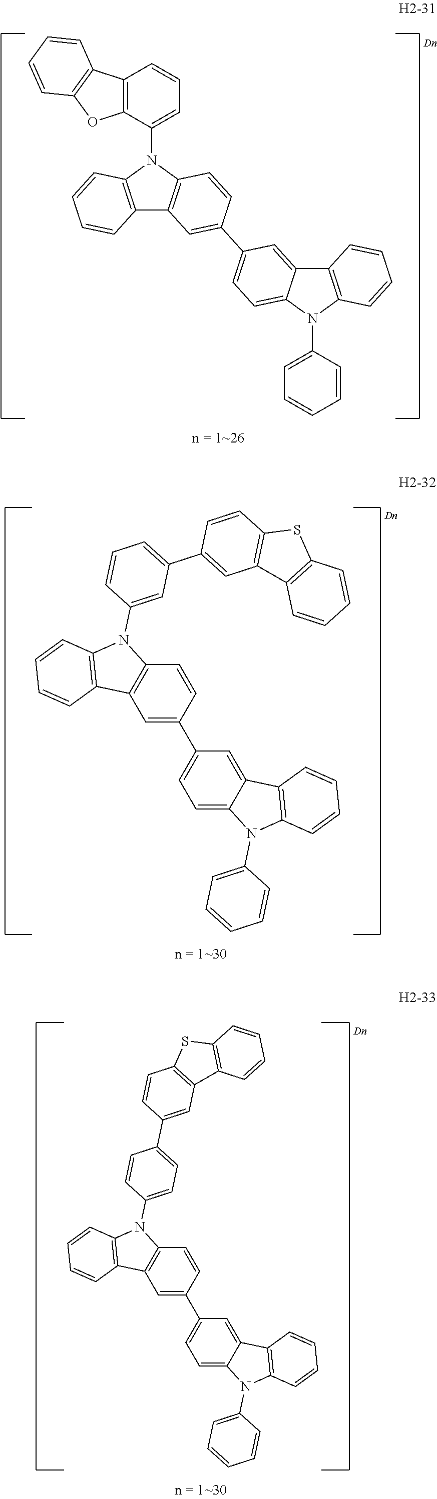 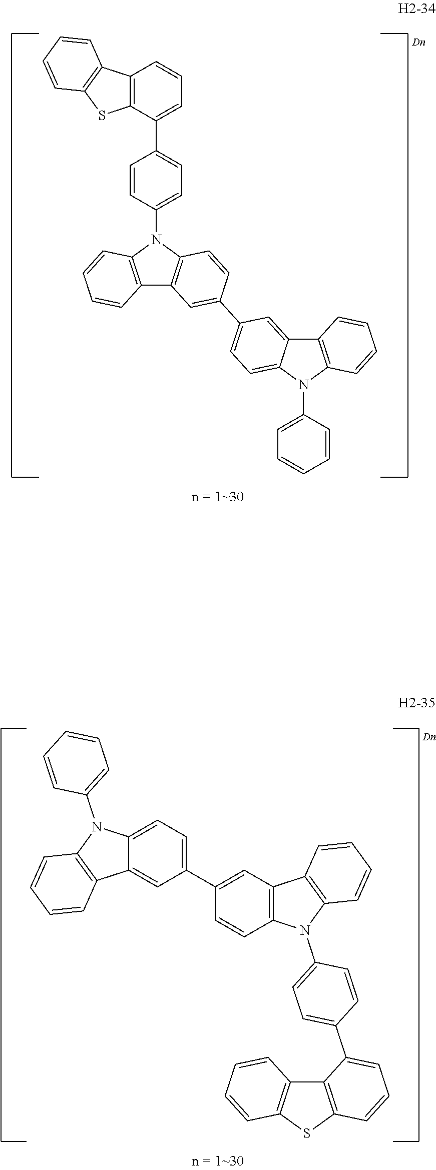 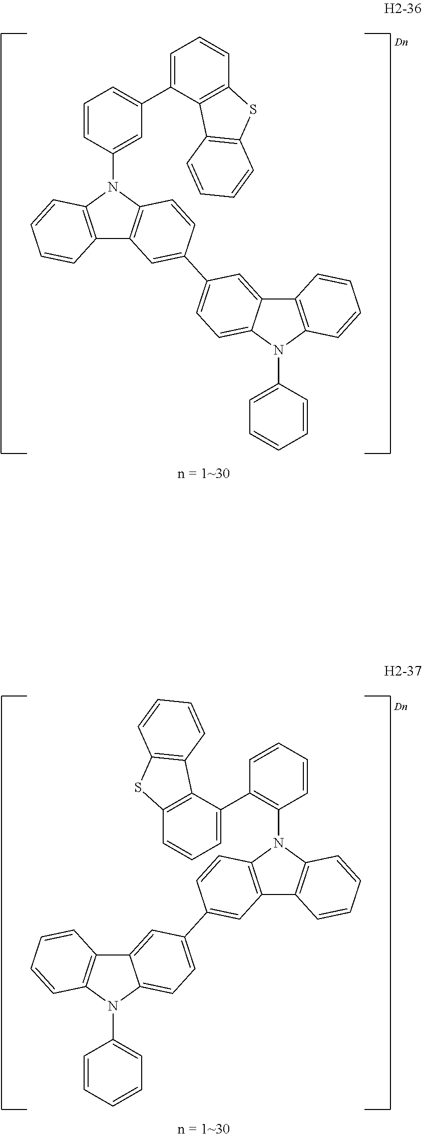 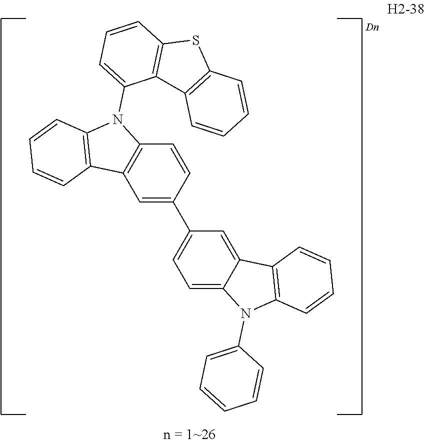 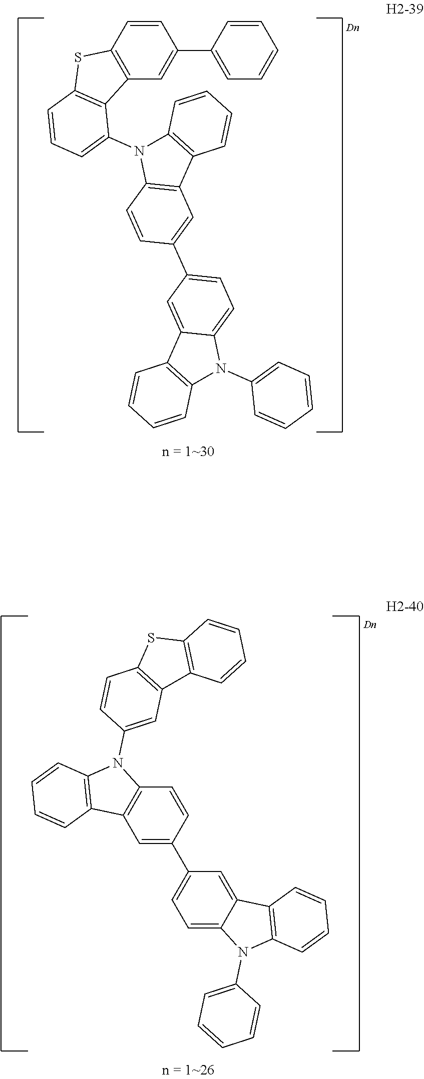 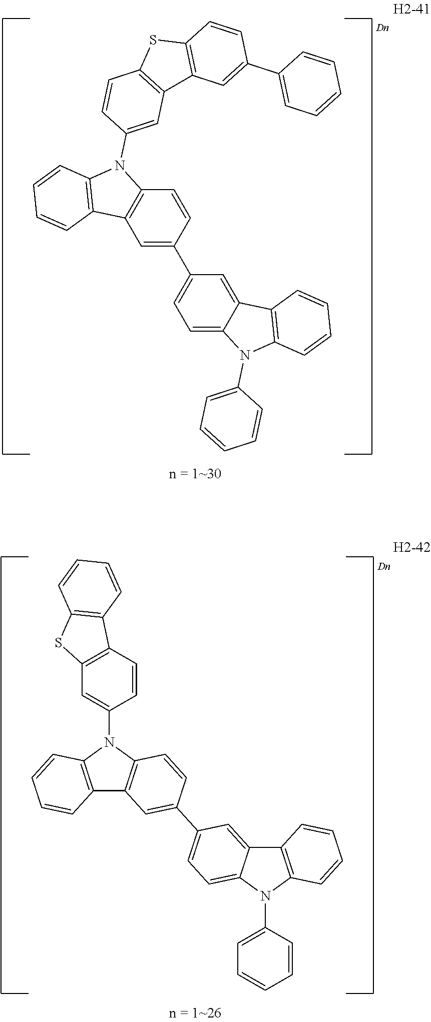 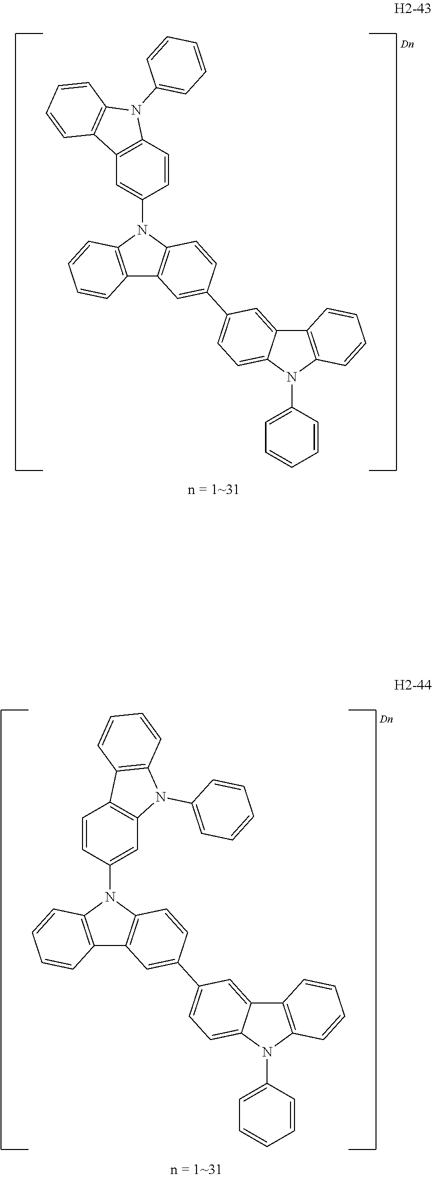 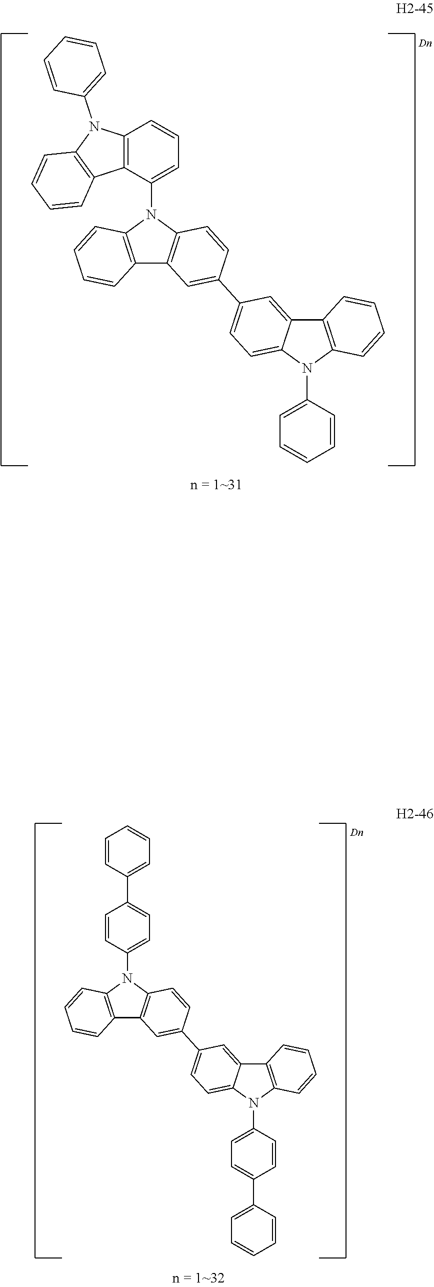 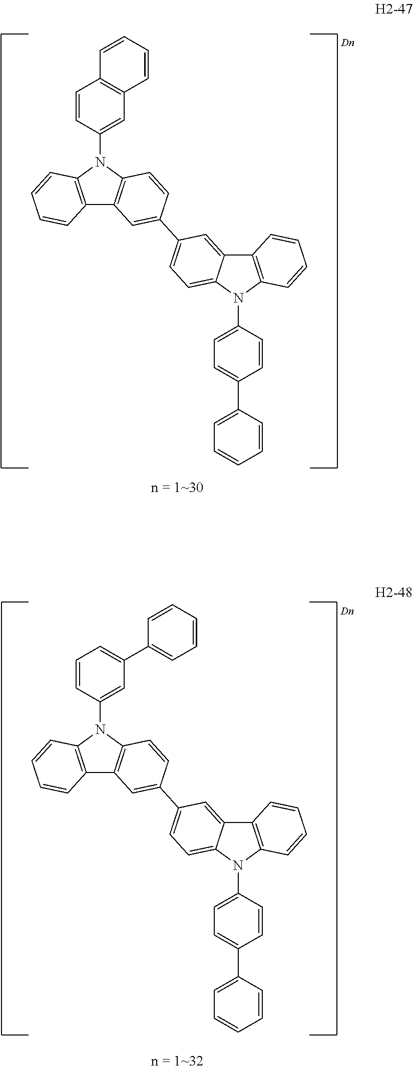 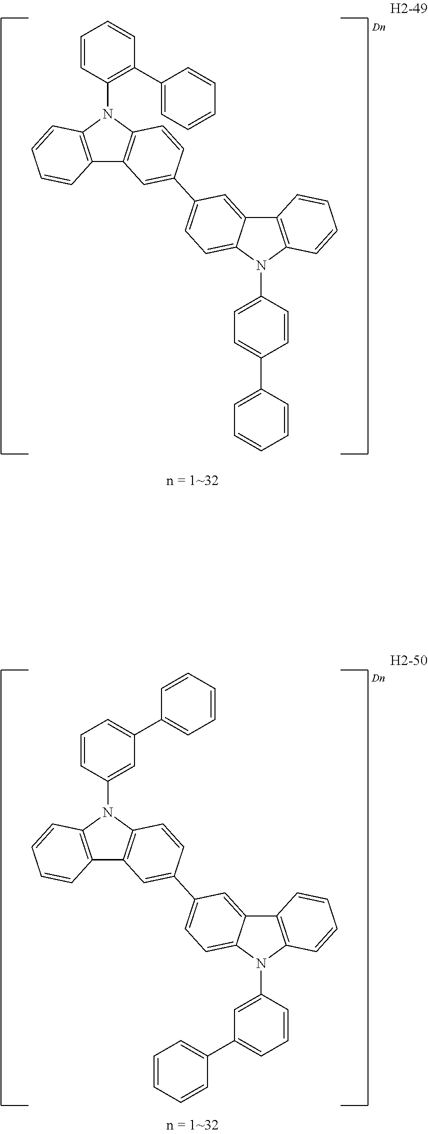 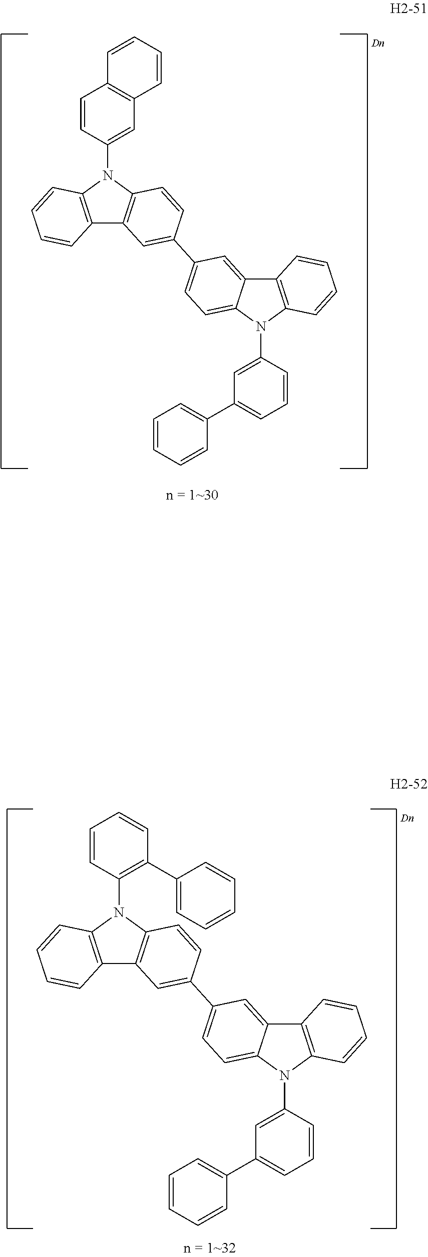 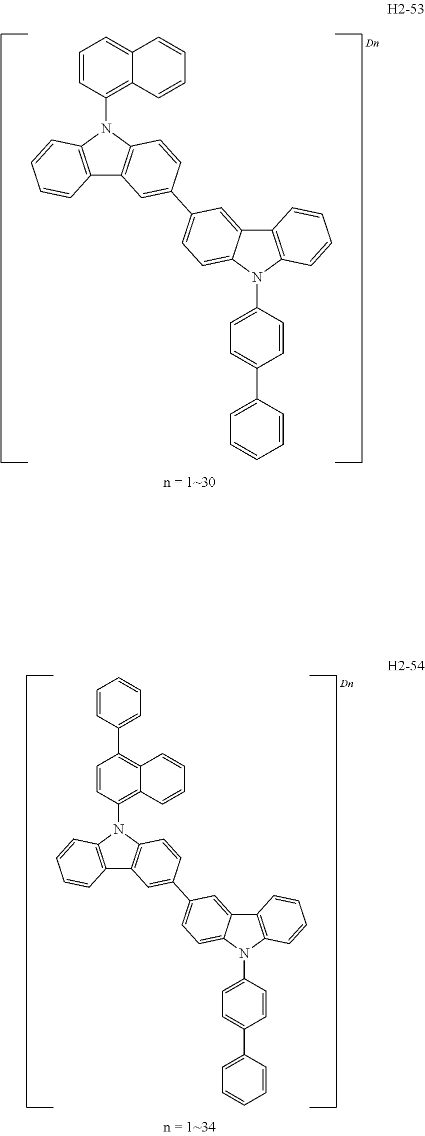 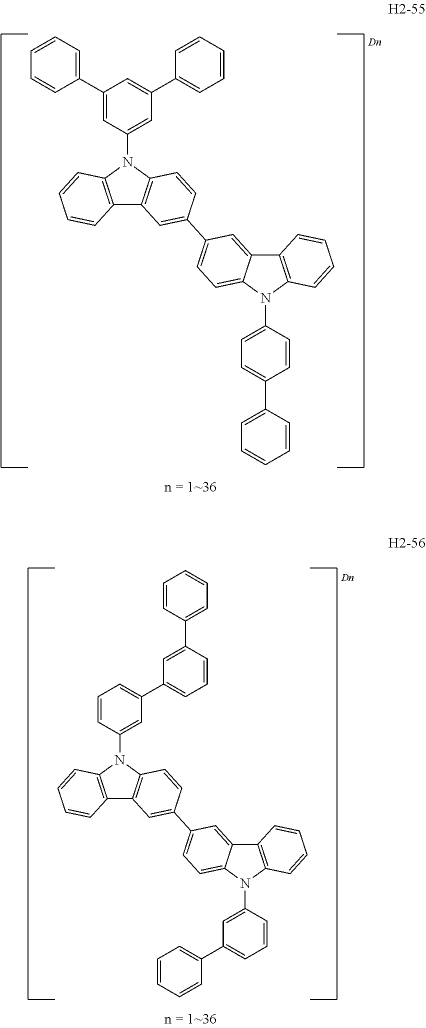 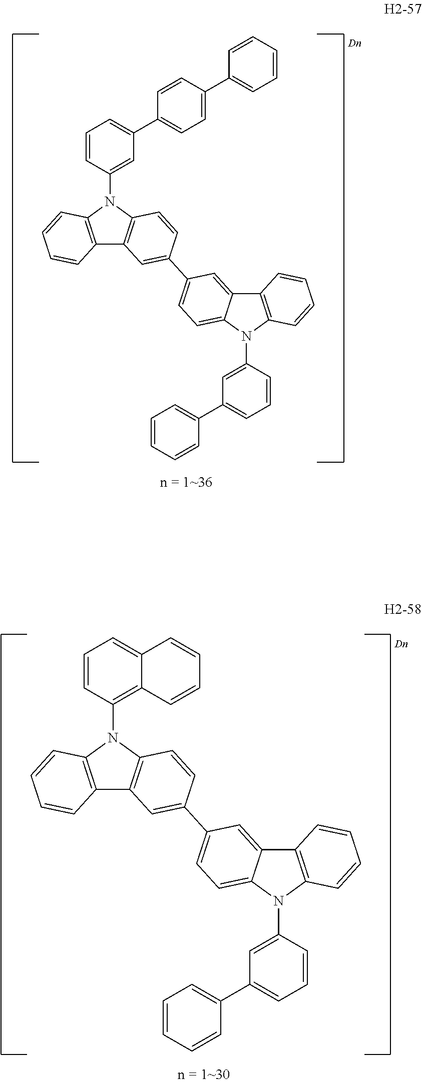 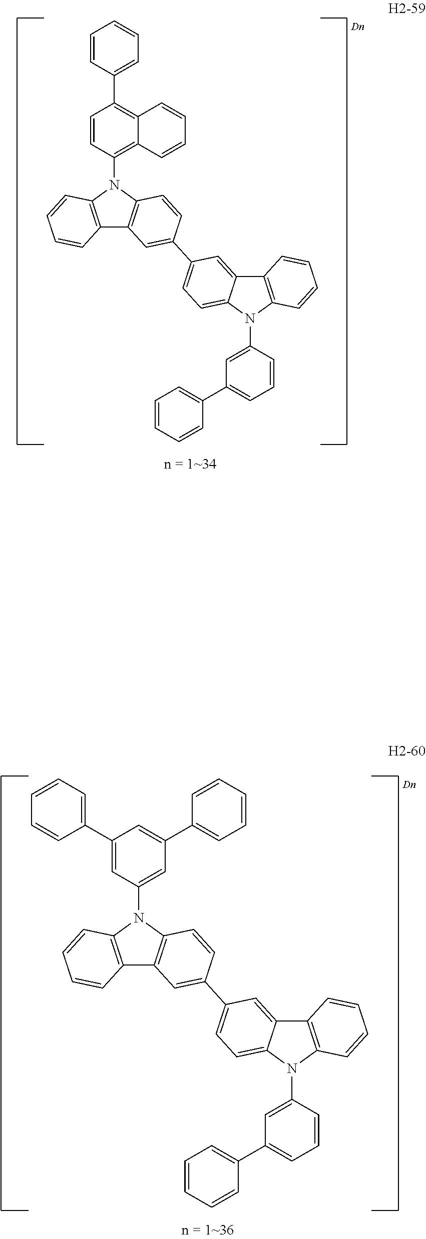 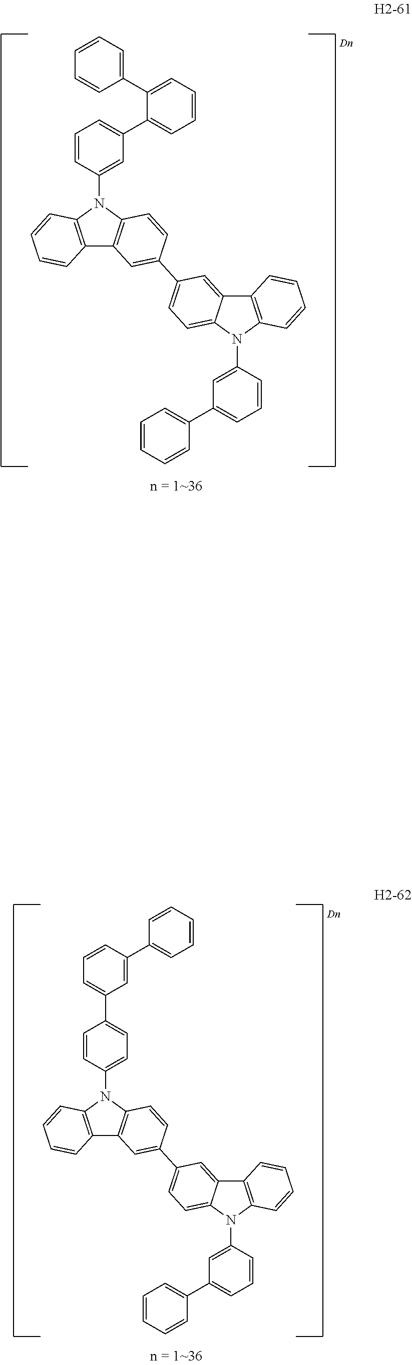 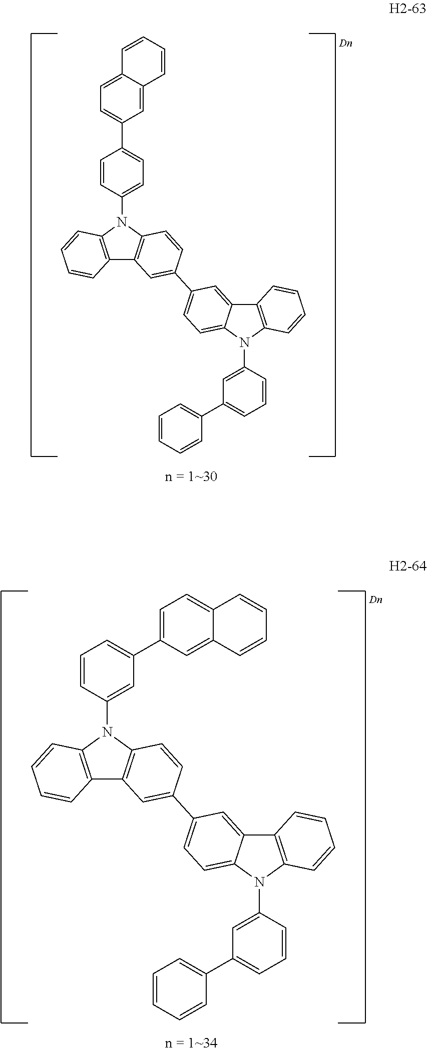 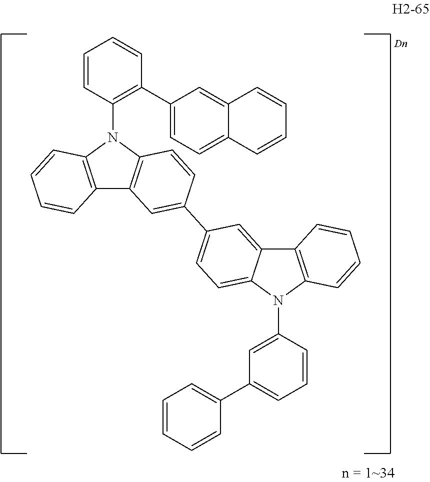 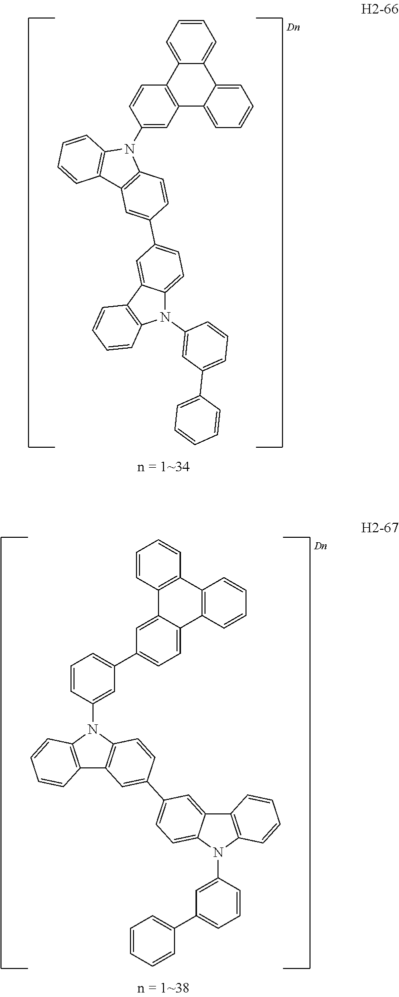 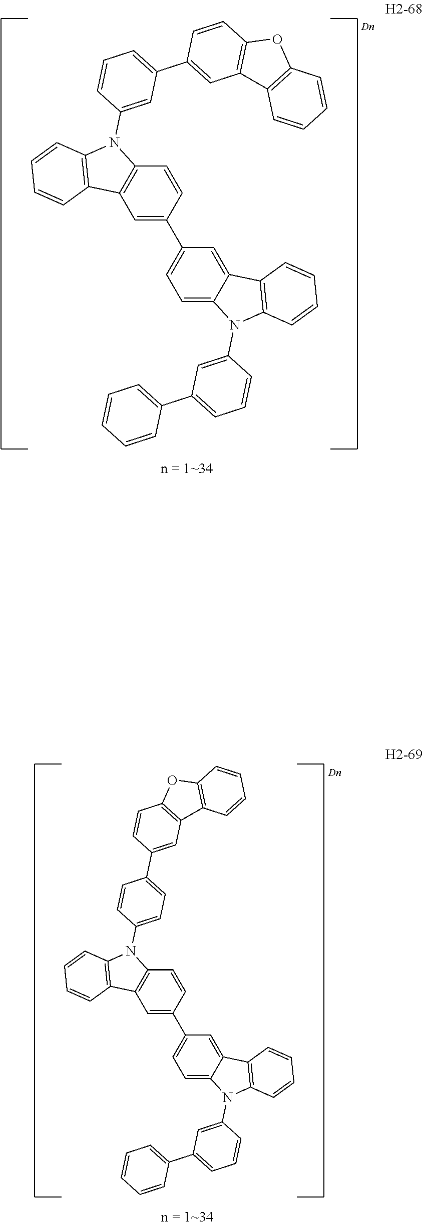 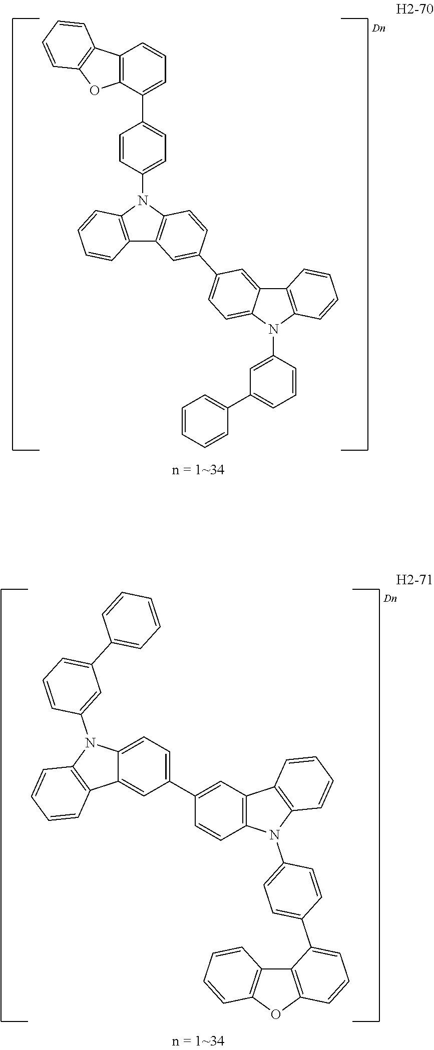 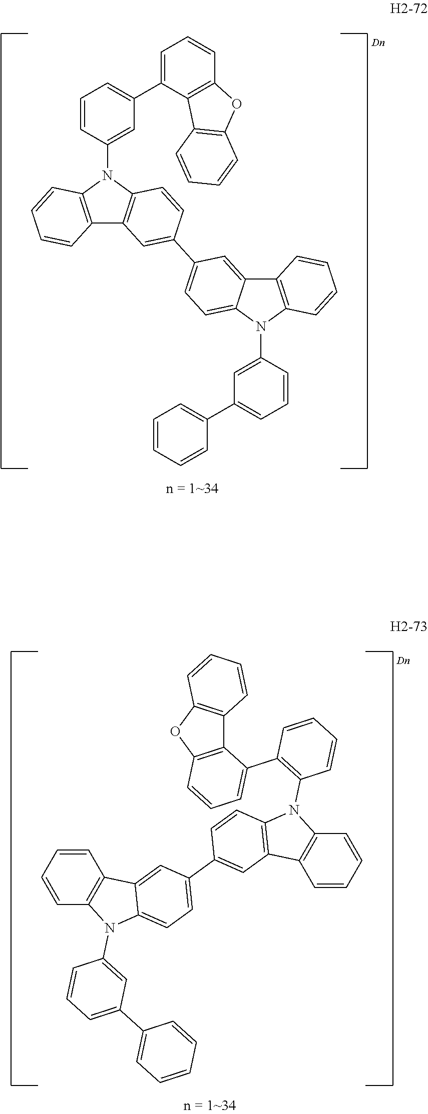 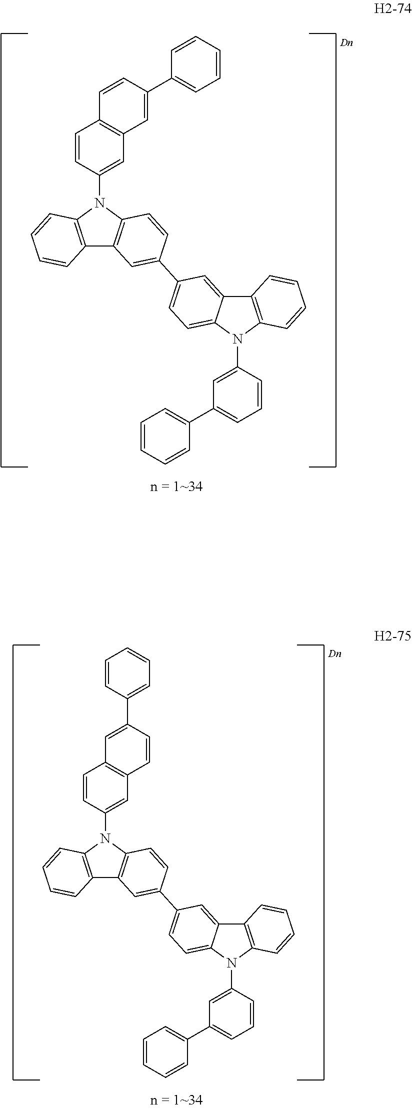 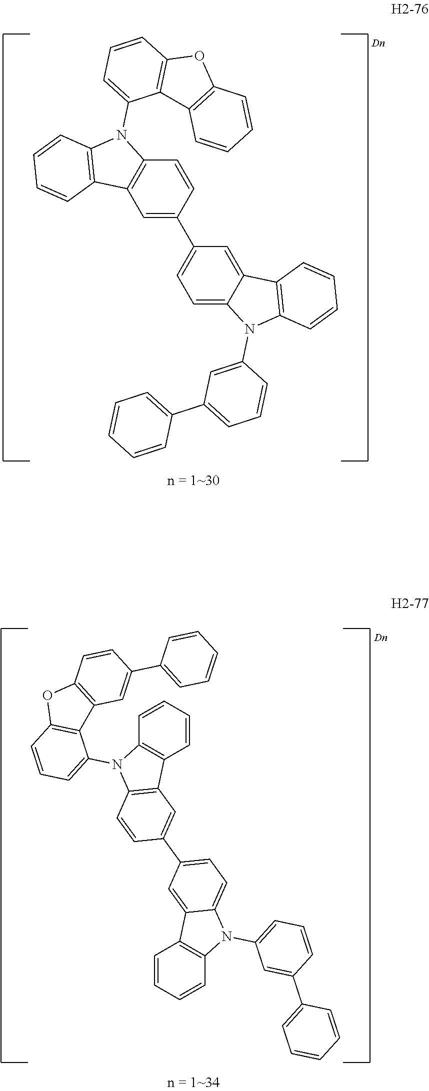 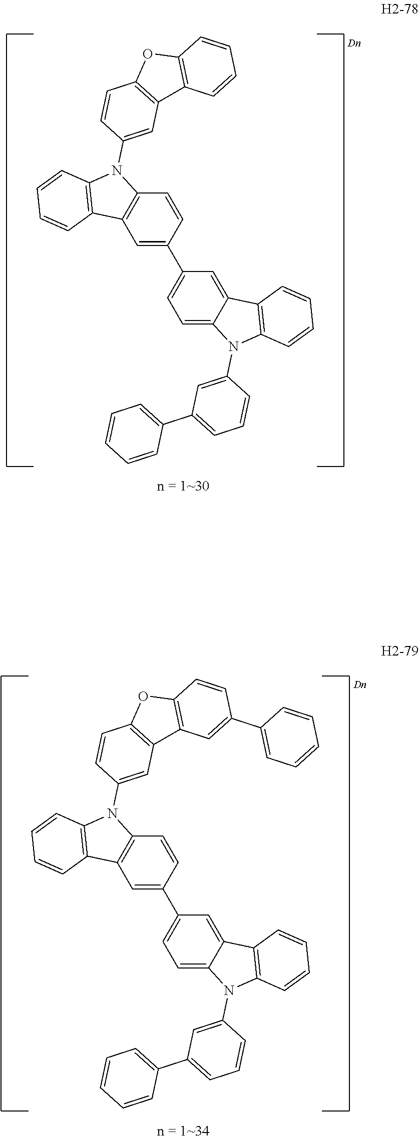 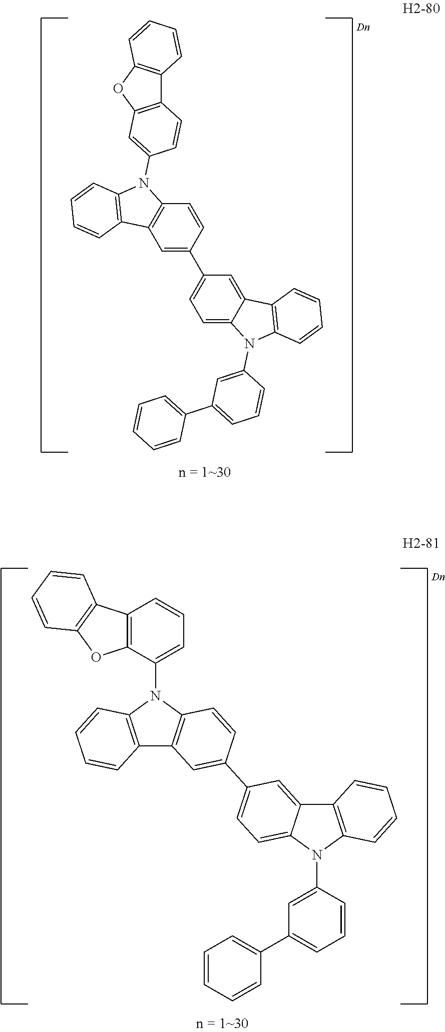 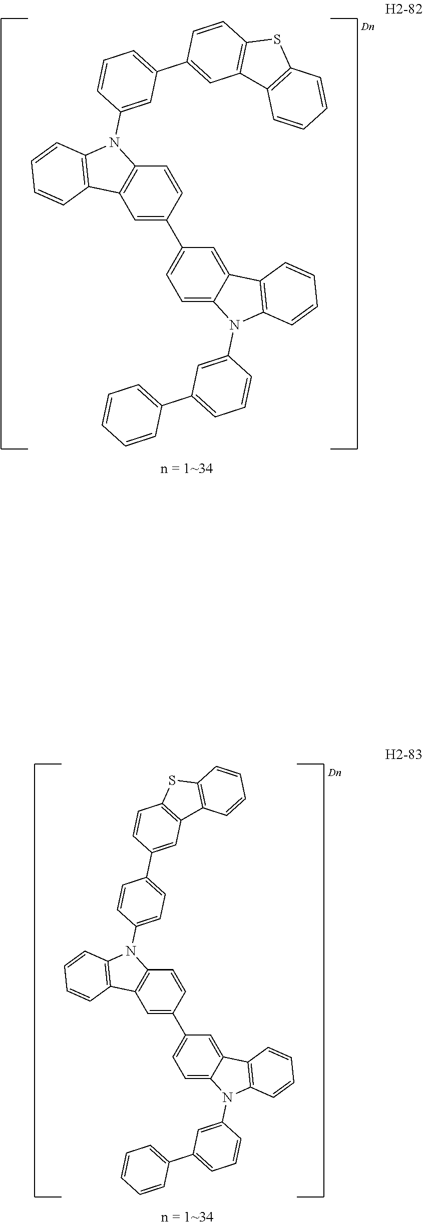 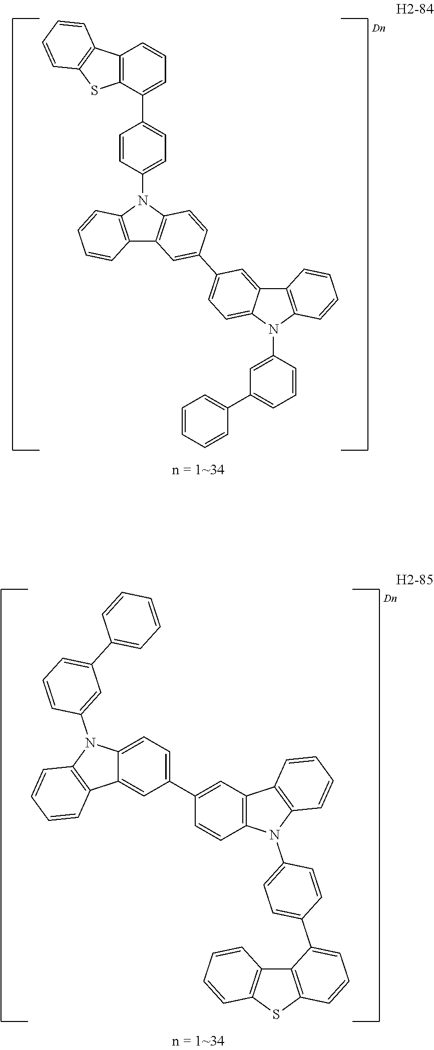 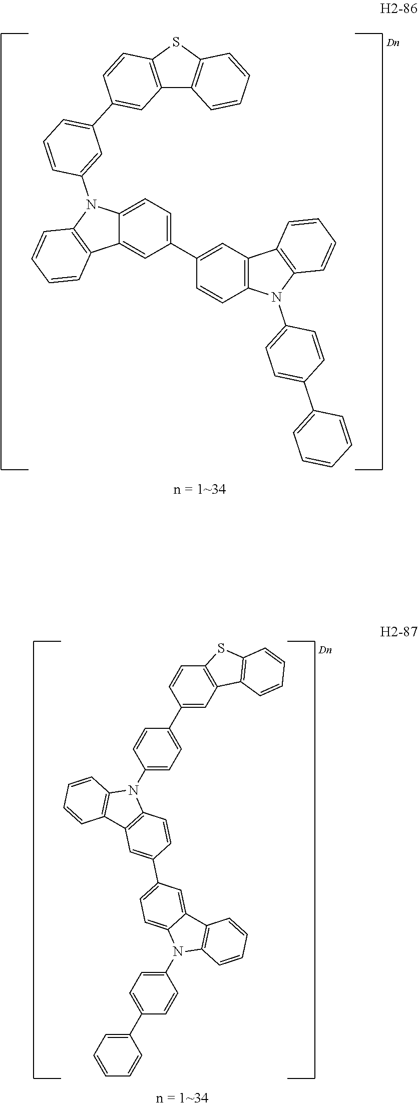 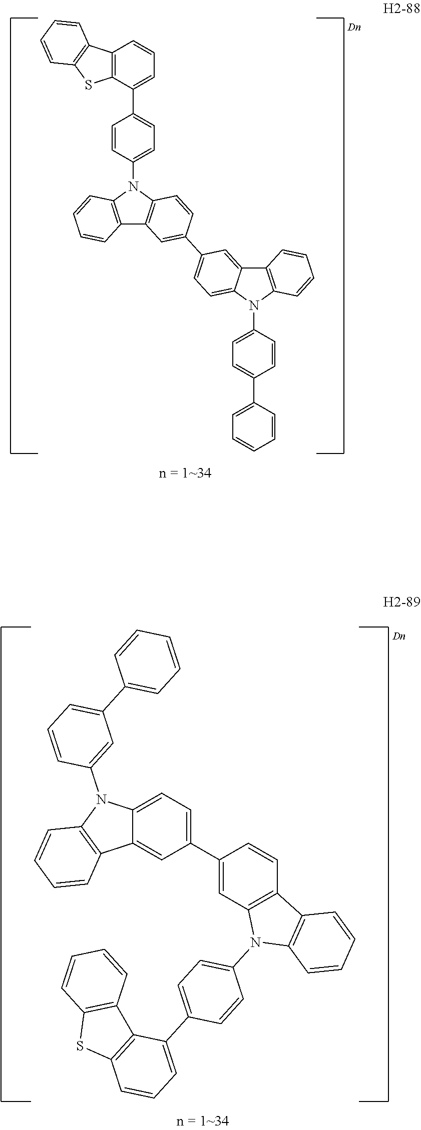 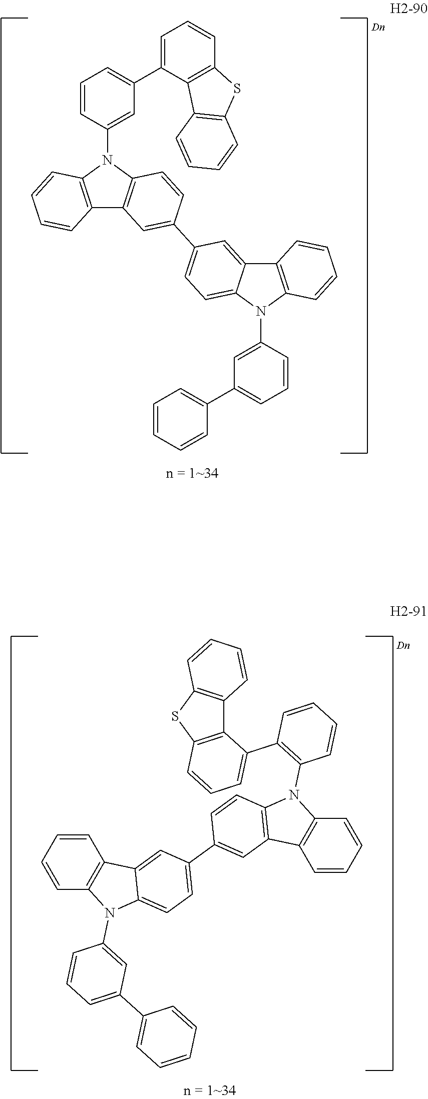 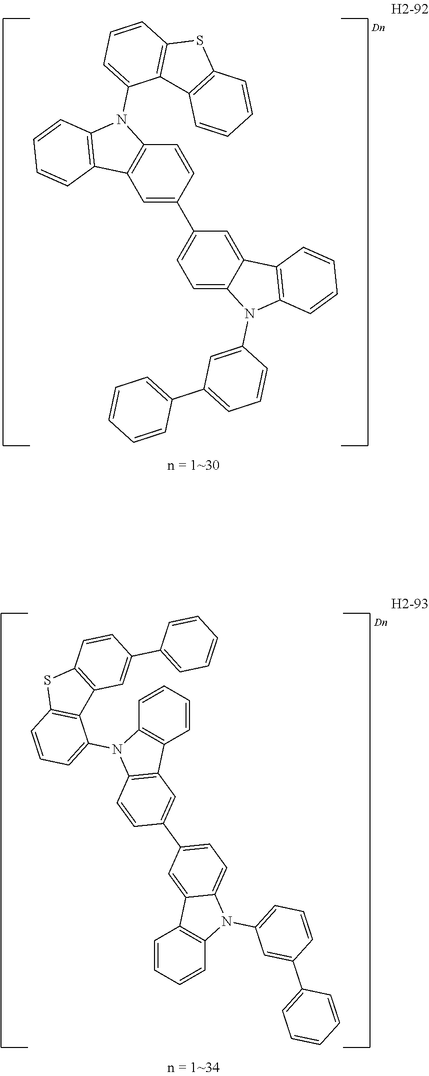 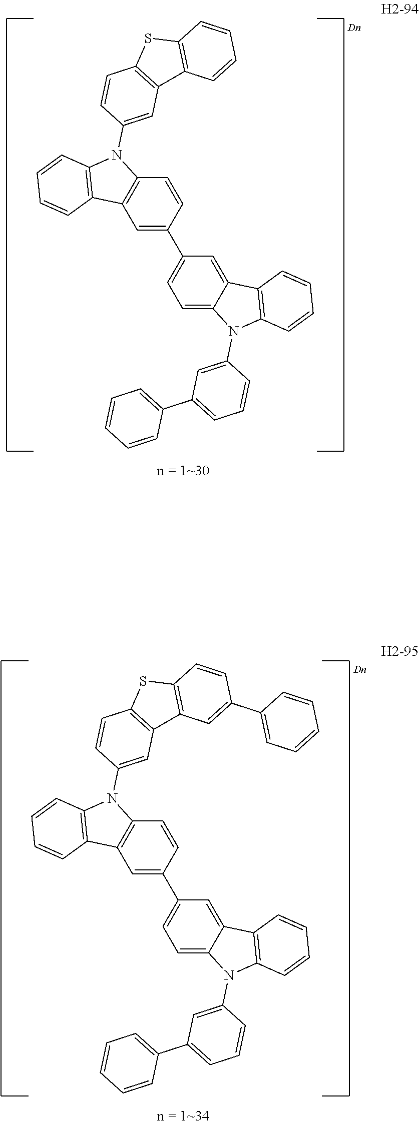 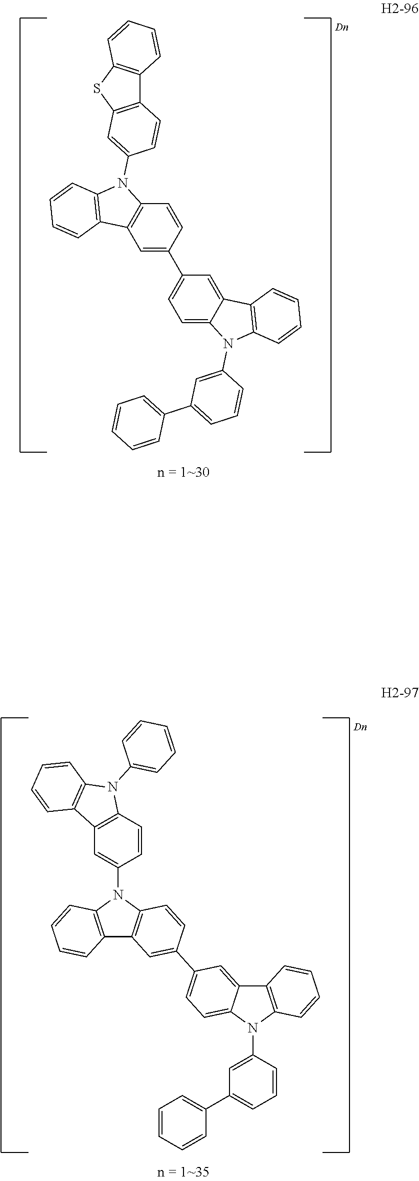 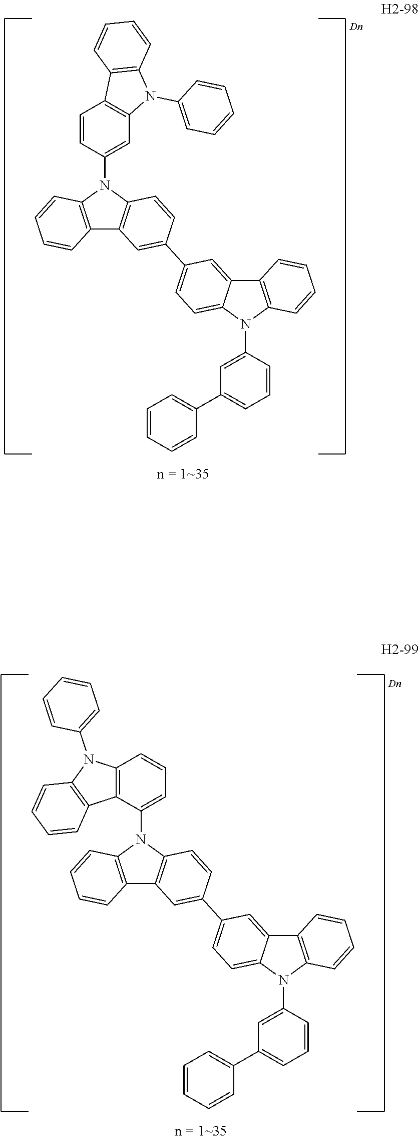 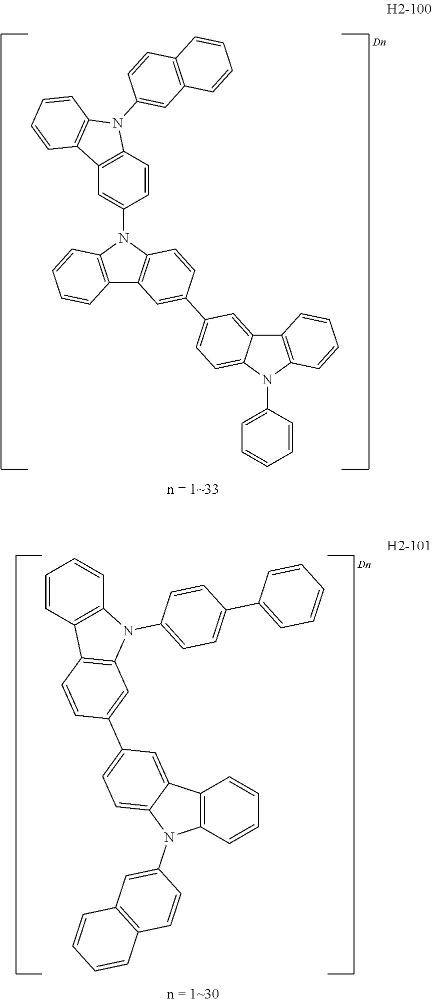 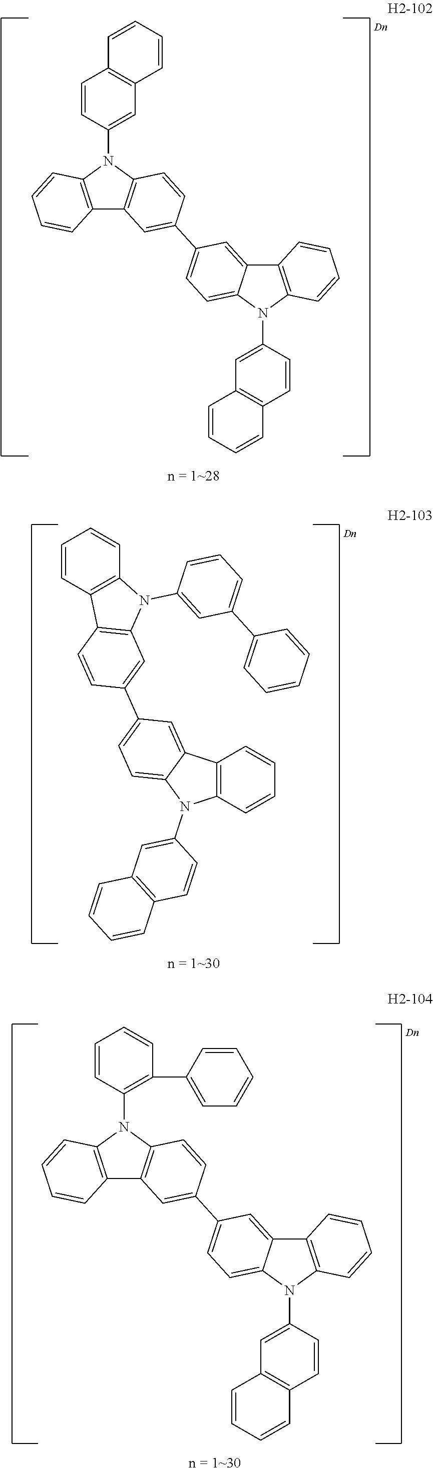 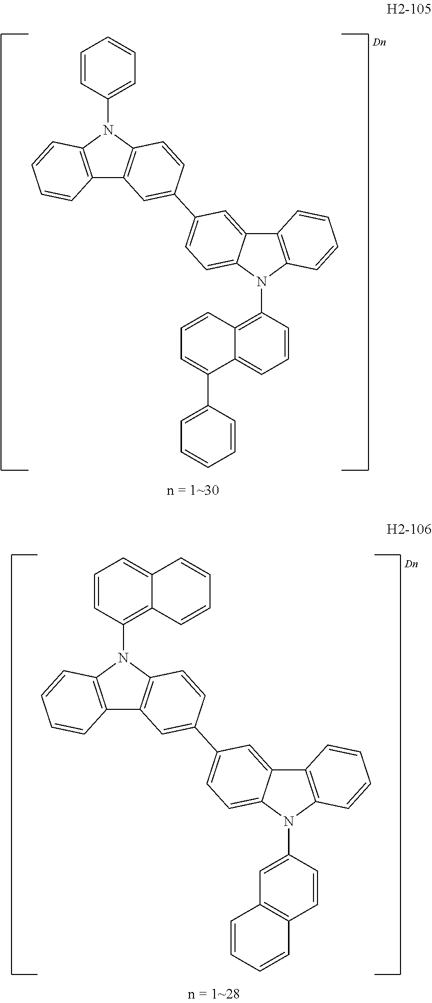 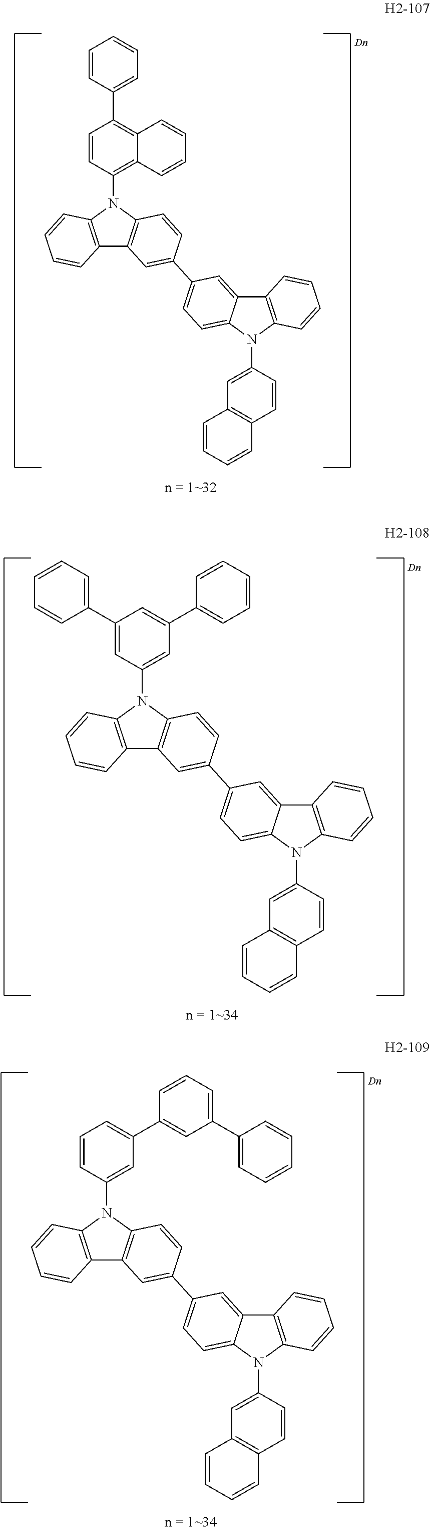 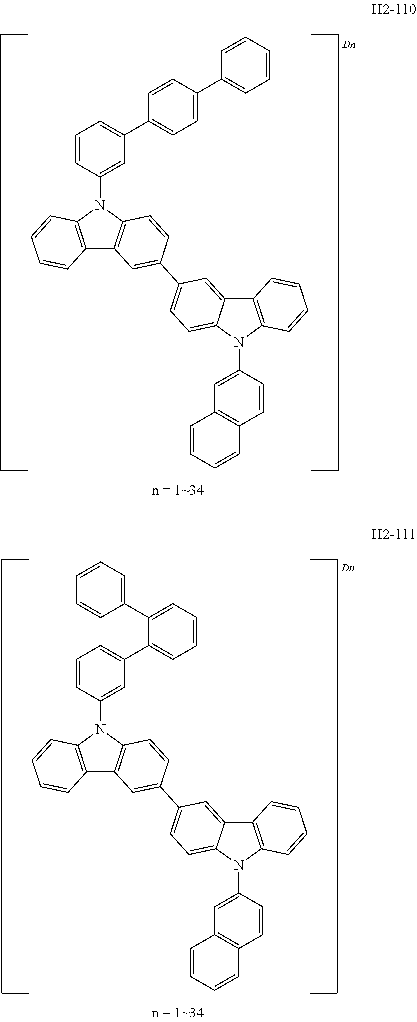 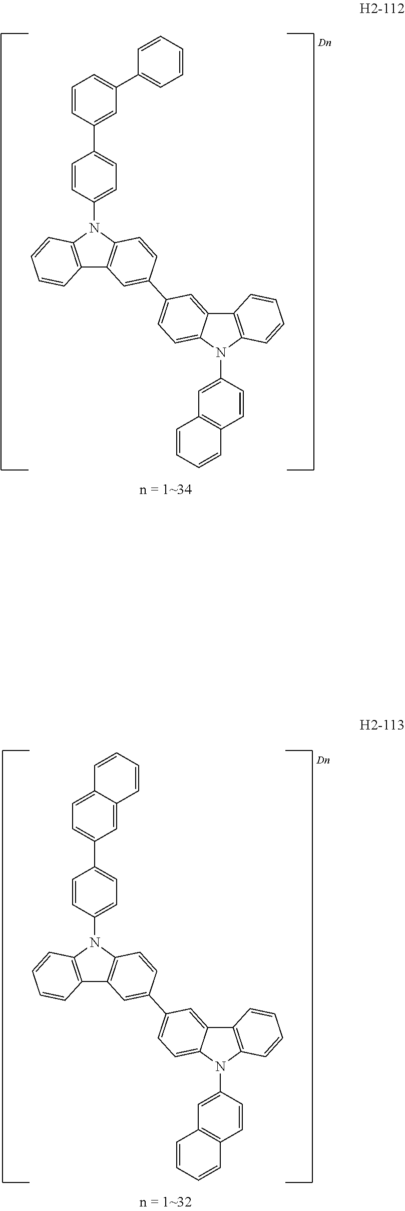 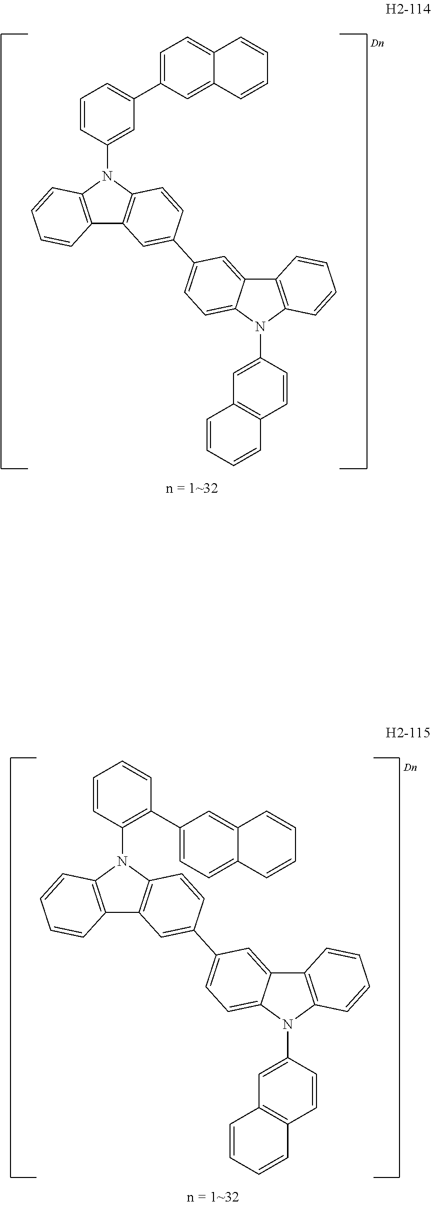 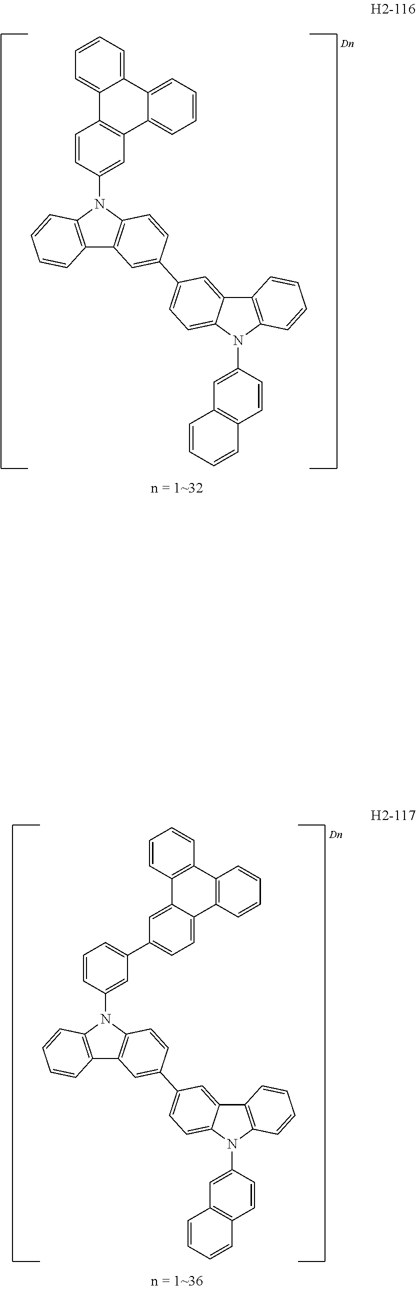 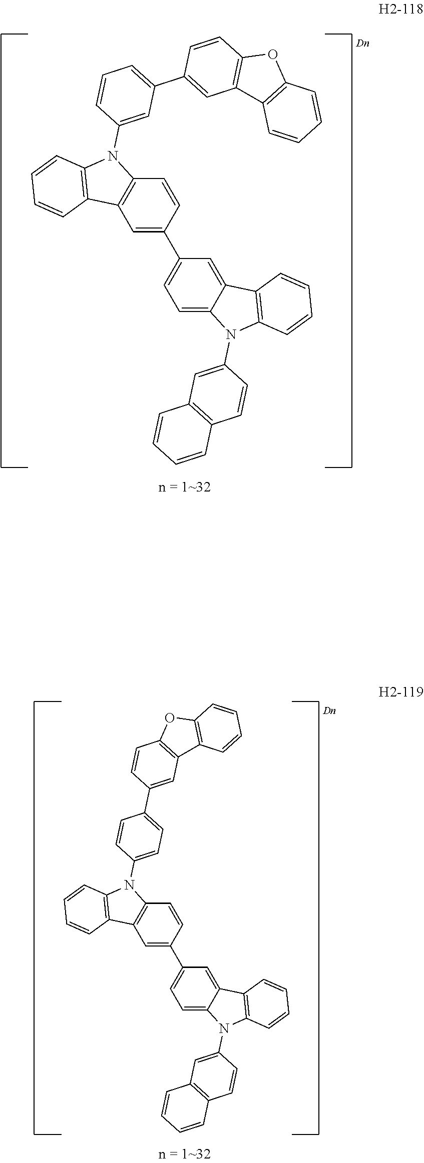 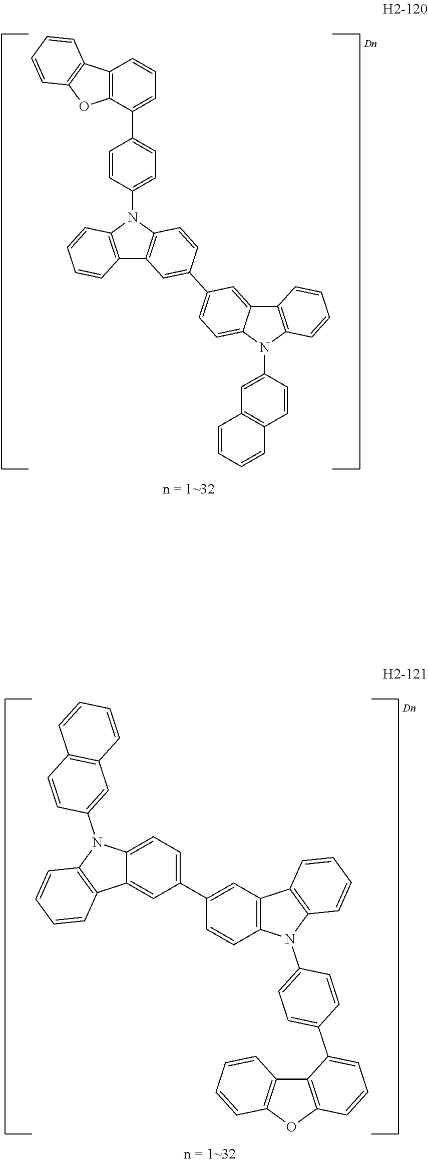 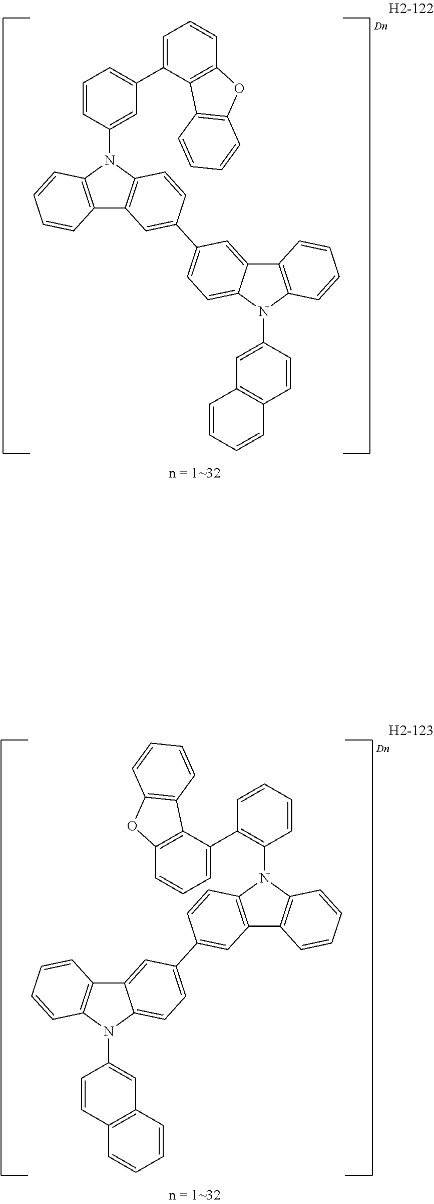 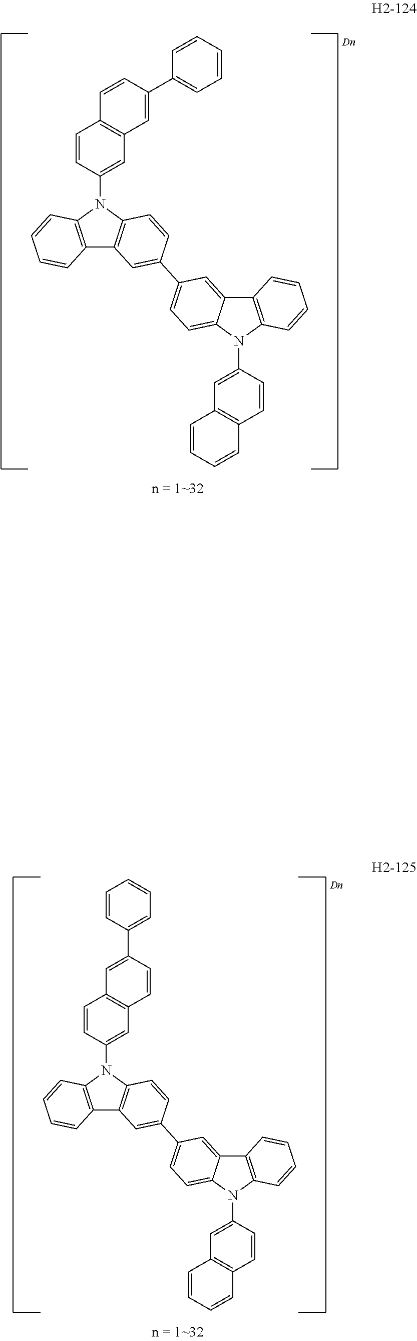 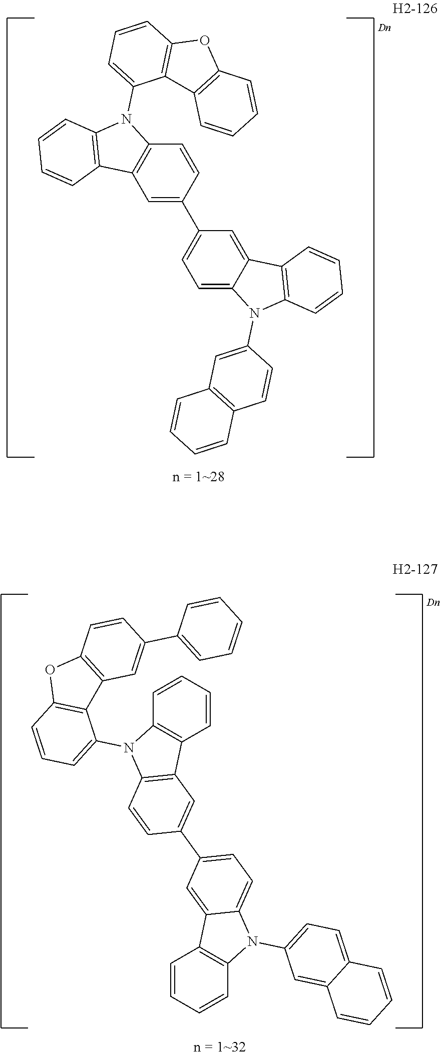 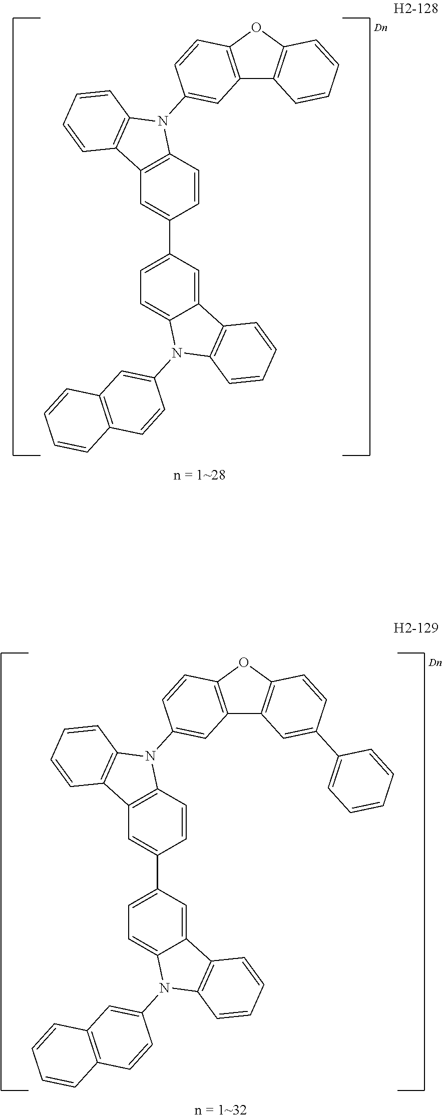 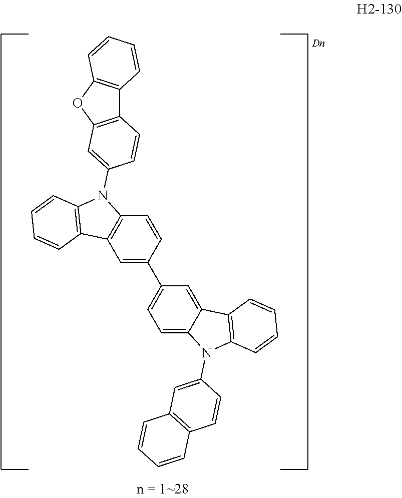 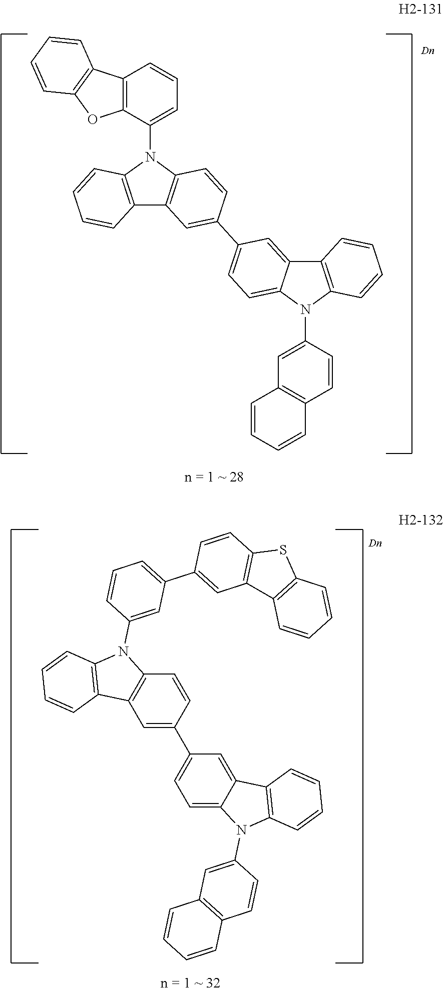 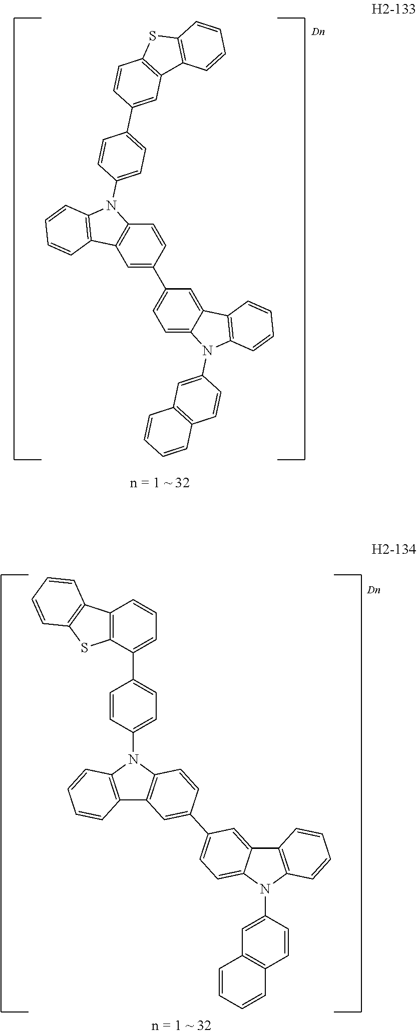 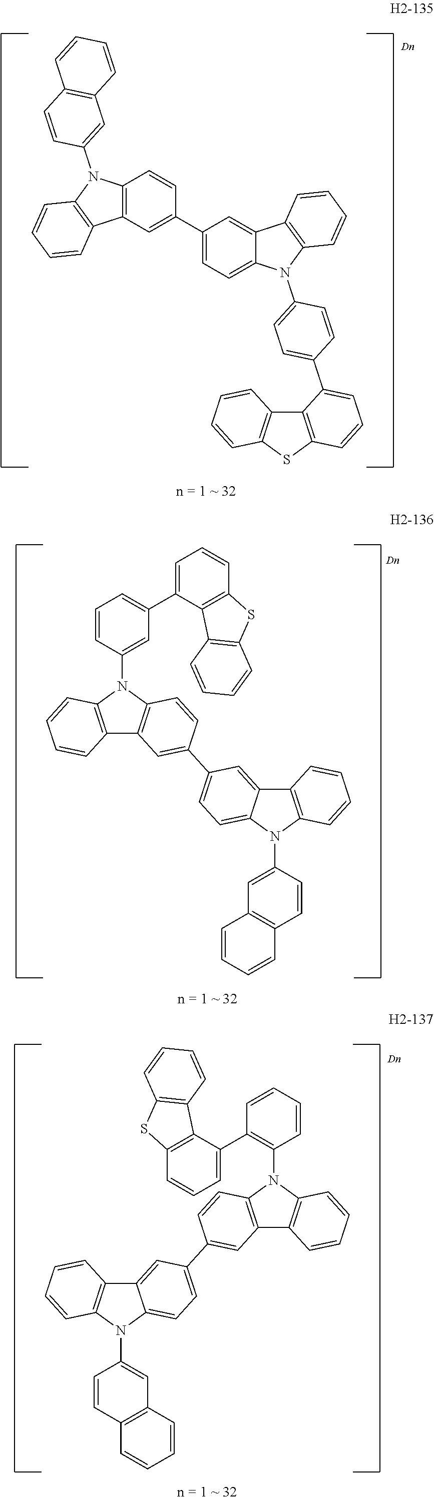 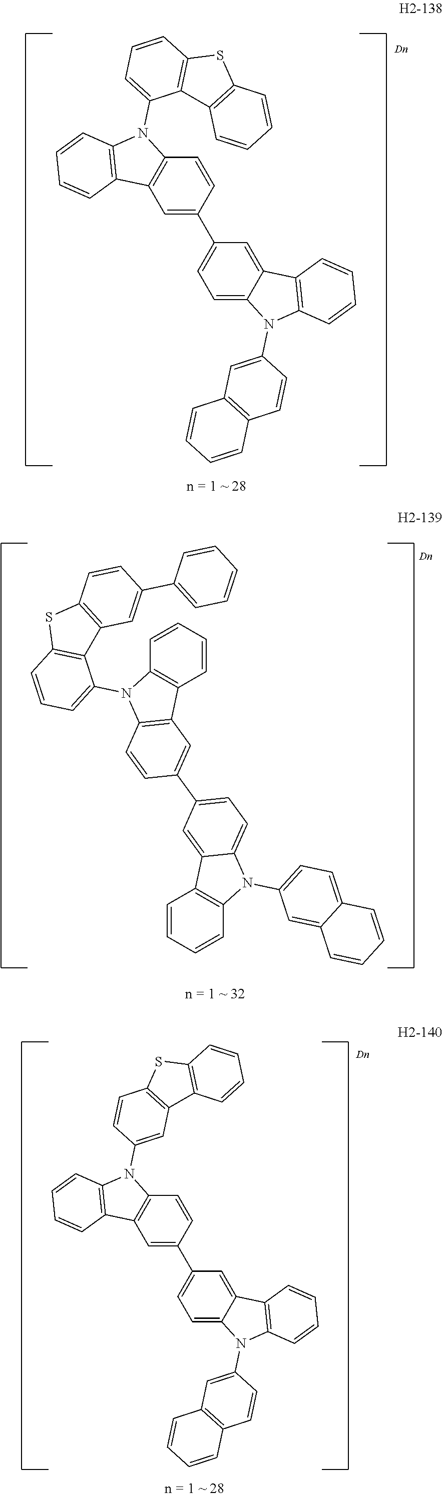 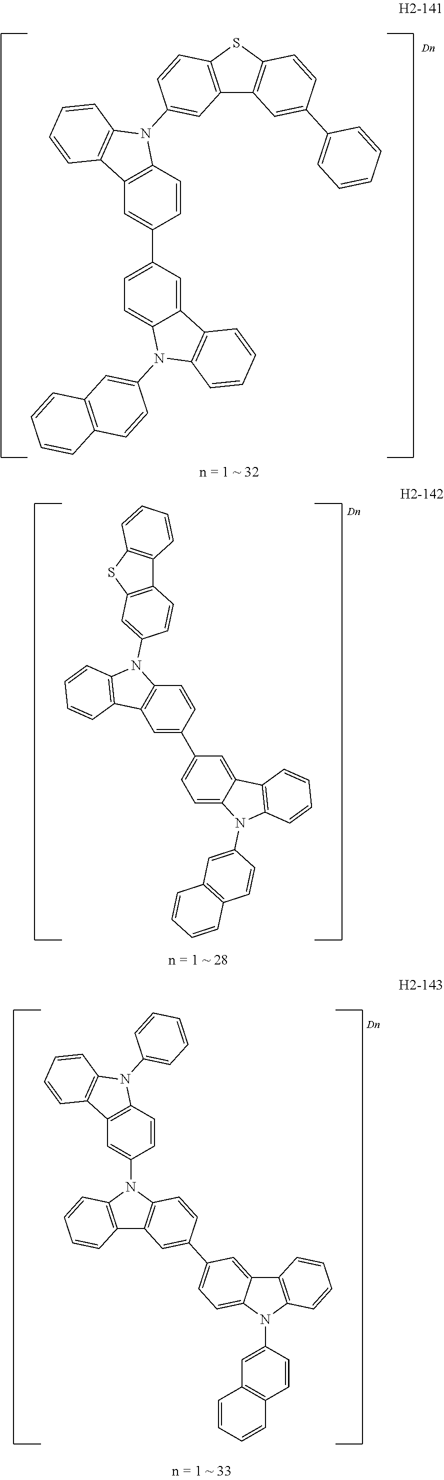 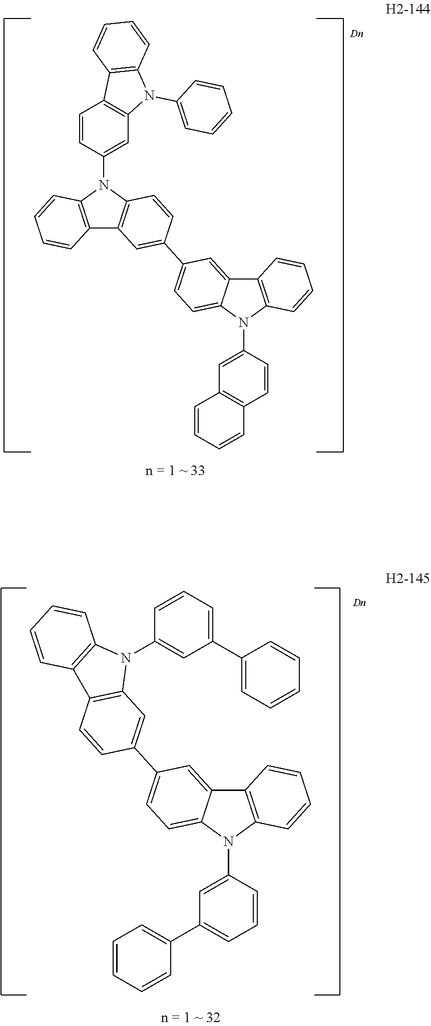 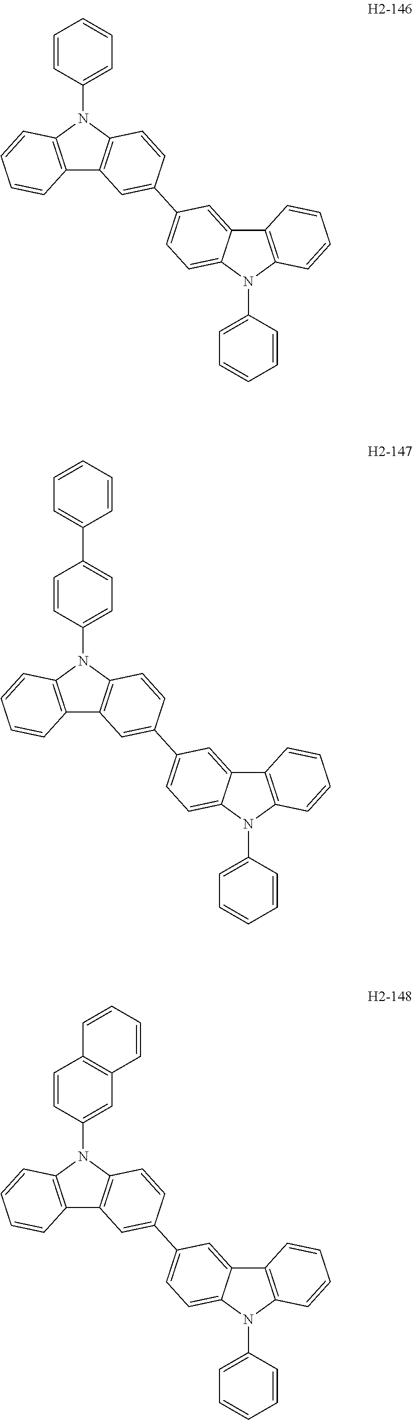 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1] 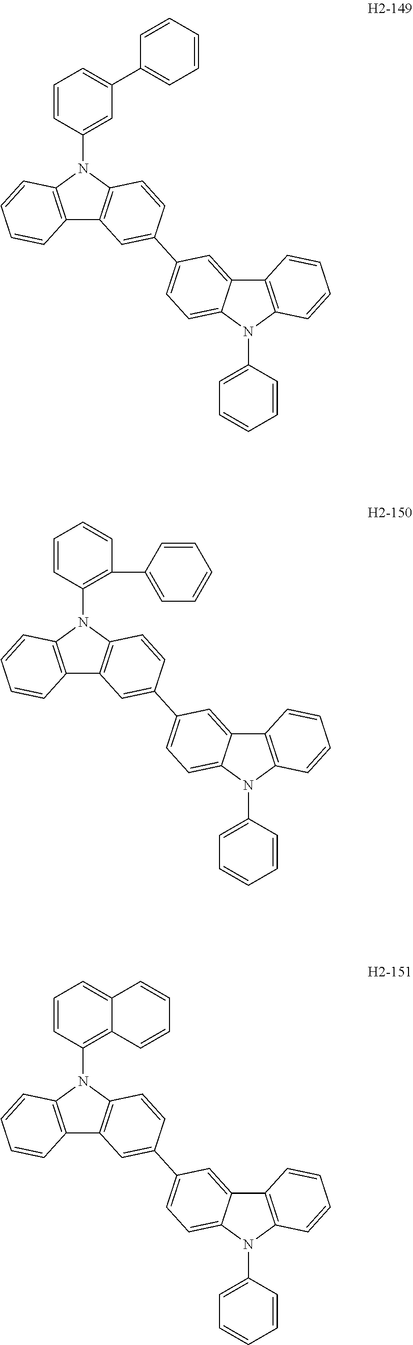 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1] 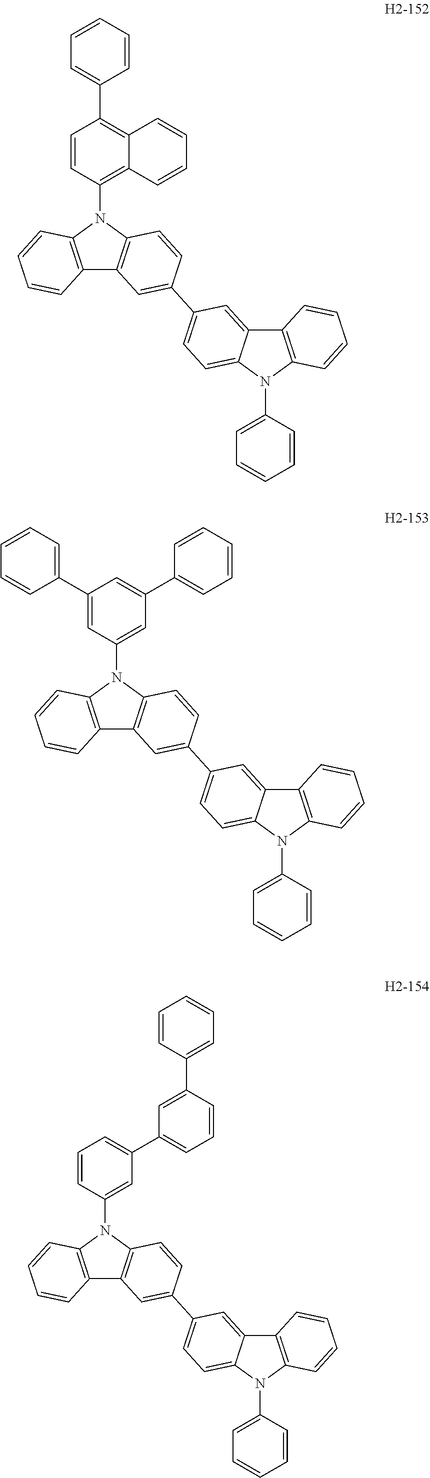 [SMILES: c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1] 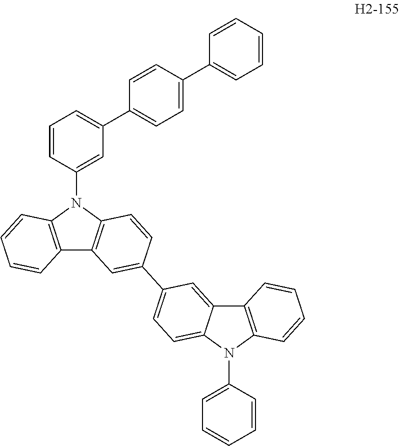 [SMILES: c1ccc(-c2ccc(-c3cccc(-n4c5ccccc5c5cc(-c6ccc7c(c6)c6ccccc6n7-c6ccccc6)ccc54)c3)cc2)cc1] 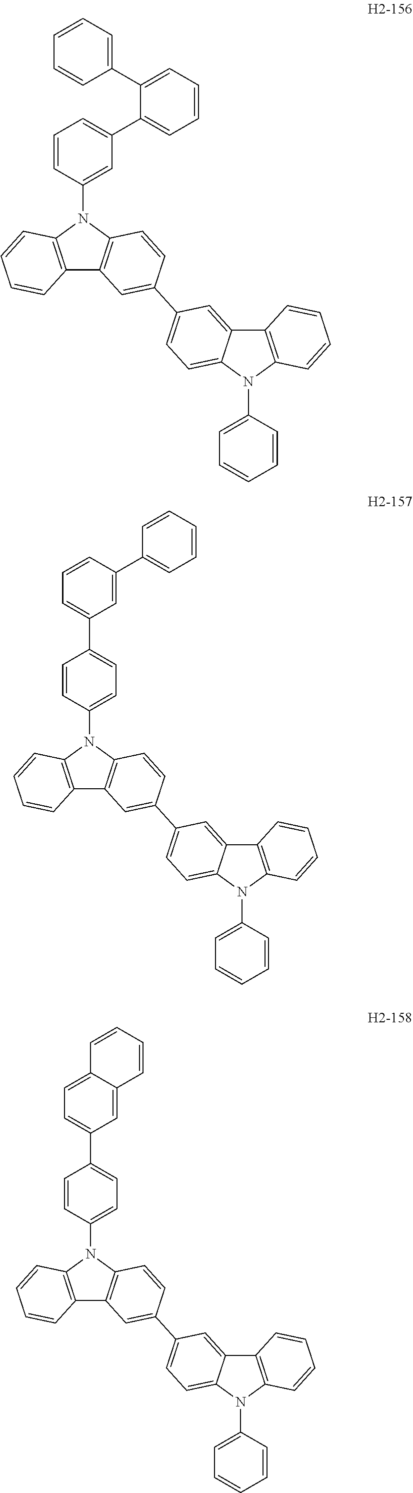 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5ccc6ccccc6c5)cc4)ccc32)cc1] 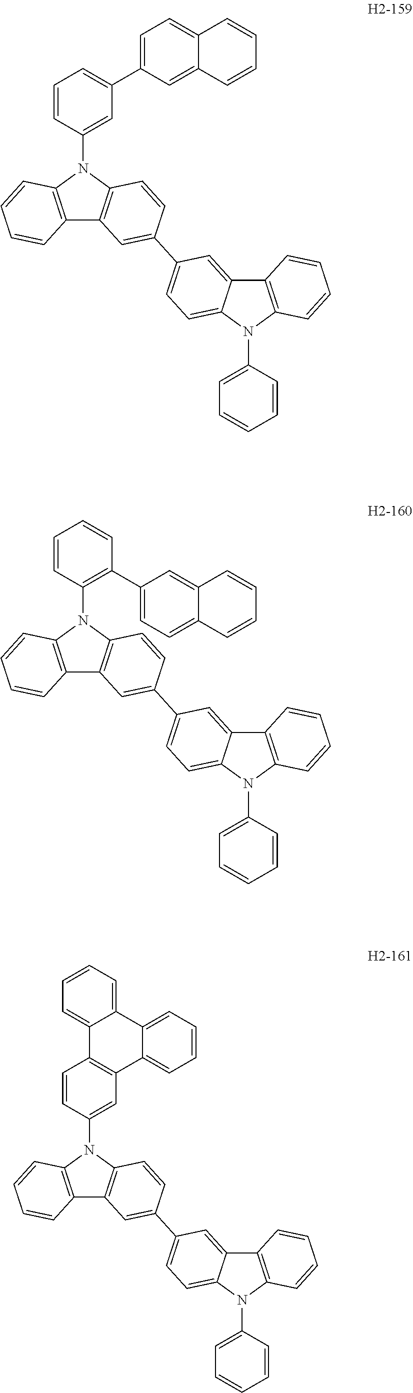 [SMILES: c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1] 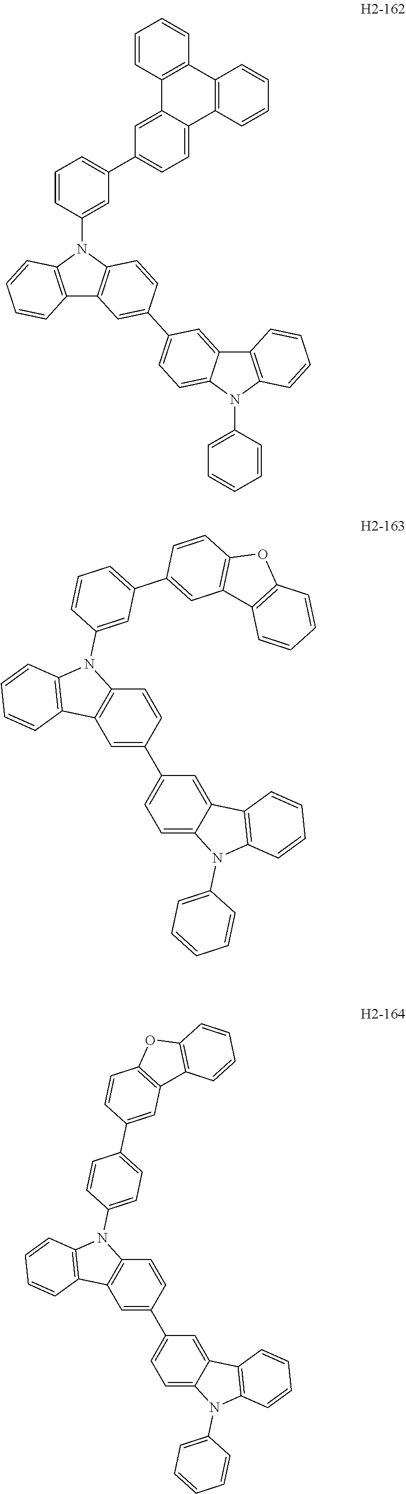 [SMILES: c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1] 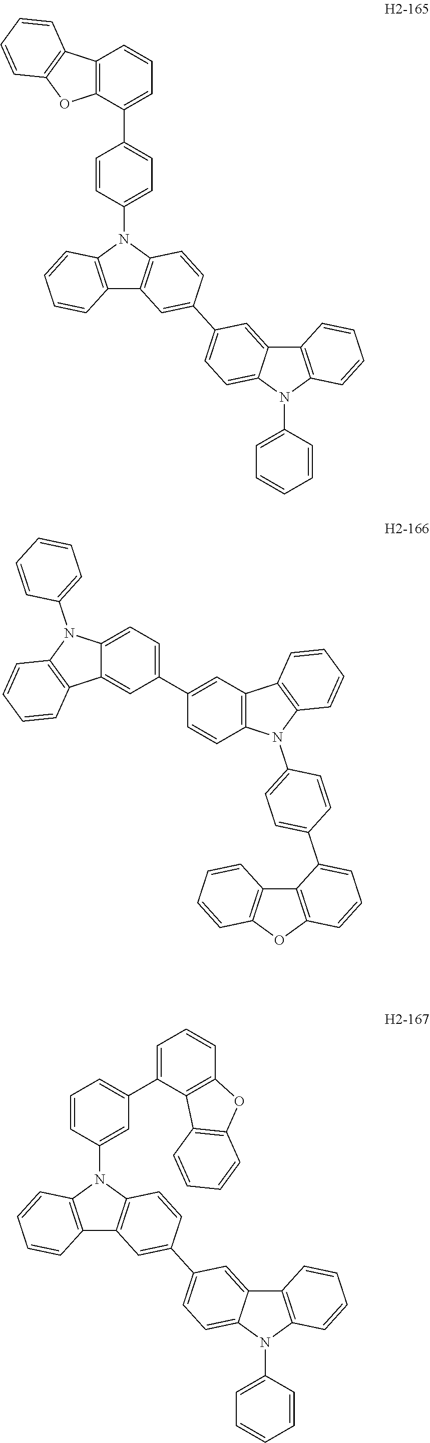 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1] 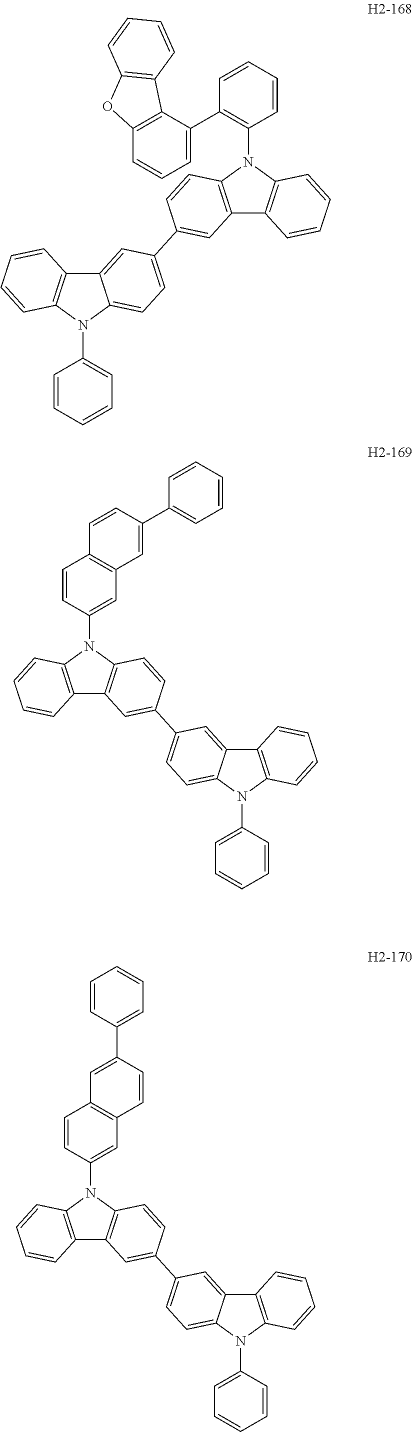 [SMILES: c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1 ; c1ccc(-c2ccc3cc(-n4c5ccccc5c5cc(-c6ccc7c(c6)c6ccccc6n7-c6ccccc6)ccc54)ccc3c2)cc1] 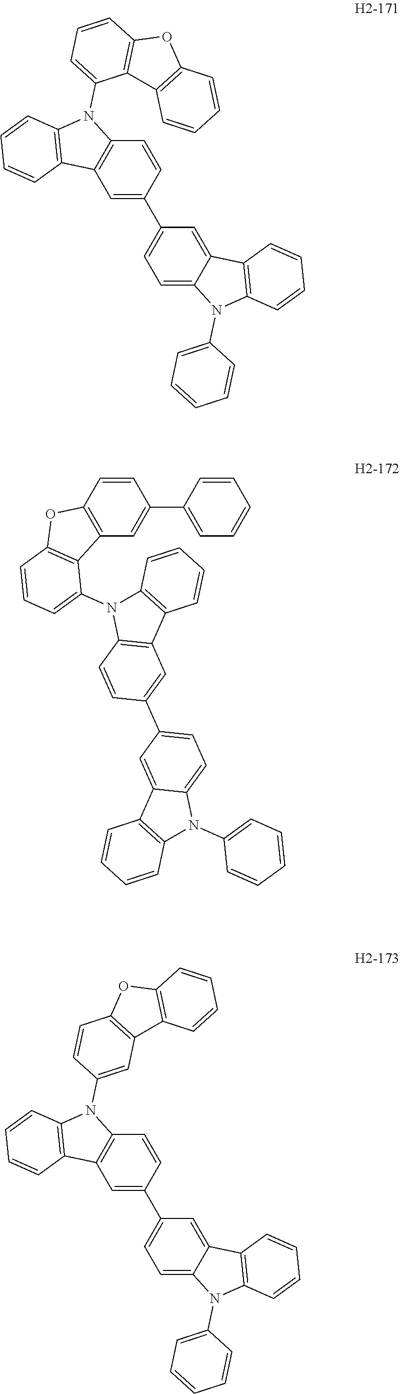 [SMILES: c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5oc6ccccc6c5c4)ccc32)cc1] 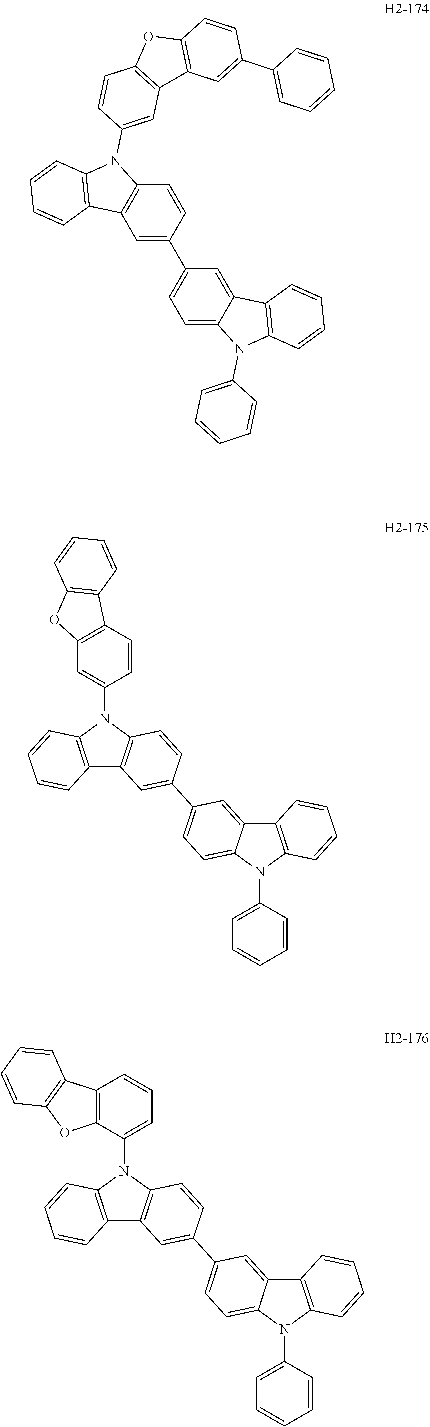 [SMILES: c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5c4oc4ccccc45)ccc32)cc1] 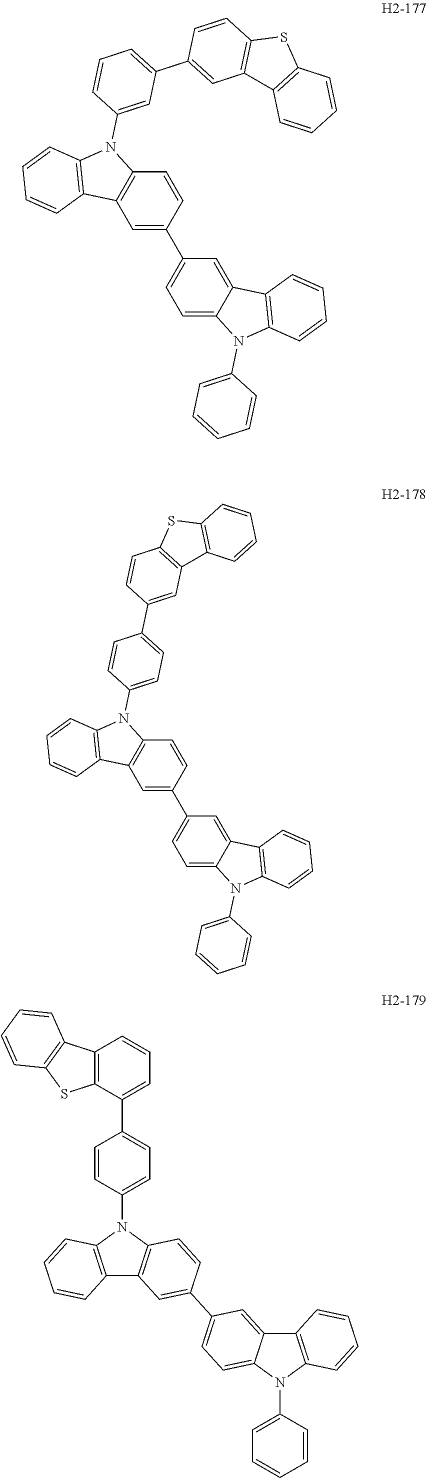 [SMILES: c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1] 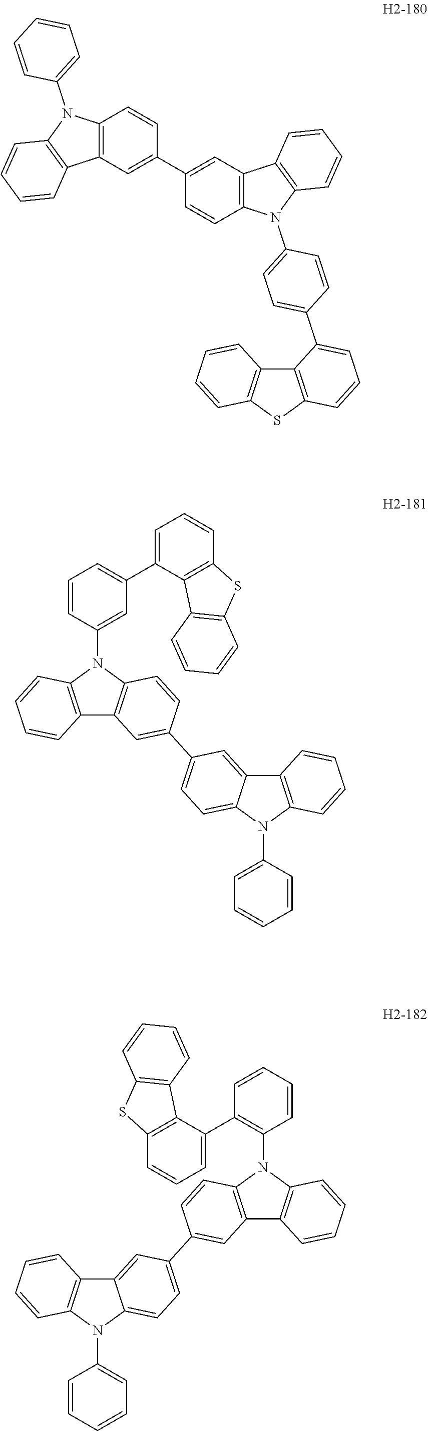 [SMILES: c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1] 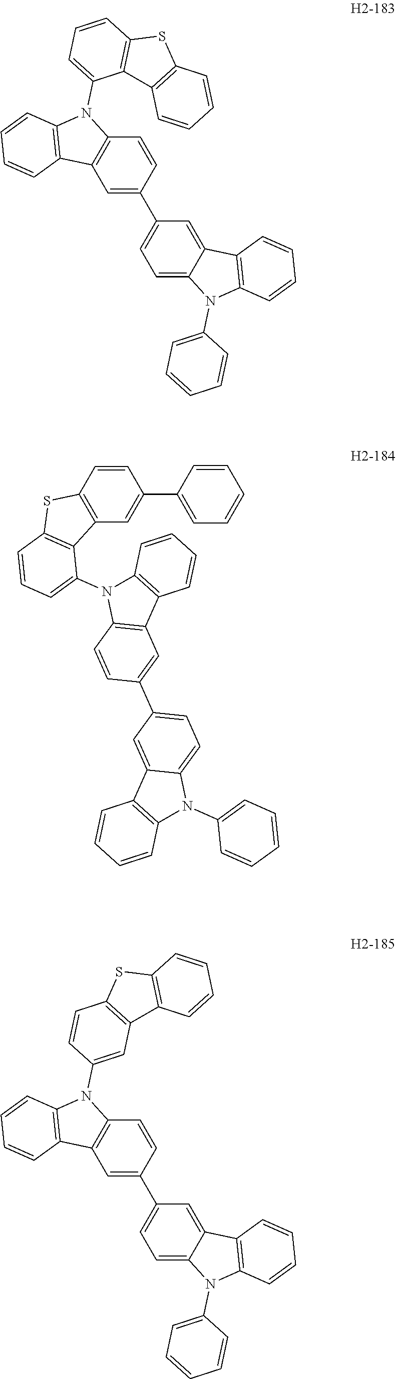 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc3sc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5sc6ccccc6c5c4)ccc32)cc1] 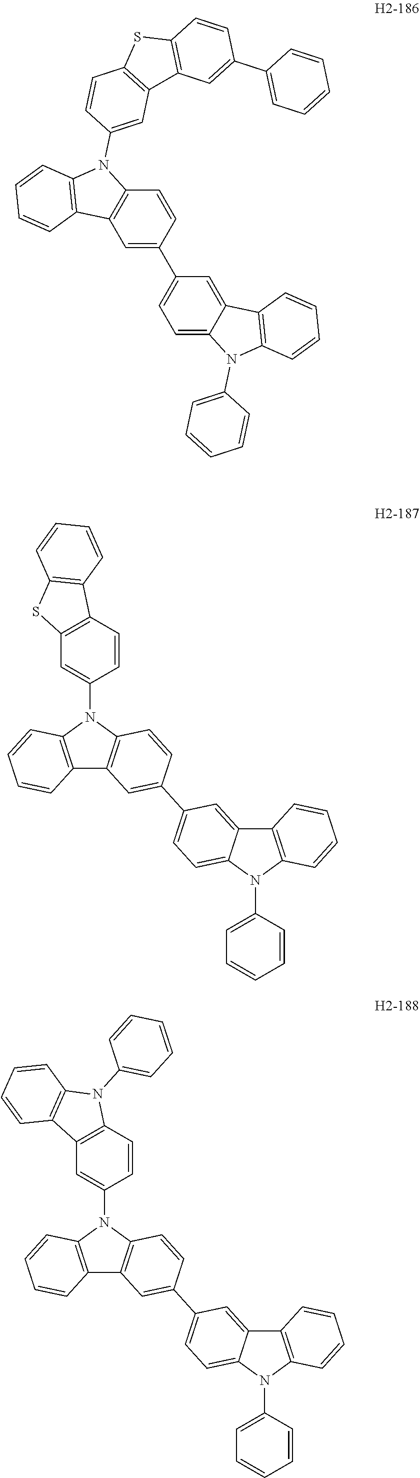 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1] 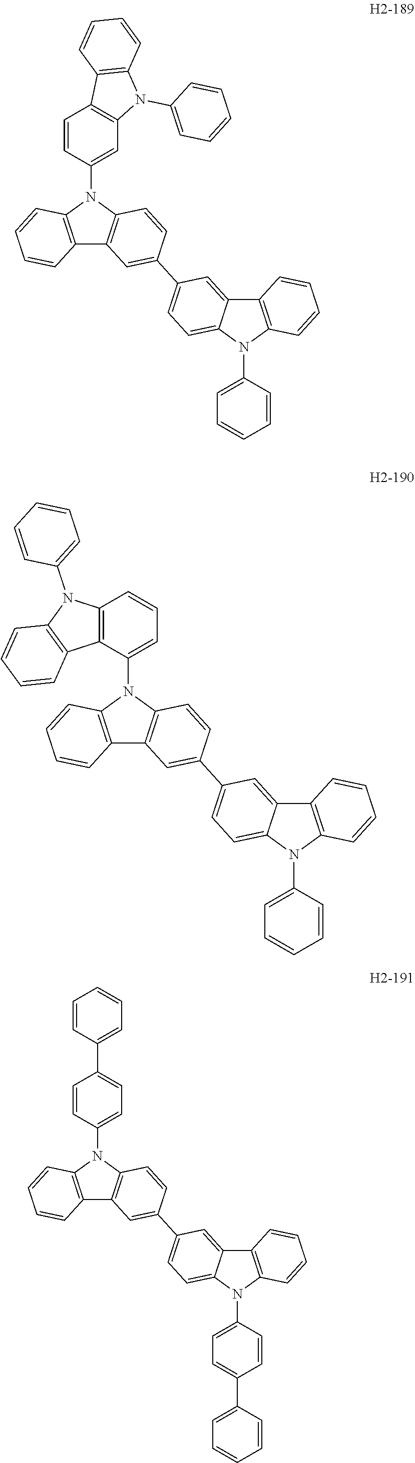 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1] 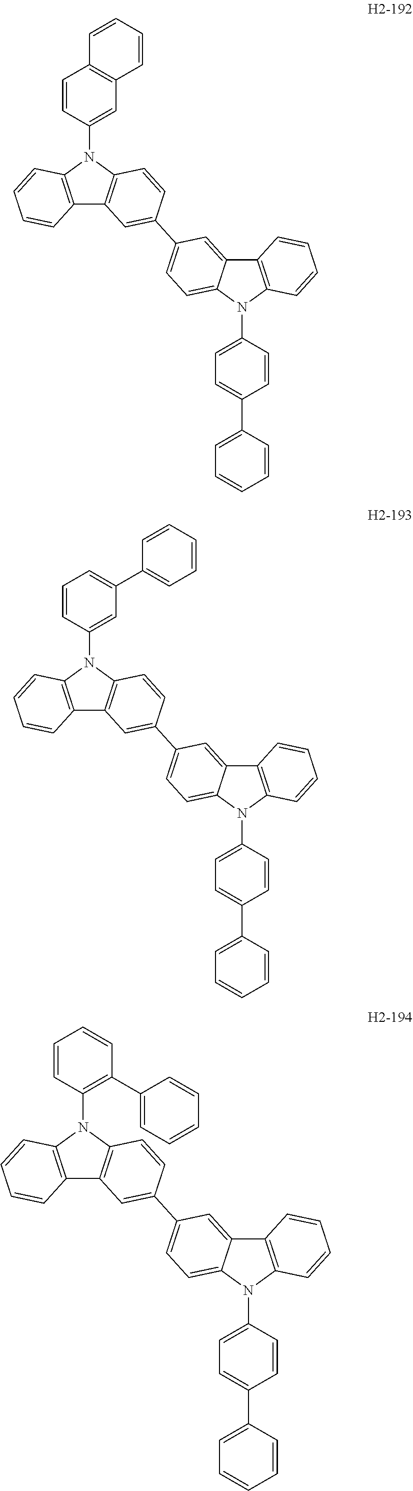 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1] 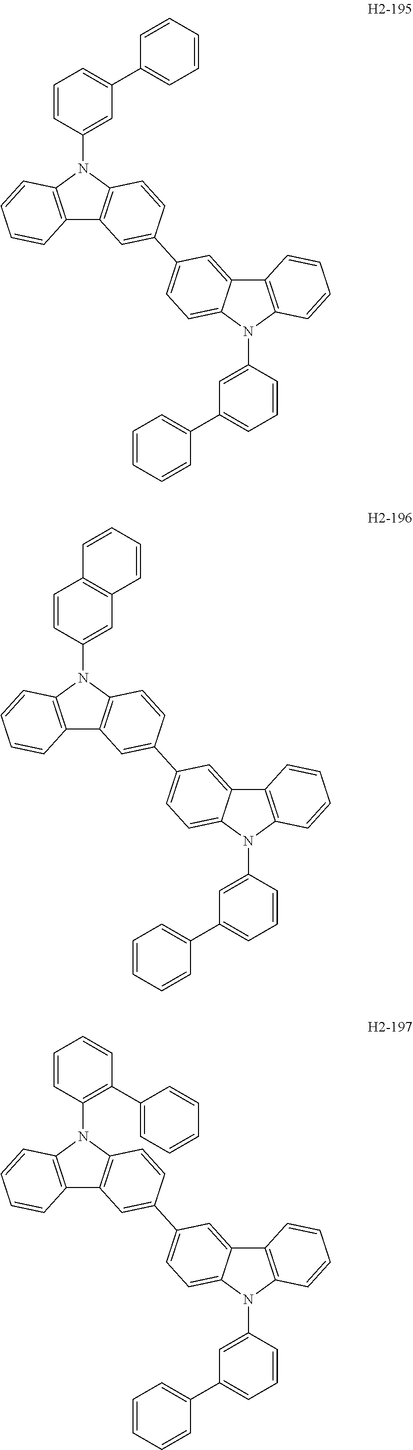 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1] 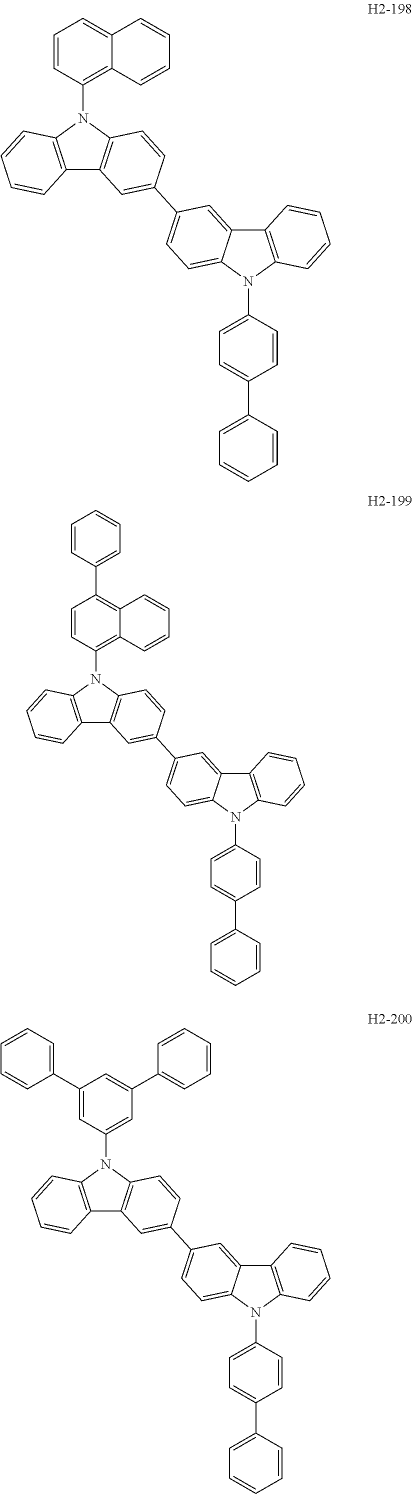 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1] 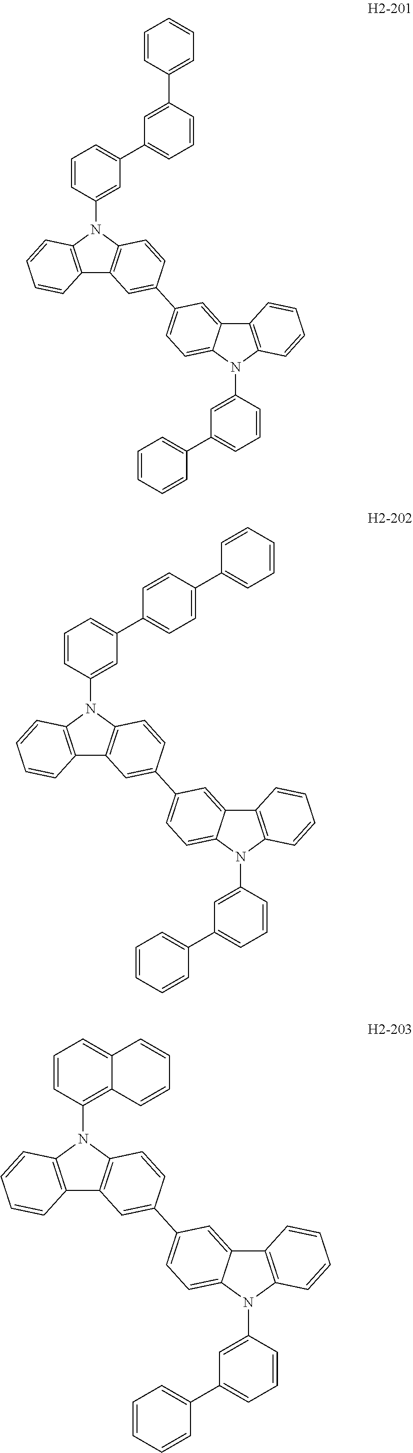 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1] 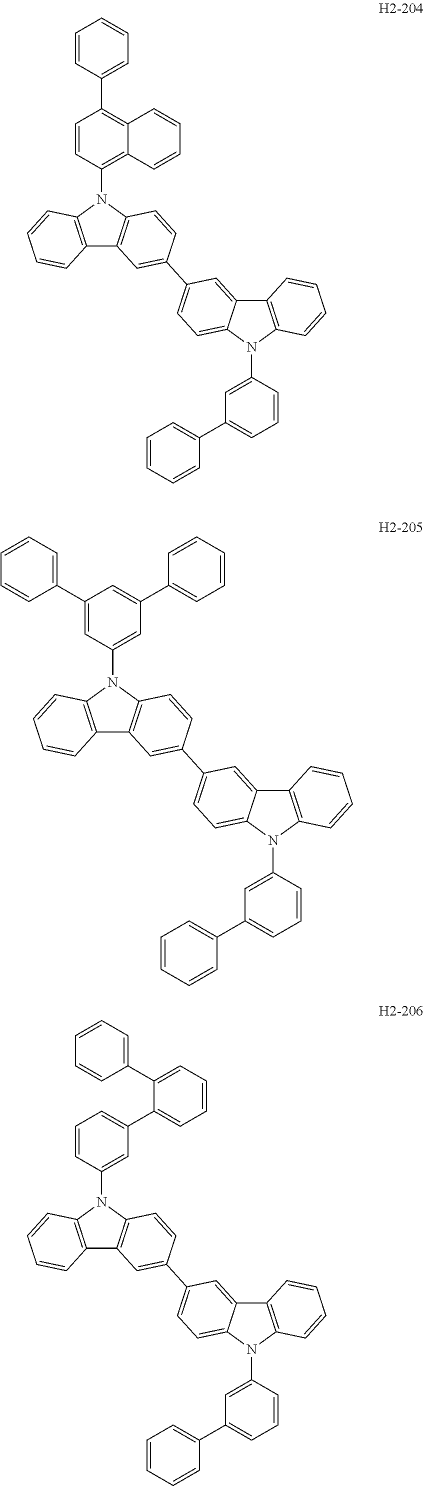 [SMILES: c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5cccc(-c6ccccc6-c6ccccc6)c5)ccc43)c2)cc1] 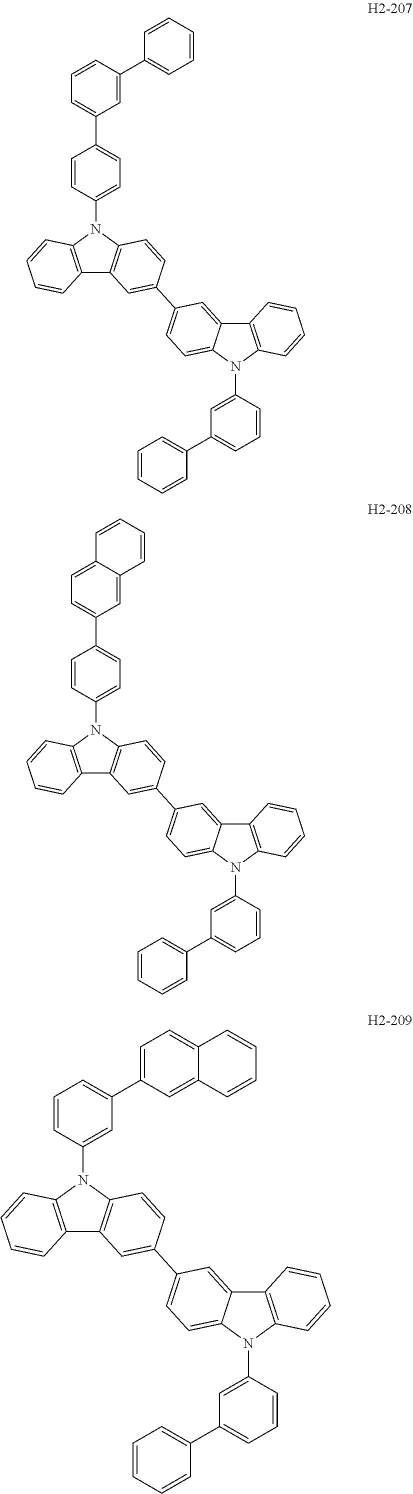 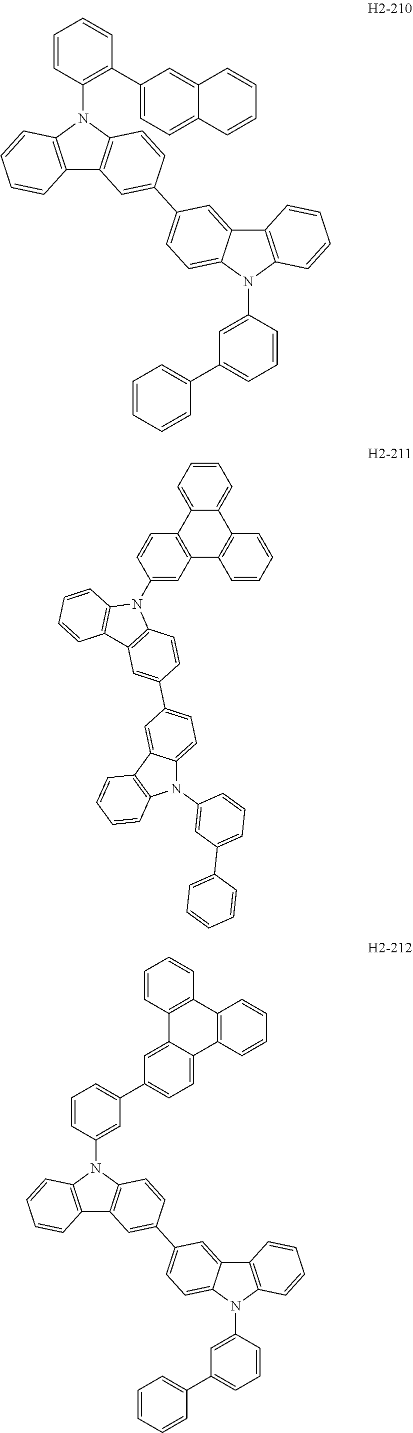 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1] 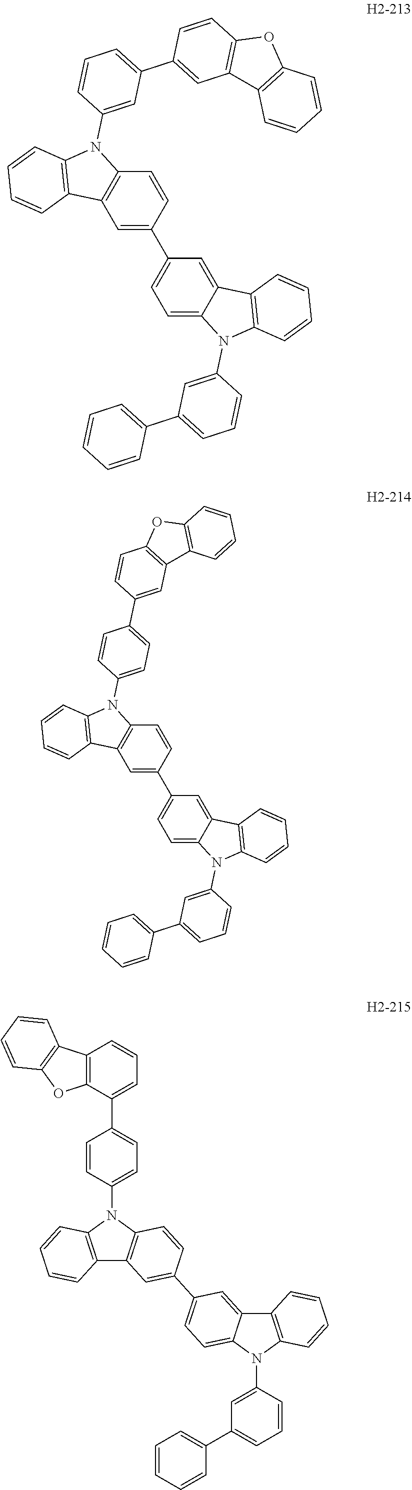 [SMILES: c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1] 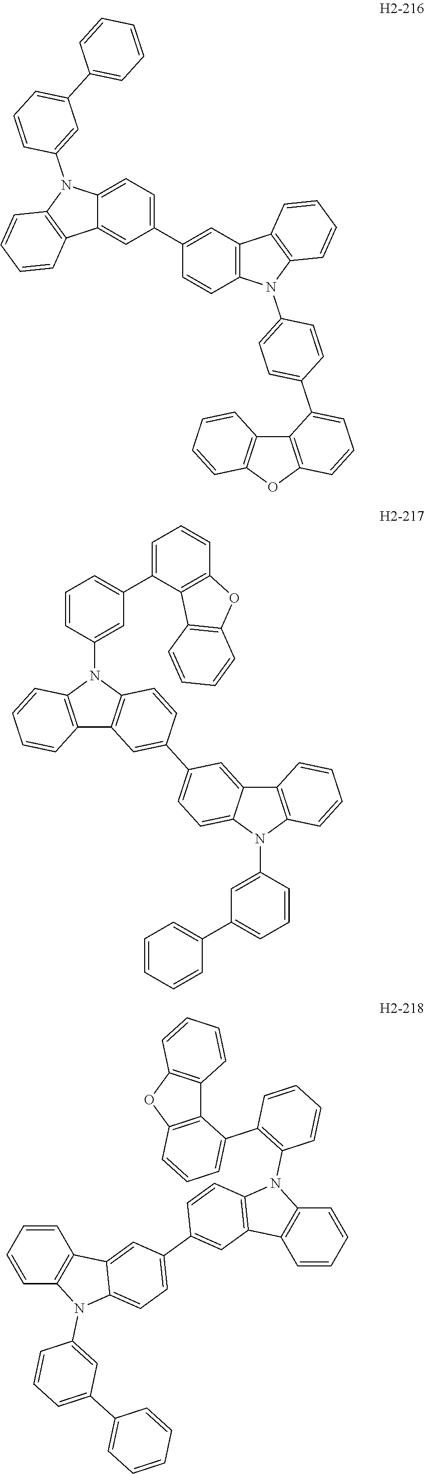 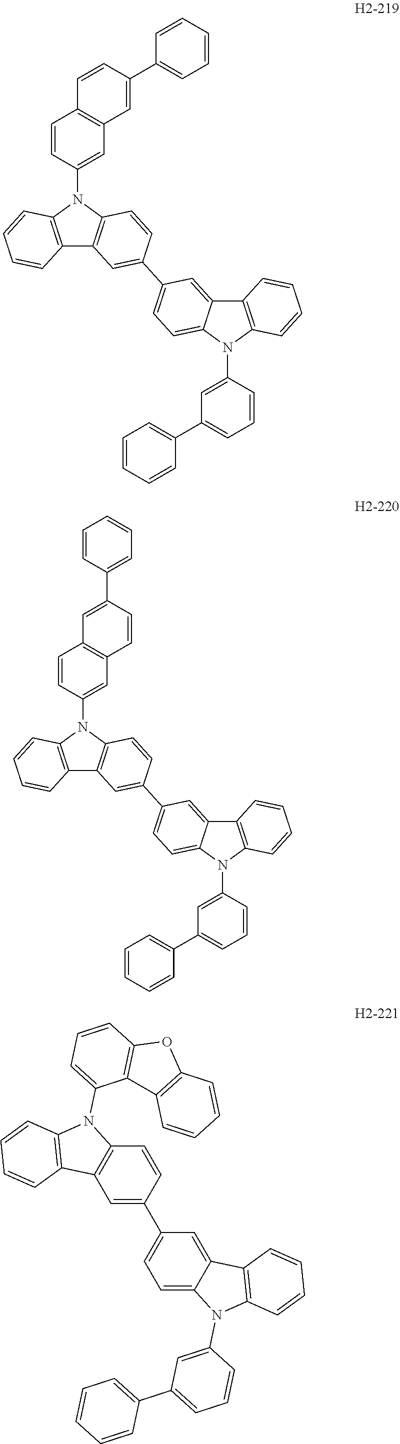 [SMILES: c1ccc(-c2cccc(-n3c4ccccc4c4cc(-c5ccc6c(c5)c5ccccc5n6-c5ccc6ccc(-c7ccccc7)cc6c5)ccc43)c2)cc1 ; c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5cccc6oc7ccccc7c56)ccc43)c2)cc1] 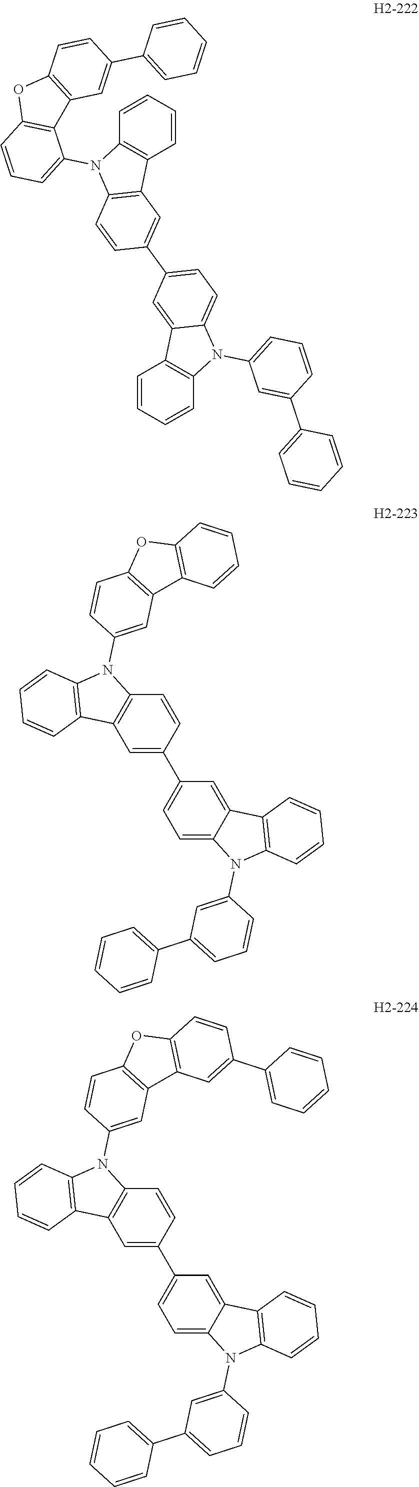 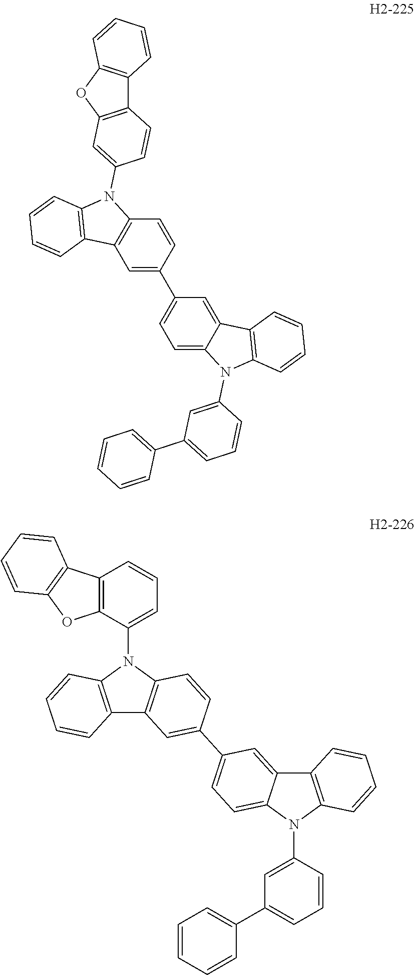 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cccc6c5oc5ccccc56)ccc43)c2)cc1] 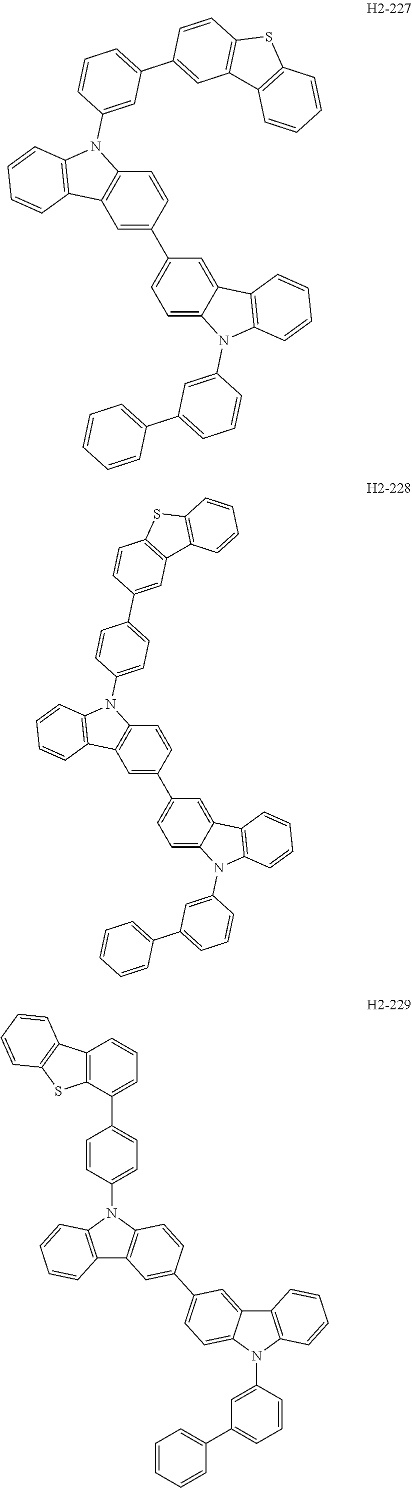 [SMILES: c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1] 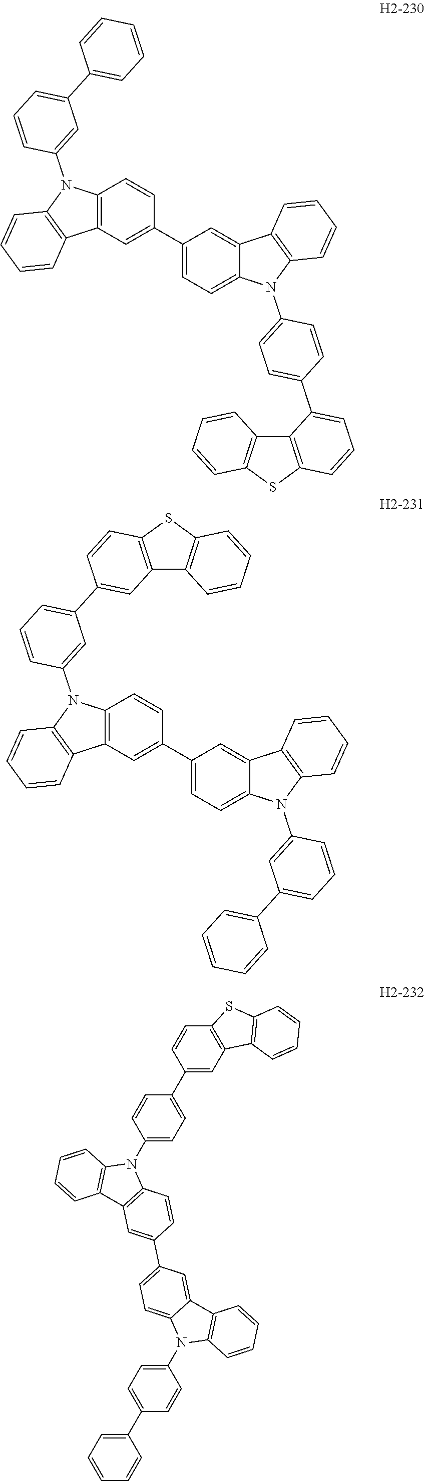 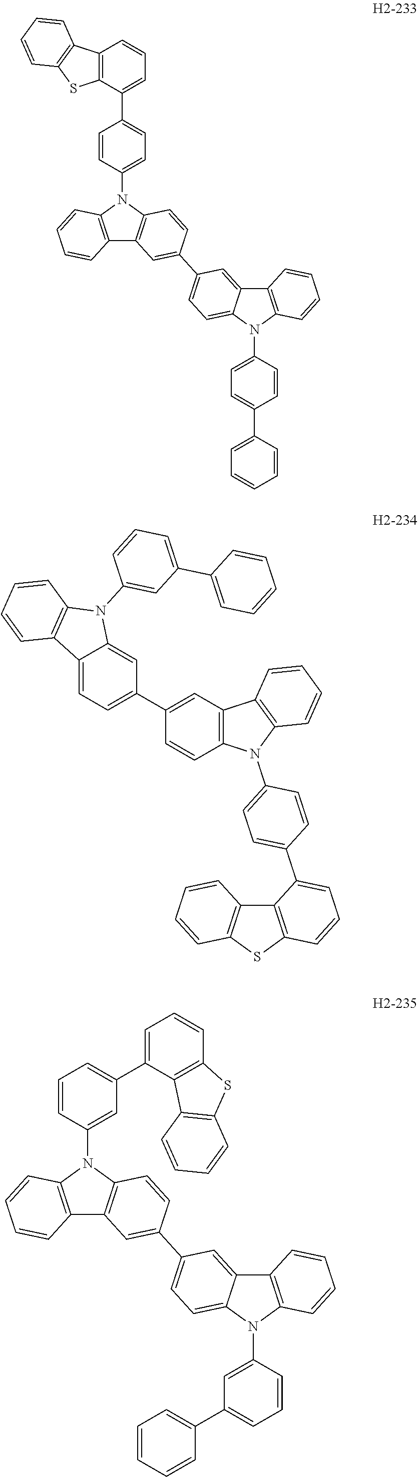 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4ccc(-c5ccc6c(c5)c5ccccc5n6-c5ccc(-c6cccc7sc8ccccc8c67)cc5)cc43)c2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1] 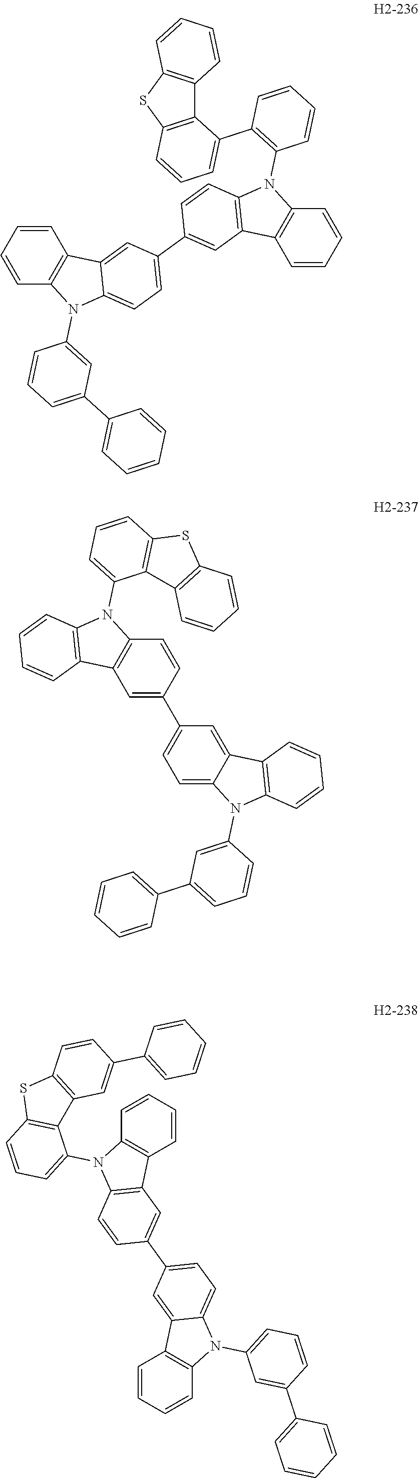 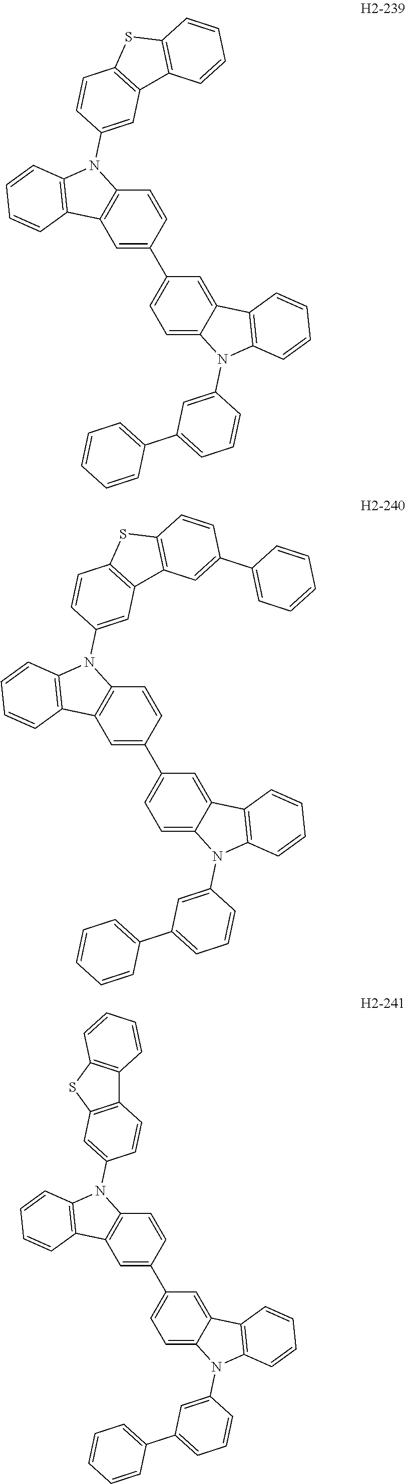 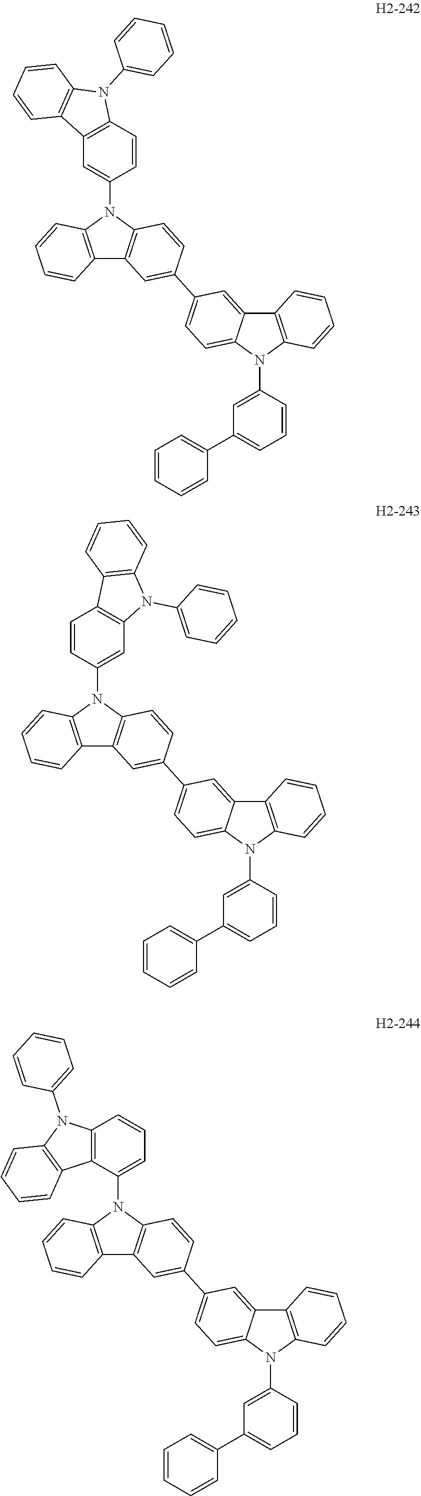 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1 ; c1ccc(-c2cccc(-n3c4ccccc4c4cc(-c5ccc6c(c5)c5ccccc5n6-c5cccc6c5c5ccccc5n6-c5ccccc5)ccc43)c2)cc1] 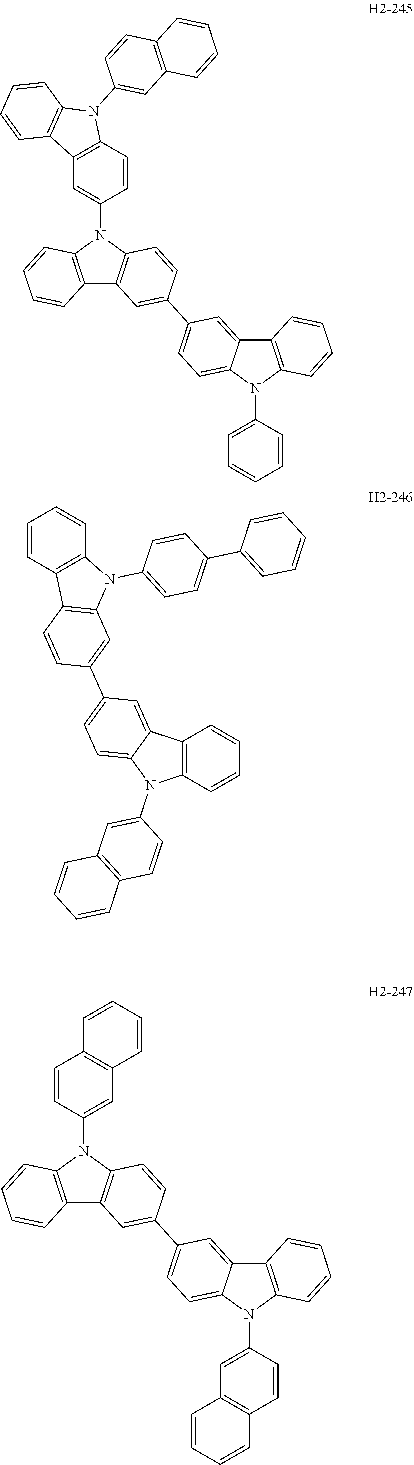 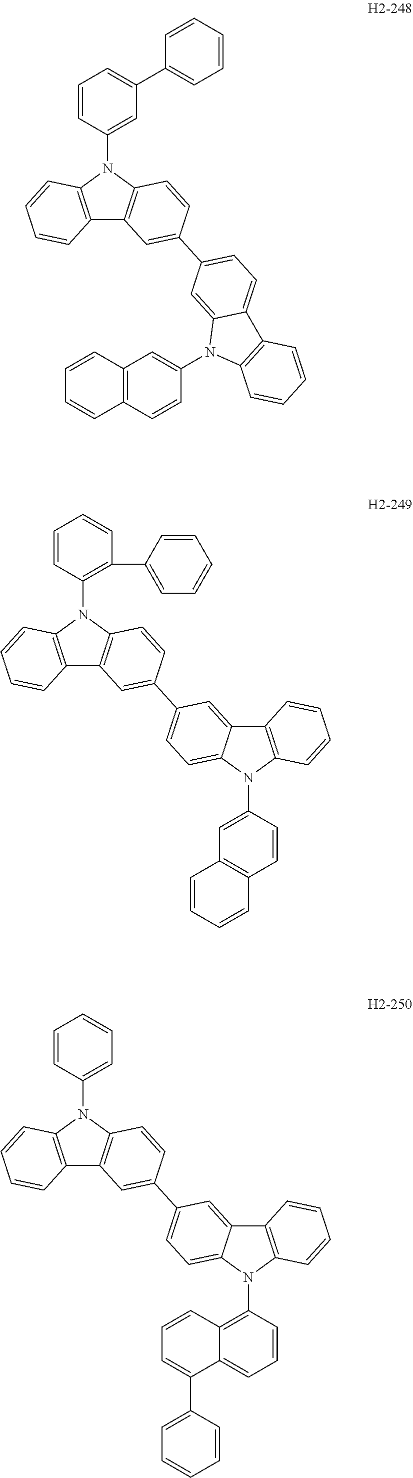 [SMILES: c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1] 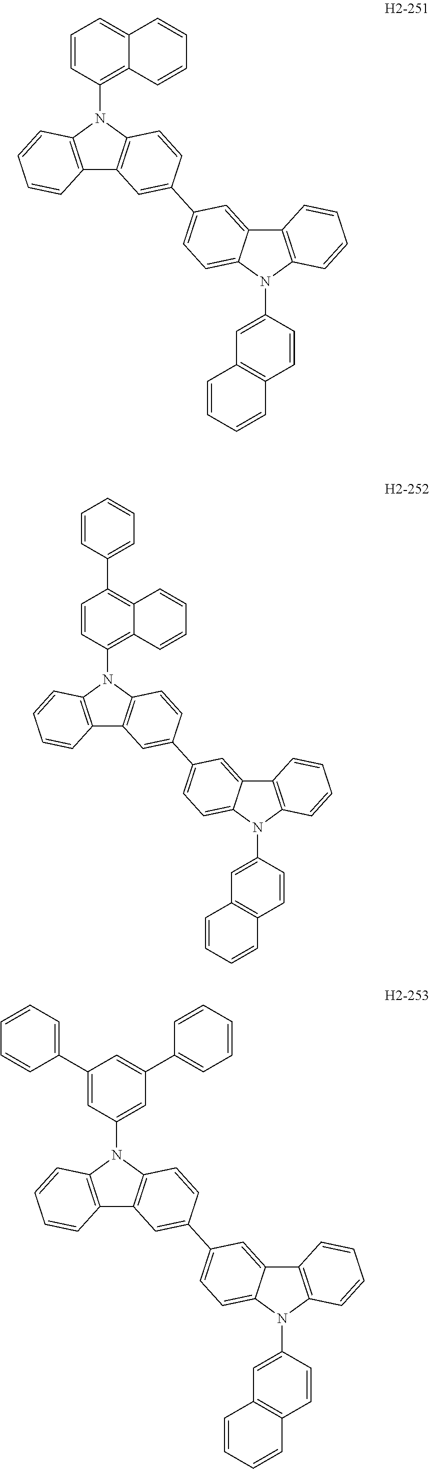 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1] 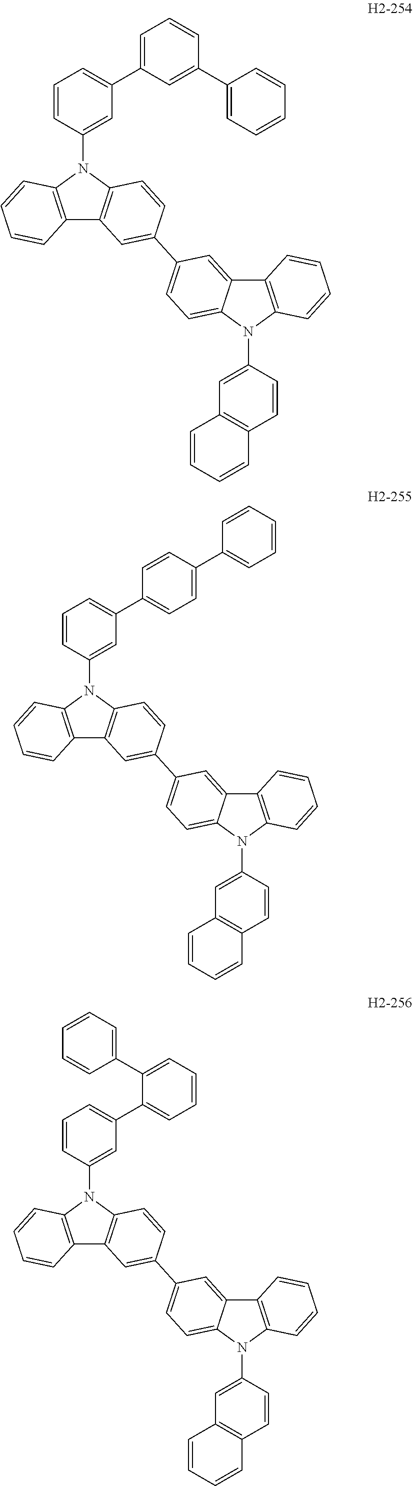 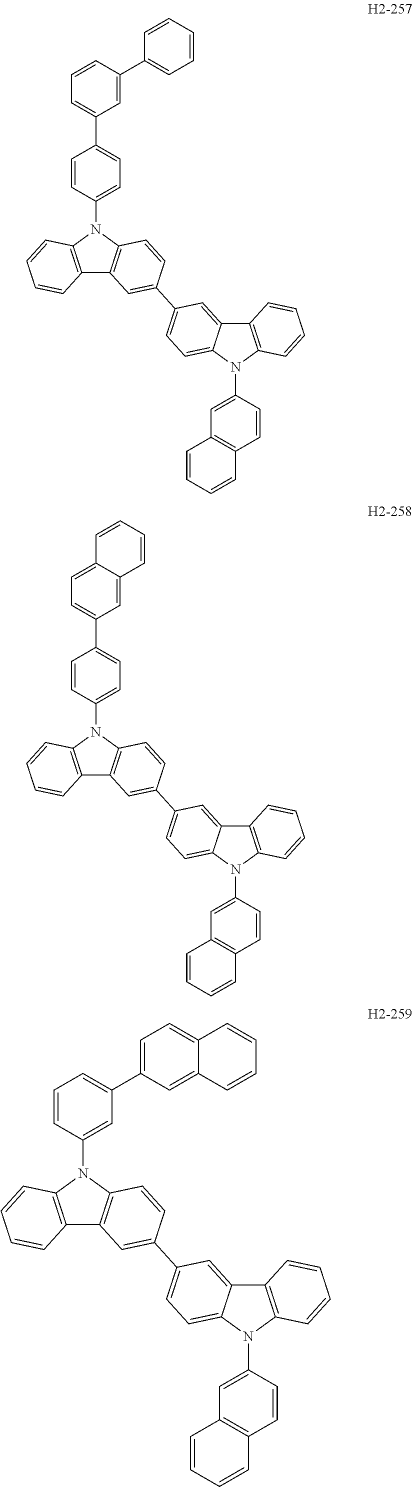 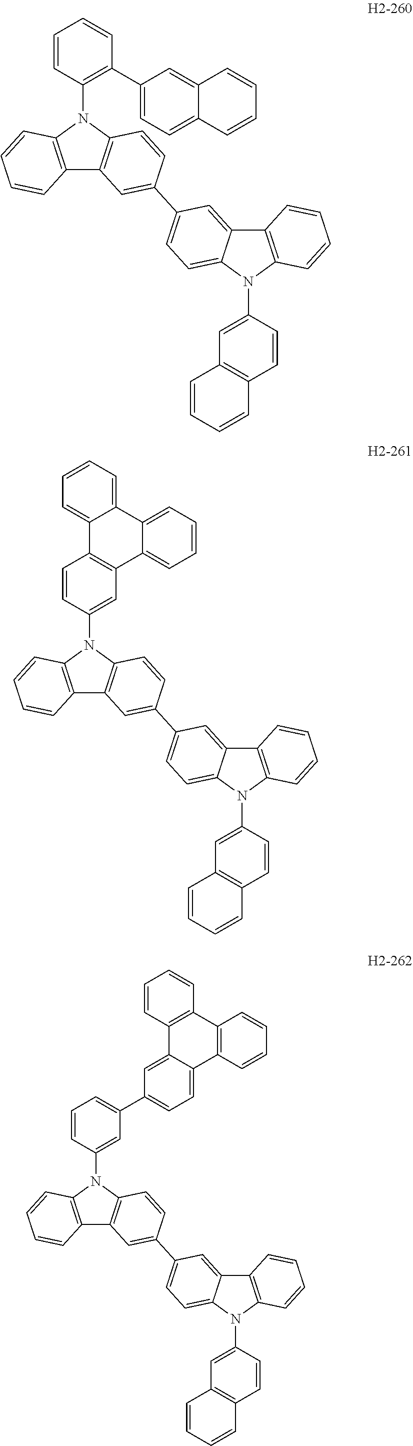 [SMILES: c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1] 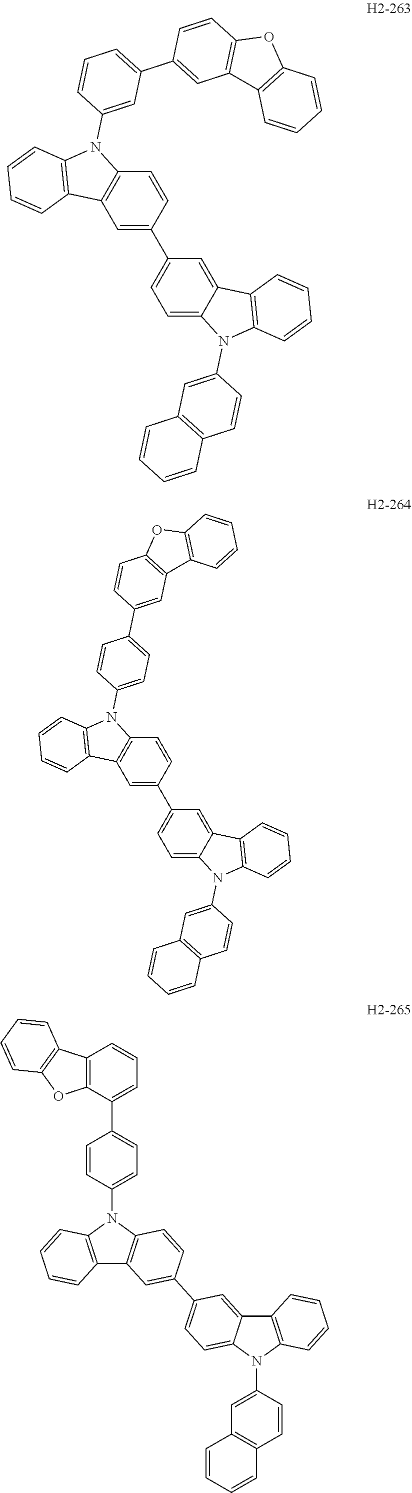 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6oc6ccccc67)cc5)ccc43)ccc2c1] 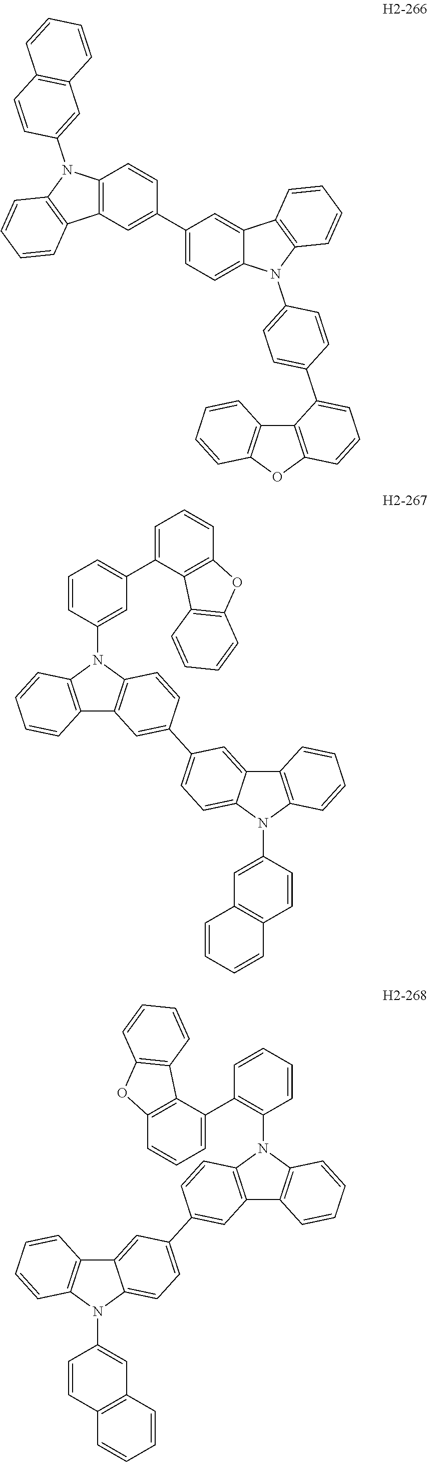 [SMILES: c1ccc2cc(-n3c4ccccc4c4cc(-c5ccc6c(c5)c5ccccc5n6-c5ccc(-c6cccc7oc8ccccc8c67)cc5)ccc43)ccc2c1 ; c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1] 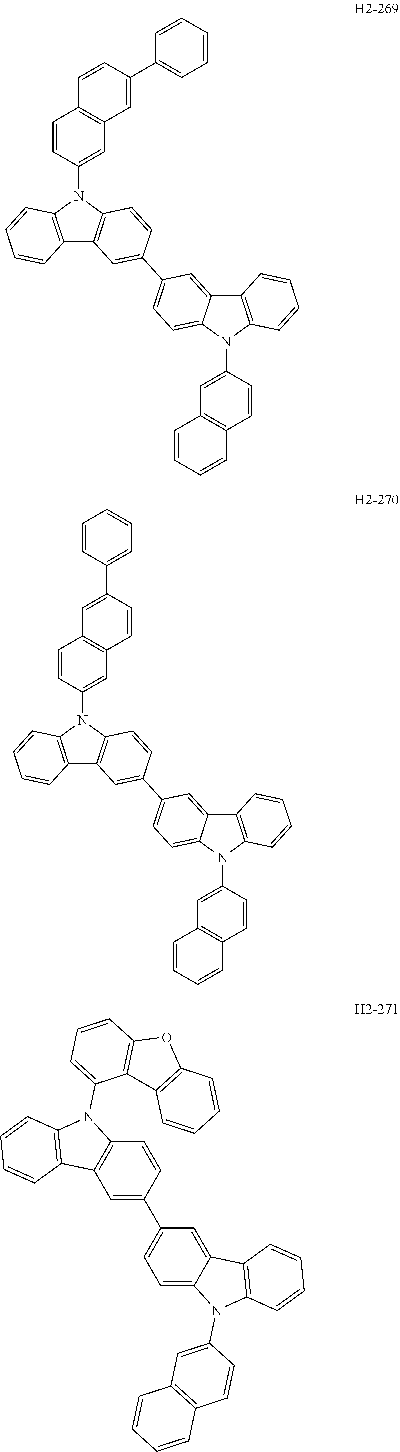 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1 ; c1ccc2cc(-n3c4ccccc4c4cc(-c5ccc6c(c5)c5ccccc5n6-c5cccc6oc7ccccc7c56)ccc43)ccc2c1] 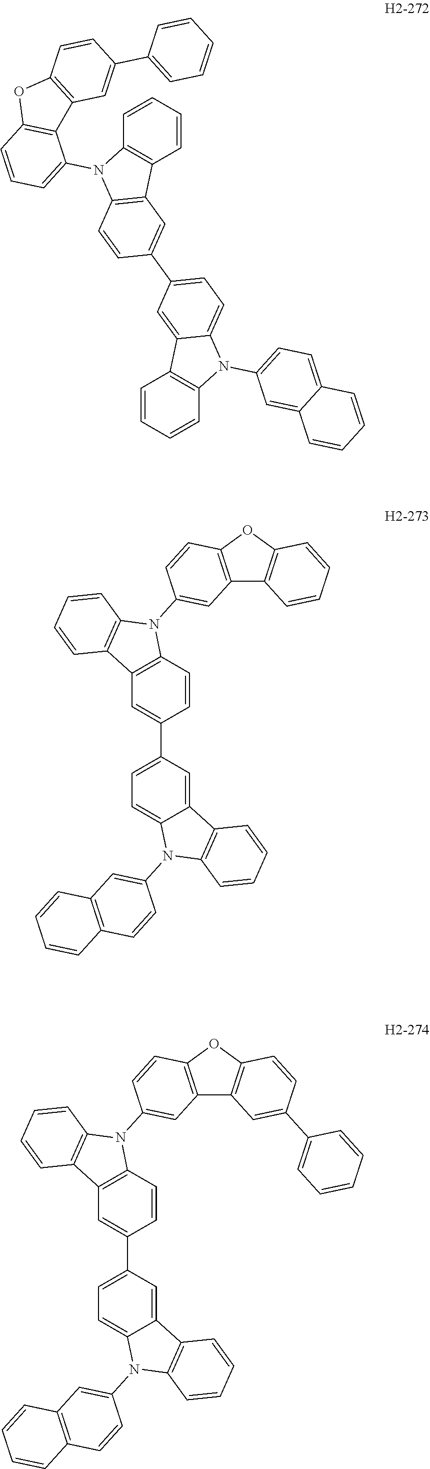 [SMILES: c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1] 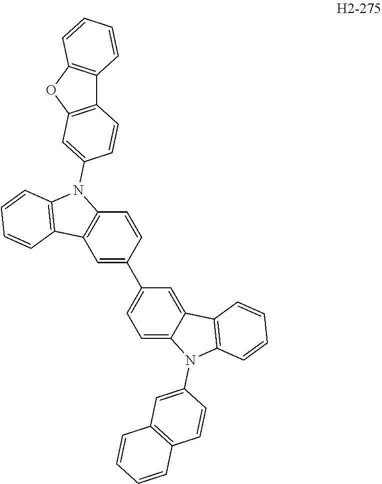 [SMILES: c1ccc2cc(-n3c4ccccc4c4cc(-c5ccc6c(c5)c5ccccc5n6-c5ccc6c(c5)oc5ccccc56)ccc43)ccc2c1] 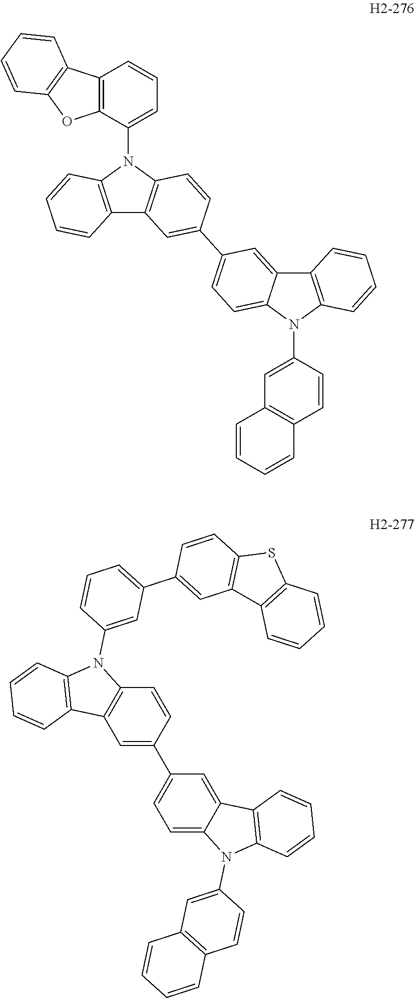 [SMILES: c1ccc2cc(-n3c4ccccc4c4cc(-c5ccc6c(c5)c5ccccc5n6-c5cccc6c5oc5ccccc56)ccc43)ccc2c1 ; c1cc(-c2ccc3sc4ccccc4c3c2)cc(-n2c3ccccc3c3cc(-c4ccc5c(c4)c4ccccc4n5-c4ccc5ccccc5c4)ccc32)c1] 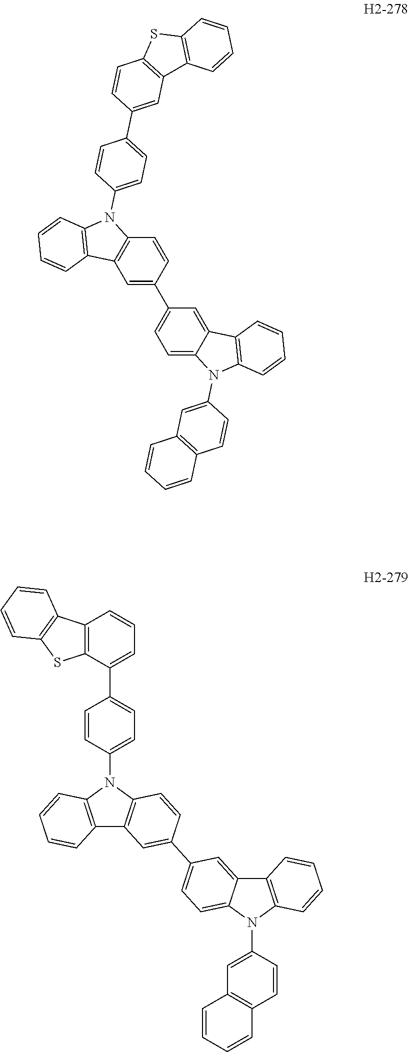 [SMILES: c1ccc2cc(-n3c4ccccc4c4cc(-c5ccc6c(c5)c5ccccc5n6-c5ccc(-c6ccc7sc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6sc6ccccc67)cc5)ccc43)ccc2c1] 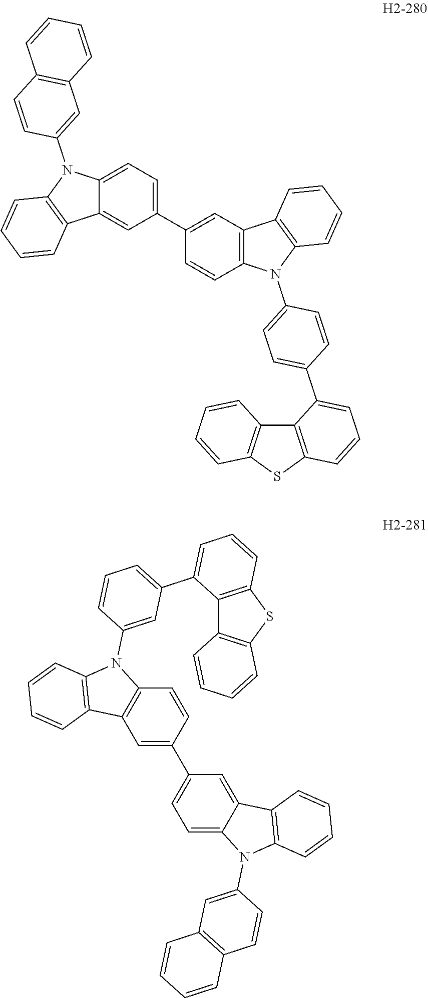 [SMILES: c1ccc2cc(-n3c4ccccc4c4cc(-c5ccc6c(c5)c5ccccc5n6-c5ccc(-c6cccc7sc8ccccc8c67)cc5)ccc43)ccc2c1 ; c1cc(-c2cccc3sc4ccccc4c23)cc(-n2c3ccccc3c3cc(-c4ccc5c(c4)c4ccccc4n5-c4ccc5ccccc5c4)ccc32)c1] 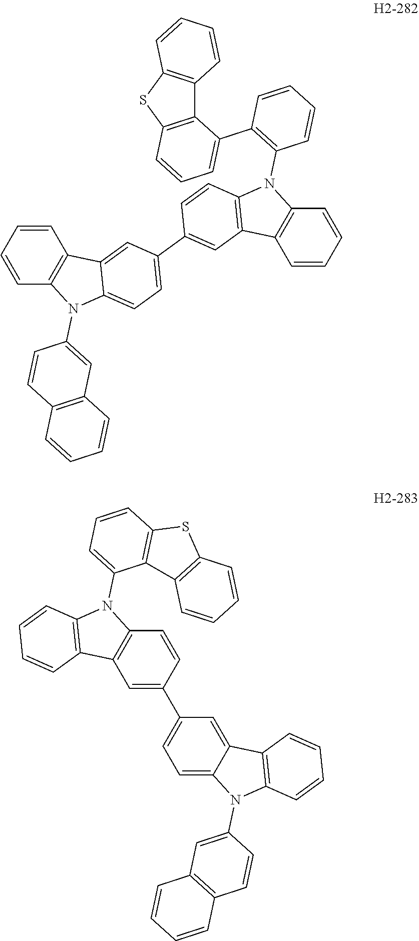 [SMILES: c1ccc(-n2c3ccccc3c3cc(-c4ccc5c(c4)c4ccccc4n5-c4ccc5ccccc5c4)ccc32)c(-c2cccc3sc4ccccc4c23)c1 ; c1ccc2cc(-n3c4ccccc4c4cc(-c5ccc6c(c5)c5ccccc5n6-c5cccc6sc7ccccc7c56)ccc43)ccc2c1] 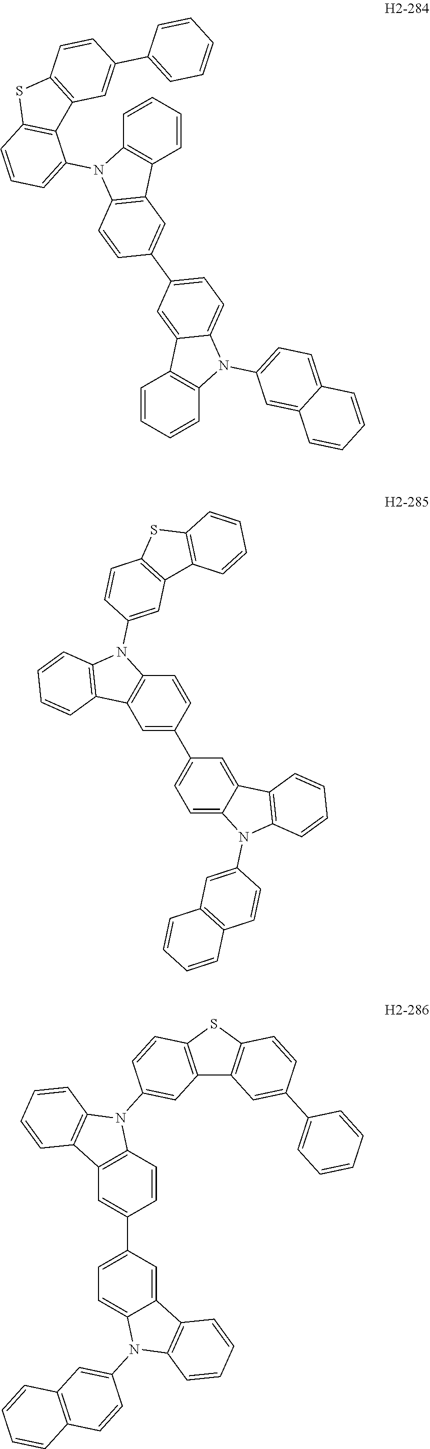 [SMILES: c1ccc(-c2ccc3sc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6sc7ccccc7c6c5)ccc43)ccc2c1 ; c1ccc(-c2ccc3sc4ccc(-n5c6ccccc6c6cc(-c7ccc8c(c7)c7ccccc7n8-c7ccc8ccccc8c7)ccc65)cc4c3c2)cc1] 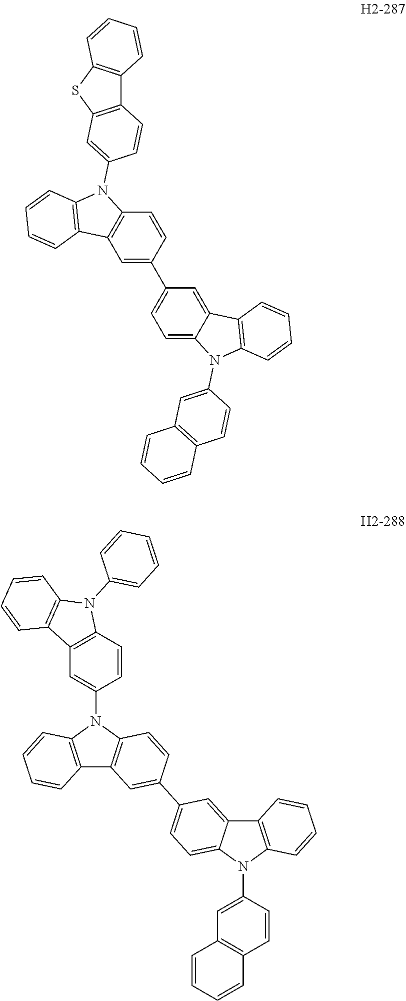 [SMILES: c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1] 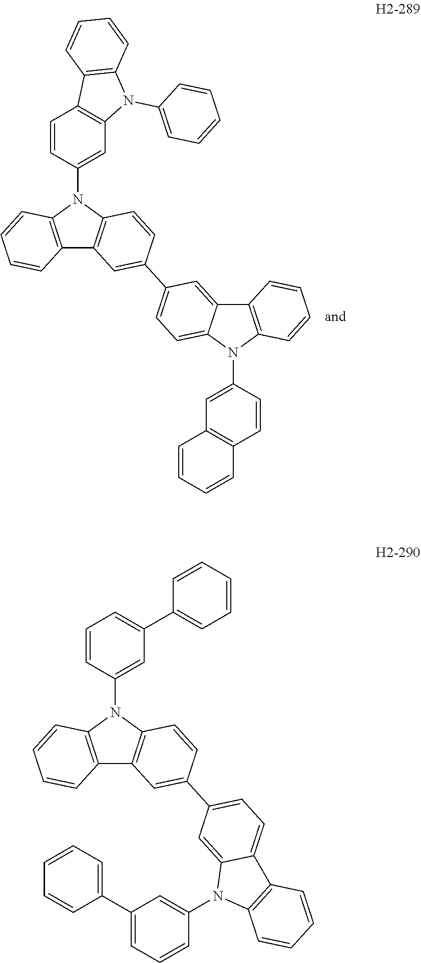 [SMILES: c1ccc(-n2c3ccccc3c3ccc(-n4c5ccccc5c5cc(-c6ccc7c(c6)c6ccccc6n7-c6ccc7ccccc7c6)ccc54)cc32)cc1 ; c1ccc(-c2cccc(-n3c4ccccc4c4cc(-c5ccc6c7ccccc7n(-c7cccc(-c8ccccc8)c7)c6c5)ccc43)c2)cc1]
wherein “Dₙ” in the above compounds means that n number of hydrogens is replaced by deuterium.

13. The organic electroluminescent material according to claim 5, wherein the compound represented by Formula 3 is selected from the following compounds: 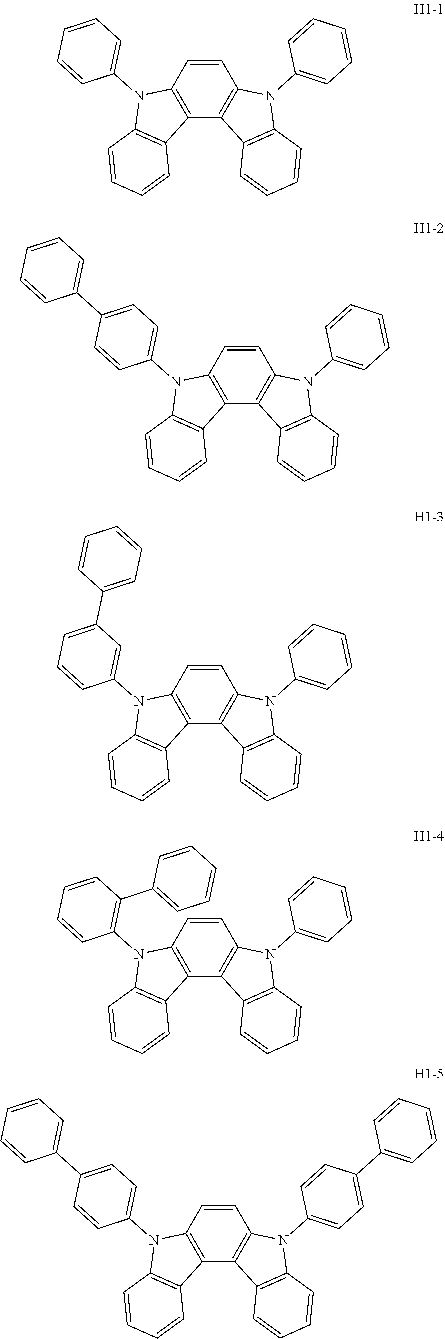 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccccc2-n2c3ccccc3c3c4c5ccccc5n(-c5ccccc5)c4ccc32)cc1 ; c1ccc(-c2ccc(-n3c4ccccc4c4c5c6ccccc6n(-c6ccc(-c7ccccc7)cc6)c5ccc43)cc2)cc1] 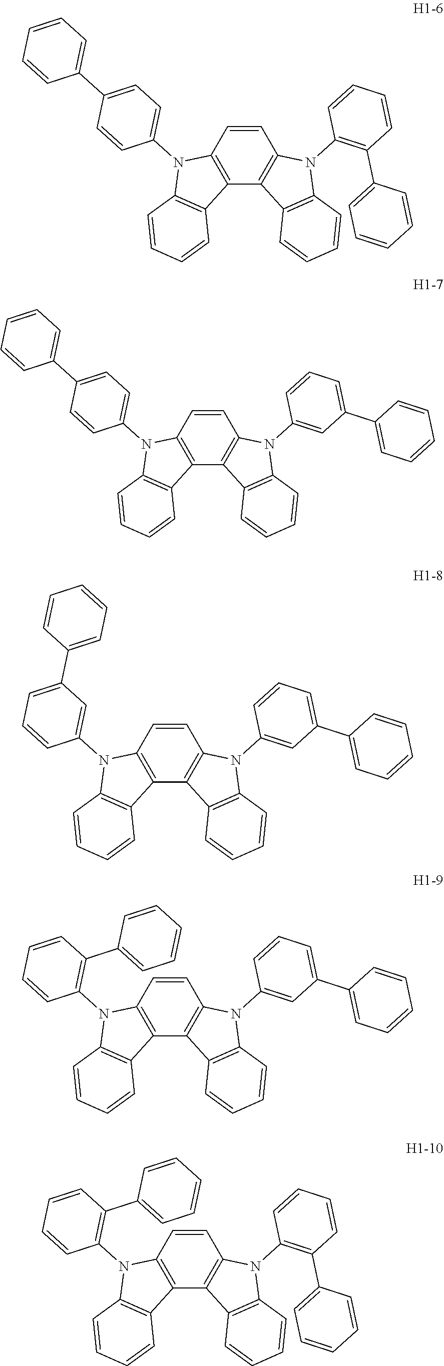 [SMILES: c1ccc(-c2ccc(-n3c4ccccc4c4c5c6ccccc6n(-c6ccccc6-c6ccccc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2cccc(-n3c4ccccc4c4c5c6ccccc6n(-c6cccc(-c7ccccc7)c6)c5ccc43)c2)cc1 ; c1ccc(-c2cccc(-n3c4ccccc4c4c5c6ccccc6n(-c6ccccc6-c6ccccc6)c5ccc43)c2)cc1 ; c1ccc(-c2ccccc2-n2c3ccccc3c3c4c5ccccc5n(-c5ccccc5-c5ccccc5)c4ccc32)cc1] 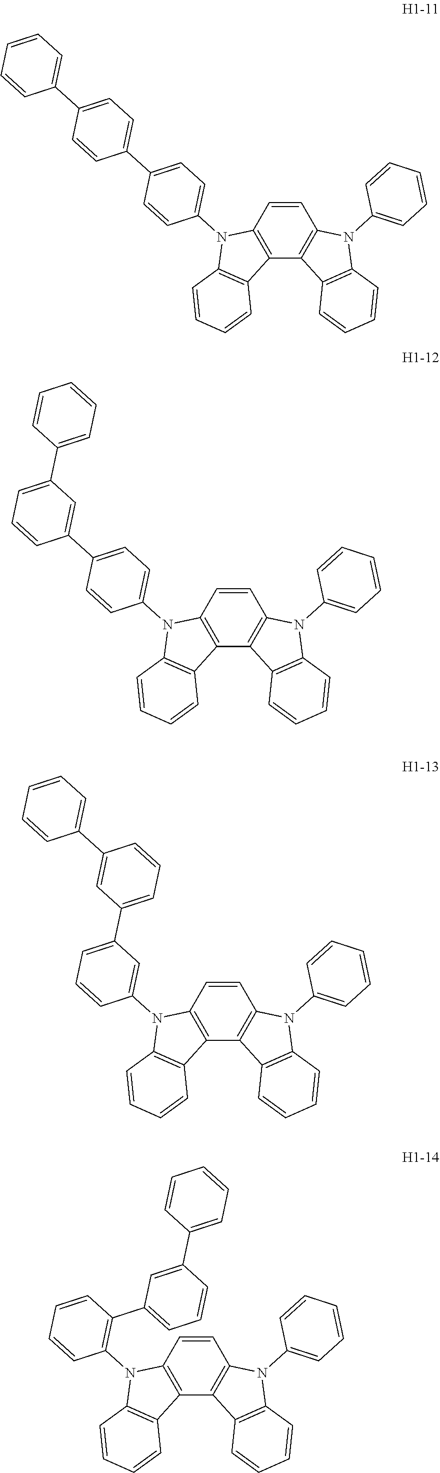 [SMILES: c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2cccc(-c3ccccc3-n3c4ccccc4c4c5c6ccccc6n(-c6ccccc6)c5ccc43)c2)cc1] 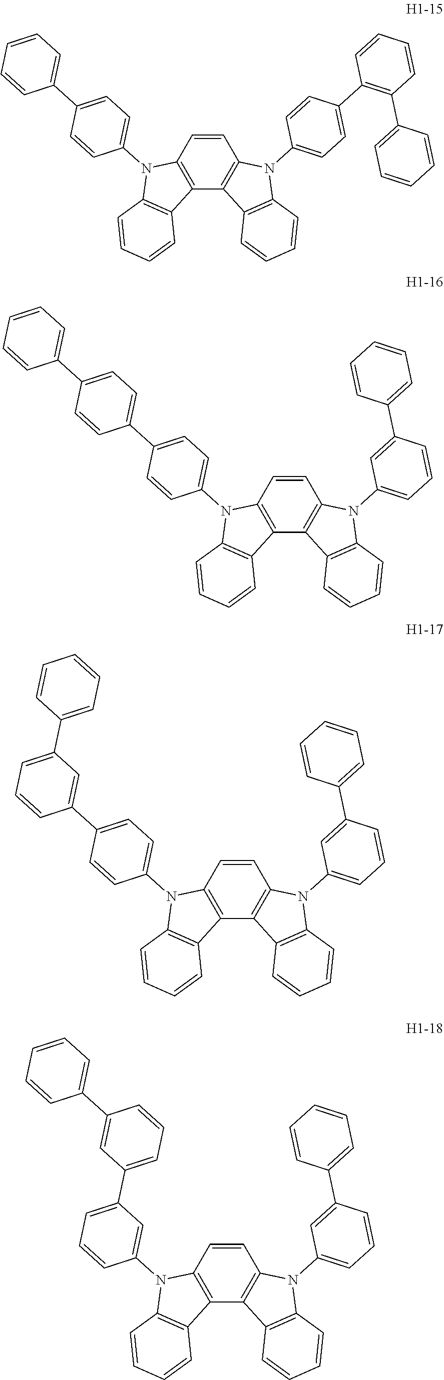 [SMILES: c1ccc(-c2ccc(-n3c4ccccc4c4c5c6ccccc6n(-c6ccc(-c7ccccc7-c7ccccc7)cc6)c5ccc43)cc2)cc1 ; c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2cccc(-c3cccc(-n4c5ccccc5c5c6c7ccccc7n(-c7cccc(-c8ccccc8)c7)c6ccc54)c3)c2)cc1] 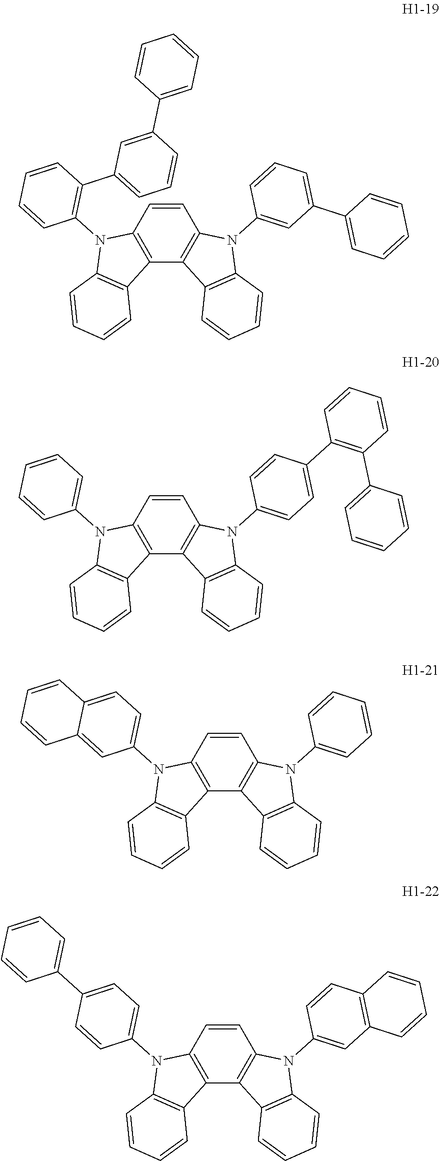 [SMILES: c1ccc(-c2cccc(-c3ccccc3-n3c4ccccc4c4c5c6ccccc6n(-c6cccc(-c7ccccc7)c6)c5ccc43)c2)cc1 ; c1ccc(-c2ccccc2-c2ccc(-n3c4ccccc4c4c5c6ccccc6n(-c6ccccc6)c5ccc43)cc2)cc1 ; c1ccc(-n2c3ccccc3c3c4c5ccccc5n(-c5ccc6ccccc6c5)c4ccc32)cc1 ; c1ccc(-c2ccc(-n3c4ccccc4c4c5c6ccccc6n(-c6ccc7ccccc7c6)c5ccc43)cc2)cc1] 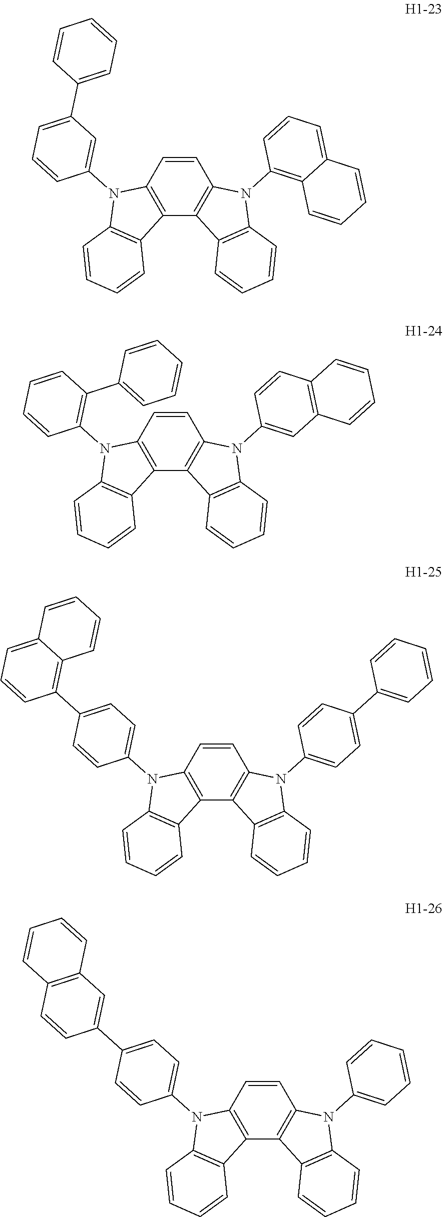 [SMILES: c1ccc(-c2cccc(-n3c4ccccc4c4c5c6ccccc6n(-c6cccc7ccccc67)c5ccc43)c2)cc1 ; c1ccc(-c2ccccc2-n2c3ccccc3c3c4c5ccccc5n(-c5ccc6ccccc6c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-n2c3ccccc3c3c4c5ccccc5n(-c5ccc(-c6ccc7ccccc7c6)cc5)c4ccc32)cc1] 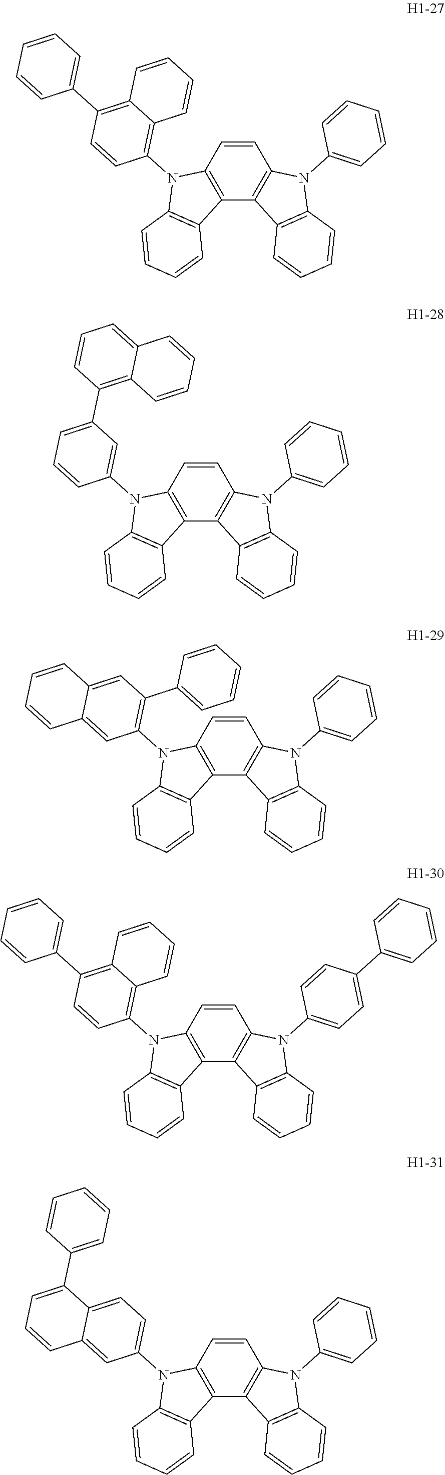 [SMILES: c1ccc(-c2ccc(-n3c4ccccc4c4c5c6ccccc6n(-c6ccccc6)c5ccc43)c3ccccc23)cc1 ; c1ccc(-n2c3ccccc3c3c4c5ccccc5n(-c5cccc(-c6cccc7ccccc67)c5)c4ccc32)cc1 ; c1ccc(-c2cc3ccccc3cc2-n2c3ccccc3c3c4c5ccccc5n(-c5ccccc5)c4ccc32)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc3cc(-n4c5ccccc5c5c6c7ccccc7n(-c7ccccc7)c6ccc54)ccc23)cc1] 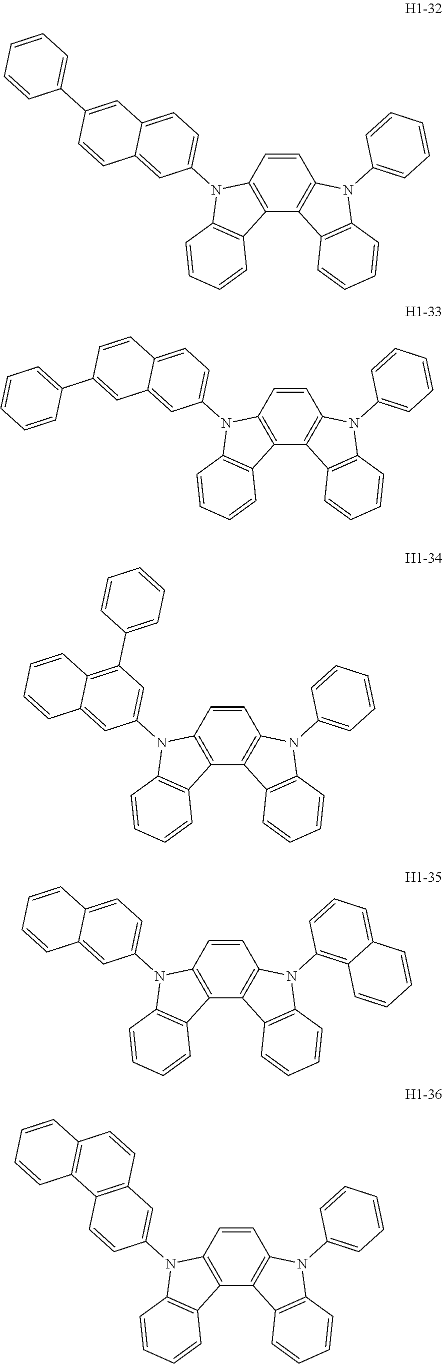 [SMILES: c1ccc(-c2ccc3cc(-n4c5ccccc5c5c6c7ccccc7n(-c7ccccc7)c6ccc54)ccc3c2)cc1 ; c1ccc(-c2ccc3ccc(-n4c5ccccc5c5c6c7ccccc7n(-c7ccccc7)c6ccc54)cc3c2)cc1 ; c1ccc(-c2cc(-n3c4ccccc4c4c5c6ccccc6n(-c6ccccc6)c5ccc43)cc3ccccc23)cc1 ; c1ccc2cc(-n3c4ccccc4c4c5c6ccccc6n(-c6cccc7ccccc67)c5ccc43)ccc2c1 ; c1ccc(-n2c3ccccc3c3c4c5ccccc5n(-c5ccc6c(ccc7ccccc76)c5)c4ccc32)cc1] 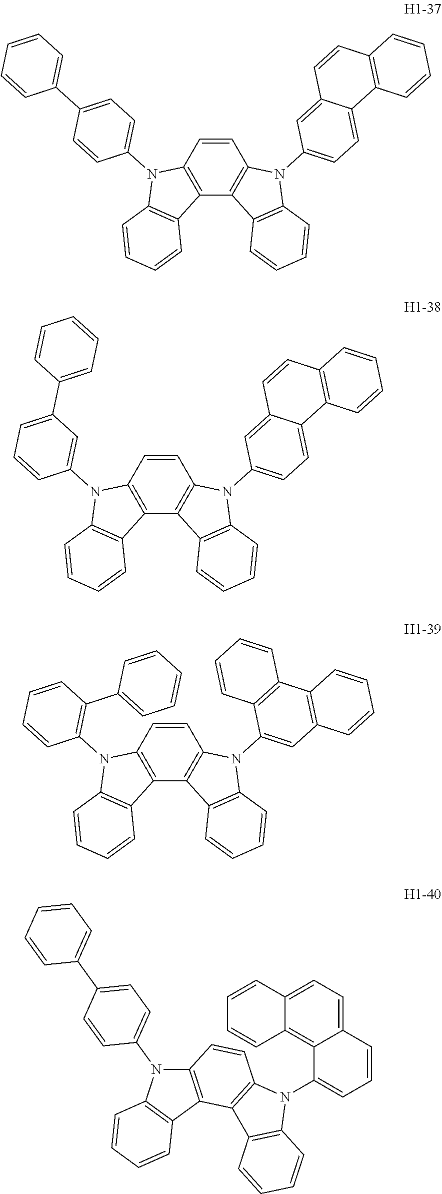 [SMILES: c1ccc(-c2ccc(-n3c4ccccc4c4c5c6ccccc6n(-c6ccc7c(ccc8ccccc87)c6)c5ccc43)cc2)cc1 ; c1ccc(-c2cccc(-n3c4ccccc4c4c5c6ccccc6n(-c6ccc7c(ccc8ccccc87)c6)c5ccc43)c2)cc1 ; c1ccc(-c2ccccc2-n2c3ccccc3c3c4c5ccccc5n(-c5cc6ccccc6c6ccccc56)c4ccc32)cc1 ; c1ccc(-c2ccc(-n3c4ccccc4c4c5c6ccccc6n(-c6cccc7ccc8ccccc8c67)c5ccc43)cc2)cc1] 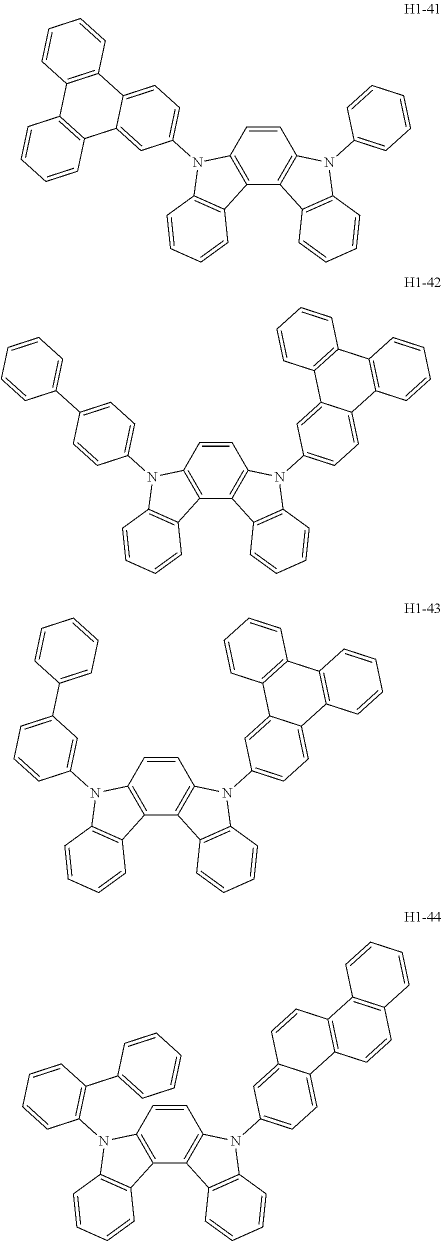 [SMILES: c1ccc(-n2c3ccccc3c3c4c5ccccc5n(-c5ccc6c7ccccc7c7ccccc7c6c5)c4ccc32)cc1 ; c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6ccc7c8ccccc8c8ccccc8c7c6)c5ccc43)c2)cc1 ; c1ccc(-c2ccccc2-n2c3ccccc3c3c4c5ccccc5n(-c5ccc6c(ccc7c8ccccc8ccc67)c5)c4ccc32)cc1] 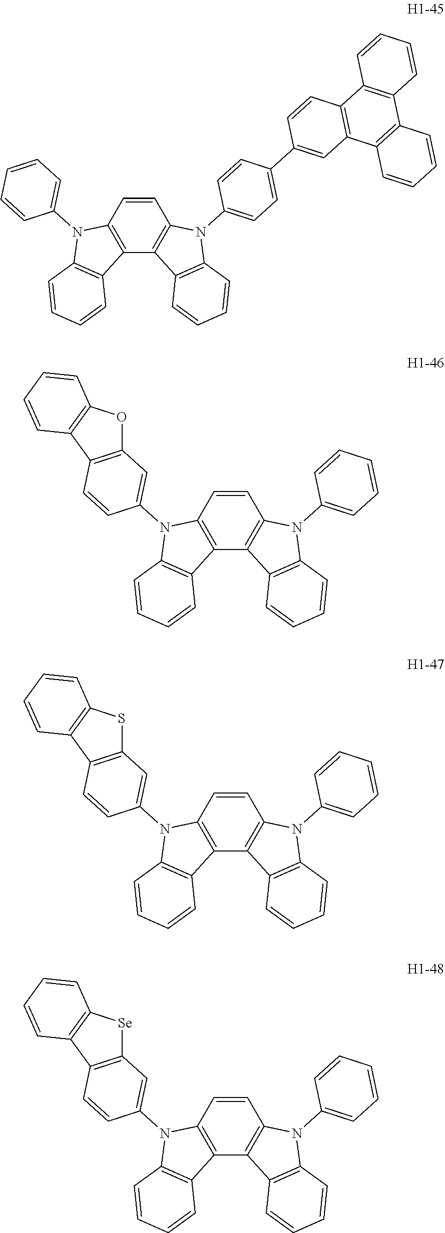 [SMILES: c1ccc(-n2c3ccccc3c3c4c5ccccc5n(-c5ccc(-c6ccc7c8ccccc8c8ccccc8c7c6)cc5)c4ccc32)cc1 ; c1ccc(-n2c3ccccc3c3c4c5ccccc5n(-c5ccc6c(c5)oc5ccccc56)c4ccc32)cc1 ; c1ccc(-n2c3ccccc3c3c4c5ccccc5n(-c5ccc6c(c5)sc5ccccc56)c4ccc32)cc1 ; c1ccc(-n2c3ccccc3c3c4c5ccccc5n(-c5ccc6c(c5)[se]c5ccccc56)c4ccc32)cc1] 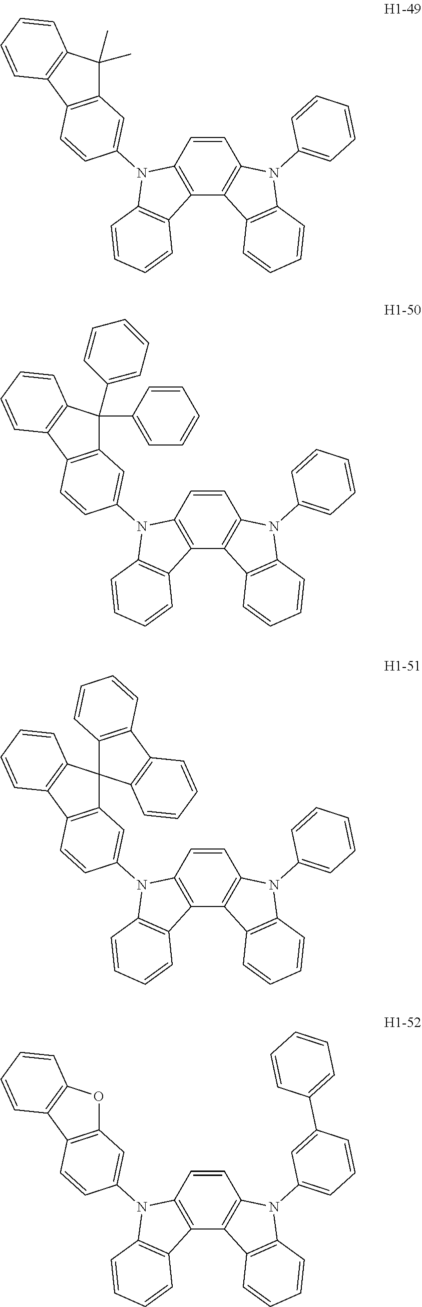 [SMILES: CC1(C)c2ccccc2-c2ccc(-n3c4ccccc4c4c5c6ccccc6n(-c6ccccc6)c5ccc43)cc21 ; c1ccc(-n2c3ccccc3c3c4c5ccccc5n(-c5ccc6c(c5)C(c5ccccc5)(c5ccccc5)c5ccccc5-6)c4ccc32)cc1 ; c1ccc(-n2c3ccccc3c3c4c5ccccc5n(-c5ccc6c(c5)C5(c7ccccc7-c7ccccc75)c5ccccc5-6)c4ccc32)cc1 ; c1ccc(-c2cccc(-n3c4ccccc4c4c5c6ccccc6n(-c6ccc7c(c6)oc6ccccc67)c5ccc43)c2)cc1] 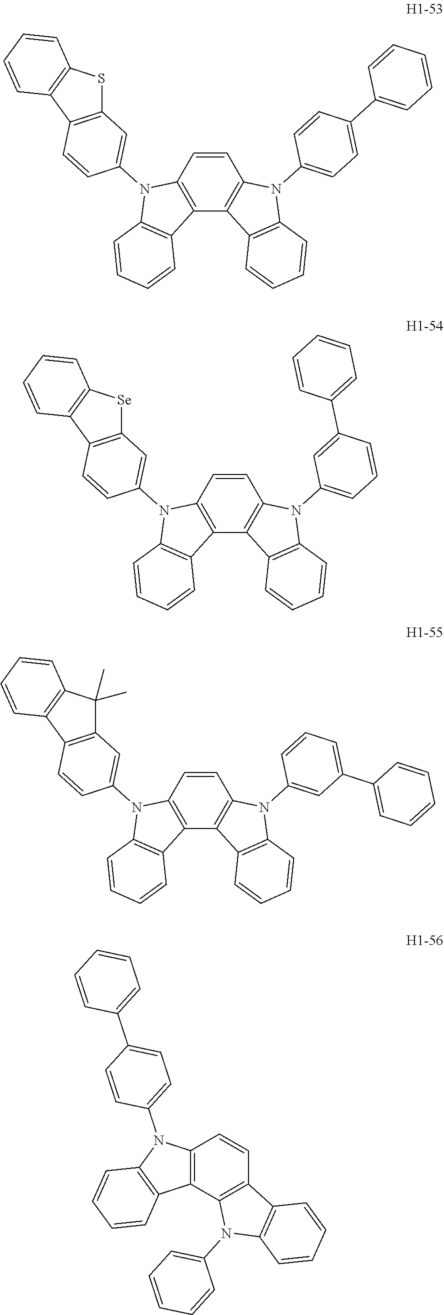 [SMILES: c1ccc(-c2ccc(-n3c4ccccc4c4c5c6ccccc6n(-c6ccc7c(c6)sc6ccccc67)c5ccc43)cc2)cc1 ; c1ccc(-c2cccc(-n3c4ccccc4c4c5c6ccccc6n(-c6ccc7c(c6)[se]c6ccccc67)c5ccc43)c2)cc1 ; CC1(C)c2ccccc2-c2ccc(-n3c4ccccc4c4c5c6ccccc6n(-c6cccc(-c7ccccc7)c6)c5ccc43)cc21 ; c1ccc(-c2ccc(-n3c4ccccc4c4c3ccc3c5ccccc5n(-c5ccccc5)c34)cc2)cc1] 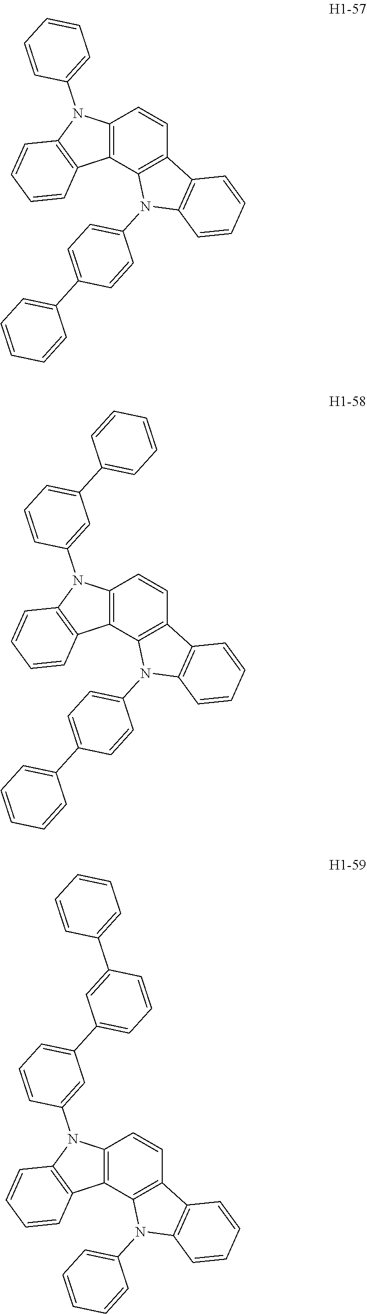 [SMILES: c1ccc(-c2ccc(-n3c4ccccc4c4ccc5c(c6ccccc6n5-c5ccccc5)c43)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2cccc(-c3cccc(-n4c5ccccc5c5c4ccc4c6ccccc6n(-c6ccccc6)c45)c3)c2)cc1] 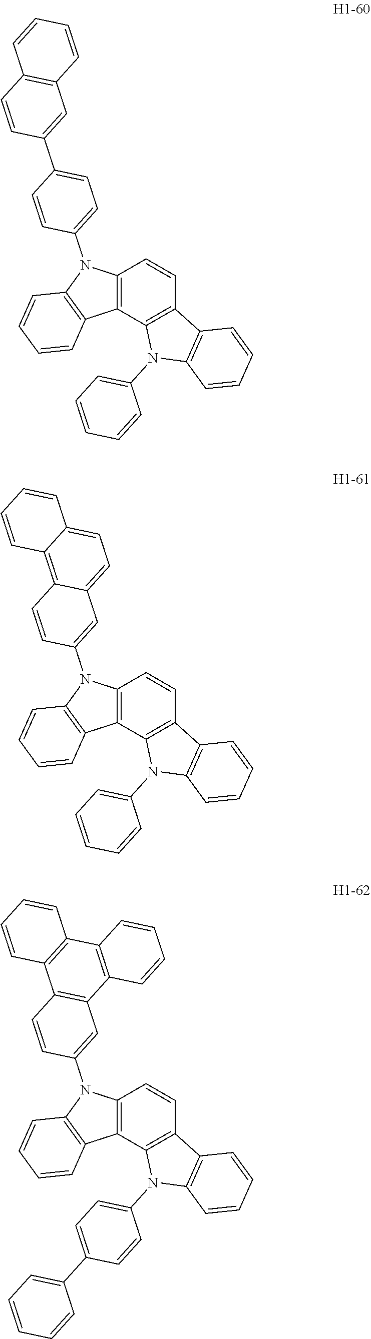 [SMILES: c1ccc(-n2c3ccccc3c3ccc4c(c5ccccc5n4-c4ccc(-c5ccc6ccccc6c5)cc4)c32)cc1 ; c1ccc(-n2c3ccccc3c3ccc4c(c5ccccc5n4-c4ccc5c(ccc6ccccc65)c4)c32)cc1 ; c1ccc(-c2ccc(-n3c4ccccc4c4ccc5c(c6ccccc6n5-c5ccc6c7ccccc7c7ccccc7c6c5)c43)cc2)cc1] 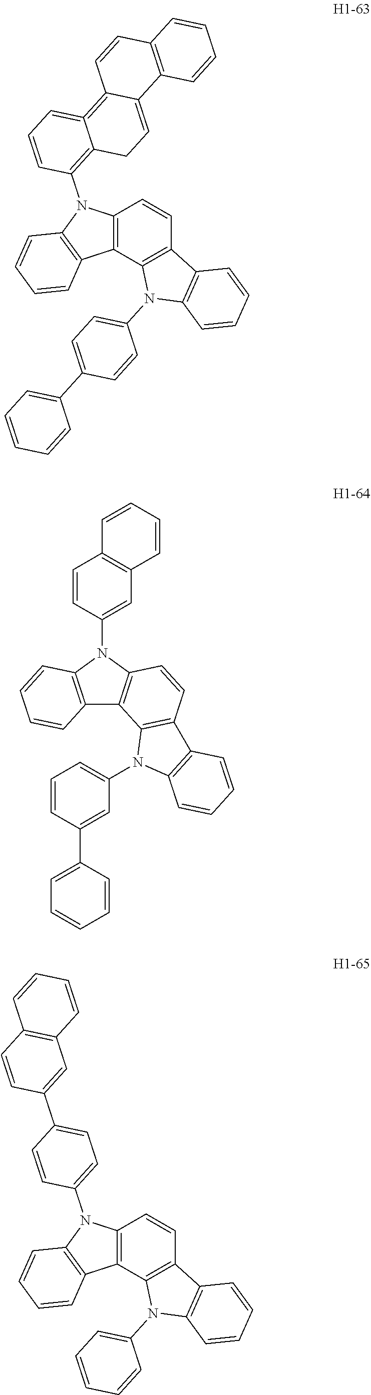 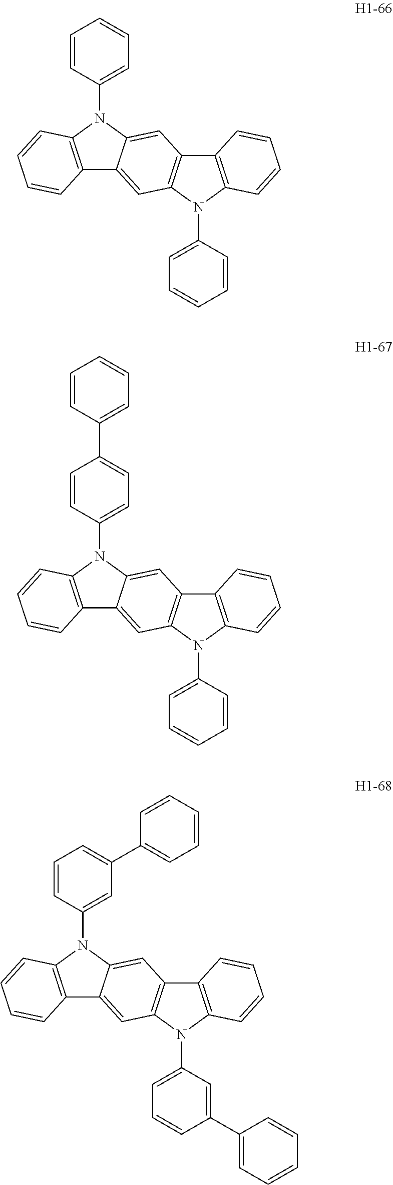 [SMILES: c1ccc(-n2c3ccccc3c3cc4c(cc32)c2ccccc2n4-c2ccccc2)cc1 ; c1ccc(-c2ccc(-n3c4ccccc4c4cc5c(cc43)c3ccccc3n5-c3ccccc3)cc2)cc1 ; c1ccc(-c2cccc(-n3c4ccccc4c4cc5c(cc43)c3ccccc3n5-c3cccc(-c4ccccc4)c3)c2)cc1] 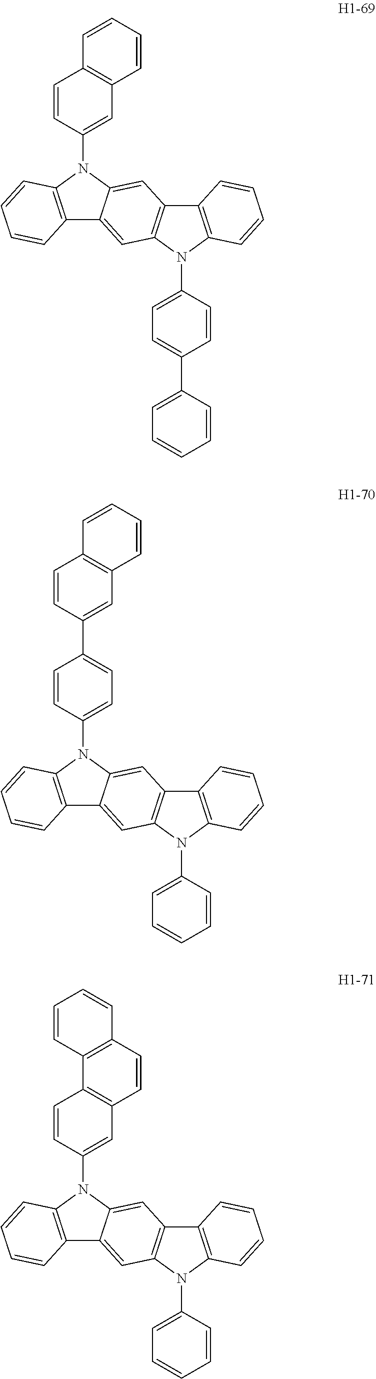 [SMILES: c1ccc(-c2ccc(-n3c4ccccc4c4cc5c(cc43)c3ccccc3n5-c3ccc4ccccc4c3)cc2)cc1 ; c1ccc(-n2c3ccccc3c3cc4c(cc32)c2ccccc2n4-c2ccc(-c3ccc4ccccc4c3)cc2)cc1 ; c1ccc(-n2c3ccccc3c3cc4c(cc32)c2ccccc2n4-c2ccc3c(ccc4ccccc43)c2)cc1] 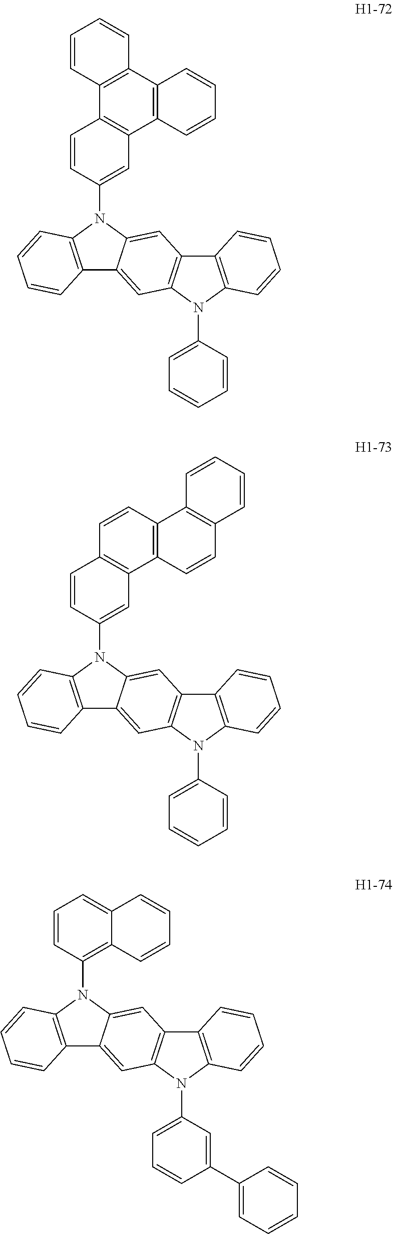 [SMILES: c1ccc(-n2c3ccccc3c3cc4c(cc32)c2ccccc2n4-c2ccc3c4ccccc4c4ccccc4c3c2)cc1 ; c1ccc(-n2c3ccccc3c3cc4c(cc32)c2ccccc2n4-c2ccc3ccc4c5ccccc5ccc4c3c2)cc1 ; c1ccc(-c2cccc(-n3c4ccccc4c4cc5c(cc43)c3ccccc3n5-c3cccc4ccccc34)c2)cc1] 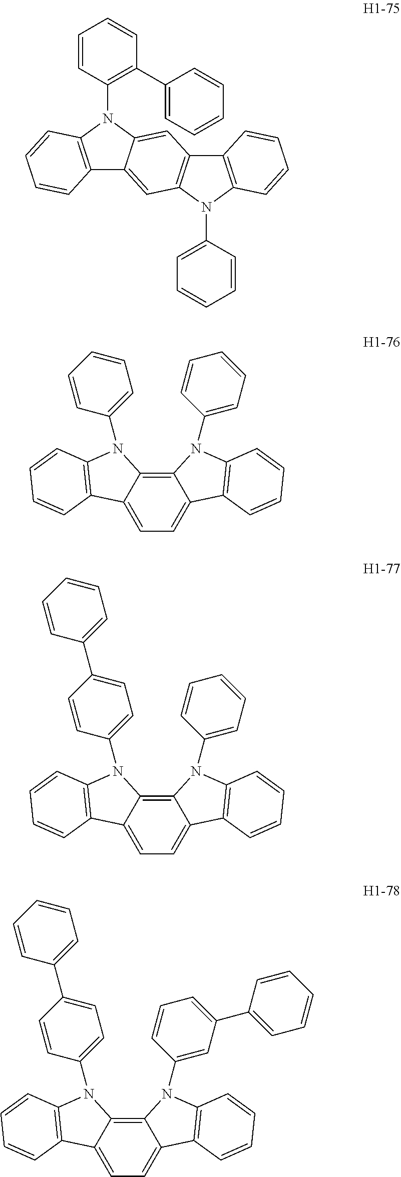 [SMILES: c1ccc(-c2ccccc2-n2c3ccccc3c3cc4c(cc32)c2ccccc2n4-c2ccccc2)cc1 ; c1ccc(-n2c3ccccc3c3ccc4c5ccccc5n(-c5ccccc5)c4c32)cc1 ; c1ccc(-c2ccc(-n3c4ccccc4c4ccc5c6ccccc6n(-c6ccccc6)c5c43)cc2)cc1 ; c1ccc(-c2ccc(-n3c4ccccc4c4ccc5c6ccccc6n(-c6cccc(-c7ccccc7)c6)c5c43)cc2)cc1] 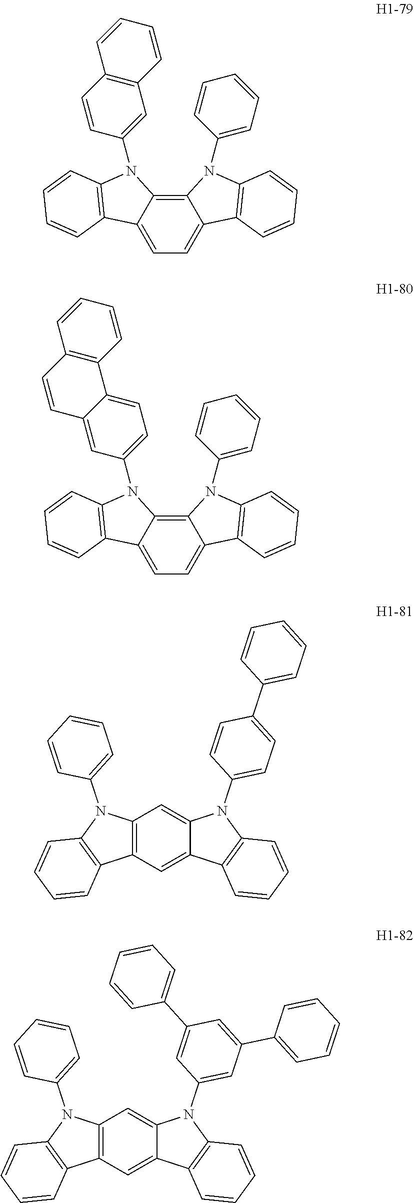 [SMILES: c1ccc(-n2c3ccccc3c3ccc4c5ccccc5n(-c5ccc6ccccc6c5)c4c32)cc1 ; c1ccc(-n2c3ccccc3c3ccc4c5ccccc5n(-c5ccc6c(ccc7ccccc76)c5)c4c32)cc1 ; c1ccc(-c2ccc(-n3c4ccccc4c4cc5c6ccccc6n(-c6ccccc6)c5cc43)cc2)cc1 ; c1ccc(-c2cc(-c3ccccc3)cc(-n3c4ccccc4c4cc5c6ccccc6n(-c6ccccc6)c5cc43)c2)cc1] 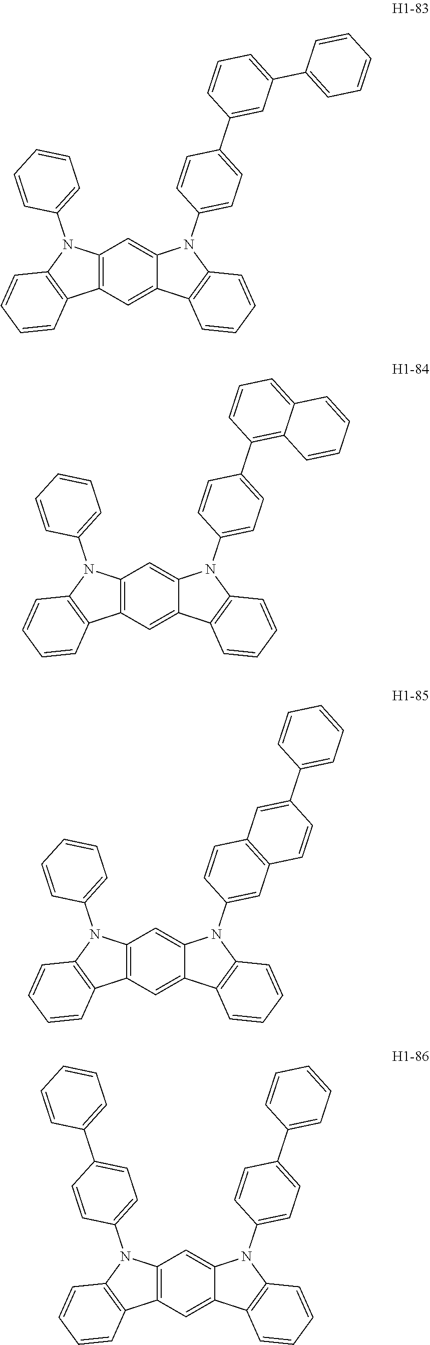 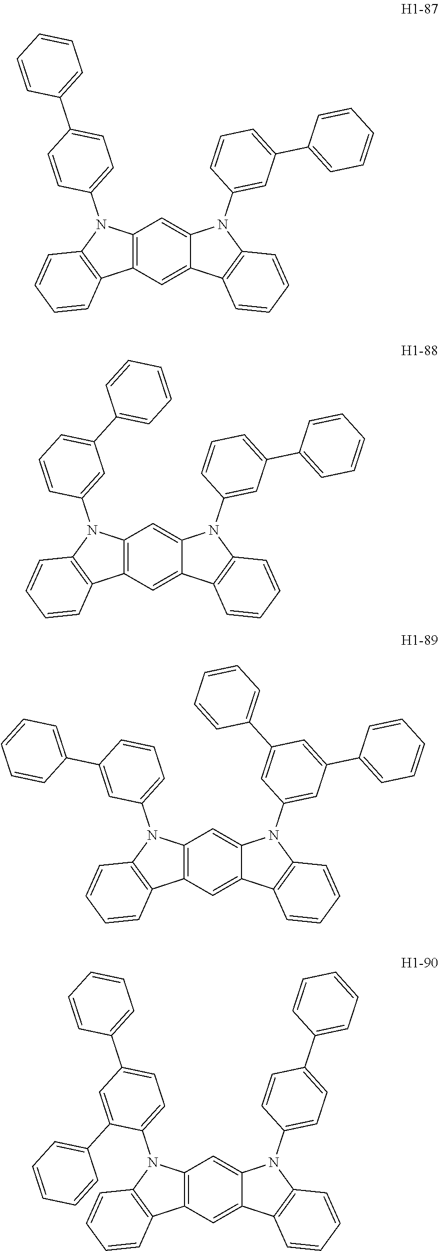 [SMILES: c1ccc(-c2ccc(-n3c4ccccc4c4cc5c6ccccc6n(-c6cccc(-c7ccccc7)c6)c5cc43)cc2)cc1 ; c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c6ccccc6n(-c6ccc(-c7ccccc7)cc6-c6ccccc6)c5cc43)cc2)cc1] 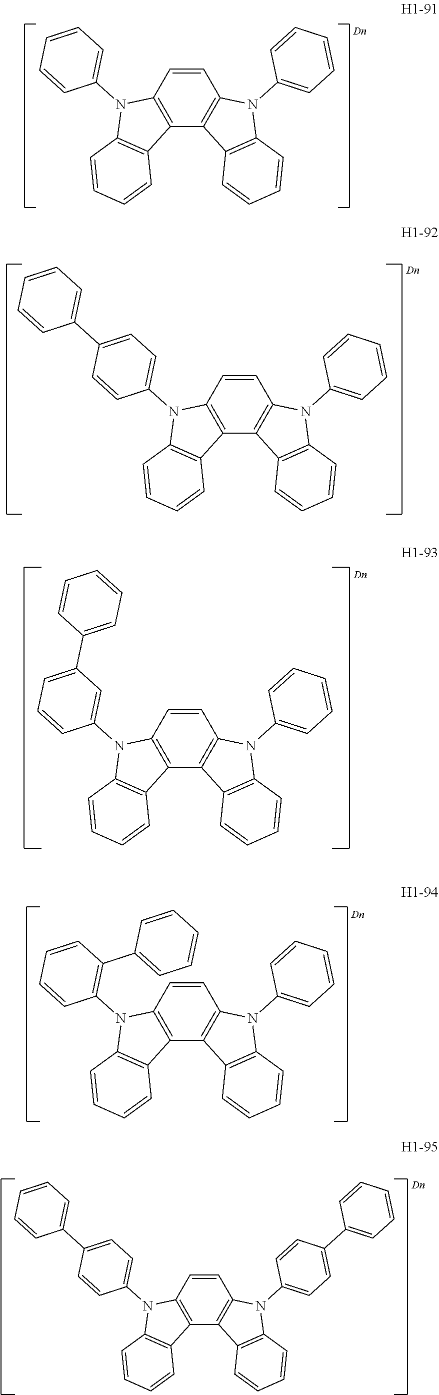 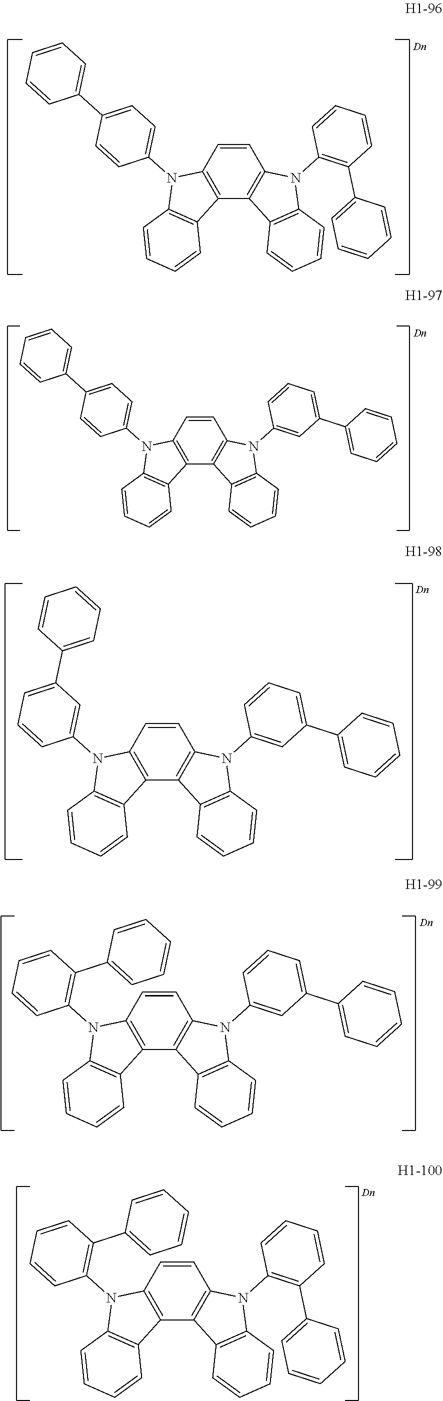 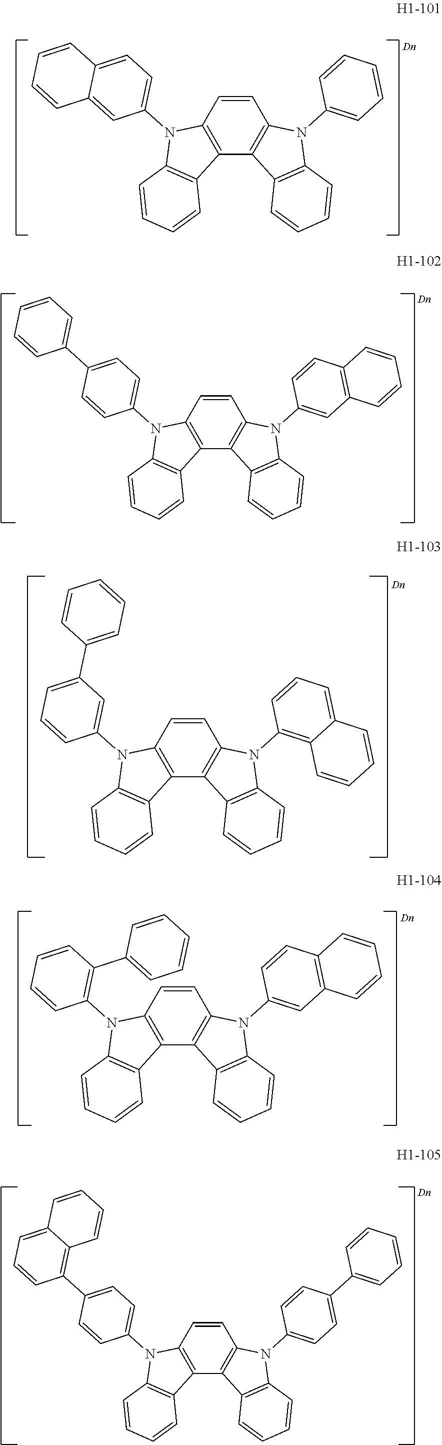 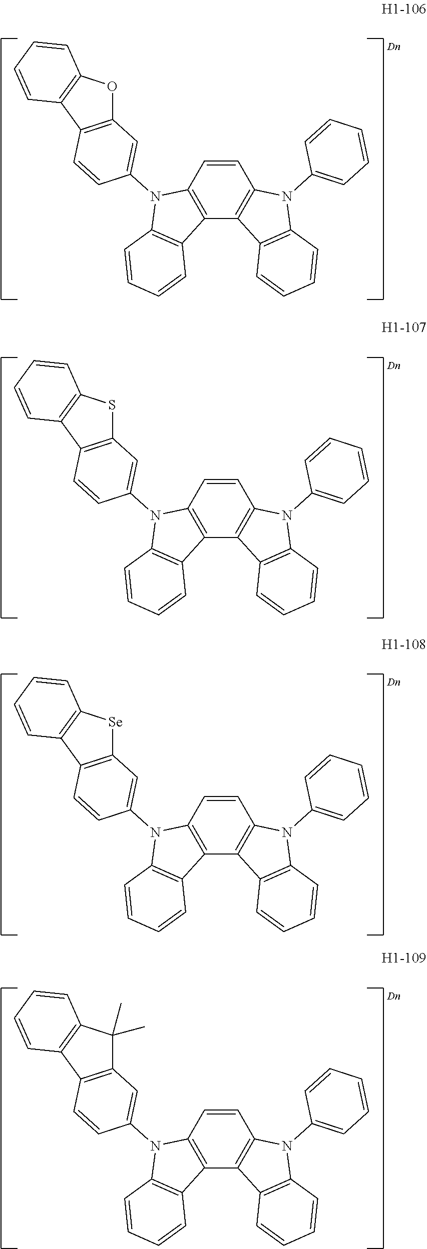 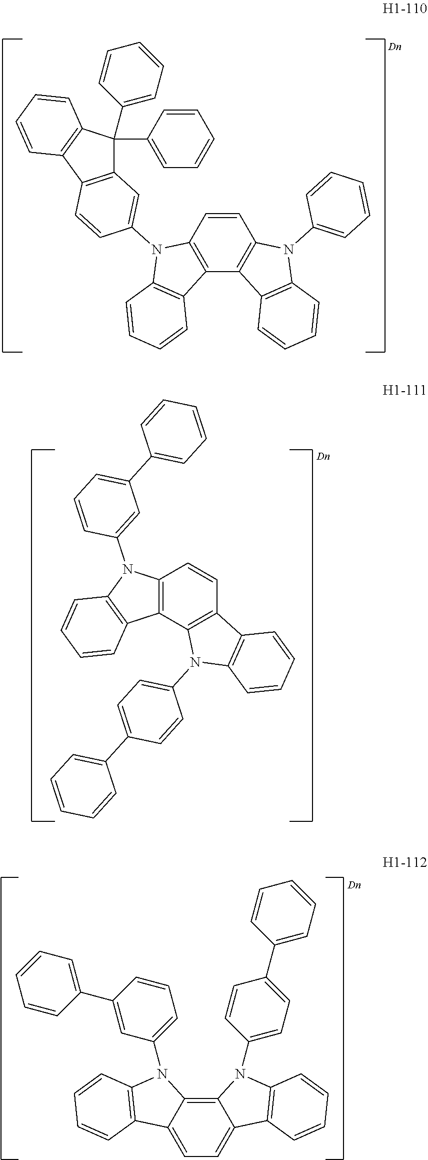 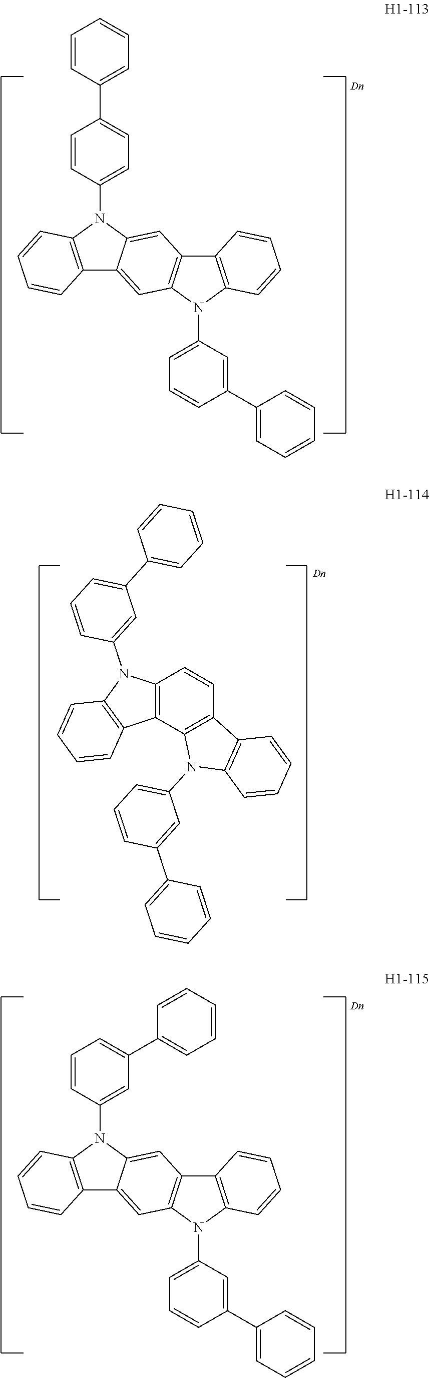 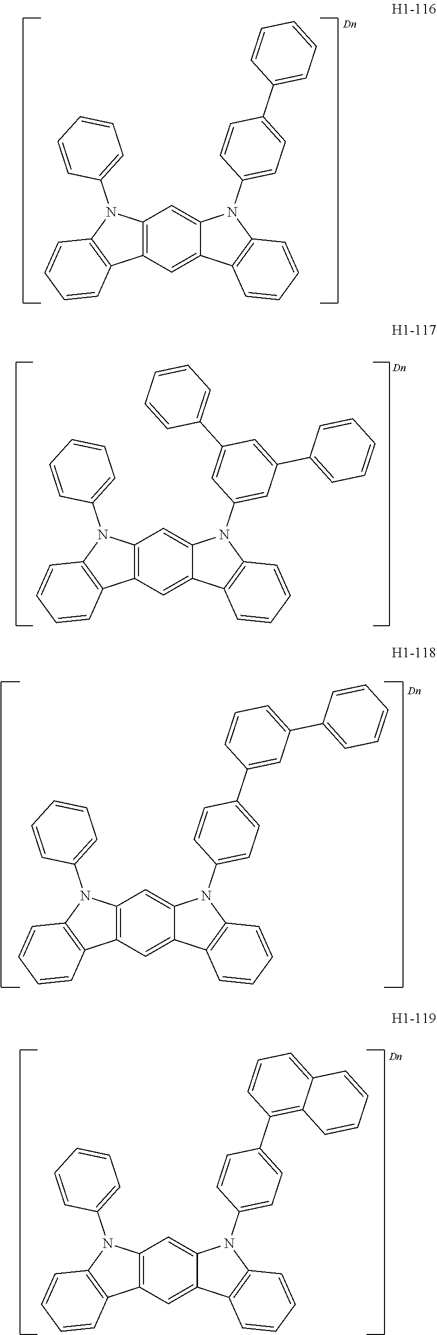 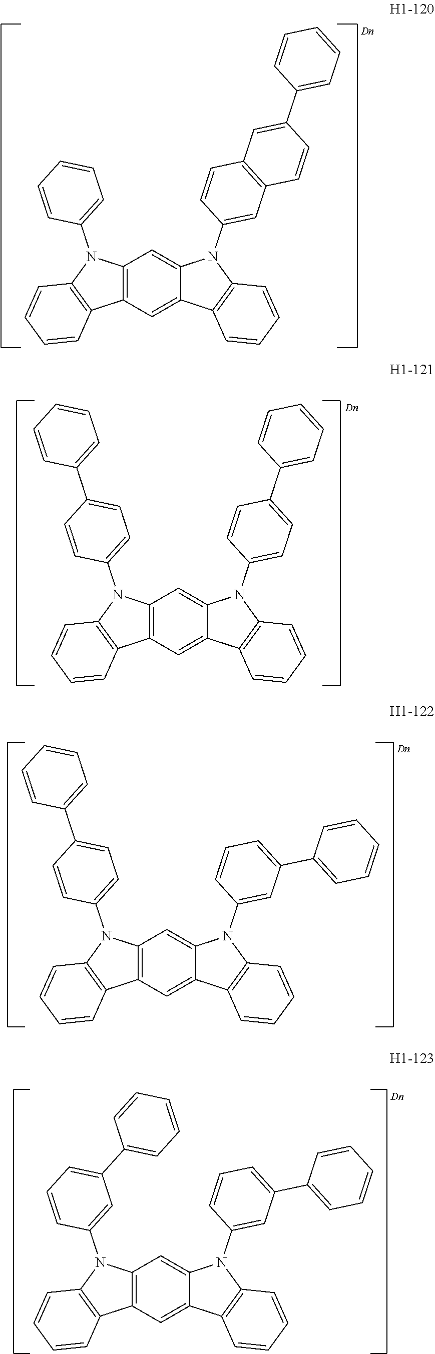 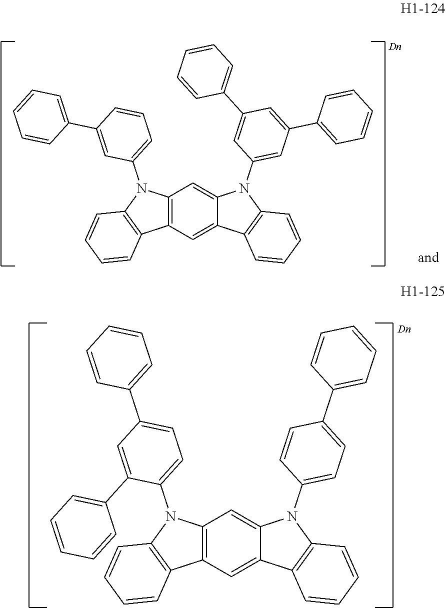
wherein “Dₙ” in the above compounds means that n number of hydrogens is replaced by deuterium, wherein n represents an integer of 1 or more, and the upper limit of n is determined by the number of hydrogens that can be substituted in each compound.

14. An organic electroluminescent device, comprising a first electrode; a second electrode; and at least one light-emitting layer interposed between the first electrode and the second electrode, wherein the at least one light-emitting layer comprises a compound according to claim 1.

15. An organic electroluminescent device, comprising a first electrode; a second electrode; and at least one light-emitting layer interposed between the first electrode and the second electrode, wherein the at least one light-emitting layer comprises an organic electroluminescent material according to claim 5.
